SEQUENCE LISTING (299 sequences)

SEQ ID NO: 1 - DNA, 43942 bp, Homo sapiens
  source 1..43942: mol_type = genomic DNA
ctatttagcattgctgctgccagccccaaccacatttctgattgcctaggaagaacagcctttcaaatttgacttctgcatgtggataggtgagtgtcttttgtggataccgacagtgggaatggctcctctcaatttgagaaattctgaaggaatttccattggcaagttcagcaaggccaactgacggagagaggaagcaccctgactgttccagttcagacactcttggggcttgttagtaattgagtatgtgtctggataagtgagtgtcatttgtgggtccagacagcaggatctgcttttctcaatttgagaaattccgaaggaatctccatttgcagattgaactagcccaacaaacagggagaaagcaactcaactgtttcagtttggactctcttgggacttgcttattgttgcagcagttagattgtattctggtggttgtttgtgtgttgatgtagttgtgggagatcagggtttgatctgagtgcagaccgcttgggtgtattcttcagggttggtctgcatgtggctgtgggccagtggagtgagggaacatctctacccgtatcacgctgaacatgcccaatctcatctgatctcagacagtctttgaattgtagcactgccttatatttggatttattttgctgttgaatgtgaatatgggatggaagtccatgtctccaggcttctatgctgctgctctaaggacaggcaggcctgattagtatgtgctgctgtttggctacagagttctttggagtctggagagatttggcctttaaaaatcaaactgtcagaccctgtctcaaggaaaaaaaaaaaaacaggatgtgagcgctctccctctccctctccctctccctctccgtctccctctccctctccctctccgtctccctctccctctccctctccgtctccctctccctctccccacggtctccctctcatgtggagccgaagctggactgtactgctgccatctcggctcactgcaacctccctgcctgattctcctgcctcagcctgccgagtgcctgcgattgcaggcacgcaccaccacgcctgactggttttggtggagacggggtttcgctgtgttggccgggccggtctccagcccctaaccgcgagtgatccgccagccttggcctcccgaggtgccgggattgcagacggagtctcgttcactcagtgctcaatggtgcccaggctggagtgcagtggcgtgatctcggctcactacaacctacacctcccagcaacctgccttggcctcccaaagtgccgagattgcagcctctgcccggccgccaccccgtctgggaagtgaggagtgtctccgcctggccacccatcgtctgggatgtgaggagcccctctgcctggctgcccagtctggaaagtgaggagcgtctccgcccggccgccatcccatctaggaagtgaggagcgcctcttcccagccgccatcacatctaggaagtgaggagcgtctctgcccggccgcccatcgtctgagatgtggggagcgcctctgccccgccgccccatctgggatgtgaggagcgcctctgcccggccgagaccccgtctgggaggtgaggagcgtctctgcccggccgccccgtctgagaagtgaggagaccctctgcctggcaaccaccccgtctgagaagtgaggagcccctccgcccggcagccgccccgtctgagaagtgaggagcctctccgcccggcagccaccccatctgggaagtgaggagcgtctccgcccagcagccaccccgtccgggagggaggtggggggggttagccctccgcccggccagccgccccatccgggagggaggtggggggtcagccccccgcctggccagccatgccgtccgggagggaggtgggggggtcagccccctgcccggccagccgccccgtccaggaggtgaggggcgcctctgcccgcccccccctactgggaagtgaggagcccctcagcccggccagccaccccgtctgggacggagatggggggtcagcccccccgcccagccagctgccccgtccgggagggaggtgggggggtcagccccccgcctggccagccgccccgtccgggagggaggtgggggggtcagccccccgcctggccagccgccccgtccgggagggaggtggggggggtcagcccccccgcccagccagccgccccgtccgggaggtgaggggcgcctctgcccggccgccccctactgggaagtgaggagcccctctgcctggccagccgccccgtccgggagggaggtgggggggtcagccccctgcccggccagccgccccgtccgggaggtgaggggcgcctctgcccggccgcccctactgggaagtgaggagcccctctgcccggccaccaccccgtctgggaggtgtgcccagcagctcattgagaacgggccaggatgacaatggcggctttgtggaatagaaaggcgggaaaggtggggaaaagattgagaaatgggatggttgccgtgtctgtgtagaaagaagtagacatgggagacttttcattttgttctgcactaagaaagattcttctgccttgggatcctgttgatctgtgaccttacccccaaccctgtgctctctgaaacatgtgctgtgtccactcagggttaaatggattaagggcggtgcaagatgtgctttgttaaacagatgcttgaaggcagcatgctcgttaagagtcatcaccaatccctaatctcaagtaatcagggacacaaacactgcggaaggccgcagggtcctctgcctaggaaaaccagagacctttgttcacttgtttatctgctgaccttccctccactattgtcccatgaccctgccaaatcccctctgtgagaaacacccaagaattatcaataaaaaaaaaaaaaaaaaaaaaaaaaaaatcaaactgtcagctgggcgcaagaaactgaggtgggcagatacttgagcccaggagttcgagaccagcctgggcaacatggtgaaaccctgtctctacaaaaaatacaaatattagctgggtgtaatggtgcatacctgtagtcccagctactcaggaggctgaggtgggaggattacctgagcctgggaggttgaggctgcagtgagccttgatcacaccactgcacttccagcctgggtgacggagatcctgtctcaaaaaaaaaaataatcaaactatcatggaaactgctttattcaaaattttgtttcacaacctttattggattacctatcagaacaaataaagttcaaccatgtgaacatgttcataaaccagtgagtttgcattgctatctcatggctggaatttaaaggtaaaagctattggatctttgtgtgtatgtaggtatatgtgtctagatgttacgtgttgtttttagagggtagcaaattggcttataaattaaaaagcgttcataaattaagtaaatgagtctaagcatttttcaagtttacatgacttaagtaaatctttaataaacaagagagcttttagatcatttgtaaaataaaaatagaaatgcctttggaattgtcagcatactttttttgtttgggttttacatttgtctctgctagatatttaaaggtgtcagggtttgacaaagagattataagactataaacccagccaagaacaaaattatctttgtgtgattttttgataaataagactaatttaatgtcgttaggttaataaaaatagctgaaacttttgcattattggtgcaagtatccatgtattttactttatgtttcttttttttaaaaacggagtctcactctgtcgcccaggctggagtgcagtggcccgatctcggctcactgcaagctctgcctcccgggttcacgccattctcctgcctcagcctcctgagtagctgggaccacaggcgcccgccaccacgcccggctaattttttgtattttttagtagagaagaggtttcaccatgttagccaggatggtcttgatctcctgacctcatgatcctcctgcctcagcctcccaaagtgctgggattacaggtgtaagcaatcatgcccagccaactttatgtttcttacttaggtgaacatctaatatatacaagttataaaaatggttaacaaggaaataatgaatagctttgatatctcttttctcataagtaatatagataattgctaaaaaagtgaatgaaatataaatggcataaatgctctaagtgaactttttgtgtaatttaatatcttgaagttattttggatgctcatgagatgtgtgggtcatttccaattatgaaagggtttcaatacagaaagttaaatgatgtaaaaaccttaattcctttaaatctcagttttcctaagcaatcaaaaacccaataacaatgacatagaaattattttgataaaatgtaaaatttgtttctcaggtcagttaccaaaaggcaaagaaaaacctcctgcagtgtgattgcttctcctatcagagtcccatctcgaaaacctgaaaatcaaacctgataaatgatgtagacatatcaagaaaagccaagaggacagaatcaagctatactggaggaaaacgttgcttctctagaattttaaaataaatatttcagcatcagaccatgacagcagttaaggcaagagggaaaaaaaagatacaagaactgacaaagttgaaggagatagtttcatttcaggttccttcaaggggagacaagctgacagcaacaaaacacaagaaagctgcactttggagatatagatctgagaagtattcaaaaagtaacagattatagagtcaaaatcaaaacctctcatccaatcaatattttaagaaaaccttgttctaacccaggggactaatctatatctctctcccctagttactggatcctttctaccctgtttcataaacaacctaagtctataatttgaattaacctttagataacttctgagttaaacaaaattatttctttaatcaataagaacacatcttctttggcacattgtacatggaatcatacaatttgctggaattcttattcttagtaatcctaaattttaatggaaacctaggaagcaagaaatcctgggctgtctatcagatattaacattttatagatgagaaacatgaatgttctcaggcccagaaaactcaagttgttttgggacctcaagaaaagaggaattcacccaattcatacaggtatctgcaagcacagatcaattattggttgtgctcgagaggcctttaaaaatcaaatctgagattccttatgaaaagttccagcaaagccaatttaaaaaagcctatatgaacaataattcttgcttcagtttatgcaaataatcaggcaaactataataagattaaaacagattttgcaaataaattggtcctactgtgatttgcctttcatggaaatggggagctggagagagaaacattatgttttggaagaaaactgcagtacacctgttattagattaatctttgacctatggcaacctggtcatccatggtatggagatacatctgtgctgtgctgcattcagttattaaaagtataagttaccagtggaatttagatatggattcatcacctcagaagttggttcactggatgcataaggaaataaaaactaatttttaaaaaagcaaaattttcaatcccttggtttttgttatcgatataaaccaaaatgtatctgaggtaggtctcaattaatttcaaagtttattttgccaagggtagggatatgtgcctgaaacacaagtctgtgcctttctccaaagatgcttttgagggcttcaatatttaaaggggaaagggcagatattggggaaagaggaagaaattttaaaaaggtgtgggtagggcggtggctcatgcctgtaatcccagtaatttgggaggccaaggtgggcagatcacctgaggtcaggagtttgagactagcctgaccaacatggacaaaccccatctctactaaaaatacaaaaattagctgggtgtggtggtgcacggctgtaatcccagctactcaggaggctgaggcaggagaatcacttgaactcgggagacagaggttgcagtgagccaaaatcgtgccactgcattccagcctgggtgatgacagagtgagacttcatctcaaaaaaaaaaaaaacaaaaaaacaaaaaaaaggtgtgggtagataagagacaaatggttgtattcttttgagattttgatcagactttcactaaatacacaattacatgtgagagtggggtaaagaaatagtcacttatgcctcagtctagatcagtgaatctgcatttttatacaaacgaaagggcagaggaagcaatcagatatgcatttgtttcagatgagcagagggttgacttcaagttctatcctttgtcccatacctgtgaagataagctatcaatttacattgccagggtgaaattcaatcgaataaaatgggagccaggtttgcctgacacaattcccagcttgacttctctctttggcttagtgatttggggaccccaagatttattttcctttcacatctgtaatagctaaaattaaagtaggagaatattgggtcaggctttgaggccaaaccaagcctagatgtgggtctgtctgaactcagatcactagcctcaaggctgctcatgaagaagaaaattatgccaggaaaccagaaagtacctctgagatctgtggtttcctggaaggtagtcaatgtggggaaggagtgaattaaagtgacttctgaaactagagggtataatgtgaaggaattgttctattttgcaaatcagtatcattagcttcctgaacctttactaaaataaattgtgagagtaattactttaggcacaatgtctttggttttaaatgctgcagagtggaagatccggttgatacagaacccacggctcacaattgaacagttgcagatgggtacatataatccgggcacacaggaggttattcccaaaagaacagcctacttggtggactggataaaacactgtgagaactatttactctgagaaggagaattgtcccactccacctataaatgccaagtggagcactccagatgaagcaacagatatgcttcaaatgtaagccacgggggactggctttataatgatgtggcaggccaggtcactaatgcaggcctccataacaactgtttcagtactgactgagtggttaagttaaatgttaaaaggtaaaaaagccagtggccttatacaaactctggattgtaacaaaagcccaccacgagttttgcctaggactttcctggggcttggacccatgacaagataatgaaggaattcttaacaggaccagattaggattaaacaagttttaccggcagtctgaagaaacttcccaggcttccacagacaagtttattggagatctcaaggaagttcccaaacctccatgatttagcaggagacaagatagggtaatcaccccagcacctggacccatatagattaagtaaatttactaagcctccagaggaaggtcttcagaactcagatctcagttatagattacaagaagttaatcactcatgtttttagatgaatgcacttacacacagacatataacttagaaggtaataagctctggaagactttgtaattttgagttggtctggcaatattttccaggccttctccctataacttgttaaagaaataaaaactcttcctccccagttcatctgcatcttgttactgggctggaagaaatagcagtctgaccctcagtttggtccaggaacaatgatagaaatattaacttggctgggcgtggtggctcactcctgtgatccctgcactttgggaggctgaggcgggtggatcacatgaggtcaggagtttgagaccagcctggccaacatgatgaaaccccatgtctactaaaaatacaaaaaattagctgggcgtggtggcgggcacctgtaaccccagctactctggaggctgaggcaggagaatctcttgaaccagtgaggcggaggttgcagtgagtagagattgtgccactgcactccagcctgggagagaagagcgaaactctgtctcaaaaaaaaaaaaaaagaagaaagaaatattaaccttactttttggtttttggctctttcgttgctttgagttttaatggtaatgagtccctggcacctctattcttgtgtggcctagaatgtttaattgactataagtcttttttgactctaagtcccttggttacaggggtcccactgacgaacaggatggactcggggcaggtagtcataccaccttggaaatgatatggaacaaaataaagtttggcaatcaatgctccctctggcatattccaaggagacctcagtttccaagagataaatcttacctgggaaaaggtcttgccccttgccctgctgagggtactggtagctcccagaaatggattagctcttagcccctttcaaaatgtttacttggagactgttttcctggactctctgatgcttgaggattttttcctcccctgaaacagaagctatgtcatacatgaaacaattagggaatatattaactagtctctccaacagacttcatttctgtatggacttgcctcttccctgtttgaaaccagggaatcgaatcatactgaagacctggaaatcccatcaggctgaagactagctgcaaccacaataggtcagtctttttaaggtgctgctgaccacccactcgtctatcaagttagccagtgttaaaccatgaattcaacatactcagtaaaactagtcactgtgggatccctccaggggaaacagtcatgctcttgtgaacctatacatggccttaagttaatattgaaagcccagccaaagatcctagataagaaactgtaaaaggaatatgcatttttcagttattattctctttaaacccacagcttttcacatataagatataatgaatcaggaatactttttggtaggcttaggattgttctcttggattccccaagggatccactccaaattctctggtattttaaaattggtttatctatattatttatcatccttgttcaatgtggcctaaaacgttgttctaaaactgaaataaaagtacccagatcatagtgctccaatgactacttctgattatagtgccacatttccattccttatcattattccacctcacacgtcctcagcatgaagcagccagaaggatcaatgaccagattctccatgattgaggaagtgataaatagaaaagagagactgaaaccggccccgttatcttatagaactaatatttacagttgtttttgtttgttttgctttggaacatagaaatggaccctgtctggtcttaaaacttgaaacttgtttgttttcctgagtttcttgcttagaaaaccgactttcaggcaaggaactgaaactcatcagagcaccacacccaggcaatgagactgcagatccttcacccagcatgattttttccctacctctgcctaatttctatttttcccctgtccacgtagcatcaaactctgtaaaatatttgaagagatttattctgagccaaatatgagtgaccatgcccgtgacacagccctcaggaggccctgcgaacatgcacccaaggtggttcaggtacacattggttttgtacattttagggagacacgaggcttcaatcaactataaacatttactgattggcaattggttgagtttatctaaaggtctggaatcaatagaaaagaaatgtctgggttgtggtaagaggttgtagagaccaaggttttatcatgcaaatgaagcctccagacagcagacttgagagagacttaagtattaatgcctgagagatataatgaagcgtgttcgacacgcacttcctgtcatggcctgaacccatctctcaggttaaattttcaaagagctgtggttgaagagggagtccattcagatggttgaggggccttagaatttcatttttggtttacactccccacacagctacatttctgcctgcacatagctacattccttcctcactatataacctcccaattttagtttgttggagaacaaagccagaataaagccaatcctagtcatagcattgcaacttgtatcagatgaaagaaacctacaaaaagataattataaaatttagataaaatgtataaaacaactgagaaggcaaaatttgaggggctccaattaatcaaggtttaccgcttatattctgtgtgactgccagaaaaaaagaaacaaaactttttggtaggaagatcatttaaaatttctgtttttaatacataaggcctgttatttaataaaaaattatgagacaacctaaaaaaaaagaattaaatatcaaaacaccaattgacaagccaaatgataaaaactggcctacatatggttcagaaaatgatttcaataaatctactcttcaccattctaatccctttgcagtgccctaggatggtgactgactctcatgtataatatccctggacactattgtcctgaagctctaggtatgtttgtgctaataggaagaataggcaaaagatttaaaggtgggagaatccttccctacagcactaggaaatggatatggagttgttctcgctgagtgtacctccagggaggcagccttcatcagctatagtttctgctgcttcagttaattgctaccagcaatgttctctcaggcctaatgtagtaatggcttccggcagtagctagccctggggtatttcactatccctcatggtttcctttggtcttatctctatcttgtaaattgtcctgttattttatcttcttaaattttccttttaatgtataaccggttttttcttttttttttttttttttggatcccaagtgtgtgtttctttttggtacctgagtatatcactgtcactgtggtctctcaacttttaaattatgtgactctgaaggtgtgtgtctccaaatttttatccctgtgcacttttcttcacctgtcttcatttctttcccttttttccgttataactttcactcacatccgtgtgaagagaccaccaaacaggctttgtgtgagcaacaaggctgtttatttcacctgggtgcaggtgggctgagtccgaaaagagagtcagcgaagggagataggggtggagctgttttataagatttgggtaggtagtgggaaattacagtcaaagggggcttgttctctggcggacaggagtgggggccacaaggtgctcagtgggggagcttttgagccaggatgagccaggagaaggaattttataatgtcatcagttaaggcaggaacagaccattttcacttcttttgtagtggaatgtcatctgttaaggcaggaaccggccatctggatgtgtatgtgcaggtcacaggggataggatggtttagcttgggctcagaggcctgacattcctgtctccttatattaataagaaaataaaaatgaaatagtggtaaagtgttgggacggcaaaaattttgggggcttggtatggagagataacgggcgatatttctcagggctgcttccagcgggattaggggcgatgtgggaacctagagtgggagagattaagctgaaggaagatttcatgctaaggggtgatattgtggagttgctagaagaaacatttgtcatttagaattattcgtgatggcctggatacggttttgtatgaattgaaaaactaaacagaataagagaaggagaaaaacaggtattaaaggactaagaattgggaggacctaggacatctaattagagagtgtccaagggggttcagcataattacttacttggttggcaagtttttgggctctatccttgagttttttatgttgtcatacaccaggccagattgatttaggtagaaacaacactcttcatttaagaatatacagagtcctcctttttcagcagtgactaagtcaaggcctcagcggttttggaggacaactgcagctaaagagtcaacttgggcctggaggactgataaagtttgtgatatgtctgtgatgctagcagagaagacactagagaggctatggaaggtcgtgacagaggttgaaatgcctcctattccagtaccgagagcaatagtggaggcagaaagtcttaaactgataagcaagggaattagtggaataactcttttttgtcgtgtcggtgtcatgaggggaacagggagctcttcggtcctatttgcaaattgaattttaggagtaaggaaaactagtgtgcatgtacctgtccaattagcaggtagacacatggaggtagaggatccacagaggaagaagagaccttgtgcgaggcaaaactggagaagcaaagtaaaaagatgagaaggagtgctgaaaggggtgtcttgtacccagactcctggggatccagctagggcagcagccatcagaggttgtaatggggactggtggggtaactgcgtagagggggaggttcgattttcatggtgtatgagaaaatgttgagtgtctatgagctacctttcactgttatttatggggctgggtataagtaaacaacaagagggctttggagatgaagagtaaaggaacatcaaagtgaaaggttacccaggggaattccagtgggtctttgctgagagatacataaaggagcagccacaggaatagtagtttgtgttgtgagaggtaaatacggggggagtagagttgatacaaggagaaaggttttttaagtaagtgtggaggagggcggcagcttgctgacgtgaaatgtctggggaggtctagctggacctgtctagaaagtaaatgagttcatcaggagggtaaaggtgagggctgttaaaggaagtttggaggtgtaggagacaggagatgttgcctagtctgcatgtaaggtggggacagctgtgtaggcattggaagaaagggaaatgcaaagctagagtttgttcgctaaggagggattagaaacagctaggagagaatgagtaatgtttatagtgtggtggagatagctggggagaggtagagggtggcataagaatgggaatgagaataagattgagtataaaagtaaagaatagaacttcatcagggtgaaagtattggagggtcccctgccagcaaagattatctatcccctctgagagggagttaagagttgccagtcctgggtgggggcaaatcctcgagcttgatgtgtagggaaaggagggggcctgaataatctatgaggaggagtagaatagtagatggaacagaagttatttccttgaggatagatttccacgatggaaaggaaatgagaggttctaagaggtgggctggtgacttgtactatagcatagcctgcctttgctggtgtgtggcgattaggcctggtggaactactatgaataaactaagtgtgttcagggtgaggaacacgaaagaaggaaatatgaggaaaaggggtgaatgtcaggtgaatcagagagatacagtcataggggtcaggtgtggtatccagaatactgtgggaggctggattgaagtctgggccaggaacaatggtaattgtcagagacttaacaaaaagggagtgtagctgaaggagccagggagcagaaagtgtatgcgtcaggtgggagaaagaaaatagattttggaaattatgagaaccgtagagaatgagttgggcatagtttgtgattttttgggcctctaaaagtattaaagcagcggcagccactgcatgcagacatgagggctaggctaaaatggtaaggtcaagttgtttggacagaaaggctacagggtgcggtcccagctcttgtgtaagaattctgactgccctaaccatgcctaggaaggaaaggagttgttgttttgtagaagggattggggtttgggaaattagccggacatgatcagcagggagagcacatgtgtttttatgagaattatgctgagataggtaagagatgaggaagaaatttgggcttgactgaagtaatgggggctgtctgtgaagccttgcggcaatacagcctaggtaatttgctgagtctgatgggtgtcagggtcagtccaagtgaaagtgaagagaggctgggatgacgggtgcaaaggaatagtaaagaaagcatgtttgagacccagaacagaataatgggttgtggagggagatattgaggataggagagtatatggatttggcaccatggggtggttaggcaaaacaatttggttgataaggcacagatcctgaactaacctgtaagccttgtctggttttaggacaggtaaaatgggggaattgtaaggggagtttataggctttaaaaggccatgctgtagcaggagagtgataataggctgtaatccttttaaagcgtgctgtgggataggatattggcattgagcggggtaaggtgattaggttttaatgggatggtaaggggtgcaggatcagtcgctaaggaggcagtagaggtgtcctatacttgtgggttaaggtggggagatacaaggggaggatgtgaaggaggctttgacctgggggaaaaggtggcaatgaggtgtggctgtagcctaggaatagttagggaagcagataatttagttaaagtgtctcagcctaataagggaactaggcaggtggggataactaaaaaggagtgcttaaaagagtattgtctaagttggcacaagagttggggagttttaagaggtttagaagcctggccatcagtacttacaacagttatgaaggcaagggaaacaggcctttgaaaagaaggtaatgtggagtgggtagcctccgtattgattaagaaggggacgggcttaccctccactgtgagagttacctagagtgtctgtgatggtcctgtaggcttctgaggtgattgggcagtgacagtcttcagttgttaagccgagaagatctgggaaggagtcagtcagagagccttggggcagagttccaggggctatgggagtggctgccggtgagatggacagtctgatttccagtggggtcttgcacagatgggacatggcttaggaggaatcttgggctgcgggcattctctggccaggtggccagatttctggcacttttagcaagctcctgggggaggcaggcctggaggaatgcctggccactgtggtttaggcgtttggaagttcttgcgtgctggagatgtggctggggtttgtctcacagtggaggcaaggaattgcaactcagaaatatgttgctactttgctgcctctactttattattgtacactttgaaggcgaggttaattaagtcctgttgtggggtttgaggactggaatttaatttttggagttttatttaatgtcgggagcagattgggtaataaaatgtatattgagaataagacggccttttgaccttttaggatctagggctgtaaagcgtctcagggttgctgccaaacgagccatgaactgggctgggttattatatttgatgaaaaagagcctaaacgctatctgatttgggataaagaaaaaggaacattaaccttgactatgcctttagctccagccacctttttaagaggaaattgctgaacaggttggggagggctagtcatggaatgaaactgtaagctgggccaggtgtgaggagaggaggtgataaaaggattacagggtggagaagtggaggctgaggaagaattgggacctagctcggcctggtgaggaggcgagaggtcacatgggtttgtagaaaaggaagattagaaagactcagcgatgcttggggttgggactgaggggacaggcgggagggaaagaaggaagatttgggatgagttgcattgggaacagagactagggagggaccaatgtgtaaaagaatgcctggacgtcaggcacctcagaccgtttgcctattttacaacaagaattatttagatcttgtaggatggaaaaatggaaagtgacattttctggctatgtggaactactgtcgagtttgtattggggtcaagtggcattgcagaagaaaataaggcatttaggttttaggtcagctgtgaattgaagaggttttaggtttttaagagcacaggctaagggagaagaagggggaatggagggcggaagcttgcccatagtgaaggaggcaagcccagagagaagagagagtagagacatggagagaaggggttgggggttcttgcccccaagaagagcggtacttgccgctaagggtgaaggagaaggggttggggggttcttgccccccagaaaagcggagaaggggtagagacatggagaaaagcggtggggttcttgccccccagaaaagcagagaaggagtagagacacggagagaaggggttgggggttcttgctcccaagaaaagcagtacttgtcgctaagggtgaaggaccaaggcaggtgtccccacatggtcagacacctctgaaacatgggtgaataatcaggcaggcatccctgcctgattccaaagggaagactgtcttcccaattccgtgactggtgccggagttttgggtctatgaataaaacgcgtctccttcatctctgtcagaaaaggaaaggaactgaaattaagagagagattgaagggtgatgccaagattgaaaggagaaagaggttgagggatagtgagagaggttggagaagagagtaaaaagaggccacttactggatttaaaattggtgagatgttccttgggctagttggtctgaggaccagaggtcataggtggatctttctcatggaataaagagcaggaggacaggggattgatctcccaagggaggtcccctgatccgagtcacagcaccaaatttcactcgcatccgtgtgaagagaccacaaaacaggctttgtgtgagcaacaaggctgtttatttcacctgggtgcaggtgggctgagtctgcaaagagagtcagcgaagggagatggggtggggctgttttataagatttgggtaggtaaaggaaaattacatcaaaggggggttgttctctggcgggcaggagtgggggtaacaaggtgctcaataggggagcttttgagccaagatgagccaggagaaggaatttcagaagataatgtcatcagttaaggcaggaacaggccattttcacttcttttgtggtggaatgtcatcagttaacgcaggaaccggccatctggatgtgtacatgcaggtcataggcatatacacaggcatatgatggcttagcttgggctcagaggcctgacaataacaacccacaaaaaatatataatttaggtaatggataatgtatcacttatgtttaatattggaagacatctgtaattatatttgcagattgtctcttagtattcaactagaatgaaaattgatataggttgatttcatatcttgtctattgcaaatagtgctgcaataaacatgggagtgcaggtatctatagtatatatacacaatggaatattatttggccatagaaggaaatgaaatcctgtcatttgcagaaacaggagtggaactgaaggtcattatgttaactgaaataagccaggcacagaaagacaaatatcacatgtttccactcatatgtgagagctaaaagaatgaatctcatggaagcagagagtagaattgtggttaccagaggcttagaaaggggtggtgggggataaagataagttggttgggtacaaaaataaaattagatagaaggaacaaattctagtatttgatagtacagtgggaaaactatagttgacaataatttattgtatatttcaaaatagctaaaagagaagaaatgtactattcccaacagaaggaaaaggtaagtgtttgacggaatgaatattcttattaccctgatttggtcattacacattgtatacaggtttcaaaatatcacatgtacctccaaaatacatacaactattttgtatcaataacaaaatttttaaaaaactgatataggctgtgaagggcaaaccatgtagtcatctcagagctcagcttattcttgtggatataaatgttttagctggctctaaaccaaggattttgtatgaagtaatacttattcaaatattttcaacaaaatagagtaccttgcaaacaggactttgagatttttcatttgtttccctttacagttcgtcatatacgcttcatcttctagtgttgattgatgaaacaacgttccacatgagacagcagagcaatagaacacagaacaggactctgcctgtgtggtggccggtttccatcctttcacaaacaaagagatatttataaacaaatatttatatgcaaatatttgatccacctaggaaaaatagagggctactctttcccaccaggagaggccttatagcctgtgcacatatttgaacaaggcaaccagagactttgatggtctcaactctaccactcaccaactgtgtgatgttggatacaaacttaaccactttgcaactcacttccgcatctgtaaaataagggtgataataatagcacctggtttagagagttgtaaagataaaagcaacattagtaaagtacttaggatgaagtaatcatgttttgaatatattttcctttaaatgtggctgccaatattttcatggtaatattttcaaatgtattcaatagctttttctttatcaggagaaaatcagttgttttaattaaattgacattgaatttttgatacatttaggagaatttagtttctttttatacagtattgacgcttctaatcccacagcatgtttttctatttttttcttttcttcaatagttttaaaatttttttgttcattttggttatgcaaatatctagttaatttctatctgtaatattttcctatttatttttagtagctcttataaagggagcattttgtttttcctgtctatcctgacataataaagcaattagtttttaattgatttttaaaaatatttatgcattctctttcatatgtataataataatacctaaccattttatgatttacatgtggcaatgtcaagaagaattttaaagtaagatttactaacgaatattagagaacatgctcaaaaacagtcctctccaccccctcacttccacttgcattcttgatcaagaagtgaaataaaaaatactaagactattgagtcaaactaataagacacaaatctggactttggagactcatttccctcccacttttgatagcttagctgaggaggagatcaaatggttgagtcagtctgtagagagatagcaaggctgattatagttttcctggcctgcaaagctgtttccagagccctcagtatacagaccttggccatcagtatgcatactctctggtgtggtttctgggcatcagatcccttaatatataggttggcgctaaagtaattttgtaaacttttaatggcaaaaaccacaattacttttgcttttaatggcaaaaaccagaattactttagcaccaacctatatattgttatttagagaaactaggcattccctacctctcttaggcaaacctatagctatgtaacatggcaacggaatgacactaactgtctcattctccttgggagtgctctctgtatgttcagtctcccagtgaagagaacccttctatcaggaccttcatcatcatacaaaaggaatatctatgccaggccacagctgcagattagtgagtgtctgagaactgcatcttttctgttatttttctgcttgaaagtttttcaccaataaatcctacactatgtctgcaccatgtggtgctagttatttgtgttctcttgcatgacactgatgatgttatccaaacacaatttcaaaaactactttctgtagttggtaaatatatagaaattgagaaaaaggaagaaatatgaggaagaaagatgttatctgttgcaacagttcaatgttcttaactgaagccacagatgagatttggctcatttgcagacatatgggtgattggtacagtaggtttataaacagaagtttaaacttgtaagcttaagcttccgtttataaacagaagtttaaaattataggtcctgtttaacattcagctctgttaactcactcatctttttgtgtttttacactttgtcaagatttctttacatattcatcaatgtctgaagaagttacttatgcagatcttcaattccagaactccagtgagatggaaaaaatcccagaaattggcaaatttggggaaaaaggtaagattttgagttatggatgtgttgtaagtttgtataatagaagtggttatagacttgcatcttcaatattagtgatatagttactattcaacttaaattttcaattaagtctcttatgtttaagcaaaggaacaaaaagtgtatgaacaaaatgattagaaaaaagggttttgtaaaaacacttatctctgcattgaataatcaatcttttacatttttgaaattttttattatcacactcaatataattgggacagaatttttcaaatgattgggttgtatgaagattttatttctttatgagatgaagccatggaaaaagaaacatgttcaggaaagtcatgatttcatatgtgtgtgtgtgtgtgtgtttttttttttataagaaaatgctttgtgtcagacagagagacctttataggatttcatttgactatctaccaaactaagcataaatcaccctaaaatttctcctacttcttttggagattttttactaacttactaaaaaataaaacatgttaagttaaatgaaatgaatcattaaggaataaaggaagaaaagaggtaaagaaatatcagaagacataatatattcataatattcaaaaccacattatatcaagtaactttgtatttttcaattcaaattaaaatcatgaaataagtggtccatctgtgttgactttcacttaaacaaaactactcttcatcttactcagaatcctatagttttcctattatgtatggttcttgccttctggctcctagccttttgggagtgaaatctgccctagcgaatccatagtttaaatcagtgtatttgagttaaaaataacttaaacagtgatagtggagaacacaaagaagggtgatagaaacagaaaaataaggagagaagagtgccaaaattctcacatattatttaagttgttgtcctctctcctctccccacacagaatataacatctttaatattctagcattgctataaacagaaatttcactgttacatcttggatccatttatgatgagcattgtaaatcttgagaaaggatagcagattaattctgttggcttcttttttaatactgaaatttttttttagaaagaagaagggtttaagaattttacaatattttctgtctttatttccttgtttatatttcttcttttatacactgaaagagaatgggcatatcatgagcctaatattaccaacaattgccctttacttgtcctctcacaccctttcccacctcttttcctgccaatccaccttaagttcccaccacaagaattccctgaattcacggaaatattgaatccatgtcttcttcatgttcttaatctcatctaattttctcattatcgagcttagaatctacattatgtaatataacattgtattatgtaatgtctactcttagtctcttacacttctcctcttcaatgtgtgttaatttttccttaaaaactccagctctggctaggtgcagtggctcatacccatagtcctagcactttgagaggccaagatgggaggattatttgaggccagaagtttgagaccagcctggggaaagtagagatatgccatgtctataaaaaaaaaatttttttaattaactgggcacccatagtctcagctgcttgggaggctgaggaaagaggattacttgagtccaggagttggaggctctagtaagctaagatcaggcgagtgtactccagtctgggtgacacagcaagatcctgcctcaaacaaatcaaaacaaaacaagacaaaaaccccaaaaacaaacaaataaaaacaactccagttctggttaaacccgaatatccacccagtctgccccagagttgaatattgctggtgaaaaatacacaaattttcaaccccagttactctcacctttcctctggtaagaaaaaagcatccatctacacttttttttgttgttatttaaatgtctatcctaccatgtccttactgatactctgctttccttccagatagtatttacgataagtatgtacttatgaagggacggctcctcattttgctctgaatttcagtccatgtgtcgttttcaaagacagacttccgtaattttttcctttctctcctgtgtcattattctctcttccttctctgcttttcctttttggtgcaccaacatactctactactcaccaatatactctgctactccttttataacaaaacacaatccttgcttgaccacttaagcctttccattattatttaacttctttgtttcctttcacaactcaacttttgaaagcattttctttggccactgactctcagttcttcacctctcattctgttctcaaccaactctattaggcttctgcccccttggtgccatagagactgagctttaggtcactgatgtcctgcctgtttcatcccaatagtcacatgtctcttccattacttcacttccctgcagcattgaaaaagttgaacagtcctctaaatgctttctttgcacatccctcctgacacatcctcatggcttctcctgtcaccatgcctgcctctcctcattctttgttgattcctcctcctagatgaaactctacaagatggaatactctgaagccctgtcctgggtcccgttttattgcatcactcagtacactcttctaatctacctcattcagttgtttgactttaaatagagtgactccccaatatctctgtatgacctttccctgaatctcaagactagaaaatacatgtgccactttcacatttccataaggagttctaatctcattttaaacttaaaatgtcaaaaatagtaatatttatttttcttctaaaattgtctctctctccagacagcctcatctcattaaatacacatataaatatatatttaaatacaattttcaagtgttttctctgtgccagttctgttctaattgcattataaatattaaattatttagtgtctgatactttgcagtctttctgtctcccatgctttaaccatattgaaactcagacacagagatgttaaaaggtgttaaataattttcccgaggtttcacaagcaataagcagcagaaccagaattcaaaccaaaacatactgatatggtttgactgtgtctccacccaaatctcatcttgaattgtagctcccataagtcccacatgctgtaggagggacatggtgggaggtaattgaatcatgggagcgggtttttcccatgctgttcttgtgatagtgaataagtctcatgagatctgatggttttataaagaggagttttcctgcacaagttctctcttgtctgtggccatgtgagatgtgcctttcaccttccaccatgagtgtgaggcctctccagccacatggaaccatgagtccattaaacctctttttacttataaattacccagttgtgggtatgtctttatcagtcgtgtgaaaatgaactaatgcagtaaattggtaccaatagagtagggcgctgctgtaaagatacccaaaaatctgaaagagactttgggactgggcaacaggcagaggttggaatactttggagggctcagaagaagacagaaaaatgtgggacagtttggcacttcctagagacttcttgaatggctttgacaaaaatggtgataaggctgaggtggtctcagatggagatgaggaatttgttgggaacgggagcaaaggtgactcttgttatgttttagcaaagtgactggtagcattttgctcctgccctagaaatttgtggaacattgaacttgagagggatgatttaggatatctggcagaagaaatttctaagcagcaaagcattaaagacgtgactcgggtgctgttaaaagcattcagttttataagggaagcagagcataaaagttcagaaaatttgtggcttgacaatgataaataaaagaaaatcccattttctgaggagaaattcaagccagctgaagaaatttctaaagtcatgggaagccaaatgataatcgccaaggcaatggggaaaatgactccagggcatatcagaggtcttcccagcagcccctcccatcacatgcccagaggcctaggaggaataaatggtattatggactgagcccagggtccctctgctgtgtgcagtctaggaacttggtgccctgcatcccagctgctccagccatgactaaaagaggctaaggtacagctcaggtcatggcttcggagagggtgcaaaccccaagccttggtagcttccatgtggtgttgagcctttgagtgcacagaagtcaagagttgggttttgggaacctctgtctagatttcagaggatgtatggaaatgcctggatgtccaggcagaagtttgctgcaggcatggggtgctcatggataacctctgctaggacagtgcagaagggaaatgtgaggtcagaagctccacacagagtccccactggggtactgtccagtggagcagtgagaagtggaccaccgtcctccagctctcagaatggtagatccactgacagcatgcactgtccacttggaaaagctgcagacactcaatatcagcccatgaaagcagccatgagggagactgtaccttgcaaagccacaggagcagagctgcccaaagacatgggagcctacctcttgcatcagcatgacctggatgtgagatatagagccaaaggagatgattttggagctttaagatttggtgccctttggacttgcatggggcctgtagcccctttgttttggccaatttctcccatttggaatgaccgtatttatccaatgcctgtactctcattgtatctaggaagtaactaactttcttttgaatttactggctcataggtggaagggacttgccttgtctcagatggactgtggacttttgagttaatgatgaaatgagttaagtcttttggggactgttgggaaggcatgattggttttgaaatgtgagacatgagatttgggagaggtcagggtggaatgatatggtttggctgtgtccacacccaaatctcatcttgaattatagctcccacaatttccacgtgttgttggagggacctcgtgggaggtaattgaatcatatggtaggggtgtttctcgtgctgttcctgtgatagtgaataagtgtcatgagatctgatggttttataaaggggagtttccctgcacaagttctttcttctcttgtctgctgccatatgaggtgtgcctttcaccttccaccatgattgtgaggcctccccagccgcgtgaaactgtgaatccattaaacctctctttccttataaattgcccagtcttgggtatgtcttattagcaatgtgaaaatggactaatacacatgctaaccctaaagatggtgttttgctttttatcactatgagatactgcctacaaatgtgcatatctaccaactttctctaaagaaaaatttcagaaatttttgcaatagattttagcttagaaatgtatacttaatttatccatgggtcatcaaattccctagtactagctagtcaaacttctgttctctttcacatgtcatactgtaacagatttcttactgataaaacaactctgttattttgccctcctagattaaattcttccagtggtttccccaaaaatctaacttattctggctagaaaaggctgtaaataatgtctcctccttgttattttaaaaacaaaacaaaagaaaacaaataaaacctttaacatcatttttcacattggccacaattcttctgcccctctgaatatattctgtttttcaaatagaaatcttttccttaaattattgtctttgaacttcatgtaattgctacctgtaatgttcttcctacttattttgcatgctggttcctattcataagatttgaactgcctgctctctataacagcttagttttaaatctctgcacaacatcgccaaatttgagatcttttctccctttctttctttctccctgtctcacttatatatctatttatctatttcctcctacaattaaaatttatagctgtgaaaacaggaacttgtacatcttgctcactttgatattgttagcacccaggataatgtctaataaatacaaggcactcaaaagataacttttgaataattgaactttttaataattgaataagttcattgaataaataaacttgaataattgaatacacatataaataagaaaataattttaagaaagatagcactggcatttataattctaatcttagataaaatcttgaacatgtagttctttcagtagtaactattttctaaagtctatataagttaagttttactatttattagtaattgtctctggatttcacaatattattgcaccatcaactatgagatgtaagacatagctcagaagtatagtgagattattatggtatccctgaattggattttcttgaaatatctagctagtctttacctaatgtataatagctttatacataatgatgatttaaataacatagtcacaataataattcactcacaaagtttataattcctgttttttttttaaatagagaaatgatctattctgggcttcactattcattaaaaaggaagtattcaataaaagtttcttgcaaaaacacatgaaaatttttttgaaaaattgcaataaaaatggtgtagatctggactgattaagatcaaaagctaacgggaggacacccaaagacattatgaaaagacagcctatcttgttcattctgcagctaaattctcataattgcctggggggatacataaacaactgaaactgtttttcttgacatgccacaattgtctactcaagcaaagacttctcctattggcttctctgttaatttccagctactcacttttcccaaatttgtacaaacaaaatacactatatctaggttgactggttttctccattctctattataaattctgtttttgcttatctgtttttttaaatcagtctaccagttaacagcttctcctgccagaatcaatttacatacaaaacaattaagtgattttgatttattcttgacatattcttcaaaaatattttttgaatattcttgaatggctagcaaatccatttctttccaataggcatattaggaaataaaggtaaaatgaatgaatgaatgaatgagtaaataatccaaatgaaagttataccatttttaggcctctctgttaattttgctttccacagcacctccagctccctctcatgtatggcgtccagcagccttgtttctgactcttctgtgccttctgttgctcattggattgggagtcttggcaagcatgtgtatgtactgcccctgtttttgtcaagaggaagtatctagaatttcaaatatttagcaacaaaagtttattccttcagtggaagatttataataatgataggtgataggcccacaaggaggacttacaatgtggaacttagtctctttccctcactcctgtagagtaattgctttccctacccctttaatctttatactacttctgcaaatgataaaggatgatggctctattgagaatttacaattttttgtcatttttttaatgtggagttcacgtaactttgaagatagaaatgaaaaaaatgaacaaactacaaaacatcagtgaagagctccagagaaatatttctctacaactgatgagtaacatgaatatctccaacaagatcaggaacctctccaccacactgcaaacaatagccaccaaattatgtcgtgagctatatagcaaagaacaaggtaatcttgtattctcttggagttatttattggacaataaactaaaccactgagtctcttaagaatcattttataattagctagcagcctttctctagggagtcataatttgggggaaagaattacatgtggttgatggagaattcattgatatttccacgcagttagaagaaaaacagaaaaagtagtaacaacaataggcccccaaattatcaggagataatggttacataataaaatgttagacaagacagtttctctaatctgtcacttactataattattctgggcactgaggttaatagaagacagggaggaagtgacacctctttacaggtgacttcattcttaaatttcagtcagtgattgataacaaagtccatagaaaaaaataaattgagacttcacctttgggatcggggagttggtcttttagtgtctccataagtttctatagtcatagttgctaaaacatattccttttgctcctgagaaacctcaaagtttcaacatttcagagaacaaaaattaaagaagataagattggtatagataggtggggttaattcttactatacagaatgtttccagtaattagtttttattgtctttaaagaatatacaaaataacaaagaggaattgacataggaagacacacttgaatccaagtgcttaatttgaaatttttttgttcaagttatgtgtctgacacattatggtggtatataaggaactagaatacaggattcacttacttccccttcgatgtaaaaaaaaaagaaacggcatgtaatcccagctacttgggtggctgaggcaggagagtcacttgaatccaggaggtggaggctgctgtgagctgagatcacgccactgctctccagtttgggcaatagagcaagactctgtcaaaaaaaaaaaaagggggaaagagagaaagaaaaagaaagaagaataaacagcaaatgtgatcaatgtcacacagagaggaaaggaaataataagaattattatgactatcaacaaaacccaaataaaactatgttcttcttccagagcacaaatgtaagccttgtccaaggagatggatttggcataaggacagctgttatttcctaagtgatgatgtccaaacatggcaggagagtaaaatggcctgtgctgctcagaatgccagcctgttgaagataaacaacaaaaatgcattggtaaagcccaggtgtttctaccatcatgggatagagaggggtgtggcatgttggagaagacttcaatcaacccactcatgattctggtttatttcagttggggtgtgaagtgactgaaactttgtacacttatctcaagatatcataggcaaggtaaaacatacaagtttaggaccttagcataagagaaatattactagctgacaggtagatgagtattacagggcacccagaatctataggtctgtaagcatttccaaattagaatctagaaccaaatctcagaacttaatttataaacatttatctaaaaacagaggatacagagattattccccaggattagcaattccataagaactacatgggagaatatgttttgtttctaccattttgtattttttaattcactaagagaaataggcaagttatagtaggttagcataaaatatgtacaaacaagaagaactgaagtaagatggcacaggcaagtgtcagatatattgtactgagtccttgaatcccaggaaaaatgtgttttctggtagctttattgatttattcattttcttagttttccatgtctaaataatgtttccatgttacacttattacatagcatttttggtctctcacatagcaatcatatatttttaagcaaaattaccttttctaaaattctctgcttccccacaataagaaactttcatcatcctttatagaagagagataaatcaacccacaatctcttcctcatcagatcattgtcaagtgacccattcacagttacttactctagaaaaaatttttaaaggctagaataagaaaaaaacctgttatttggctacaaaattctggactggttctgaattctgttctgtaaccactcacatctgagtgtgtggcatttttttcctactctttctgtttctattaactcttgtttatagccattacccacctcctttggccctggacacaatatttactgctcaaattttttaatcttgaacttgtcattgtgaactcttcactgactgtaaattaaggttgtacatggattagtagggcctctcttcttgtgagaccagcaatgatattgttaaaaatattgaaattttgtagtaaggagtactttctttcaatttcttataatgacatttaatagtagtagtgcaatgtccagtatgttaccttaaaacagcaatttgtaagaaattacattataatgtaaaatacaaaagtaggagactgtttcttaaacagactatctgtatttcctgtaggaatttataaaatcccagagtagatcatatgactattggctgggattatctcctgaagaagattccactcgtggtatgagagtggataatataatcaactcctctgcctggtaagtgtctattcttgttagaattttatagctgggtttgagtagaggtattttccttaggagcactgtagattcaaattaaaaccagcttcaattgaaattgcatgctaaaagaacccttttctatcatgcttctttgatgtattttctaatacataaagttaccataaatgccttctcataattttgacaagtaactaacaaaattacagcagccaaattatggagaactctgtaaataattttatctaagaaaaattgtggaagcccagaaaagtcattggactaacaagtacttaatattgtgataagttctagggttcaaagatgaatgaaatattgattcatcattaaagttacttaaccatcaattttatgtgcagactgattgaattatgctagagataaagagaatatagtgtgataaatgcttcagtaagaatacacagactaacaaaacataaatttttgcaatgatacttttgttataaatgacacttcaaatgggcattttaaaaagtttatggatttagggggtacgattgcaattttgttatataaatatattgcataatgatgaagtcagtgttttcagtgtaaccataacctggatagtgtacactgtacccaacaggtaatttctcattctttattttcttcccaccctcctactttttggagtctccaatgtctattattccactttttatgtgcatgtgtacatattatttagctcccacttaagtgagaatatgcagtatttaaatttacgttcctgagttatttcatttaagagaatggtctccagctccatccattttgctgcaaagtcatgatttccttcttttttatacttgagtagtatttcatggtgtatacactctaaaagaaagcatggtttcatgttttcttatgcaataatccgttgatggacacttaggacagttccatgactttgctattgtaaatagttctacaacaaacatttaagtgtaggtgtcttttttttaatataataattcctttccctttggttagatacccaatagtggaattgctggatcaaatggtaattctatttttagttctttgaaaaattgccatagagtttacactaacttccattctgaaatgggcatttttgaatgaaaaatttaaagactggtttagaagagcacttcaggtgaaagaatataaacttcagcttaatatatcacatatatagtatagataaaataatttgttgtgtagtataatgaggaatgtggttataaatatactattttaagtggatttatacgttgtatttgttctgcttataatgggaaaaaaagtctttacatataaaaatagtggcaaattccatttgtattttttaatctctgaaatttgggaagcagaaaatatcaaacgaagaaagaaaccagagtctcaacctgctggacactattggaagtccatcatttaacacgtttttagtatatacttttagcaggagacagctctgagtcaactgtgttgaggtgccaccacagcgagtttaggcactcagatccctgcatactcatcacattgggccataatggcaaatagaattttttgttttgttttgtttgtttgctttttctttcacatagaaatagtaagtgtaggagtgtgggtcagaaagaaaaggtggccctacctctgatggttggcaatgataggatacaatgggagataagctatctacaaatggagtggagaaggatatatatttcaaaggcctaatttgtagtgaaagactagagacaaaggtaatgtgtgtgtcaggagagagtacagatggaatcttgttttgcaaacgtagaatatgtatgtgtttgtaattattgcaaatggaatggtaatctataatggaatggaaaacattgtagatattttcagttatcaaaaagaaaactgaaaaagtatataataattgtatgtatgatatatatatgtgtgtgtgtgtgtatatatatcttcactttataactctgtgttgttttggggtttgtttctgaaagggggttgtaataaatgacatctgtactatgtcaccacaaataaatctcattcttaaacatttaattgatgaacttatttactggttgatatcattatttttttacccaagtattgatgctaaattaatctaaaaggtaatatttttaatttataaatttgtttttagtgcatagtccctgatcactaatctagtatttatgggctggtcattagaacttcagagtaaaatcttttgccaagtgtcacttagagatgatgttgaacttcaggtgtcatatatctcaggtgaattcctgactttgatgattataaaaagcacttgagggagaaagggtccaagtcttactactcctagccagttagactaggagaatcttatggaatggtattattccaaattatattatatatgaaggggctggggtgctaactctgtaagcaaaggacagcaaatatcagatgttagaaagtggaaaaagcagggagaaatgaagcagtatttgacattgcatatgtgttaacataaacaattaactcggacattcttgatactctgaacattaaattagatttagtttaaacaacacagagtctagggcaataatatcatgttggaagtgtaatttttttttctgtgaatatgtttcaaatatctgacttgccaagtgtaattctttactttctctttcagggttataagaaacgcacctgacttaaataacatgtattgtggatatataaatagactatatgttcaatattatcactgcacttataaaaaaagaatgatatgtgagaagatggccaatccagtgcagcttggttctacatattttagggaggcatgaggcatcaatcaaatacatttaaggagtgtagggggtgggggttctaggctataggtaaatttaaatattttctggttgacaattagttgagtttgtctgaagacctgggattttatcatgcagatgaaacatccaggtagcaagcttcagagagaatagactgtgaatgttaatgccagagaggtataatgaagcatgtcccacctcccactttccatcatggcctgaaccctggaggaagaggaagtccattcagatagttgtggggggccttcgaattttcattttcatttacgttcttccccttctggccaagatttgccagaggcaacatcaaaaaccagcaaattttaattttgtcccacagcgttgctagggtggcatggctccccatctcgggtccatcctatacttccatgggactccctatggctgaaggccttatgagtcaaaggacttatagccaattgattgttctaggccaggtaagaatggatatggacatgcatttattacctcttaaaattattattttaagtaaaagccaataaacaaaaacgaaaaggcaagttacgagactgacttatttttaacttctgtgtgttgagctactgtaagcttggcttttgttaaagacatacagcaattagctatgcaaacataagcattgttctgaaaaaaaatatatagatagatatgtttatctccataactcataactgggagtattatacccaagaggctttgttacaaggtattttttatcctgttagtaaatattttcctgtaattctataataagcaaataaattttcatggttggggtagatgcaaaagtgacacataatcgtttagaaagcaactaaacttgttttaccagctgtttaggcatttttgtaacccctccttgatttggaggtttgaccttgacctaatttttttctttaaaaccagcccttacaatcttctgccccctcctcttctgtgacagtagctgggcctagagggaagggggcttgtatagtttgtgcagcagagcattctcagtgaaatacagatcccggcccagtgggatgccaaaatgacggagagccacatctctgatcttcaaaatatcatgatttgggttttcctggaagtaaaacaagaagagataaataacatttacagtttgatcattaaaagagcaatttgtgcgtcagaacagaaaaaggaacctattccattagggtaccaactaaaaatacgaagaaaaattataatctggtacttgctagtggattaccatagccaaaaaatgatgtttcagtctccactgaaaaaaaaaaaaaaaaagattaggactgaaattgagtattaaatgttttcctttgaagcaattcctttatccccccccccctttttttctattaaagagaaattatagcaagaccaatttgtgtgcaaaataagttttcggcttattatacttggcctggtaattttgcataaaaggcagcaagggttgggtatggtggcttatgcctgtaatcccagcacgttgggaggccaaggcaggtggattgcctgaggtcaggagttcaagaccagactggctaacatggtgaaatcccatttctactaaaaatacaaaaatcagccaggcgtggtgacacacgcctacagtcccagctactcgggaggctgaggcaggagaatcgcttgaacccgggaggcaaaggtgccagtgaggtgagattgcaccactgcactccagcctgggtgacagagcaagattccatctccaaaaaaaattaattaattaatttaaaatgcagcaagaattgattggccatataggcttctttaagttgaccttgactgaactttacctaaaaatatgctattttagttaaagtcttagtaaaataaccagtgtctcaaattcttttattttaaaagatgctttttgaatttatgcaaatgactatactgtcataaaatcacaatccaaattttagaactcaaagagaaatataaatttgcttacaaaaacatacttcactcaaataactcaaaagaaaaagattttgttgaccctgctttaaccagagccagagctttcaaacaatgtttatttgggaaatgccatttacaagccaacagctcatgaaagctgtctatcaggcatggtagaatctaggaaagaggccccctacttatcagtattttctcctaatatccccaggtagcaagattctatgtaaatcatttttatttttttatggaactcttttgggcaccatcatttttattagcataggggtggcttcagttaaaattccatagcaaggcagtacatggctctcaggtggaaattctctgttcaatattgtcattgggaaatatttacagttttttgccatcagccccaataaatgtgccacaaatgccacaaatgaaatggaggatttgtcctgactagcgctctagcttctacccaatactttgtgggctgaggcaattttactagttcccattcagcatgttcaattaacatttactaaaagagcagatttttatgccttcagctttatagtactagaaaggggaaacatctcccagtcagatatagtacccattttcacatgacatttaggtaaagggcttacaactaccttccataaagtttgtttaaacatcttaaattttataattctgttaacctgtatgtttttgtttgtttttttgttttgttttgtttttagccccagatcattttaccttttctttctttcttttatttatttatttttttttgagacagagtctcgctcttgtcacccaggctggagtgcagtggcatgatcttggctcactgcaacctccgcctccttggttcaagtgattctcctgcctcagcctaccaactagctgggattacaggtgcccaccatcacacctggctaatttttgtattttttgggctgtggactgttgagttaatgctaaaatgagttaagactttgggggacttttgggaagacatgattgattttgaaatgtgaggacatgagatttgggaggtccagggatggagtgatatggtttggctgtgtctccacccaaatctaatcttgaattgtagctcccataattcaacatgatgcaggaggggacctggtgggagataattaaatcatgagggtggttcccctatactgttctcatgttagacctaaaaccataaaaatcctagaagaaaacctaggcattaccattcaggacacaggcatgggcaaggacttcatgtctaaaacaccaaaagcaatggcatcaaaagccaaaattgacaaatgggatctaattaaactcaagagcttctgcacagcaaaagaaactaccatcagagtgtacaggcaacctacaaaatgggagaaagtttttgcaatctactcatctgacaaagggctaatatccagaatctacgaagaactcaaacaaatttacaagaaaaaacaaacaaccccatcaaaaagtgggcaaaggatatgaacagacacttctcaaaagaagacatttatgcagccaaaagacacatgtaaaagtgctcatcatcactggccatcagagaaatgcaaatcaaaaccacaatgagataccatcccacaccagttagaatggtgatcattaaaaagtcaggagacaacaggtgctggagaggatgtggagaaataggaacacttttacactgttggtgggacgtaaactagttcaaccattatggaagtcagtgtggcgattcctcagggatctagaactagaaataccatttgacccagccatcccattgctgggtatatacccaaaggattataaaacatgttgctataaagacacatgcacatgtatgtttattgcagcactattcacaatagcaaagacttggaaccaacccgaatgtccaacagtgatagactggattaagaaaatgtggcacatatacaccatggaatactatgcagccataaaaaaggatgagttcatgtcctttgtagggacatggatgaagctggaaaccatcattctcagcaaagtattgcaaggacaaaaaaccaaacaccgcatgttctcactcataggtgggaattgaacaatgagaacacctggacacaggaaggggaacatcacacaccggggcctgttgtgggatggggggaggggggagggatagcattaggagatacacctaatgttaaatgacgagttaatgggtgcagcacaccaacatggcacatgtataaatatgtaactaacctgcacgttgtgcacatgtaccctaaaacttaaagtataataataaaaaaaaagaaattacaagtgctgctccaatgaacacacaaatagtaagaaaacaagatagccttattgctgatatggagaaagtttggtggtctggatacaagacgaaaccaatcacaacgttctcttaagtcaaagcctaatccagagcaaggccctaactcttgtcaattctgtgatgggtgagagaggtgaagaagctacagaagaaatgttggaagctagcagaggttggttcatcaggtttaaggaaagaagccttctctctaacataaaaatgaaagatgaagcagcaagttgtgatgtagaaactggagcaagttatccagaagatatagctaagatagctgaaggtgtctagatgaaacagctttgtattggaaagacatgtcatctaggatttttatacctagagaggaaaagtcaatggctggcttgaaagcttcaaaggacaagctgactctcttgttagggactaatgcagctgatgactttaagttaaagccagtagtcttttatcatttcaaacgtcctagggaccttaataattatgctaaatgtacctgccaatgctctatgaatgaaacaatgacgtctagatgaaagcacatttgtttatagcattgtttactgagtattttaagcccattgttgagaaacactgctcagaaaaaaaaaaggtctttttttcaaaatattacagctcattgacaatgcacctagtcacccaagagctcttatagacatgtaccgggaggttaatgttttcagtcctgctaacacagcatccttttctgcaaccaacaaatcaaggagtaattttgactttcaagttttattctttaagaaatacacatcataaggctataactcccacagatactgattcttcttatatatcagagcaaagtaaattgaaaaccacctggagacaacttaccattctagatgccattaaaaagatttgtgattcatggaaggaagacaaagtgtcaacattaacagacacttggaagaagttgaatcaaaccctcatggataacttgcaggcgttcaagacttcagtggtggaagtaactgcagatgtggtggaaatagcaagagaactagaactggaagtggagcctgaagatgtggctgaattgatgcaattttatcaggaaactttaacaaatgaggagttgctttttatggatgagcaaagaaaatggttcttgagacaaatattattcctggtgaacatgctgtgaacattgttgaaatgctaataaagtattctgaatattgcataaacttagttgataaagcagtagcaggatttgagaggactgacttcaattttgaaagaagtttacgatgagtaaaatgctatttaacagcatcgcattctacagataaaaatttcataaaaggaagactaaattgatgctacaaacttcaccattgtcttattttaaaaatagccacatctaccccatccttcagcaaccactgctgtgatccatcagcagccatcaacatcaaggcaagacgctacaccagcaaaaagattttcactcactaaagactcaaataatcagcatttttagcaataaactatttttaataaaactatgtacattttccagacataatgctgttgtatacttaatagaagacgttgtagctcaaacataacttttacacgaattgggaaagcaaataattaatgtgactcactttattgcaataattgctttattgtggtggtctggaactgaacccacaatatttccgaggcatgcctgtaagttagttttaaattgaagaagacagaagtcaaattttgctggagttcacttagatgacatgtagaagatttagtatttgaaatgaggtggtctgatctgtgctactaacgtttgtgctattttgattttatgtataagatactttagatatgaataccagaaaaagaatcacagtgattggcctacatttttaattgtattgttgccatgcactaagatattattcttctaagtttatgtgtgctatagggatgtatacacatatgtttttccattttagggagatgaaacaaagttgagttgctaattttgtacagttttctgaattattggttcctatttagtttctctaaatagacaattcaaaataaaaaacatactgataccatcacccaaatccaagatctccagacatttgaagtaacaagtaaaatttgaggtatttctattatttttcctaagccctccaaatctgcttaattattgataggctagaaaaaagataaagattcaagtgtctttcagtgttagcgcacaaagctttaagagtaacacatatttattgagtatgtattacatacttggtattctgatcgatactttatataatcatattaaatgagcttcacaattaacttatgagcaaggtaataatattaaacccatttcaaggtgagaacattacttatccaaagccagatagagagtaagttctaaagtagtgatatttctctaactgtattaaagactacttgtatgagaattgtgtcgtgatgctggtaaaaacccaggtctctgtgtctcctcagtcttcccactgagtaacatcagcatttgagtttcctatttggtttctctatgttttaaccaaaacctccgttgagatgtttgagaatcatgttcgtaaatggtagccactagttaaaaaagggaagctgtgggaaagaaatgcaattttttcctattatcctgtaaaagggtgcctttccacatattctgtaaatacttcctgagttactcagcaaaaataaagcacagaactgttaggaaaatactaattaaagcatttttacttgtccttctgtctagtagcctctgaaacttaagaagcatttattcattttacaaacccagtgagttttcttgcaaactttcttttccttctctagcgcagaaaattactactgttgaatctaaccagaggtagacttggaatagtaattctatttgcgtaatgttttaatgattgagctatttttctcatgaactatcttatacaaagagaaacaaatatctctctgttttgtgagccagggtttcgggttggtgctggtaagagaatagtgaatggggaagtcatagtgttcgctttcatggagtttctggtacagttaacactctaaagttctattttaggttgtagaactgtgagatataaaagatggttgttattattcctattttaaatatgagaaaagggaagttaaagaggttatgaaacttgttcatgattgtaatttagttaattgacatctaaattattttgctgattctaagtttaatattctttttactatcttacaccgcctttcaaacttagatattttggacagatttcaaacatagatattttgcacaacttatattaattaagccctaacctatattggcctgattttgatgttatatgtgtatatctgaagatgcagtaaatcataccatgttcaggtaattagaaatgaaggacaagaagaaagaaaattaaaggaaaatagaacaattgggaagcaaatgataaactcacgtgatggcttctgtatattcaaagaagggactaggtaattctgataggaaagataatattaaaaataactccagtgagaagaaagggagaaataagcaattttcagctactgatgcattcatacatatcttttattgtacaataaagcccttatctgtgttatcctgtccacctctttgcattaaggtagttggtccaccttggtcatgccagccttcctctcacacattaaataatgtttgttctcacagaaggtagggtgcattttcccattatgaaaataagcacaattcatatttccttttccatcttccaaaaactcaaacctgtgaagggaaagttagaataaattcctttgaaaactgagcaaacaatcagtaatcagactctgcgtatagtagtttgatgcaccaaatagagactgaggaaaataggaaaaaaaaacaaaaaaaaaaacgattctcattgttgagaaagttccaccctgcttggcagtacaagatatgcatatttaaaaagcccaatatcaatttcatttagcataaaattaggagcaagggaacagtaggtacaataggagccctgaagaagtgatggagatggctccaaaataatgggaaaacgcattatctacaagatcagactttagttcggtattgatggaaggtcaggactcagatgaactgacaggagatggaagagccctacaggacaagtagctgcactagatgggaatacgctcatggaaaagtgagttgattcagtagttttgaaatcaggatttatgttgcaagataaatcatctaacacagcaggctgtattgtaattgtctgttaagttatatttctttctttctaaactgcttataatgagatgacaaggaatatatacttattggtttatttggagaatacctgcagaactatgtgacaaagttgttctatgtgctgaggatacactgataaagaaagacaagaaagctaatgtctttcttgttggggaactttcatttaagtggggcgataccggtaagaagcccacaaaaaatagacaagaaaaattgagacagtggtaagagttacaaagaaacaaaacagtggtggactgtatggatatgggtttaagggaggctaatttagtttgggtagagttaggttcactgaagaagtgatgtttaaatagagacttgagtctcagtggagagatggtcatggagttgtggaggcagagcattctagaagaaaatagcaagacaaaagccttaaggtaacaaaacgagtttgatttgttggaagaatgtaaagaacactaagttgcctgcaatattgtaaatgaggcagcaagcaataggagataggagataaagcccaaggtctgtgaagaggcttgcttgtagttgtagtatcctaagatcctatctatagtaaatacttaataaatatttgtttaaaaatttaattcataatttgagtaataatcagattatataagttttggttatcagtttaaattttaattgaagagagactgcagttttgatatacaggatgaacaaaagatgttgagactgcagattggaaagccatataggaagcaactccactcaatgcaatcagtccctttgcctggattgtttgtagaaggacaaagcttcgttaatggacattggaattatcttgtgaaatacaatgtatcgaattcttggagactcccagaatagaggaataggcaaaacaatattgaaaactcaaaaggactctcaagataacaactttcaactgtaaaataatgatatgaaattttgttttaaaaatatgtgaagataatcagaacaagcaaaacacaaaaacacacacacatgtgcatgcgcgcacacacacacacacactcacacatacacatacatgcacacagtgttccatggtaaggttgaatgtaaaaatgaatgggaataatatcagagataaaggtggattgtggcttagataaagagcaaagtaatgggagagaggggaaagaattgtcttatgaccagcgctgtcttgtttgaattagcaggtaagtgacccagctgctgcttctataacccatagtagtgacttggactgagagaagagggaatctcaagaataagagtttccagtactccctcctattctaagtgtccagtgacttacatattttctgagataaccgagccatcctcccacttccagacctcattcgacttctggcgagataatccga

SEQ ID NO: 2 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 3 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 4 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 5 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 6 - protein, 7 aa, synthetic construct
  VARIANT 2: note = X is K or R
  source 1..7: note = CDR1
SXTFTIS

SEQ ID NO: 7 - protein, 17 aa, synthetic construct
  VARIANT 5: note = X is L or I
  source 1..17: note = CDR2
GIIPXFGTITYAQKFQG

SEQ ID NO: 8 - protein, 9 aa, synthetic construct
  VARIANT 7: note = X is L or F
  source 1..9: note = CDR3
RGNWNPXDP

SEQ ID NO: 9 - protein, 5 aa, synthetic construct
  VARIANT 2: note = X is F or Y
  source 1..5: note = CDR1
RXWIG

SEQ ID NO: 10 - protein, 17 aa, synthetic construct
  VARIANT 1: note = X is H or N
  VARIANT 6: note = X is D or V
  VARIANT 15: note = X is L or M
  source 1..17: note = CDR3
XIRYFXWSEDYHYYXDV

SEQ ID NO: 11 - protein, 17 aa, synthetic construct
  VARIANT 6: note = X is S or G
  VARIANT 8: note = X is S or T
  VARIANT 9: note = X is I or T
  VARIANT 10: note = X is G or Y
  source 1..17: note = CDR2
DISWSXGXXXYADSVKG

SEQ ID NO: 12 - protein, 15 aa, synthetic construct
  VARIANT 3: note = X is R or M
  VARIANT 15: note = X is H or Y
  source 1..15: note = CDR3
DHXGYGDYEGGGFDX

SEQ ID NO: 13 - protein, 267 aa, Homo sapiens
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVMDSNYQLIWGAGTKLIIKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 14 - protein, 310 aa, Homo sapiens
MRIRLLCCVAFSLLWAGPVIAGITQAPTSQILAAGRRMTLRCTQDMRHNAMYWYRQDLGLGLRLIHYSNTAGTTGKGEVPDGYSVSRANTDDFPLTLASAVPSQTSVYFCASSEAGGNTGELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 15 - DNA, 321 bp, synthetic construct
  source 1..321: mol_type = other DNA; note = IGKV1-39/jk1
  CDS <1..>321: protein_id = 16; translation = DIQMTQSPSSLSASVGDRVTITCRASQSISSYLNWYQQKPGKAP KLLIYAASSLQSGVPSRFSGSGSGTDFTLTISSLQPEDFATYYCQQSYSTPPTFGQGT KVEIK
gacatccagatgacccagtctccatcctccctgtctgcatctgtaggagacagagtcaccatcacttgccgggcaagtcagagcattagcagctacttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccaacgttcggccaagggaccaaggtggagatcaaa

SEQ ID NO: 16 - protein, 107 aa, synthetic construct
  source 1..107: note = Synthetic Construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 17 - DNA, 324 bp, synthetic construct
  source 1..324: mol_type = other DNA; note = IGKV1-39/jk5
  CDS <1..321: protein_id = 18
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 18 - protein, 107 aa, synthetic construct [sequence omitted]

SEQ ID NO: 19 - protein, 108 aa, synthetic construct
  source 1..108: note = IGKV1-39/jk5 common light chain variable domain translation
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 20 - protein, 95 aa, synthetic construct
  source 1..95: note = V-region IGKV1-39A
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 21 - protein, 6 aa, synthetic construct
  source 1..6: note = CDR1 common light chain
QSISSY

SEQ ID NO: 22 - skipped

SEQ ID NO: 23 - protein, 7 aa, synthetic construct
  source 1..7: note = CDR3 common light chain
QQSYSTP

SEQ ID NO: 24 - DNA, 294 bp, synthetic construct
  source 1..294: mol_type = other DNA; note = IgG heavy chain CH1 region
  CDS <1..>294: protein_id = 25; translation = ASTKGPSVFPLAPSSKSTSGGTAALGCLVKDYFPEPVTVSWNSG ALTSGVHTFPAVLQSSGLYSLSSVVTVPSSSLGTQTYICNVNHKPSNTKVDKRV
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagagagtt

SEQ ID NO: 25 - protein, 98 aa, synthetic construct
  source 1..98: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 26 - DNA, 45 bp, synthetic construct
  source 1..45: mol_type = other DNA; note = IgG heavy chain hinge region
  CDS <1..>45: protein_id = 27; translation = EPKSCDKTHTCPPCP
gagcccaaatcttgtgacaaaactcacacatgcccaccgtgccca

SEQ ID NO: 27 - protein, 15 aa, synthetic construct
  source 1..15: note = Synthetic Construct
EPKSCDKTHTCPPCP

SEQ ID NO: 28 - DNA, 330 bp, synthetic construct
  source 1..330: mol_type = other DNA; note = IgG heavy chain CH2 region
  CDS <1..>330: protein_id = 29; translation = APELLGGPSVFLFPPKPKDTLMISRTPEVTCVVVDVSHEDPEVK FNWYVDGVEVHNAKTKPREEQYNSTYRVVSVLTVLHQDWLNGKEYKCKVSNKALPAPI EKTISKAK
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaa

SEQ ID NO: 29 - protein, 110 aa, synthetic construct
  source 1..110: note = Synthetic Construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 30 - DNA, 330 bp, synthetic construct
  source 1..330: mol_type = other DNA; note = IgG heavy chain CH2 domain containing L235G and G236R silencing substitutions
  CDS <1..>330: protein_id = 31; translation = APELGRGPSVFLFPPKPKDTLMISRTPEVTCVVVDVSHEDPEVK FNWYVDGVEVHNAKTKPREEQYNSTYRVVSVLTVLHQDWLNGKEYKCKVSNKALPAPI EKTISKAK
gcacctgaactcggcaggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaa

SEQ ID NO: 31 - protein, 110 aa, synthetic construct
  source 1..110: note = Synthetic Construct
APELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 32 - DNA, 321 bp, synthetic construct
  source 1..321: mol_type = other DNA; note = IgG heavy chain CH3 domain containing substitutions L351K and T366K (KK)
  CDS <1..318: protein_id = 33; translation = GQPREPQVYTKPPSREEMTKNQVSLKCLVKGFYPSDIAVEWESN GQPENNYKTTPPVLDSDGSFFLYSKLTVDKSRWQQGNVFSCSVMHEALHNHYTQKSLS LSPG
gggcagccccgagaaccacaggtgtacaccaagcccccatcccgggaggagatgaccaagaaccaggtcagcctgaagtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 33 - protein, 106 aa, synthetic construct
  source 1..106: note = Synthetic Construct
GQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - DNA, 321 bp, synthetic construct
  source 1..321: mol_type = other DNA; note = IgG heavy chain CH3 region containing substitutions L351D and L368E (DE)
  CDS <1..318: protein_id = 35; translation = GQPREPQVYTDPPSREEMTKNQVSLTCEVKGFYPSDIAVEWESN GQPENNYKTTPPVLDSDGSFFLYSKLTVDKSRWQQGNVFSCSVMHEALHNHYTQKSLS LSPG
gggcagccccgagaaccacaggtgtacaccgaccccccatcccgggaggagatgaccaagaaccaggtcagcctgacctgcgaggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 35 - protein, 106 aa, synthetic construct
  source 1..106: note = Synthetic Construct
GQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 4 aa, synthetic construct
  source 1..4: note = CH# sequence
DEKK

SEQ ID NO: 37 - DNA, 373 bp, synthetic construct
  source 1..373: mol_type = other DNA; note = MF8057
ggcccagccggccatggccgaggtgcagctggtgcagtctggagctgaggtgaagaagcctgggtcctcggtgaaggtctcctgcaaggcttctggaggcaccttccgcagctttggtatcagttgggtgcgacaggcccctggacaaggacttgagtggatgggaggattcatccctgtccttggtacagcaaactacgcacagaaattccagggcagagtgacgattatcgcggacaaatccacgaacacagcctacatggagctgagcagcctgagatctgaggacacggccgtgtattactgtgcgcgaaggggtaactggaacccgttcgacccctggggccagggaaccctggtcaccgtctcgagt

SEQ ID NO: 38 - protein, 30 aa, synthetic construct
  source 1..30: note = MF8057 FW1
EVQLVQSGAEVKKPGSSVKVSCKASGGTFR

SEQ ID NO: 39 - protein, 14 aa, synthetic construct
  source 1..14: note = MF8057 FW2
WVRQAPGQGLEWMG

SEQ ID NO: 40 - protein, 32 aa, synthetic construct
  source 1..32: note = MF8057 FW3 -1AA in FW4
RVTIIADKSTNTAYMELSSLRSEDTAVYYCAR

SEQ ID NO: 41 - protein, 11 aa, synthetic construct
  source 1..11: note = MF8057 FW4
WGQGTLVTVSS

SEQ ID NO: 42 - protein, 5 aa, synthetic construct
  source 1..5: note = MF8057 CDR1
SFGIS

SEQ ID NO: 43 - protein, 17 aa, synthetic construct
  source 1..17: note = MF8057 CDR2
GFIPVLGTANYAQKFQG

SEQ ID NO: 44 - protein, 9 aa, synthetic construct
  source 1..9: note = MF8057 CDR3
RGNWNPFDP

SEQ ID NO: 45 - protein, 118 aa, synthetic construct
  source 1..118: note = MF8057 VH
EVQLVQSGAEVKKPGSSVKVSCKASGGTFRSFGISWVRQAPGQGLEWMGGFIPVLGTANYAQKFQGRVTIIADKSTNTAYMELSSLRSEDTAVYYCARRGNWNPFDPWGQGTLVTVSS

SEQ ID NO: 46 - DNA, 379 bp, synthetic construct
  source 1..379: mol_type = other DNA; note = MF8058
ggcccagccggccatggcccaggtgcagctggtgcagtctggggctgaggtgaagaagcctgggtcctcggtgaaggtctcctgcaaggcttctggagacgccttcaaaagcaaaacctttactatcagctgggtgcgacaggcccctggacaggggcttgagtggctgggggggatcatccctctttttggtacaattacctacgcacagaagttccagggcagagtcacgattaccgcggacaaatccacgaacactgccttcatggaactgagcagcctgagatctgaggacacggccatgtattattgtacgcgacgggggaactggaaccccttcgacccctggggccagggaaccctggtcaccgtctcgagt

SEQ ID NO: 47 - protein, 14 aa, synthetic construct
  source 1..14: note = MF8058 FW2
WVRQAPGQGLEWLG

SEQ ID NO: 48 - protein, 30 aa, synthetic construct
  source 1..30: note = MF8058 FW1
QVQLVQSGAEVKKPGSSVKVSCKASGDAFK

SEQ ID NO: 49 - protein, 32 aa, synthetic construct
  source 1..32: note = MF8058 FW3
RVTITADKSTNTAFMELSSLRSEDTAMYYCTR

SEQ ID NO: 50 - protein, 11 aa, synthetic construct
  source 1..11: note = MF8058 FW4
WGQGTLVTVSS

SEQ ID NO: 51 - protein, 7 aa, synthetic construct
  source 1..7: note = MF8058 CDR1
SKTFTIS

SEQ ID NO: 52 - protein, 17 aa, synthetic construct
  source 1..17: note = MF8058 CDR2
GIIPLFGTITYAQKFQG

SEQ ID NO: 53 - protein, 9 aa, synthetic construct
  source 1..9: note = MF8058 CDR3
RGNWNPFDP

SEQ ID NO: 54 - protein, 120 aa, synthetic construct
  source 1..120: note = MF8058 VH
QVQLVQSGAEVKKPGSSVKVSCKASGDAFKSKTFTISWVRQAPGQGLEWLGGIIPLFGTITYAQKFQGRVTITADKSTNTAFMELSSLRSEDTAMYYCTRRGNWNPFDPWGQGTLVTVSS

SEQ ID NO: 55 - DNA, 379 bp, synthetic construct
  source 1..379: mol_type = other DNA; note = MF8078
ggcccagccggccatggccgaggtgcagctggtgcagtctgggtctgagttgaagaagcctgggtcctcggtgaaggtctcctgcaaggcttctggagtcaccttcaacagcagaacctttactatcagttgggtgcgacaggcccctggacaagggcttgagtggctggggagtatcatccctatttttggtacaataacctacgcacagaagttccagggcagagtcacgattaccgcggacaaatccacgagcactgccttcatggaactgaccagcctgagatctgaggacacggccatatattattgtacgagacgggggaactggaaccccttcgacccctggggccagggcaccctggtcaccgtctcgagt

SEQ ID NO: 56 - protein, 30 aa, synthetic construct
  source 1..30: note = MF8078 FW1
EVQLVQSGSELKKPGSSVKVSCKASGVTFN

SEQ ID NO: 57 - protein, 14 aa, synthetic construct
  source 1..14: note = MF8078 FW2
WVRQAPGQGLEWLG

SEQ ID NO: 58 - protein, 32 aa, synthetic construct
  source 1..32: note = MF8078 FW3
RVTITADKSTSTAFMELTSLRSEDTAIYYCTR

SEQ ID NO: 59 - protein, 11 aa, synthetic construct
  source 1..11: note = MF8078 FW4
WGQGTLVTVSS

SEQ ID NO: 60 - protein, 7 aa, synthetic construct
  source 1..7: note = MF8078 CDR1
SRTFTIS

SEQ ID NO: 61 - protein, 17 aa, synthetic construct
  source 1..17: note = MF8078 CDR2
SIIPIFGTITYAQKFQG

SEQ ID NO: 62 - protein, 9 aa, synthetic construct
  source 1..9: note = MF8078 CDR3
RGNWNPFDP

SEQ ID NO: 63 - protein, 120 aa, synthetic construct
  source 1..120: note = MF8078 VH
EVQLVQSGSELKKPGSSVKVSCKASGVTFNSRTFTISWVRQAPGQGLEWLGSIIPIFGTITYAQKFQGRVTITADKSTSTAFMELTSLRSEDTAIYYCTRRGNWNPFDPWGQGTLVTVSS

SEQ ID NO: 64 - protein, 30 aa, synthetic construct
  source 1..30: note = FW1
QVQLVQSGAEVKKPGSSVKVSCKASGGTFR

SEQ ID NO: 65 - protein, 14 aa, synthetic construct
  source 1..14: note = FW2
WVRQAPGQGLEWVG

SEQ ID NO: 66 - protein, 32 aa, synthetic construct
  source 1..32: note = FW3
RVTITADKSATTAYMELSSLRSEDTAIYYCAK

SEQ ID NO: 67 - protein, 11 aa, synthetic construct
  source 1..11: note = FW4
WGQGTLVTVSS

SEQ ID NO: 68 - protein, 5 aa, synthetic construct
  source 1..5: note = CDR1
GSGIS

SEQ ID NO: 69 - protein, 17 aa, synthetic construct
  source 1..17: note = CDR2
GFIPFFGSANYAQKFRD

SEQ ID NO: 70 - protein, 9 aa, synthetic construct
  source 1..9: note = CDR3
RGNWNPLDP

SEQ ID NO: 71 - protein, 118 aa, synthetic construct
  source 1..118: note = VH
QVQLVQSGAEVKKPGSSVKVSCKASGGTFRGSGISWVRQAPGQGLEWVGGFIPFFGSANYAQKFRDRVTITADKSATTAYMELSSLRSEDTAIYYCAKRGNWNPLDPWGQGTLVTVSS

SEQ ID NO: 72 - DNA, 90 bp, synthetic construct
  source 1..90: mol_type = other DNA; note = FW1
caggtgcagctggtgcagtctggggctgaggtgaagaagcctgggtcctcggtgaaggtctcctgcaaggcttctggaggcaccttcaga

SEQ ID NO: 73 - DNA, 42 bp, synthetic construct
  source 1..42: mol_type = other DNA; note = FW2
tgggtgcgacaggcccctggacaagggcttgagtgggtgggc

SEQ ID NO: 74 - DNA, 96 bp, synthetic construct
  source 1..96: mol_type = other DNA; note = FW3
agagtcacgattaccgcggacaaatccgcgaccacagcctacatggaactgagcagcctgagatctgaggacacggccatatattactgtgcgaaa

SEQ ID NO: 75 - DNA, 33 bp, synthetic construct
  source 1..33: mol_type = other DNA; note = FW4
tggggccagggcaccctggtcaccgtctcgagt

SEQ ID NO: 76 - DNA, 15 bp, synthetic construct
  source 1..15: mol_type = other DNA; note = CDR1
ggttctggtatcagc

SEQ ID NO: 77 - DNA, 51 bp, synthetic construct
  source 1..51: mol_type = other DNA; note = CDR2
gggttcatccctttctttggttcagcaaactacgcacagaagttccgggac

SEQ ID NO: 78 - DNA, 27 bp, synthetic construct
  source 1..27: mol_type = other DNA; note = CDR3
agaggaaattggaacccgctcgacccc

SEQ ID NO: 79 - DNA, 354 bp, synthetic construct
  source 1..354: mol_type = other DNA; note = VH
caggtgcagctggtgcagtctggggctgaggtgaagaagcctgggtcctcggtgaaggtctcctgcaaggcttctggaggcaccttcagaggttctggtatcagctgggtgcgacaggcccctggacaagggcttgagtgggtgggcgggttcatccctttctttggttcagcaaactacgcacagaagttccgggacagagtcacgattaccgcggacaaatccgcgaccacagcctacatggaactgagcagcctgagatctgaggacacggccatatattactgtgcgaaaagaggaaattggaacccgctcgacccctggggccagggcaccctggtcaccgtctcgagt

SEQ ID NO: 80 - protein, 30 aa, synthetic construct
  source 1..30: note = FW1
QVQLVQSGAEVKKPGSSVKVSCKASGVTFK

SEQ ID NO: 81 - protein, 14 aa, synthetic construct
  source 1..14: note = FW2
WVRQAPGQGLEWLG

SEQ ID NO: 82 - protein, 32 aa, synthetic construct
  source 1..32: note = FW3
RVSITADKSTNTAYLELNSLRSEDTAIYYCAR

SEQ ID NO: 83 - protein, 11 aa, synthetic construct
  source 1..11: note = FW4
WGQGTLVTVSS

SEQ ID NO: 84 - protein, 7 aa, synthetic construct
  source 1..7: note = CDR1
SKTLTIS

SEQ ID NO: 85 - protein, 17 aa, synthetic construct
  source 1..17: note = CDr2
GIIPIFGSITYAQKFQD

SEQ ID NO: 86 - protein, 9 aa, synthetic construct
  source 1..9: note = CDR3
RGNWNPFDP

SEQ ID NO: 87 - protein, 120 aa, synthetic construct
  source 1..120: note = VH
QVQLVQSGAEVKKPGSSVKVSCKASGVTFKSKTLTISWVRQAPGQGLEWLGGIIPIFGSITYAQKFQDRVSITADKSTNTAYLELNSLRSEDTAIYYCARRGNWNPFDPWGQGTLVTVSS

SEQ ID NO: 88 - DNA, 90 bp, synthetic construct
  source 1..90: mol_type = other DNA; note = FW1
caggtgcagctggtgcagtctggggctgaggtgaagaagcctgggtcctcggtgaaggtctcctgcaaggcttctggagtcaccttcaaa

SEQ ID NO: 89 - DNA, 42 bp, synthetic construct
  source 1..42: mol_type = other DNA; note = FW2
tgggtgcgacaggcccctggacaagggcttgagtggctggga

SEQ ID NO: 90 - DNA, 96 bp, synthetic construct
  source 1..96: mol_type = other DNA; note = FW3
agagtctcgattaccgcggacaaatccacgaacacagcctacttggaactgaacagcctgaggtctgaggacacggccatttattattgtgcgaga

SEQ ID NO: 91 - DNA, 33 bp, synthetic construct; identical to SEQ ID NO: 75
  source 1..33: mol_type = other DNA; note = FW4

SEQ ID NO: 92 - DNA, 21 bp, synthetic construct
  source 1..21: mol_type = other DNA; note = CDR1
agcaaaaccttaactatcagc

SEQ ID NO: 93 - DNA, 51 bp, synthetic construct
  source 1..51: mol_type = other DNA; note = CDR2
gggatcatccctatttttggttcaataacctacgcacagaagttccaggac

SEQ ID NO: 94 - DNA, 27 bp, synthetic construct
  source 1..27: mol_type = other DNA; note = CDR3
cgggggaactggaaccccttcgacccc

SEQ ID NO: 95 - DNA, 360 bp, synthetic construct
  source 1..360: mol_type = other DNA; note = VH
caggtgcagctggtgcagtctggggctgaggtgaagaagcctgggtcctcggtgaaggtctcctgcaaggcttctggagtcaccttcaaaagcaaaaccttaactatcagctgggtgcgacaggcccctggacaagggcttgagtggctgggagggatcatccctatttttggttcaataacctacgcacagaagttccaggacagagtctcgattaccgcggacaaatccacgaacacagcctacttggaactgaacagcctgaggtctgaggacacggccatttattattgtgcgagacgggggaactggaaccccttcgacccctggggccagggcaccctggtcaccgtctcgagt

SEQ ID NO: 96 - protein, 30 aa, synthetic construct; identical to SEQ ID NO: 38
  source 1..30: note = FW1

SEQ ID NO: 97 - protein, 14 aa, synthetic construct
  source 1..14: note = FW2
WVRQAPGQGLEWVG

SEQ ID NO: 98 - protein, 32 aa, synthetic construct; identical to SEQ ID NO: 66
  source 1..32: note = FW3

SEQ ID NO: 99 - protein, 11 aa, synthetic construct
  source 1..11: note = FW4
WGQGTLVTVSS

SEQ ID NO: 100 - protein, 5 aa, synthetic construct
  source 1..5: note = CDR1
GSGIS

SEQ ID NO: 101 - protein, 17 aa, synthetic construct
  source 1..17: note = CDR2
GFIPFFGSANYAQKFRD

SEQ ID NO: 102 - protein, 9 aa, synthetic construct
  source 1..9: note = CDR3
RGNWNPFDP

SEQ ID NO: 103 - protein, 118 aa, synthetic construct
  source 1..118: note = VH
EVQLVQSGAEVKKPGSSVKVSCKASGGTFRGSGISWVRQAPGQGLEWVGGFIPFFGSANYAQKFRDRVTITADKSATTAYMELSSLRSEDTAIYYCAKRGNWNPFDPWGQGTLVTVSS

SEQ ID NO: 104 - DNA, 90 bp, synthetic construct
  source 1..90: mol_type = other DNA; note = FW1
gaggtgcagctggtgcagtctggagctgaggtgaagaagcctgggtcctcggtgaaggtctcctgcaaggcttctggaggcaccttcaga

SEQ ID NO: 105 - DNA, 42 bp, synthetic construct; identical to SEQ ID NO: 73
  source 1..42: mol_type = other DNA; note = FW2

SEQ ID NO: 106 - DNA, 96 bp, synthetic construct; identical to SEQ ID NO: 74
  source 1..96: mol_type = other DNA; note = FW3

SEQ ID NO: 107 - DNA, 33 bp, synthetic construct; identical to SEQ ID NO: 75
  source 1..33: mol_type = other DNA; note = FW4

SEQ ID NO: 108 - DNA, 15 bp, synthetic construct
  source 1..15: mol_type = other DNA; note = CDR1
ggttctggtatcagc

SEQ ID NO: 109 - DNA, 51 bp, synthetic construct; identical to SEQ ID NO: 77
  source 1..51: mol_type = other DNA; note = CDR2

SEQ ID NO: 110 - DNA, 27 bp, synthetic construct
  source 1..27: mol_type = other DNA; note = CDR3
agaggaaattggaacccgttcgacccc

SEQ ID NO: 111 - DNA, 354 bp, synthetic construct
  source 1..354: mol_type = other DNA; note = FW
gaggtgcagctggtgcagtctggagctgaggtgaagaagcctgggtcctcggtgaaggtctcctgcaaggcttctggaggcaccttcagaggttctggtatcagctgggtgcgacaggcccctggacaagggcttgagtgggtgggcgggttcatccctttctttggttcagcaaactacgcacagaagttccgggacagagtcacgattaccgcggacaaatccgcgaccacagcctacatggaactgagcagcctgagatctgaggacacggccatatattactgtgcgaaaagaggaaattggaacccgttcgacccctggggccagggcaccctggtcaccgtctcgagt

SEQ ID NO: 112 - DNA, 379 bp, synthetic construct
  source 1..379: mol_type = other DNA; note = MF8397
caggtgcagctggtgcagtctgggggaggcctggtacagcctggggggtccctgagactctcctgtgcaacctctggattcaaattcagtagctatgccctgagctgggtccgccaggcaccagggaagggactggagtgggtctcaggtattagtggtagtggtcgcaccacatggtatgcagactccgtgaagggccggttcaccatctccagagacaattccaagaacacgctgtatctgcaaatgaacagcctgagagccgaggacacggccgtatattactgtgcgagagacggtggatacagctatggcccttactggtacttcgatctctggggccgtggaaccctggtcacccctggtcaccgtctccagt

SEQ ID NO: 113 - protein, 30 aa, synthetic construct
  source 1..30: note = MF8397 FW1
QVQLVQSGGGLVQPGGSLRLSCATSGFKFS

SEQ ID NO: 114 - protein, 14 aa, synthetic construct
  source 1..14: note = MF8397 FW2
WVRQAPGKGLEWVS

SEQ ID NO: 115 - protein, 32 aa, synthetic construct
  source 1..32: note = MF8397 FW3
RFTISRDNSKNTLYLQMNSLRAEDTAVYYCAR

SEQ ID NO: 116 - protein, 11 aa, synthetic construct
  source 1..11: note = MF8397 FW4
WGRGTLVTVSS

SEQ ID NO: 117 - protein, 5 aa, synthetic construct
  source 1..5: note = MF8397 CDR1
SYALS

SEQ ID NO: 118 - protein, 17 aa, synthetic construct
  source 1..17: note = MF8397 CDR2
GISGSGRTTWYADSVKG

SEQ ID NO: 119 - protein, 14 aa, synthetic construct
  source 1..14: note = MF8397 CDR3
DGGYSYGPYWYFDL

SEQ ID NO: 120 - protein, 123 aa, synthetic construct
  source 1..123: note = MF8397 VH
QVQLVQSGGGLVQPGGSLRLSCATSGFKFSSYALSWVRQAPGKGLEWVSGISGSGRTTWYADSVKGRFTISRDNSKNTLYLQMNSLRAEDTAVYYCARDGGYSYGPYWYFDLWGRGTLVTVSS

SEQ ID NO: 121 - protein, 30 aa, synthetic construct
  source 1..30: note = FW1
QVQLVESGGGLVQPGGSLRLSCATSGFTFI

SEQ ID NO: 122 - protein, 14 aa, synthetic construct
  source 1..14: note = FW2
WVRQAPGKGLEWVS

SEQ ID NO: 123 - protein, 32 aa, synthetic construct
  source 1..32: note = FW3
RFTISRDNSKNTLFLQMNSLRAEDTAVYYCAR

SEQ ID NO: 124 - protein, 11 aa, synthetic construct
  source 1..11: note = FW4
WGRGTLVTVSS

SEQ ID NO: 125 - protein, 5 aa, synthetic construct
  source 1..5: note = CDR1
SYALS

SEQ ID NO: 126 - protein, 17 aa, synthetic construct
  source 1..17: note = CDR2
AISGSGRTTWYADSVKG

SEQ ID NO: 127 - protein, 14 aa, synthetic construct
  source 1..14: note = CDR3
DGGYTYGPYWYFDL

SEQ ID NO: 128 - protein, 123 aa, synthetic construct
  source 1..123: note = VH
QVQLVESGGGLVQPGGSLRLSCATSGFTFISYALSWVRQAPGKGLEWVSAISGSGRTTWYADSVKGRFTISRDNSKNTLFLQMNSLRAEDTAVYYCARDGGYTYGPYWYFDLWGRGTLVTVSS

SEQ ID NO: 129 - DNA, 90 bp, synthetic construct
  source 1..90: mol_type = other DNA; note = FW1
caggtgcagctggtggagtctgggggaggcctggtacagcctggggggtccctgagactctcctgtgcaacctctggattcacctttatc

SEQ ID NO: 130 - DNA, 42 bp, synthetic construct
  source 1..42: mol_type = other DNA; note = FW2
tgggtccgccaggctccagggaaggggctggaatgggtctca

SEQ ID NO: 131 - DNA, 96 bp, synthetic construct
  source 1..96: mol_type = other DNA; note = FW3
cggttcaccatctccagagacaattccaagaacacgctgtttctgcaaatgaacagcctgagagccgaggacacggccgtatattactgtgcgaga

SEQ ID NO: 132 - DNA, 33 bp, synthetic construct
  source 1..33: mol_type = other DNA; note = FW4
tggggccgtggcaccctggtcaccgtctcgagt

SEQ ID NO: 133 - DNA, 15 bp, synthetic construct
  source 1..15: mol_type = other DNA; note = CDR1
agctatgccctgagc

SEQ ID NO: 134 - DNA, 51 bp, synthetic construct
  source 1..51: mol_type = other DNA; note = CDR2
gctattagtggtagtggtcgtaccacatggtacgcagactccgtgaagggc

SEQ ID NO: 135 - DNA, 42 bp, synthetic construct
  source 1..42: mol_type = other DNA; note = CDR3
gacggtggatacacctatggcccttactggtacttcgatctc

SEQ ID NO: 136 - DNA, 369 bp, synthetic construct
  source 1..369: mol_type = other DNA; note = VH
caggtgcagctggtggagtctgggggaggcctggtacagcctggggggtccctgagactctcctgtgcaacctctggattcacctttatcagctatgccctgagctgggtccgccaggctccagggaaggggctggaatgggtctcagctattagtggtagtggtcgtaccacatggtacgcagactccgtgaagggccggttcaccatctccagagacaattccaagaacacgctgtttctgcaaatgaacagcctgagagccgaggacacggccgtatattactgtgcgagagacggtggatacacctatggcccttactggtacttcgatctctggggccgtggcaccctggtcaccgtctcgagt

SEQ ID NO: 137 - DNA, 391 bp, synthetic construct
  source 1..391: mol_type = other DNA; note = MF8508
ggcccagccggccatggccgaggtgcagctggtggagtctgggggaggattggtacagcctggcaggtccctgagactctcctgtgcaacctctggattcaactttgatgattataccatgcactgggtccggcaagctccagggaagggcctggagtgggtctcagatattagttggagtagtggtagcataggctatgcggactctgtgaagggccgattcaccatctccagagacaacgccaagaactccctgtggctgcaaatgaacagtctgagaactgaggacacggccttgtatttctgtgcaaaagatcatagggggtacggtgactacgagggtggtggctttgactactggggccagggaaccctggtcaccgtctccagt

SEQ ID NO: 138 - protein, 30 aa, synthetic construct
  source 1..30: note = MF8508 FW1
EVQLVESGGGLVQPGRSLRLSCATSGFNFD

SEQ ID NO: 139 - protein, 14 aa, synthetic construct
  source 1..14: note = MF8508 FW2
WVRQAPGKGLEWVS

SEQ ID NO: 140 - protein, 32 aa, synthetic construct
  source 1..32: note = MF8508 FW3
RFTISRDNAKNSLWLQMNSLRTEDTALYFCAK

SEQ ID NO: 141 - protein, 11 aa, synthetic construct
  source 1..11: note = MF8508 FW4
WGQGTLVTVSS

SEQ ID NO: 142 - protein, 5 aa, synthetic construct
  source 1..5: note = MF8508 CDR1
DYTMH

SEQ ID NO: 143 - protein, 17 aa, synthetic construct
  source 1..17: note = MF8508 CDR2
DISWSSGSIGYADSVKG

SEQ ID NO: 144 - protein, 15 aa, synthetic construct
  source 1..15: note = MF8508 CDR3
DHRGYGDYEGGGFDY

SEQ ID NO: 145 - protein, 124 aa, synthetic construct
  source 1..124: note = MF8508 VH
EVQLVESGGGLVQPGRSLRLSCATSGFNFDDYTMHWVRQAPGKGLEWVSDISWSSGSIGYADSVKGRFTISRDNAKNSLWLQMNSLRTEDTALYFCAKDHRGYGDYEGGGFDYWGQGTLVTVSS

SEQ ID NO: 146 - protein, 30 aa, synthetic construct
  source 1..30: note = FW1
EVQLVESGGGLVQPGRSLRLSCATSGFTFD

SEQ ID NO: 147 - protein, 14 aa, synthetic construct
  source 1..14: note = FW2
WVRQAPGKGLEWVS

SEQ ID NO: 148 - protein, 32 aa, synthetic construct
  source 1..32: note = FW3
RFTISRDNAKNSLYLQMNSLRAEDTALYFCAK

SEQ ID NO: 149 - protein, 11 aa, synthetic construct
  source 1..11: note = FW4
WGQGTLVTVSS

SEQ ID NO: 150 - protein, 5 aa, synthetic construct
  source 1..5: note = CDR1
DYTMH

SEQ ID NO: 151 - protein, 17 aa, synthetic construct
  source 1..17: note = CDR3
DISWSSGSIGYADSVKG

SEQ ID NO: 152 - protein, 15 aa, synthetic construct
  source 1..15: note = CDR3
DHRGYGDYEGGGFDH

SEQ ID NO: 153 - protein, 124 aa, synthetic construct
  source 1..124: note = VH
EVQLVESGGGLVQPGRSLRLSCATSGFTFDDYTMHWVRQAPGKGLEWVSDISWSSGSIGYADSVKGRFTISRDNAKNSLYLQMNSLRAEDTALYFCAKDHRGYGDYEGGGFDHWGQGTLVTVSS

SEQ ID NO: 154 - DNA, 90 bp, synthetic construct
  source 1..90: mol_type = other DNA; note = FW1
gaggtgcagctggtggagtctgggggaggcttggtacagcctggcaggtccctgagactctcctgtgcaacctctggattcacctttgat

SEQ ID NO: 155 - DNA, 42 bp, synthetic construct
  source 1..42: mol_type = other DNA; note = FW2
tgggtccggcaagctccagggaagggcctggagtgggtctca

SEQ ID NO: 156 - DNA, 96 bp, synthetic construct
  source 1..96: mol_type = other DNA; note = FW3
cgattcaccatctccagagacaacgccaagaactccctgtatctgcaaatgaacagtctgagagctgaggacacggccttgtatttctgtgcaaaa

SEQ ID NO: 157 - DNA, 33 bp, synthetic construct
  source 1..33: mol_type = other DNA; note = FW4
tggggccagggaaccctggtcaccgtctccagt

SEQ ID NO: 158 - DNA, 15 bp, synthetic construct
  source 1..15: mol_type = other DNA; note = CDR1
gattataccatgcac

SEQ ID NO: 159 - DNA, 51 bp, synthetic construct
  source 1..51: mol_type = other DNA; note = CDR2
gatattagttggagtagtggtagcataggctatgcggactctgtgaagggc

SEQ ID NO: 160 - DNA, 45 bp, synthetic construct
  source 1..45: mol_type = other DNA; note = MF8998_16
gatcatagggggtacggtgactacgagggtggtggctttgaccac

SEQ ID NO: 161 - DNA, 372 bp, synthetic construct
  source 1..372: mol_type = other DNA; note = VH
gaggtgcagctggtggagtctgggggaggcttggtacagcctggcaggtccctgagactctcctgtgcaacctctggattcacctttgatgattataccatgcactgggtccggcaagctccagggaagggcctggagtgggtctcagatattagttggagtagtggtagcataggctatgcggactctgtgaagggccgattcaccatctccagagacaacgccaagaactccctgtatctgcaaatgaacagtctgagagctgaggacacggccttgtatttctgtgcaaaagatcatagggggtacggtgactacgagggtggtggctttgaccactggggccagggaaccctggtcaccgtctccagt

SEQ ID NO: 162 - protein, 30 aa, synthetic construct
  source 1..30: note = FW1
EVQLVESGGVVVQPGGSLRLSCAASGFTFD

SEQ ID NO: 163 - protein, 14 aa, synthetic construct
  source 1..14: note = FW2
WVRQAPGKGLEWVS

SEQ ID NO: 164 - protein, 32 aa, synthetic construct
  source 1..32: note = FW3
RFTISRDNSKNSLYLQMNSLRTEDTALYYCAK

SEQ ID NO: 165 - protein, 11 aa, synthetic construct
  source 1..11: note = FW4
WGRGTLVTVSS

SEQ ID NO: 166 - protein, 5 aa, synthetic construct
  source 1..5: note = CDR1
DYTMH

SEQ ID NO: 167 - protein, 17 aa, synthetic construct
  source 1..17: note = CDR2
DISWSGGSIYYADSVKG

SEQ ID NO: 168 - protein, 15 aa, synthetic construct
  source 1..15: note = CDR3
DHRGYGDYEGGGFDY

SEQ ID NO: 169 - protein, 124 aa, synthetic construct
  source 1..124: note = VH
EVQLVESGGVVVQPGGSLRLSCAASGFTFDDYTMHWVRQAPGKGLEWVSDISWSGGSIYYADSVKGRFTISRDNSKNSLYLQMNSLRTEDTALYYCAKDHRGYGDYEGGGFDYWGRGTLVTVSS

SEQ ID NO: 170 - DNA, 90 bp, synthetic construct
  source 1..90: mol_type = other DNA; note = FW1
gaggtgcagctggtggagagcggcggcgtggtggtgcagcccggcggcagcctgagactgagctgcgccgccagcggcttcaccttcgac

SEQ ID NO: 171 - DNA, 42 bp, synthetic construct
  source 1..42: mol_type = other DNA; note = FW2
tgggtgagacaggcccccggcaagggcctggagtgggtgagc

SEQ ID NO: 172 - DNA, 96 bp, synthetic construct
  source 1..96: mol_type = other DNA; note = FW3
agattcaccatcagcagagacaacagcaagaacagcctgtacctgcagatgaacagcctgagaaccgaggacaccgccctgtactactgcgccaag

SEQ ID NO: 173 - DNA, 33 bp, synthetic construct
  source 1..33: mol_type = other DNA; note = FW4
tggggcagaggcaccctggtcaccgtctcgagc

SEQ ID NO: 174 - DNA, 15 bp, synthetic construct
  source 1..15: mol_type = other DNA; note = CDR1
gactacaccatgcac

SEQ ID NO: 175 - DNA, 51 bp, synthetic construct
  source 1..51: mol_type = other DNA; note = CDR2
gacatcagctggagcggcggcagcatctactacgccgacagcgtgaagggc

SEQ ID NO: 176 - DNA, 45 bp, synthetic construct
  source 1..45: mol_type = other DNA; note = CDR3
gaccacagaggctacggcgactacgagggcggcggcttcgactac

SEQ ID NO: 177 - DNA, 372 bp, synthetic construct
  source 1..372: mol_type = other DNA; note = VH
gaggtgcagctggtggagagcggcggcgtggtggtgcagcccggcggcagcctgagactgagctgcgccgccagcggcttcaccttcgacgactacaccatgcactgggtgagacaggcccccggcaagggcctggagtgggtgagcgacatcagctggagcggcggcagcatctactacgccgacagcgtgaagggcagattcaccatcagcagagacaacagcaagaacagcctgtacctgcagatgaacagcctgagaaccgaggacaccgccctgtactactgcgccaaggaccacagaggctacggcgactacgagggcggcggcttcgactactggggcagaggcaccctggtcaccgtctcgagc

SEQ ID NO: 178 - protein, 30 aa, synthetic construct
  source 1..30: note = FW1
EVQLVESGGGLVQPGRSLRLSCVTSGFTFD

SEQ ID NO: 179 - protein, 14 aa, synthetic construct
  source 1..14: note = FW2
WVRQAPGKGLEWVS

SEQ ID NO: 180 - protein, 32 aa, synthetic construct
  source 1..32: note = FW3
RFTISRDNAKDSLYLQMNSLRTEDTALYYCAK

SEQ ID NO: 181 - protein, 11 aa, synthetic construct
  source 1..11: note = FW4
WGQGTLVTVSS

SEQ ID NO: 182 - protein, 5 aa, synthetic construct
  source 1..5: note = CDR1
DYTMH

SEQ ID NO: 183 - protein, 17 aa, synthetic construct
  source 1..17: note = CDR2
DISWSSGTTGYADSVKG

SEQ ID NO: 184 - protein, 15 aa, synthetic construct
  source 1..15: note = CDR3
DHMGYGDYEGGGFDY

SEQ ID NO: 185 - protein, 124 aa, synthetic construct
  source 1..124: note = VH
EVQLVESGGGLVQPGRSLRLSCVTSGFTFDDYTMHWVRQAPGKGLEWVSDISWSSGTTGYADSVKGRFTISRDNAKDSLYLQMNSLRTEDTALYYCAKDHMGYGDYEGGGFDYWGQGTLVTVSS

SEQ ID NO: 186 - DNA, 90 bp, synthetic construct
  source 1..90: mol_type = other DNA; note = FW1
gaagtgcagctggtggagtctgggggaggcttggtacagcctggcaggtccctgagactctcctgtgtaacctctggattcacctttgat

SEQ ID NO: 187 - DNA, 42 bp, synthetic construct
  source 1..42: mol_type = other DNA; note = FW2
tgggtccggcaagctccggggaagggcctggagtgggtctca

SEQ ID NO: 188 - DNA, 96 bp, synthetic construct
  source 1..96: mol_type = other DNA; note = FW3
cgattcaccatctccagagacaatgccaaggactccctgtatctgcaaatgaacagtctgagaactgaggacacggccttgtattactgtgcaaaa

SEQ ID NO: 189 - DNA, 33 bp, synthetic construct
  source 1..33: mol_type = other DNA; note = FW4
tggggccagggaaccctggtcaccgtctcgagc

SEQ ID NO: 190 - DNA, 15 bp, synthetic construct
  source 1..15: mol_type = other DNA; note = CDR1
gattataccatgcac

SEQ ID NO: 191 - DNA, 51 bp, synthetic construct
  source 1..51: mol_type = other DNA; note = CDR2
gatattagttggagtagtggcaccactggctatgcggactctgtgaagggc

SEQ ID NO: 192 - DNA, 45 bp, synthetic construct
  source 1..45: mol_type = other DNA; note = CDR3
gatcatatggggtacggtgactacgagggtggtggctttgactac

SEQ ID NO: 193 - DNA, 372 bp, synthetic construct
  source 1..372: mol_type = other DNA; note = VH
gaagtgcagctggtggagtctgggggaggcttggtacagcctggcaggtccctgagactctcctgtgtaacctctggattcacctttgatgattataccatgcactgggtccggcaagctccggggaagggcctggagtgggtctcagatattagttggagtagtggcaccactggctatgcggactctgtgaagggccgattcaccatctccagagacaatgccaaggactccctgtatctgcaaatgaacagtctgagaactgaggacacggccttgtattactgtgcaaaagatcatatggggtacggtgactacgagggtggtggctttgactactggggccagggaaccctggtcaccgtctcgagc

SEQ ID NO: 194 - DNA, 397 bp, synthetic construct
  source 1..397: mol_type = other DNA; note = MF9249
ggcccagccggccatggccgaggtgcagctggtgcagtctggagcagaagtgaaaaagcccggggagtctctgaagatctcctgtaagggttctggatacagttttactaggttttggatcggctgggtgcgccagatgcccgggaaaggcctggagtggatggggatcatctatcctggtgactctgataccagatacagcccgtccttccaaggccaggtcaccatctcagccgacaagtccaccagtaccgcctacctgcagtggagcagcctgaaggcctcggacaccggcatgtattattgtgtgagacatatacgatattttgactggtcggaagactaccactattacctggacgtctggggcaaagggaccacggtcaccgtctccagt

SEQ ID NO: 195 - protein, 30 aa, synthetic construct
  source 1..30: note = MF9249 FW1
EVQLVQSGAEVKKPGESLKISCKGSGYSFT

SEQ ID NO: 196 - protein, 14 aa, synthetic construct
  source 1..14: note = MF9249 FW2
WVRQMPGKGLEWMG

SEQ ID NO: 197 - protein, 32 aa, synthetic construct
  source 1..32: note = MF9249 FW3
QVTISADKSTSTAYLQWSSLKASDTGMYYCVR

SEQ ID NO: 198 - protein, 11 aa, synthetic construct
  source 1..11: note = MF9249 FW4
WGKGTTVTVSS

SEQ ID NO: 199 - protein, 5 aa, synthetic construct
  source 1..5: note = MF9249 CDR1
RFWIG

SEQ ID NO: 200 - protein, 17 aa, synthetic construct
  source 1..17: note = MF9249 CDR2
IIYPGDSDTRYSPSFQG

SEQ ID NO: 201 - protein, 17 aa, synthetic construct
  source 1..17: note = MF9249 CDR3
HIRYFDWSEDYHYYLDV

SEQ ID NO: 202 - protein, 126 aa, synthetic construct
  source 1..126: note = MF9249 VH
EVQLVQSGAEVKKPGESLKISCKGSGYSFTRFWIGWVRQMPGKGLEWMGIIYPGDSDTRYSPSFQGQVTISADKSTSTAYLQWSSLKASDTGMYYCVRHIRYFDWSEDYHYYLDVWGKGTTVTVSS

SEQ ID NO: 203 - DNA, 378 bp, synthetic construct
  source 1..378: mol_type = other DNA; note = MF9267
gaggtgcagctggtggagtctggggcagaggtgaaaaagcccggggagtctctgaagatctcctgtaagggttctggatacagttttactaggtattggatcggctgggtgcgccagatgcccgggaaaggcctggagtggatggggatcatctatcctggtgactctgataccagatacagcccgtccttccaaggccaggtcaccatctcagccgacaagtccatcagtactgcctacctgcagtggagcagcctgaaggcctcggacaccgccatgtattattgtgtgagaaatatacgatattttgtctggtcggaggactaccactactacatggacgtctggggcaaagggaccacggtcaccgtctccagt

SEQ ID NO: 204 - protein, 30 aa, synthetic construct
  source 1..30: note = MF9267 FW1
EVQLVESGAEVKKPGESLKISCKGSGYSFT

SEQ ID NO: 205 - protein, 14 aa, synthetic construct
  source 1..14: note = MF9267 FW2
WVRQMPGKGLEWMG

SEQ ID NO: 206 - protein, 32 aa, synthetic construct
  source 1..32: note = MF9267 FW3
QVTISADKSISTAYLQWSSLKASDTAMYYCVR

SEQ ID NO: 207 - protein, 11 aa, synthetic construct
  source 1..11: note = MF9267 FW4
WGKGTTVTVSS

SEQ ID NO: 208 - protein, 5 aa, synthetic construct
  source 1..5: note = MF9267 CDR1
RYWIG

SEQ ID NO: 209 - protein, 17 aa, synthetic construct
  source 1..17: note = MF9267 CDR2
IIYPGDSDTRYSPSFQG

SEQ ID NO: 210 - protein, 17 aa, synthetic construct
  source 1..17: note = MF9267 CDR3
NIRYFVWSEDYHYYMDV

SEQ ID NO: 211 - protein, 126 aa, synthetic construct
  source 1..126: note = MF9267 VH
EVQLVESGAEVKKPGESLKISCKGSGYSFTRYWIGWVRQMPGKGLEWMGIIYPGDSDTRYSPSFQGQVTISADKSISTAYLQWSSLKASDTAMYYCVRNIRYFVWSEDYHYYMDVWGKGTTVTVSS

SEQ ID NO: 212 - DNA, 363 bp, synthetic construct
  source 1..363: mol_type = other DNA; note = MF5377
gaggtgcagctggtgcagtctggagctgaggtgaagaagcctgggtcctcggtgaaggtctcctgcaaggcttctggaggcatcttcagcacctatgctatcagctgggtgcgacaggcccctggacaagggcttgagtggatgggagggatcatccctatctttgatacaccaaactacgcacagaagttccagggcagagtcacgattaccgcggacaaatccacgagcacagcctacatggacctgagcagcctgagatctgaggacacggccgtatattactgtgcgaaaaacgtgcgtggatatagtgcctacgaccttgactactggggccagggcaccctggtcaccgtctccagt

SEQ ID NO: 213 - protein, 30 aa, synthetic construct
  source 1..30: note = MF5377 FW1
EVQLVQSGAEVKKPGSSVKVSCKASGGIFS

SEQ ID NO: 214 - protein, 14 aa, synthetic construct
  source 1..14: note = MF5377 FW2
WVRQAPGQGLEWMG

SEQ ID NO: 215 - protein, 32 aa, synthetic construct
  source 1..32: note = MF5377 FW3
RVTITADKSTSTAYMDLSSLRSEDTAVYYCAK

SEQ ID NO: 216 - protein, 11 aa, synthetic construct
  source 1..11: note = MF5377 FW4
WGQGTLVTVSS

SEQ ID NO: 217 - protein, 5 aa, synthetic construct
  source 1..5: note = MF5377 CDR1
TYAIS

SEQ ID NO: 218 - protein, 17 aa, synthetic construct
  source 1..17: note = MF5377 CDR2
GIIPIFDTPNYAQKFQG

SEQ ID NO: 219 - protein, 12 aa, synthetic construct
  source 1..12: note = MF5377 CDR3
NVRGYSAYDLDY

SEQ ID NO: 220 - protein, 121 aa, synthetic construct
  source 1..121: note = MF5377 VH
EVQLVQSGAEVKKPGSSVKVSCKASGGIFSTYAISWVRQAPGQGLEWMGGIIPIFDTPNYAQKFQGRVTITADKSTSTAYMDLSSLRSEDTAVYYCAKNVRGYSAYDLDYWGQGTLVTVSS

SEQ ID NO: 221 - DNA, 363 bp, synthetic construct
  source 1..363: mol_type = other DNA; note = MF5444
caggtgcagctggtgcaatctgggtctgagttgaagaagcctggggcctcagtgaaggtttcctgcaaggcttctggatacaccttcactagctattctatgaattgggtgcgacaggcccctggacaagggcttgagtggatgggatggatcaacaccaacactgggaacccaacgtatgcccagggcttcacaggacggtttgtcttctccttggacacctctgtcagcacggcatatctgcagatcagcagcctaaaggctgaggacactgccgtgtattactgtgcgagagatcacgattttcggactgggagggcttttgatatctggggccaagggaccacggtcaccgtctccagt

SEQ ID NO: 222 - protein, 30 aa, synthetic construct
  source 1..30: note = MF5444 FW1
QVQLVQSGSELKKPGASVKVSCKASGYTFT

SEQ ID NO: 223 - protein, 14 aa, synthetic construct
  source 1..14: note = MF5444 FW2
WVRQAPGQGLEWMG

SEQ ID NO: 224 - protein, 32 aa, synthetic construct
  source 1..32: note = MF5444 FW3
RFVFSLDTSVSTAYLQISSLKAEDTAVYYCAR

SEQ ID NO: 225 - protein, 11 aa, synthetic construct
  source 1..11: note = MF5444 FW4
WGQGTTVTVSS

SEQ ID NO: 226 - protein, 5 aa, synthetic construct
  source 1..5: note = MF5444 CDR1
SYSMN

SEQ ID NO: 227 - protein, 17 aa, synthetic construct
  source 1..17: note = MF5444 CDR2
WINTNTGNPTYAQGFTG

SEQ ID NO: 228 - protein, 12 aa, synthetic construct
  source 1..12: note = MF5444 CDR3
DHDFRTGRAFDI

SEQ ID NO: 229 - protein, 121 aa, synthetic construct
  source 1..121: note = MF5444 VH
QVQLVQSGSELKKPGASVKVSCKASGYTFTSYSMNWVRQAPGQGLEWMGWINTNTGNPTYAQGFTGRFVFSLDTSVSTAYLQISSLKAEDTAVYYCARDHDFRTGRAFDIWGQGTTVTVSS

SEQ ID NO: 230 - DNA, 372 bp, synthetic construct
  source 1..372: mol_type = other DNA; note = MF5380
gaggtgcagctggtggagtctgggggagacgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaactccaagagcacgctgtttctgcaaatgaacagcctgagagctgaggacacggctgtgtatttctgtgtgagaggcctccccataactatggttcggggagcgtactcctttgactactggggccagggcaccctggtcaccgtctccagt

SEQ ID NO: 231 - protein, 30 aa, synthetic construct
  source 1..30: note = MF5380 FW1
EVQLVESGGDVVQPGRSLRLSCAASGFTFS

SEQ ID NO: 232 - protein, 14 aa, synthetic construct
  source 1..14: note = MF5380 FW2
WVRQAPGKGLEWVA

SEQ ID NO: 233 - protein, 32 aa, synthetic construct
  source 1..32: note = MF5380 FW3
RFTISRDNSKSTLFLQMNSLRAEDTAVYFCVR

SEQ ID NO: 234 - protein, 11 aa, synthetic construct
  source 1..11: note = MF5380 FW4
WGQGTLVTVSS

SEQ ID NO: 235 - protein, 5 aa, synthetic construct
  source 1..5: note = MF5380 CDR1
SYGMH

SEQ ID NO: 236 - protein, 17 aa, synthetic construct
  source 1..17: note = MF5380 CDR2
VISYDGSNKYYADSVKG

SEQ ID NO: 237 - protein, 15 aa, synthetic construct
  source 1..15: note = MF5380 CDR3
GLPITMVRGAYSFDY

SEQ ID NO: 238 - protein, 124 aa, synthetic construct
  source 1..124: note = MF5380 VH
EVQLVESGGDVVQPGRSLRLSCAASGFTFSSYGMHWVRQAPGKGLEWVAVISYDGSNKYYADSVKGRFTISRDNSKSTLFLQMNSLRAEDTAVYFCVRGLPITMVRGAYSFDYWGQGTLVTVSS

SEQ ID NO: 239 - DNA, 366 bp, synthetic construct
  source 1..366: mol_type = other DNA; note = MF8233
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatgccaacacaaactatgcacagaagctccagggcagagtcaccatgaccacagacacatccacgagcacagcctacatggagctgaggagcctgagatctgacgacacggctgtgtattactgtgcaaaagatcgtcattggcattggtggctggacgcctttgattattggggccaaggtaccctggtcaccgtctccagt

SEQ ID NO: 240 - protein, 30 aa, synthetic construct
  source 1..30: note = MF8233 FW1
QVQLVQSGAEVKKPGASVKVSCKASGYTFT

SEQ ID NO: 241 - protein, 14 aa, synthetic construct
  source 1..14: note = MF8233 FW2
WVRQAPGQGLEWMG

SEQ ID NO: 242 - protein, 32 aa, synthetic construct
  source 1..32: note = MF8233 FW3
RVTMTTDTSTSTAYMELRSLRSDDTAVYYCAK

SEQ ID NO: 243 - protein, 11 aa, synthetic construct
  source 1..11: note = MF8233 FW4
WGQGTLVTVSS

SEQ ID NO: 244 - protein, 5 aa, synthetic construct
  source 1..5: note = MF8233 CDR1
SYGIS

SEQ ID NO: 245 - protein, 17 aa, synthetic construct
  source 1..17: note = MF8233 CDR2
WISAYNANTNYAQKLQG

SEQ ID NO: 246 - protein, 13 aa, synthetic construct
  source 1..13: note = MF8233 CDR3
DRHWHWWLDAFDY

SEQ ID NO: 247 - protein, 122 aa, synthetic construct
  source 1..122: note = MF8233 VH
QVQLVQSGAEVKKPGASVKVSCKASGYTFTSYGISWVRQAPGQGLEWMGWISAYNANTNYAQKLQGRVTMTTDTSTSTAYMELRSLRSDDTAVYYCAKDRHWHWWLDAFDYWGQGTLVTVSS

SEQ ID NO: 248 - DNA, 366 bp, synthetic construct
  source 1..366: mol_type = other DNA; note = MF9891
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatgccaacacaaactatgcacagaagctccagggcagagtcaccatgaccacagacacatccacgagcacagcctacatggagctgaggagcctgagatctgacgacacggctgtgtattactgtgcaaaagatctgtacggtcattggtggctggatgcctttgattattggggccaaggtaccctggtcaccgtctccagt

SEQ ID NO: 249 - protein, 30 aa, synthetic construct; identical to SEQ ID NO: 240
  source 1..30: note = MF9891 FW1

SEQ ID NO: 250 - protein, 14 aa, synthetic construct
  source 1..14: note = MF9891 FW2
WVRQAPGQGLEWMG

SEQ ID NO: 251 - protein, 32 aa, synthetic construct; identical to SEQ ID NO: 242
  source 1..32: note = MF9891 FW3

SEQ ID NO: 252 - protein, 11 aa, synthetic construct
  source 1..11: note = MF9891 FW4
WGQGTLVTVSS

SEQ ID NO: 253 - protein, 5 aa, synthetic construct
  source 1..5: note = MF9891 CDR1
SYGIS

SEQ ID NO: 254 - protein, 17 aa, synthetic construct
  source 1..17: note = MF9891 CDR2
WISAYNANTNYAQKLQG

SEQ ID NO: 255 - protein, 13 aa, synthetic construct
  source 1..13: note = MF9891 CDR3
DLYGHWWLDAFDY

SEQ ID NO: 256 - protein, 122 aa, synthetic construct
  source 1..122: note = MF9891 VH
QVQLVQSGAEVKKPGASVKVSCKASGYTFTSYGISWVRQAPGQGLEWMGWISAYNANTNYAQKLQGRVTMTTDTSTSTAYMELRSLRSDDTAVYYCAKDLYGHWWLDAFDYWGQGTLVTVSS

SEQ ID NO: 257 - DNA, 366 bp, synthetic construct
  source 1..366: mol_type = other DNA; note = MF9886
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatgccaacacaaactatgcacagaagctccagggcagagtcaccatgaccacagacacatccacgagcacagcctacatggagctgaggagcctgagatctgacgacacggctgtgtattactgtgcaaaaggtccaggttctcattggtggctggatgcctttgattattggggccaaggtaccctggtcaccgtctccagt

SEQ ID NO: 258 - protein, 30 aa, synthetic construct; identical to SEQ ID NO: 240
  source 1..30: note = MF9886 FW1

SEQ ID NO: 259 - protein, 14 aa, synthetic construct
  source 1..14: note = MF9886 FW2
WVRQAPGQGLEWMG

SEQ ID NO: 260 - protein, 32 aa, synthetic construct; identical to SEQ ID NO: 242
  source 1..32: note = MF9886 FW3

SEQ ID NO: 261 - protein, 11 aa, synthetic construct
  source 1..11: note = MF9886 FW4
WGQGTLVTVSS

SEQ ID NO: 262 - protein, 5 aa, synthetic construct
  source 1..5: note = MF9886 CDR1
SYGIS

SEQ ID NO: 263 - protein, 17 aa, synthetic construct
  source 1..17: note = MF9886 CDR2
WISAYNANTNYAQKLQG

SEQ ID NO: 264 - protein, 13 aa, synthetic construct
  source 1..13: note = MF9886 CDR3
GPGSHWWLDAFDY

SEQ ID NO: 265 - protein, 122 aa, synthetic construct
  source 1..122: note = MF9886 VH
QVQLVQSGAEVKKPGASVKVSCKASGYTFTSYGISWVRQAPGQGLEWMGWISAYNANTNYAQKLQGRVTMTTDTSTSTAYMELRSLRSDDTAVYYCAKGPGSHWWLDAFDYWGQGTLVTVSS

SEQ ID NO: 266 - DNA, 366 bp, synthetic construct
  source 1..366: mol_type = other DNA; note = MF9873
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatgccaacacaaactatgcacagaagctccagggcagagtcaccatgaccacagacacatccacgagcacagcctacatggagctgaggagcctgagatctgacgacacggctgtgtattactgtgcaaaagatagaggttggcattggtggctggatgcctttgattattggggccaaggtaccctggtcaccgtctccagt

SEQ ID NO: 267 - protein, 30 aa, synthetic construct; identical to SEQ ID NO: 240
  source 1..30: note = MF9873 FW1

SEQ ID NO: 268 - protein, 14 aa, synthetic construct
  source 1..14: note = MF9873 FW2
WVRQAPGQGLEWMG

SEQ ID NO: 269 - protein, 32 aa, synthetic construct; identical to SEQ ID NO: 242
  source 1..32: note = MF9873 FW3

SEQ ID NO: 270 - protein, 11 aa, synthetic construct
  source 1..11: note = MF9873 FW4
WGQGTLVTVSS

SEQ ID NO: 271 - protein, 5 aa, synthetic construct
  source 1..5: note = MF9873 CDR1
SYGIS

SEQ ID NO: 272 - protein, 17 aa, synthetic construct
  source 1..17: note = MF9873 CDR2
WISAYNANTNYAQKLQG

SEQ ID NO: 273 - protein, 13 aa, synthetic construct
  source 1..13: note = MF9873 CDR3
DRGWHWWLDAFDY

SEQ ID NO: 274 - protein, 122 aa, synthetic construct
  source 1..122: note = MF9873 VH
QVQLVQSGAEVKKPGASVKVSCKASGYTFTSYGISWVRQAPGQGLEWMGWISAYNANTNYAQKLQGRVTMTTDTSTSTAYMELRSLRSDDTAVYYCAKDRGWHWWLDAFDYWGQGTLVTVSS

SEQ ID NO: 275 - DNA, 375 bp, synthetic construct
  source 1..375: mol_type = other DNA; note = MF9988
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatgccaacacaaactatgcacagaagctccagggcagagtcaccatgaccacagacacatccacgagcacagcctacatggagctgaggagcctgagatctgacgacacggctgtgtattactgtgcaaaagatcgtcattggcattggtggctggacggctttgattattggggccaaggtaccctggtcaccgtctcgagtgtctccagt

SEQ ID NO: 276 - protein, 30 aa, synthetic construct; identical to SEQ ID NO: 240
  source 1..30: note = MF9988 FW1

SEQ ID NO: 277 - protein, 14 aa, synthetic construct
  source 1..14: note = MF9988 FW2
WVRQAPGQGLEWMG

SEQ ID NO: 278 - protein, 32 aa, synthetic construct; identical to SEQ ID NO: 242
  source 1..32: note = MF9988 FW3

SEQ ID NO: 279 - protein, 11 aa, synthetic construct
  source 1..11: note = MF9988 FW4
WGQGTLVTVSS

SEQ ID NO: 280 - protein, 5 aa, synthetic construct
  source 1..5: note = MF9988 CDR1
SYGIS

SEQ ID NO: 281 - protein, 17 aa, synthetic construct
  source 1..17: note = MF9988 CDR2
WISAYNANTNYAQKLQG

SEQ ID NO: 282 - protein, 13 aa, synthetic construct
  source 1..13: note = MF9988 CDR3
DRHWHWWLDGFDY

SEQ ID NO: 283 - protein, 122 aa, synthetic construct
  source 1..122: note = MF9988 VH
QVQLVQSGAEVKKPGASVKVSCKASGYTFTSYGISWVRQAPGQGLEWMGWISAYNANTNYAQKLQGRVTMTTDTSTSTAYMELRSLRSDDTAVYYCAKDRHWHWWLDGFDYWGQGTLVTVSS

SEQ ID NO: 284 - DNA, 369 bp, synthetic construct
  source 1..369: mol_type = other DNA; note = MF6744 anti-CD127 antibody sequence
gaggtgcagctggtgcagtctggagcagaggtgaaaaagcccggggagtcactgaaaatctcctgtaagggttctggatacagctttaccagctactggatcggctgggtgcgccagatgcccgggaaaggcctggagtggatggggatcatctttcctgatgactctgataccagatacagtccgtccttccaaggccaggtcaccatctcagccgacaagtccatcagtaccgcctacctgcagtggagcagcctgaaaccctcggacaccgccatgtattactgtgtgagacttggtggatatagtggctacgctgaggattttgttgacttctggggccagggaaccctggtcaccgtctccagt

SEQ ID NO: 285 - protein, 30 aa, synthetic construct; identical to SEQ ID NO: 195
  source 1..30: note = MF6744 FW1

SEQ ID NO: 286 - protein, 14 aa, synthetic construct
  source 1..14: note = MF6744 FW2
WVRQMPGKGLEWMG

SEQ ID NO: 287 - protein, 32 aa, synthetic construct
  source 1..32: note = MF6744 FW3
QVTISADKSISTAYLQWSSLKPSDTAMYYCVR

SEQ ID NO: 288 - protein, 11 aa, synthetic construct
  source 1..11: note = MF6744 FW4
WGQGTLVTVSS

SEQ ID NO: 289 - protein, 5 aa, synthetic construct
  source 1..5: note = MF6744 CDR1
SYWIG

SEQ ID NO: 290 - protein, 17 aa, synthetic construct
  source 1..17: note = MF6744 CDR2
IIFPDDSDTRYSPSFQG

SEQ ID NO: 291 - protein, 14 aa, synthetic construct
  source 1..14: note = MF6744 CDR3
LGGYSGYAEDFVDF

SEQ ID NO: 292 - protein, 123 aa, synthetic construct
  source 1..123: note = MF6744 VH
EVQLVQSGAEVKKPGESLKISCKGSGYSFTSYWIGWVRQMPGKGLEWMGIIFPDDSDTRYSPSFQGQVTISADKSISTAYLQWSSLKPSDTAMYYCVRLGGYSGYAEDFVDFWGQGTLVTVSS

SEQ ID NO: 293 - protein, 118 aa, synthetic construct
  source 1..118: note = MF4327 CLEC12A
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAKGTTGDWFDYWGQGTLVTVSS

SEQ ID NO: 294 - DNA, 393 bp, synthetic construct
  source 1..393: mol_type = other DNA; note = H1H7232B (1129) VH-MF6955 in combination with common light chain, VH gene: IGHV3-9
gcggcccagccggccatggccgaggtgcagctggtggagtcggggggaggcttggttcaccctggcaggtccctgagactctcctgtgcagcctctggattcacctttgatgattataccatgcactgggtccggcaagctccagggaagggcctggaatgggtctcagatattagttggaatagtggtagcagaggctatgcggactctgtgaagggccgattcaccatctccagagataatgccgagaactccctgtacctgcaaatgaacagtctgagagctgaggacacggccttgtattactgtgcaaaagataaaagtggctacggccactactactactacgctatggacgtctggggccaagggaccacggtcaccgtctcctca

SEQ ID NO: 295 - protein, 131 aa, synthetic construct
  source 1..131: note = H1H7232B (1129) VH - MF6955 in combination with common light chain VH gene: IGHV3-9
AAQPAMAEVQLVESGGGLVHPGRSLRLSCAASGFTFDDYTMHWVRQAPGKGLEWVSDISWNSGSRGYADSVKGRFTISRDNAENSLYLQMNSLRAEDTALYYCAKDKSGYGHYYYYAMDVWGQGTTVTVSS

SEQ ID NO: 296 - DNA, 390 bp, synthetic construct
  source 1..390: mol_type = other DNA; note = H1H7241B (1145) VH - MF6964 in combination with common light chain VH gene: IGHV3-9
gcggcccagccggccatggccgaggtgcagctggtggagtctgggggaggcttggttcagcctggcaggtccctgagactctcctgtgcagcctctggatttacctttgctgactataccatgcactgggtccggcaaggtccagggaagggcctggagtgggtctcagatattggttggaatagtaatactataggctatgcggactctgtgaagggccgattcgccatctccagagacaacgccaagaactccctgtatcttcaaatgaacagtctgcgacctgaggacacggccttatattactgtgtaaaggataaaagtggctacgggaaattccaatacggtttggacgtctggggccaagggaccacggtcaccgtctcctca

SEQ ID NO: 297 - protein, 130 aa, synthetic construct
  source 1..130: note = H1H7241B (1145) VH - MF6964 in combination with common light chain VH gene: IGHV3-9
AAQPAMAEVQLVESGGGLVQPGRSLRLSCAASGFTFADYTMHWVRQGPGKGLEWVSDIGWNSNTIGYADSVKGRFAISRDNAKNSLYLQMNSLRPEDTALYYCVKDKSGYGKFQYGLDVWGQGTTVTVSS

SEQ ID NO: 298 - protein, 126 aa, synthetic construct; identical to SEQ ID NO: 202
  source 1..126: note = VH

SEQ ID NO: 299 - protein, 9 aa, synthetic construct
  source 1..9: note = CDR3 of the common light chain
QQSYSTPPT

(19) United States
(12) Patent Application Publication
(10) Pub. No.: US 2026/0308254 A1
(43) Pub. Date: Oct. 8, 2026

(54) CD3 BINDING MOLECULES

(71) Applicant: Merus N.V., Utrecht (NL)

(72) Inventor: Pieter Fokko VAN LOO, Utrecht (NL)

(73) Assignee: Merus N.V., Utrecht (NL)

(21) Appl. No.: 19/344,218

(22) Filed: Sep. 29, 2025

Related U.S. Application Data
(63) Continuation of application No. 17/438,768, filed on Mar. 27, 2020, now Pat. No. 12,460,002.

(30) Foreign Application Priority Data
Mar. 29, 2019 (EP) 19166345.9
Oct. 1, 2019 (EP) 19200931.4

(51) Int. Cl. C07K 16/28; A61P 35/00

(52) U.S. Cl. CPC C07K 16/2809; A61P 35/00; C07K 16/283; C07K 16/2863; C07K 2317/31; C07K 2317/524; C07K 2317/526; C07K 2317/53; C07K 2317/55; C07K 2317/565; C07K 2317/92

(57) ABSTRACT

The invention relates to heavy chain variable regions, binding domains and antibodies specific for human CD3, and CD3 binding proteins. The invention further relates to the use of a CD3 binding protein, preferably an antibody, of the invention in the treatment of cancer or autoimmune disease.

### CROSS-REFERENCE TO RELATED APPLICATIONS

This application is a continuation of U.S. application Ser. No. 17/438,768 filed Sep. 13, 2021, which is the 35 U.S.C. § 371 National Phase of International Application No. PCT/NL2020/050214, filed Mar. 27, 2020, which claims priority to EP 19200931.4, filed Oct. 1, 2019 and EP 19166345.9, filed Mar. 29, 2019. The entire contents of International Application No. PCT/NL2020/050214 are hereby incorporated by reference.

### REFERENCE TO A SEQUENCE LISTING SUBMITTED ELECTRONICALL VIA EFS WEB

This application includes a Sequence Listing submitted electronically via EFS-web (Name: 4096_0390003_SequenceListing_ST26.xml, File Size: 383,696 bytes, and Created On: Jun. 17, 2026), which is hereby incorporated by reference in its entirety.

### FIELD OF THE INVENTION

The invention relates to the field of antibodies, in particular to the field of therapeutic antibodies. The antibodies can be used in the treatment of humans. More in particular the invention relates to antibodies and preferably bispecific or multispecific antibodies for the treatment of a tumor.

### BACKGROUND TO THE INVENTION

Monoclonal antibodies that bind to human CD3 were among the first antibodies developed for therapeutic use in humans. Monoclonal CD3 binding antibodies are typically used for their immune suppressive qualities, for instance in transplant rejection. Antibodies which are bispecific for CD3 on T cells and for a surface target antigen on cancer cells, are capable of connecting any kind of T cell to a cancer cell, independently of T-cell receptor specificity, costimulation, or peptide antigen presentation. Such bispecific T-cell engaging antibodies show great promise in the treatment of various cancers and neoplastic growths.

It is an object of the invention to provide new antibodies with CD3 binding properties in kind, not necessarily in amount, with improved characteristics, having relatively low affinity, with higher cytotoxicity, which are amenable for immuno-oncology applications for T-cell and effector cell engagement, and conversely, new antibodies with CD3 binding that have relatively high affinity, with lower cytotoxicity, which are amenable for auto-immune applications for T-cell and effector cell down-regulation. It is a further object of the invention to provide T-cell engaging CD3 binding proteins and antibodies with the above properties that bind at least one further membrane associated molecule.

### SUMMARY OF THE INVENTION

The invention provides an antigen-binding protein, preferably an antibody, that binds human CD3 comprising an antibody variable domain comprising a heavy chain variable region and a light chain variable region wherein the heavy chain variable region comprises a CDR1, CDR2 and CDR3 comprising the amino acid sequence:

CDR1:
SEQ ID NO 42
SFGIS;
CDR2:
SEQ ID NO 43
GFIPVLGTANYAQKFQG;
CDR3:
SEQ ID NO 44
RGNWNPFDP;
or

comprising the amino acid sequence:

CDR1:
SEQ ID NO 6
SX₁FTIS;
CDR2:
SEQ ID NO 7
GIIPX₂FGTITYAQKFQG;
CDR3:
SEQ ID NO 44
RGNWNPFDP;

wherein
X₁=Kor R;
X₂=L or I.

In a preferred embodiment X₁=K; and X₂=L;

In another preferred embodiment X₁=R; and X₂=I.

In a preferred embodiment, the invention provides an antigen-binding protein, preferably an antibody, that binds human CD3 comprising an antibody variable domain comprising a heavy chain variable region and a light chain variable region wherein the heavy chain variable region comprises a CDR1, CDR2 and CDR3 comprising the amino acid sequence:

CDR1:
SEQ ID NO 84
SKTLTIS;
CDR2:
SEQ ID NO 85
GIIPIFGSITYAQKFQD;
CDR3:
SEQ ID NO 44
RGNWNPFDP;
or

comprising the amino acid sequence:

CDR1:
SEQ ID NO 100
GSGIS;
CDR2:
SEQ ID NO 101
GFIPFFGSANYAQKFRD;
CDR3:
SEQ ID NO 8
RGNWNPX₁₃DP;

wherein
X₁₃=L or F.

The invention further provides an antigen-binding protein, preferably an antibody, that binds human CD3 comprising an antibody variable domain comprising a heavy chain variable region and a light chain variable region wherein the heavy chain variable region comprises the amino acid sequence

SEQ ID NO 45
EVQLVQSGAEVKKPGSSVKVSCKASGGTFRSFGISWVRQAPGQGLEWMG
GFIPVLGTANYAQKFQGRVTIIADKSTNTAYMELSSLRSEDTAVYYCAR
RGNWNPFDPWGQGTLVTVSS;
SEQ ID NO 54
QVQLVQSGAEVKKPGSSVKVSCKASGDAFKSKTFTISWVRQAPGQGLEW
LGGIIPLFGTITYAQKFQGRVTITADKSTNTAFMELSSLRSEDTAMYYC
TRRGNWNPFDPWGQGTLVTVSS;
SEQ ID NO 63
EVQLVQSGSELKKPGSSVKVSCKASGVTFNSRTFTISWVRQAPGQGLEW
LGSIIPIFGTITYAQKFQGRVTITADKSTSTAFMELTSLRSEDTAIYYC
TRRGNWNPFDPWGQGTLVTVSS;
SEQ ID NO 71
QVQLVQSGAEVKKPGSSVKVSCKASGGTFRGSGISWVRQAPGQGLEWVG
GFIPFFGSANYAQKFRDRVTITADKSATTAYMELSSLRSEDTAIYYCAK
RGNWNPLDPWGQGTLVTVSS;
SEQ ID NO 87
QVQLVQSGAEVKKPGSSVKVSCKASGVTFKSKTLTISWVRQAPGQGLEW
LGGIIPIFGSITYAQKFQDRVSITADKSTNTAYLELNSLRSEDTAIYYC
ARRGNWNPFDPWGQGTLVTVSS;
or
SEQ ID NO 103
EVQLVQSGAEVKKPGSSVKVSCKASGGTFRGSGISWVRQAPGQGLEWVG
GFIPFFGSANYAQKFRDRVTITADKSATTAYMELSSLRSEDTAIYYCAK
RGNWNPFDPWGQGTLVTVSS;

with 0-10, preferably 0-5 amino acid variations, insertions, deletions, substitutions, additions or a combination thereof at one or more positions other than the CDRs.

Further provided is an antigen-binding protein, preferably an antibody, that binds human CD3 comprising an antibody variable domain comprising a heavy chain variable region and a light chain variable region wherein the heavy chain variable region comprises a CDR1, CDR2 and CDR3 comprising the amino acid sequence:

CDR1:
SEQ ID NO 9
RX₃WIG;
CDR2:
SEQ ID NO 200
IIYPGDSDTRYSPSFQG;
CDR3:
SEQ ID NO 10
X₄IRYFX₅WSEDYHYYX₆DV;

wherein
X₃=F or Y;
X₄=H or N;
X₅=D or V;
X₆=L or M.

In one embodiment X₃=F; X₄=H; X₅=D; and X₆=L. In another embodiment X₃=Y; X₄=N; X₅=V; and X₆=M.

The invention further provides an antigen-binding protein, preferably an antibody, that binds human CD3 comprising an antibody variable domain comprising a heavy chain variable region and a light chain variable region wherein the heavy chain variable region comprises the amino acid sequence

SEQ ID NO 298
EVQLVQSGAEVKKPGESLKISCKGSGYSFTRFWIGWVRQMPGKGLEWMG
IIYPGDSDTRYSPSFQGQVTISADKSTSTAYLQWSSLKASDTGMYYCVR
HIRYFDWSEDYHYYLDVWGKGTTVTVSS;
or
SEQ ID NO 211
EVQLVESGAEVKKPGESLKISCKGSGYSFTRYWIGWVRQMPGKGLEWMG
IIYPGDSDTRYSPSFQGQVTISADKSISTAYLQWSSLKASDTAMYYCVR
NIRYFVWSEDYHYYMDVWGKGTTVTVSS;

with 0-10, preferably 0-5 amino acid variations, insertions, deletions, substitutions, additions or a combination thereof at one or more positions other than the CDRs.

Further provided is an antigen-binding protein, preferably an antibody, that binds human CD3 comprising an antibody variable domain comprising a heavy chain variable region and a light chain variable region wherein the heavy chain variable region comprises a CDR1, CDR2 and CDR3 comprising the amino acid sequence:

CDR1:
SEQ ID NO 117
SYALS;
CDR2:
SEQ ID NO 118
GISGSGRTTWYADSVKG;
CDR3:
SEQ ID NO 119
DGGYSYGPYWYFDL.

Further provided is an antigen-binding protein, preferably an antibody, that binds human CD3 comprising an antibody variable domain comprising a heavy chain variable region and a light chain variable region wherein the heavy chain variable region comprises a CDR1, CDR2 and CDR3 comprising the amino acid sequence:

CDR1:
SEQ ID NO 117
SYALS;
CDR2:
SEQ ID NO 126
AISGSGRTTWYADSVKG;
CDR3:
SEQ ID NO 127
DGGYTYGPYWYFDL.

Further provided is an antigen-binding protein, preferably an antibody, that binds human CD3 comprising an antibody variable domain comprising a heavy chain variable region and a light chain variable region wherein the heavy chain variable region comprises the amino acid sequence

SEQ ID NO 120
QVQLVQSGGGLVQPGGSLRLSCATSGFKFSSYALSWVRQAPGKGLEWVS
GISGSGRTTWYADSVKGRFTISRDNSKNTLYLQMNSLRAEDTAVYYCAR
DGGYSYGPYWYFDLWGRGTLVTVSS;
or
SEQ ID NO 128
QVQLVESGGGLVQPGGSLRLSCATSGFTFISYALSWVRQAPGKGLEWVS
AISGSGRTTWYADSVKGRFTISRDNSKNTLFLQMNSLRAEDTAVYYCAR
DGGYTYGPYWYFDLWGRGTLVTVSS;

with 0-10, preferably 0-5 amino acid variations, insertions, deletions, substitutions, additions or a combination thereof at one or more positions other than the CDRs.

Also provided is an antigen-binding protein, preferably an antibody, that binds human CD3 comprising an antibody variable domain comprising a heavy chain variable region and a light chain variable region wherein the heavy chain variable region comprises a CDR1, CDR2 and CDR3 comprising the amino acid sequence:

CDR1:
SEQ ID NO 142
DYTMH;
CDR2:
SEQ ID NO 143
DISWSSGSIGYADSVKG;
CDR3:
SEQ ID NO 144
DHRGYGDYEGGGFDY.

Also provided is an antigen-binding protein, preferably an antibody, that binds human CD3 comprising an antibody variable domain comprising a heavy chain variable region and a light chain variable region wherein the heavy chain variable region comprises a CDR1, CDR2 and CDR3 comprising the amino acid sequence:

CDR1:
SEQ ID NO 142
DYTMH;
CDR2:
SEQ ID NO 11
DISWSX₇GX₈X₉X₁₀YADSVKG;
CDR3:
SEQ ID NO 12
DHX₁₁GYGDYEGGGFDX₁₂;

wherein
X₇=S or G;
X₈=S or T;
X₉=I or T;
X₁₀=G or Y;
X₁₁=R or M;
X₁₂=H or Y.

In one embodiment. X₇, X₈, X₉ and X₁₀ are S, S, I and G, and X₁₁ and X₁₂ are R and H. In another embodiment, X₇, X₈, X₉ and X₁₀ are G, S, I and Y, and X₁₁ and X₁₂ are R and Y. In another embodiment, X₇, X₈, X₉ and X₁₀ are S, T, T and G, and X₁₁ and X₁₂ are M and Y.

Further provided is an antigen-binding protein, preferably an antibody, that binds human CD3 comprising an antibody variable domain comprising a heavy chain variable region and a light chain variable region wherein the heavy chain variable region comprises the amino acid sequence

(SEQ ID NO 145)
EVQLVESGGGLVQPGRSLRLSCATSGFNFDDYTMHWVRQAPGKGLEWVS
DISWSSGSIGYADSVKGRFTISRDNAKNSLWLQMNSLRTEDTALYFCAK
DHRGYGDYEGGGFDYWGQGTLVTVSS;
or
(SEQ ID NO 153)
EVQLVESGGGLVQPGRSLRLSCATSGFTFDDYTMHWVRQAPGKGLEWVS
DISWSSGSIGYADSVKGRFTISRDNAKNSLYLQMNSLRAEDTALYFCAK
DHRGYGDYEGGGFDHWGQGTLVTVSS;
or
(SEQ ID NO 169)
EVQLVESGGVVVQPGGSLRLSCAASGFTFDDYTMHWVRQAPGKGLEWVS
DISWSGGSIYYADSVKGRFTISRDNSKNSLYLQMNSLRTEDTALYYCAK
DHRGYGDYEGGGFDYWGRGTLVTVSS;
or
(SEQ ID NO 185)
EVQLVESGGGLVQPGRSLRLSCVTSGFTFDDYTMHWVRQAPGKGLEWVS
DISWSSGTTGYADSVKGRFTISRDNAKDSLYLQMNSLRTEDTALYYCAK
DHMGYGDYEGGGFDYWGQGTLVTVSS;

with 0-10, preferably 0-5 amino acid variations, insertions, deletions, substitutions, additions or a combination thereof at one or more positions other than the CDRs.

The light chain variable region in an antigen-binding protein, preferably an antibody, of the invention preferably comprises a common light chain variable region. The common light chain variable region preferably comprises an IgVκ1-39 light chain variable region. Said light chain variable region is preferably a germline IgVκ1-39*01 variable region. The light chain variable region preferably comprises the kappa light chain IgVκ1-39*01/IGJκ1*01 or IgVκ1-39*01/IGJκ5*01. In one embodiment the light chain variable region comprises the human germline kappa light chain IgVκ1-39*01/IGJκ1*01 or IgVκ1-39*01/IGJκ5*01. Said light chain variable region preferably comprises the amino acid sequence

(SEQ ID NO 16)
DIQMT QSPSS LSASV GDRVT ITCRA SQSIS SYLNW YQQKP
GKAPK LLIYA ASSLQ SGVPS RFSGS GSGTD FTLTI SSLQP
EDFAT YYCQQ SYSTP PTFGQ GTKVE IK
or
(SEQ ID NO 19)
DIQMT QSPSS LSASV GDRVT ITCRA SQSIS SYLNW YQQKP
GKAPK LLIYA ASSLQ SGVPS RFSGS GSGTD FTLTI SSLQP
EDFAT YYCQQ SYSTP PITFG QGTRL EIK

with 0-5 amino acid variations, insertions, deletions, substitutions, additions or a combination thereof.

The antigen-binding protein, is preferably an antibody, preferably a bispecific or multispecific antibody.

Said antibody preferably comprises a H/L chain combination that binds human CD3 as indicated herein and a H/L chain combination that binds a tumor-antigen. Said H/L chain combination that binds a tumor-antigen preferably binds human BCMA, CD19, CD20, CD30, CD33, CD38, CD44, CD123, CD138, CEA, CLEC12A, CS-1, EGFR, EGFRVIII, EPCAM, DLL3, LGR5, MSLN, FOLR1, FOLR3, HER2, HM1.24, MCSP, PD-L1, PSMA protein or a variant thereof, in a preferred embodiment EGFR, PD-L1 or CLEC12A.

The antibody, bispecific or multispecific antibody is preferably a human or humanized antibody.

The bispecific or multispecific antibody preferably comprises two different immunoglobulin heavy chains with compatible heterodimerization domains. Said compatible heterodimerization domains are preferably compatible immunoglobulin heavy chain CH3 heterodimerization domains.

The bispecific or multispecific antibody is preferably an IgG antibody with a mutant CH2 and/or lower hinge domain such that interaction of the bispecific or multispecific IgG antibody to a Fc-gamma receptor is reduced. The mutant CH2 and/or lower hinge domain preferably comprise an amino substitution at position 235 and/or 236 (according to EU numbering), preferably an L235G and/or G236R substitution.

The bispecific or multispecific antibody preferably comprises a common light chain.

The invention further provides an antigen-binding protein or antibody as indicated herein, for use in the treatment of a subject in need thereof. The subject preferably has cancer or is treated for cancer. An antigen-binding protein or antibody having the CDRs and/or the VH sequence of MF8057, MF8058, MF8078, or MF8508, or a variant thereof having 0-10 amino acids substitutions, variations, insertions, additions or deletions, are preferred for treatment, in particular for a treatment comprising the local administration and/or local release of the antigen-binding protein or antibody. An antigen-binding protein or antibody having the CDRs and/or the VH sequence of MF9249, MF9267, MF8397, or a variant thereof having 0-10 amino acids substitutions, variations, insertions, additions or deletions, are preferred for a treatment of a subject with an over-active immune system, such as an auto-immune disease.

An antibody of the invention is, unless otherwise specifically specified, preferably a bispecific antibody. The bispecific antibody preferably binds at least human CD3. In addition, the bispecific antibody preferably binds at least a surface molecule that is preferentially expressed on human tumor cells. In a preferred embodiment, the bispecific antibody binds to BCMA, CD19, CD20, CD30, CD33, CD38, CD44, CD123, CD138, CEA, CLEC12A, CS-1, EGFR, EGFRVIII, EPCAM, DLL3, LGR5, MSLN, PD-L1, FOLR1, FOLR3, HER2, HM1.24, MCSP, or PSMA. In a more preferred embodiment, the bispecific antibody binds to EGFR or CLEC12A. In a more preferred embodiment, the multispecific antibody binds to EGFR and PD-L1.

The invention further provides a pharmaceutical composition comprising an antibody according to the invention.

Further provided is an antibody according to the invention that further comprises a label, preferably a label for in vivo imaging.

The invention also provides a method for the treatment of a subject having a tumor or at risk of having a tumor, comprising administering to the subject a bispecific or multispecific antibody according to the invention. Also provided is a bispecific or multispecific antibody according to the invention for use in the treatment of a subject having a tumor or at risk of having a tumor. Further provided is the use of an antibody of the invention for the preparation of a medicament for the treatment of a subject having a tumor or at risk of having a tumor. In a preferred embodiment, the tumor is an EGFR or a CLEC12A positive tumor or an EGFR and PD-L1 positive tumor.

### DETAILED DESCRIPTION OF THE INVENTION

An “antibody” is a proteinaceous molecule belonging to the immunoglobulin class of proteins, containing one or more domains that bind an epitope on an antigen, where such domains are derived from or share sequence homology with the variable region of an antibody. Antibody binding has different qualities including specificity and affinity. The specificity determines which antigen or epitope thereof is specifically bound by the binding domain. The affinity is a measure for the strength of binding to a particular antigen or epitope. It is convenient to note here that the ‘specificity’ of an antibody refers to its selectivity for a particular antigen, whereas ‘affinity’ refers to the strength of the interaction between the antibody's antigen binding site and the epitope it binds. Antibodies are typically made of basic structural units—each with two heavy chains and two light chains. Antibodies for therapeutic use are preferably as close to natural antibodies of the subject to be treated as possible (for instance human antibodies for human subjects). An antibody according to the present invention is not limited to any particular format or method of producing it.

Thus, the “binding specificity” as used herein refers to the ability of an individual antibody binding site to react with an antigenic determinant. Typically, the binding site of the antibody of the invention is located in the variable domain, in the Fab portion comprising the variable domain and is constructed from the hypervariable regions of the heavy and light chains.

An antibody of the invention is preferably an IgG antibody, preferably an IgG1 antibody. Full length IgG antibodies may be preferred because of their favorable half-life and the desire to stay as close to fully autologous (human) molecules for reasons of immunogenicity. IgG1 is favored based on its long circulatory half-life in man. In order to prevent or avoid immunogenicity in humans it is preferred that a bispecific full length IgG antibody according to the invention is a human IgG1.

A “bispecific antibody” is an antibody as described herein wherein one variable domain of the antibody binds to a first antigen whereas a second variable domain of the antibody binds to a second antigen, wherein said first and second antigens are not identical. The term “bispecific antibody” also encompasses biparatopic antibodies, wherein one variable domain of the antibody binds to a first epitope on an antigen whereas a second variable domain of the antibody binds a second epitope on the antigen. The term further includes antibodies wherein at least one VH is capable of specifically recognizing a first antigen and a VL, paired with the at least one VH in an immunoglobulin variable domain, is capable of specifically recognizing a second antigen. The resulting VH/VL pair will bind either antigen 1 or antigen 2, and are called “two-in-one antibodies”, described in for instance WO 2008/027236, WO 2010/108127 and Schaefer et al (Cancer Cell 20, 472-486, October 2011). A bispecific antibody according to the present invention is not limited to any particular bispecific format or method of producing it. A bispecific antibody is a multispecific antibody.

Multispecific multimers or antibodies as referred to herein, encompass proteinaceous molecules belonging to the immunoglobulin class of proteins, containing two or more domains that bind an epitope on an antigen, where such domains are derived from or share sequence homology with the variable region of an antibody, and include proteinaceous molecules binding three antigens or more as known in the art, including as described in WO2019/190327.

An “antigen” is a molecule capable of inducing an immune response (to produce an antibody) in a host organism and/or being targeted by an antibody. At the molecular level, an antigen is characterized by its ability to be bound by the antigen-binding site of an antibody. Also mixtures of antigens can be regarded as an ‘antigen’, i.e. the skilled person would appreciate that sometimes a lysate of tumor cells, or viral particles may be indicated as ‘antigen’ whereas such tumor cell lysate or viral particle preparation exists of many antigenic determinants. An antigen comprises at least one, but often more, epitopes. For binding proteins and antibodies as disclosed herein, the antigen is typically associated with a cell membrane and present on the extracellular portion of the cell membrane.

An “epitope” or “antigenic determinant” is a site on an antigen to which an immunoglobulin or antibody specifically binds. Epitopes can be formed from contiguous amino acids or noncontiguous amino acids juxtaposed by tertiary folding of a protein (so-called linear and conformational epitopes, respectively). Epitopes formed from contiguous, linear amino acids are typically retained on exposure to denaturing solvents, whereas epitopes formed by tertiary folding, conformation is typically lost on treatment with denaturing solvents. An epitope may typically include 3, 4, 5, 6, 7, 8, 9, 10, 11, 12, 13, 14 or 15 amino acids in a unique spatial conformation.

The term “heavy chain” or “immunoglobulin heavy chain” includes an immunoglobulin heavy chain constant region sequence from any organism, and unless otherwise specified includes a heavy chain variable domain. The term heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a CH1 domain, a hinge, a CH2 domain, and a CH3 domain. A functional fragment of a heavy chain includes a fragment that is capable of specifically recognizing an antigen and that comprises at least one CDR.

The term “light chain” includes an immunoglobulin light chain variable domain, or V_{L}(, or a functional fragment thereof; and an immunoglobulin constant domain, or C_{L}, or functional fragment thereof, sequence from any organism. Unless otherwise specified, the term light chain may include a light chain selected from a human kappa, lambda, and a combination thereof. Light chain variable (V_{L}) domains typically include three light chain CDRs and four framework (FR) regions, unless otherwise specified. Generally, a full-length light chain includes, from N-terminus to C-terminus, a V_{L} domain that includes FR1-CDR1-FR2-CDR2-FR3-CDR3-FR4, and a light chain constant domain. Light chains that can be used with this invention include those, e.g., that do not selectively bind an epitope selectively bound by the heavy chains.

Suitable light chains for use in an antibody of the invention include a common light chain (cLC), such as those that can be identified by screening for the most commonly employed light chains in existing antibody libraries (wet libraries or in silico), where the light chains do not substantially interfere with the affinity and/or selectivity of the epitope-binding domains of the heavy chains, but are also suitable to pair with an array of heavy chains. For example, a suitable light chain includes one from a transgenic animal, such as MeMo® having a common light chain integrated into its genome and which can be used to generate large panels of common light chain antibodies having diversity at the heavy chain and capable of specifically binding an antigen upon exposure to said antigen.

The term “common light chain” according to the invention refers to light chains which may be identical or have some amino acid sequence differences while the binding specificity of an antibody of the invention is not affected, i.e. the differences do not materially influence the formation of functional binding regions.

It is for instance possible within the scope of the definition of common chains as used herein, to prepare or find variable chains that are not identical but still functionally equivalent, e.g., by introducing and testing conservative amino acid changes, changes of amino acids in regions that do not or only partly contribute to binding specificity when paired with a cognate chain, and the like. Such variants are thus also capable of binding different cognate chains and forming functional antigen binding domains. The term ‘common light chain’ as used herein thus refers to light chains which may be identical or have some amino acid sequence differences while retaining the binding specificity of the resulting antibody after pairing with a heavy chain. A combination of a certain common light chain and such functionally equivalent variants is encompassed within the term “common light chain”. Reference is made to WO 2004/009618 and WO2009/157771 for a detailed description of the use of common light chains.

A “Fab” means a binding domain comprising a variable region, typically a binding domain comprising a paired heavy chain variable region and light chain variable region. A Fab may comprise constant region domains, including a CH1 and a VH domain paired with a constant light domain (CL) and VL domain. Such pairing may take place, for example, as covalent linkage via a disulfide bridge at the CH1 and CL domains.

A “single-chain variable fragment” (scFv) means a binding domain comprising a VH domain and a VL domain which are connected via a linker, for example a peptide linker, for example from about 10 to about 25 amino acids in length.

The term ‘full length IgG’ or ‘full length antibody’ according to the invention is defined as comprising an essentially complete IgG, which however does not necessarily have all functions of an intact IgG. For the avoidance of doubt, a full length IgG contains two heavy and two light chains. Each chain contains constant (C) and variable (V) regions, which can be broken down into domains designated CH1, CH2, CH3, VH, and CL, VL. An IgG antibody binds to antigen via the variable region domains contained in the Fab portion, and after binding can interact with molecules and cells of the immune system through the constant domains, mostly through the Fc portion. Full length antibodies according to the invention encompass IgG molecules wherein mutations may be present that provide desired characteristics. Full length IgG should not have deletions of substantial portions of any of the regions. However, IgG molecules wherein one or several amino acid residues are deleted, without essentially altering the binding characteristics of the resulting IgG molecule, are embraced within the term “full length IgG”. For instance, such IgG molecules can have a deletion of between 1 and 10 amino acid residues, preferably in non-CDR regions, wherein the deleted amino acids are not essential for the binding specificity of the IgG.

“Percent (%) identity” as referring to nucleic acid or amino acid sequences herein is defined as the percentage of residues in a candidate sequence that are identical with the residues in a selected sequence, after aligning the sequences for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/based or amino acids. The sequence identity is the percentage of identical matches between the two sequences over the reported aligned region.

A comparison of sequences and determination of percentage of sequence identity between two sequences can be accomplished using a mathematical algorithm. The skilled person will be aware of the fact that several different computer programs are available to align two sequences and determine the identity between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley).

For the purposes of inventions and sequences set out herein, percent sequence identity between two nucleic acid sequences may be determined using the AlignX application of the Vector NTI Program Advance 10.5.2 software using the default settings, which employ a modified ClustalW algorithm (Thompson, J. D., Higgins, D. G., and Gibson T. J. (1994) Nuc. Acid Res. 22:4673-4680), the swgapdnarnt score matrix, a gap opening penalty of 15 and a gap extension penalty of 6.66. Amino acid sequences may be aligned with the AlignX application of the Vector NTI Program Advance 11.5.2 software using default settings, which employ a modified ClustalW algorithm (Thompson, J. D., Higgins, D. G., and Gibson T. J., 1994), the blosum62mt2 score matrix, a gap opening penalty of 10 and a gap extension penalty of 0.1.

The term “super-cluster” or “supercluster” is used herein to refer to a group of clones, and the binding domains they are capable of producing, based on the same VH V-gene segment usage and having at least 70% sequence identity in HCDR3 and the same HCDR3 length.

Thus, in a preferred embodiment, there is provided by the present invention, a “super-cluster” or “supercluster” comprising a group of clones, and the binding domains they are capable of producing, based on the same VH V-gene segment usage and having at least 70% sequence identity in HCDR3 and the same HCDR3 length. In a preferred embodiment, said sequence identity is 80%, more preferably 90%, most preferably 95% sequence identity, with the proviso that a clone comprising nucleic acid coding for HCDR3 sequence DGGYSYGPYWYFDL (SEQ ID NO 199) and DHRGYGDYEGGGFDY (SEQ ID NO 144), clones comprising nucleic acid coding for HCDR2 sequences GFIPVLGTANYAQKFQG, GIIPLFGTITYAQKFQG (SEQ ID NO 43), and SIIPIFGTITYAQKFQG (SEQ ID NO 52) are excluded, or with the proviso that clones comprising nucleic acid coding for VH sequences

(SEQ ID NO 120)
QVQLVQSGGGLVQPGGSLRLSCATSGFKFSSYALSWVRQAPGKGLEWVS
GISGSGRTTWYADSVKGRFTISRDNSKNTLYLQMNSLRAEDTAVYYCAR
DGGYSYGPYWYFDLWGRGTLVTVSS;
(SEQ ID NO 145)
EVQLVESGGGLVQPGRSLRLSCATSGFNFDDYTMHWVRQAPGKGLEWVS
DISWSSGSIGYADSVKGRFTISRDNAKNSLWLQMNSLRTEDTALYFCAK
DHRGYGDYEGGGFDYWGQGTLVTVSS;
(SEQ ID NO 45)
EVQLVQSGAEVKKPGSSVKVSCKASGGTFRSFGISWVRQAPGQGLEWMG
GFIPVLGTANYAQKFQGRVTIIADKSTNTAYMELSSLRSEDTAVYYCAR
RGNWNPFDPWGQGTLVTVSS;
(SEQ ID NO 54)
QVQLVQSGAEVKKPGSSVKVSCKASGDAFKSKTFTISWVRQAPGQGLEW
LGGIIPLFGTITYAQKFQGRVTITADKSTNTAFMELSSLRSEDTAMYYC
TRRGNWNPFDPWGQGTLVTVSS;
and
(SEQ ID NO 63)
EVQLVQSGSELKKPGSSVKVSCKASGVTFNSRTFTISWVRQAPGQGLEW
LGSIIPIFGTITYAQKFQGRVTITADKSTSTAFMELTSLRSEDTAIYYC
TRRGNWNPFDPWGQGTLVTVSS

are excluded or with the proviso that clones from said group comprise nucleic acid that code for a HCDR3 that is comprised by or designed to be comprised by a bispecific antibody.

The term “super-cluster 1” or “supercluster 1” is used herein to refer to a group of clones, and the binding domains they are capable of producing, based on the same VH V-gene segment usage (VH1-69) and having at least 70% sequence identity in HCDR3 and the same HCDR3 length to members of that supercluster. Included are for instance MF8048, MF8056, MF8057, MF8058, MF8078 and MF8101. In another preferred embodiment, an anti-CD3 antibody herein is based on the same VH V-gene segment usage of VH1-69 and/or having at least 80% identity in HCDR3 and the same HCDR3 length, more preferably 90% or most preferably 95% identity in the HCDR3. In another preferred embodiment, an anti-CD3 antibody herein is based on the same VH V-gene segment usage of VH1-69 and/or having at least 80% identity in HCDR3 and the same HCDR3 length compared to the encoded CDR3 segment RGNWNPFDP, preferably at least 90% sequence identity in HCDR3 and the same HCDR3 length, more preferably 95% or most preferably 98% identity and the same HCDR3 length, with the proviso that clones comprising nucleic acid coding for HCDR2 sequences GFIPVLGTANYAQKFQG (SEQ ID NO 43), GIIPLFGTITYAQKFQG (SEQ ID NO 52) and SIIPIFGTITYAQKFQG (SEQ ID NO 61) are excluded, or with the proviso that clones comprising nucleic acid coding for VH sequences

(SEQ ID NO 45)
EVQLVQSGAEVKKPGSSVKVSCKASGGTFRSFGISWVRQAPGQGLEWMG
GFIPVLGTANYAQKFQGRVTIIADKSTNTAYMELSSLRSEDTAVYYCAR
RGNWNPFDPWGQGTLVTVSS;
(SEQ ID NO 54)
QVQLVQSGAEVKKPGSSVKVSCKASGDAFKSKTFTISWVRQAPGQGLEW
LGGIIPLFGTITYAQKFQGRVTITADKSTNTAFMELSSLRSEDTAMYYC
TRRGNWNPFDPWGQGTLVTVSS;
(SEQ ID NO 63)
EVQLVQSGSELKKPGSSVKVSCKASGVTFNSRTFTISWVRQAPGQGLEW
LGSIIPIFGTITYAQKFQGRVTITADKSTSTAFMELTSLRSEDTAIYYC
TRRGNWNPFDPWGQGTLVTVSS;

are excluded or with the proviso that clones from said group comprise nucleic acid that code for a HCDR3 that is comprised by or designed to be comprised by a bispecific antibody. The term “super-cluster 3” or “supercluster 3” is used herein to refer to a group of clones, and the binding domains they are capable of producing, based on the same VH V-gene segment usage (VH3-23) and having at least 70% sequence identity in HCDR3 and the same HCDR3 length to members of that supercluster. Included are for instance MF8397, and MF8562. In another preferred embodiment, an anti-CD3 antibody herein is based on the same VH V-gene segment usage of VH3-23 and/or having at least 80% identity in HCDR3 and the same HCDR3 length, more preferably 90% or most preferable 95% identity in the HCDR3. In another preferred embodiment, an anti-CD3 antibody herein is based on the same VH V-gene segment usage of VH3-23 and/or having at least 80% identity in HCDR3 and the same HCDR3 length compared to the encoded CDR3 segment DGGYSYGPYWYFDL (SEQ ID NO 119), preferably at least 90% sequence identity in HCDR3 and the same HCDR3 length, more preferably 95% or most preferably 98% identity and the same HCDR3 length, with the proviso that a clone comprising nucleic acid encoding HCDR3 sequence DGGYSYGPYWYFDL (SEQ ID NO 119) is excluded or with the proviso that a clone comprising nucleic acid coding for VH sequence QVQLVQSGGGLVQPGGSLRLSCATSGFKFSSYALSWVRQAPGKGLEWVSGISGSGRTTWYA DSVKGRFTISRDNSKNTLYLQMNSLRAEDTAVYYCARDGGYSYGPYWYFDLWGRGTLVTVSS (SEQ ID NO 120) is excluded or with the proviso that clones from said group comprise nucleic acid that code for a HCDR3 that is comprised by or designed to be comprised by a bispecific antibody.

The term “super-cluster 4” or “supercluster 4” is used herein to refer to a group of clones, and the binding domains they are capable of producing, based on the same VH V-gene segment usage (VH3-9) and having at least 70% sequence identity in HCDR3 and the same HCDR3 length to members of that supercluster. Included are for instance MF8508, MF8998, MF10401 and MF10428. In another preferred embodiment, an anti-CD3 antibody herein is based on the same VH V-gene segment usage of VH3-9 and/or having at least 80% identity in HCDR3 and the same HCDR3 length, more preferably 90% or most preferable 95% identity in the HCDR3. In another preferred embodiment, an anti-CD3 antibody herein is based on the same VH V-gene segment usage of VH3-9 and/or having at least 80% identity in HCDR3 and the same HCDR3 length compared to the encoded CDR3 segment DHRGYGDYEGGGFDY (SEQ ID NO 144), preferably at least 90% sequence identity in HCDR3 and the same HCDR3 length, more preferably 95% or most preferably 98% identity and the same HCDR3 length, with the proviso that a clone comprising nucleic acid coding for HCDR3 sequence DHRGYGDYEGGGFDY (SEQ ID NO 168) is excluded or with the proviso that a clone comprising nucleic acid coding for VH sequence

(SEQ ID NO 145)
EVQLVESGGGLVQPGRSLRLSCATSGFNFDDYTMHWVRQAPGKGLEWVS
DISWSSGSIGYADSVKGRFTISRDNAKNSLWLQMNSLRTEDTALYFCAK
DHRGYGDYEGGGFDYWGQGTLVTVSS

is excluded or with the proviso that clones from said group comprising nucleic acid that code for a HCDR3 that is comprised by or designed to be comprised by a bispecific antibody.

The term “super-cluster 7” or “supercluster 7” is used herein to refer to a group of clones, and the binding domains they are capable of producing, based on the same VH V-gene segment usage (VH5-51) and having at least 70% sequence identity in HCDR3 and the same HCDR3 length to members of that supercluster. Included is for instance MF9249, and MF9267. In another preferred embodiment, an anti-CD3 antibody herein is based on the same VH V-gene segment usage of VH5-51 and/or having at least 80% identity in HCDR3 and the same HCDR3 length, more preferably 90% or most preferable 95% identity in the HCDR3. In another preferred embodiment, an anti-CD3 antibody herein is based on the same VH V-gene segment usage of V H5-51 and/or having at least 80% identity in HCDR3 and the same HCDR3 length compared to the encoded CDR3 segment HIRYFDWSEDYHYYLDV (SEQ ID NO 201), preferably at least 90% sequence identity in HCDR3 and the same HCDR3 length, more preferably 95% or most preferably 98% identity and the same HCDR3 length

The invention further provides a bispecific antibody comprising a variable domain with a VH encoded by
V-gene segment VH1-69; or
a variant of V-gene segment VH1-69 comprising at least 70%, preferably at least 80%, more preferably at least 90% and more preferably at least 95% sequence identity to the sequence of said V-gene segment;
   wherein the VH further comprises
a HCDR3 of MF8048, MF8056, MF8057, MF8058, MF8078 or MF8101;
or a variant of said HCDR3 comprising at least 70% sequence identity to said HCDR3 and the same length of said HCDR3.

In a preferred embodiment said variant of said HCDR3 comprises the same length as said HCDR3 and at least 80% sequence identity to said HCDR3, more preferably at least 90% and more preferably at least 95% sequence identity to the sequence of said HCDR3.

In some embodiments said bispecific antibody does not have a VH encoded by V-gene segment VH1-69; or a variant of V-gene segment VH1-69 with a HCDR2 sequence

(SEQ ID NO 43)
GFIPVLGTANYAQKFQG,
or
(SEQ ID NO 52)
GIIPLFGTITYAQKFQG
or
(SEQ ID NO 61)
SIIPIFGTITYAQKFQG.

In some embodiments said bispecific antibody does not have a VH encoded by V-gene segment VH1-69; or a variant of V-gene segment VH1-69 with a VH sequence

(SEQ ID NO 45)
EVQLVQSGAEVKKPGSSVKVSCKASGGTFRSFGISWVRQAPGQGLEWMG
GFIPVLGTANYAQKFQGRVTIIADKSTNTAYMELSSLRSEDTAVYYCAR
RGNWNPFDPWGQGTLVTVSS;
or
(SEQ ID NO 54)
QVQLVQSGAEVKKPGSSVKVSCKASGDAFKSKTFTISWVRQAPGQGLEW
LGGIIPLFGTITYAQKFQGRVTITADKSTNTAFMELSSLRSEDTAMYYC
TRRGNWNPFDPWGQGTLVTVSS;
or
(SEQ ID NO 63)
EVQLVQSGSELKKPGSSVKVSCKASGVTFNSRTFTISWVRQAPGQGLEW
LGSIIPIFGTITYAQKFQGRVTITADKSTSTAFMELTSLRSEDTAIYYC
TRRGNWNPFDPWGQGTLVTVSS.

The invention further provides a bispecific antibody comprising a variable domain with a VH encoded by
V-gene segment VH3-23; or
a variant of V-gene segment VH2-23 comprising at least 70%, preferably at least 80%, more preferably at least 90% and more preferably at least 95% sequence identity to the sequence of said V-gene segment;
   wherein the VH further comprises
a HCDR3 of MF8397; or MF8562;
or a variant of said HCDR3 comprising at least 70% sequence identity to said HCDR3 and the same length of said HCDR3.

In a preferred embodiment said variant of said HCDR3 comprises the same length as said HCDR3 and at least 80% sequence identity to said HCDR3, more preferably at least 90%, more preferably at least 93% and more preferably at least 95% sequence identity to the sequence of said HCDR3.

In some embodiments said bispecific antibody does not have a VH encoded by V-gene segment VH3-23; or a variant of V-gene segment VH3-23 with a HCDR3 sequence

(SEQ ID NO 119)
DGGYSYGPYWYFDL.

In some embodiments said bispecific antibody does not have a VH encoded by V-gene segment VH3-23; or a variant of V-gene segment VH3-23 with a VH sequence

(SEQ ID NO 120)
QVQLVQSGGGLVQPGGSLRLSCATSGFKFSSYALSWVRQAPGKGLEWVSG
ISGSGRTTWYADSVKGRFTISRDNSKNTLYLQMNSLRAEDTAVYYCARDG
GYSYGPYWYFDLWGRGTLVTVSS.

The invention further provides a bispecific antibody comprising a variable domain with a VH encoded by
V-gene segment VH3-9; or
a variant of V-gene segment VH3-9 comprising at least 70%, preferably at least 80%, more preferably at least 90% and more preferably at least 95% sequence identity to the sequence of said V-gene segment;
   wherein the VH further comprises
a HCDR3 of MF8508; MF8998; MF1041; or MF10428;
or a variant of said HCDR3 comprising at least 70% sequence identity to said HCDR3 and the same length of said HCDR3.

In a preferred embodiment said variant of said HCDR3 comprises the same length as said HCDR3 and at least 80% sequence identity to said HCDR3, more preferably at least 90% and more preferably at least 95% sequence identity to the sequence of said HCDR3.

In some embodiments said bispecific antibody does not have a VH encoded by V-gene segment VH3-9; or a variant of V-gene segment VH3-9 with a HCDR3 sequence

(SEQ ID NO 144)
DHRGYGDYEGGGFDY.

In some embodiments said bispecific antibody does not have a VH encoded by V-gene segment VH3-9; or a variant of V-gene segment VH3-9 with a VH sequence

(SEQ ID NO 145)
EVQLVESGGGLVQPGRSLRLSCATSGFNFDDYTMHWVRQAPGKGLEWVS
DISWSSGSIGYADSVKGRFTISRDNAKNSLWLQMNSLRTEDTALYFCAK
DHRGYGDYEGGGFDYWGQGTLVTVSS.

The invention further provides a bispecific antibody comprising a variable domain with a VH encoded by
V-gene segment VH5-51; or
a variant of V-gene segment VH5-51 comprising at least 70%, preferably at least 80%, more preferably at least 90% and more preferably at least 95% sequence identity to the sequence of said V-gene segment;
   wherein the VH further comprises
a HCDR3 of MF9249 or MF9267;
or a variant of said HCDR3 comprising at least 70% sequence identity to said HCDR3 and the same length of said HCDR3.

In a preferred embodiment said variant of said HCDR3 comprises the same length as said HCDR3 and at least 80% sequence identity to said HCDR3, more preferably at least 90% and more preferably at least 95% sequence identity to the sequence of said HCDR3.

A bispecific antibody as provided by the invention defined herein is preferably not a bispecific antibody comprising a CD3 binding variable domain as defined in PCT/NL2019/050199.

The invention further provides a VH encoded by
V-gene segment VH1-69; or
a variant of V-gene segment VH1-69 comprising at least 70%, preferably at least 80%, more preferably at least 90% and more preferably at least 95% sequence identity to the sequence of said V-gene segment;
   wherein the VH further comprises
a HCDR3 of MF8048, MF8056, MF8057, MF8058, MF8078 or MF8101;
or a variant of said HCDR3 comprising at least 70% sequence identity to said HCDR3 and the same length of said HCDR3.

In a preferred embodiment said variant of said HCDR3 comprises the same length as said HCDR3 and at least 80% sequence identity to said HCDR3, more preferably at least 90% and more preferably at least 95% sequence identity to the sequence of said HCDR3.

In some embodiments said VH is not a VH encoded by V-gene segment VH1-69; or a variant of V-gene segment VH1-69 with a HCDR2 sequence GFIPVLGTANYAQKFQG (SEQ ID NO 43), or GIIPLFGTITYAQKFQG (SEQ ID NO 52) or SIIPIFGTITYAQKFQG (SEQ ID NO 61).

In some embodiments said VH is not a VH encoded by V-gene segment VH1-69; or a variant of V-gene segment VH1-69 with a VH sequence

(SEQ ID NO 45)
EVQLVQSGAEVKKPGSSVKVSCKASGGTFRSFGISWVRQAPGQGLEWMG
GFIPVLGTANYAQKFQGRVTIIADKSTNTAYMELSSLRSEDTAVYYCAR
RGNWNPFDPWGQGTLVTVSS;
or
(SEQ ID NO 54)
QVQLVQSGAEVKKPGSSVKVSCKASGDAFKSKTFTISWVRQAPGQGLEW
LGGIIPLFGTITYAQKFQGRVTITADKSTNTAFMELSSLRSEDTAMYYC
TRRGNWNPFDPWGQGTLVTVSS;
or
(SEQ ID NO 63)
EVQLVQSGSELKKPGSSVKVSCKASGVTFNSRTFTISWVRQAPGQGLEW
LGSIIPIFGTITYAQKFQGRVTITADKSTSTAFMELTSLRSEDTAIYYC
TRRGNWNPFDPWGQGTLVTVSS.

The invention further provides a VH encoded by
V-gene segment VH3-23; or
a variant of V-gene segment VH2-23 comprising at least 70%, preferably at least 80%, more preferably at least 90% and more preferably at least 95% sequence identity to the sequence of said V-gene segment;
   wherein the VH further comprises
a HCDR3 of MF8397; or MF8562;
or a variant of said HCDR3 comprising at least 70% sequence identity to said HCDR3 and the same length of said HCDR3.

In a preferred embodiment said variant of said HCDR3 comprises the same length as said HCDR3 and at least 80% sequence identity to said HCDR3, more preferably at least 90%, more preferably at least 93% and more preferably at least 95% sequence identity to the sequence of said HCDR3.

In some embodiments said VH is not a VH encoded by V-gene segment VH3-23; or a variant of V-gene segment VH3-23 with a HCDR3 sequence DGGYSYGPYWYFDL. (SEQ ID NO 119)

In some embodiments said VH is not a VH encoded by V-gene segment VH3-23; or a variant of V-gene segment VH3-23 with a VH sequence

(SEQ ID NO 120)
QVQLVQSGGGLVQPGGSLRLSCATSGFKFSSYALSWVRQAPGKGLEWVS
GISGSGRTTWYADSVKGRFTISRDNSKNTLYLQMNSLRAEDTAVYYCAR
DGGYSYGPYWYFDLWGRGTLVTVSS.

The invention further provides a VH encoded by
V-gene segment VH3-9; or
a variant of V-gene segment VH3-9 comprising at least 70%, preferably at least 80%, more preferably at least 90% and more preferably at least 95% sequence identity to the sequence of said V-gene segment;
   wherein the VH further comprises
a HCDR3 of MF8508; MF8998; MF10401; or MF10428;
or a variant of said HCDR3 comprising at least 70% sequence identity to said HCDR3 and the same length of said HCDR3.

In a preferred embodiment said variant of said HCDR3 comprises the same length as said HCDR3 and at least 80% sequence identity to said HCDR3, more preferably at least 90% and more preferably at least 95% sequence identity to the sequence of said HCDR3.

In some embodiments said VH is not a VH encoded by V-gene segment VH3-9; or a variant of V-gene segment VH3-9 with a HCDR3 sequence DHRGYGDYEGGGFDY (SEQ ID NO 144).

In some embodiments said VH is not a VH encoded by V-gene segment VH3-9; or a variant of V-gene segment VH3-9 with a VH sequence

(SEQ ID NO 145)
EVQLVESGGGLVQPGRSLRLSCATSGFNFDDYTMHWVRQAPGKGLEWVS
DISWSSGSIGYADSVKGRFTISRDNAKNSLWLQMNSLRTEDTALYFCAK
DHRGYGDYEGGGFDYWGQGTLVTVSS.

The invention further provides a VH encoded by
V-gene segment VH5-51; or
a variant of V-gene segment VH5-51 comprising at least 70%, preferably at least 80%, more preferably at least 90% and more preferably at least 95% sequence identity to the sequence of said V-gene segment;
   wherein the VH further comprises
a HCDR3 of MF9249 or MF9267;
or a variant of said HCDR3 comprising at least 70% sequence identity to said HCDR3 and the same length of said HCDR3.

In a preferred embodiment said variant of said HCDR3 comprises the same length as said HCDR3 and at least 80% sequence identity to said HCDR3, more preferably at least 90% and more preferably at least 95% sequence identity to the sequence of said HCDR3.

A VH as provided by the invention defined herein is preferably not a VH of a CD3 binding variable domain as defined in PCT/NL2019/050199.

Also provided is an antigen-binding protein or antibody, preferably a bispecific antibody, wherein the CDRs have 70%, preferably 80%, more preferably 90% identity to the CDRs as claimed. In a preferred embodiment the antigen-binding protein or antibody is a bispecific antibody that comprises CDRs with at most 2, preferably at most 1 and more preferably at most 0 amino acid residue variations, insertions, substitutions, deletions or additions with respect to the CDRs as claimed.

Antigen binding by an antibody is typically mediated through the complementarity regions of the antibody and the specific three-dimensional structure of both the antigen and the variable domain allowing these two structures to bind together with precision (an interaction similar to a lock and key), as opposed to random, non-specific sticking of antibodies. As an antibody typically recognizes an epitope of an antigen, and as such epitope may be present in other proteins as well, antibodies according to the present invention that bind CD3 or CLEC12A may recognize other proteins as well, if such other proteins contain the same epitope. Hence, the term “binding” does not exclude binding of the antibodies to another protein or protein(s) that contain the same epitope. A heavy/light chain combination that binds CD3 in an antibody of the invention does not bind to other proteins on the membrane of cells in a post-natal, preferably adult human. A heavy/light chain combination that binds CLEC12A, EGFR, PD-L1 or tumor cell antigens of the invention does not bind other proteins on the membrane of cells in a post-natal, preferably adult human. Suitable tumor antigen specific arms are disclosed in PCT/NL2019/050199.

“Plurality” means two or more.

A “variant” of an antibody as described herein may comprise a functional part, derivative and/or analogue of an antibody. This includes antibody mimetics, monobodies and aptamers.

A variant typically maintains the binding specificity of the antibody, for example the specificities of a bispecific antibody. A variant may be a functional part or derivative of a binding domain, multimer or antibody as described herein.

A functional part of a binding domain, multimer or antibody as described herein is a part comprising a variable domain that binds the same target as such binding domain, multimer or antibody.

A functional derivative of an antibody as described herein is a protein comprising a variable domain that binds one target and a variable domain that binds a second target that are linked by a linking region. The variable domains may be variable domains as such, or Fab fragments or variable domain like molecules such as single chain Fv (scFv) fragments comprising a VH and a VL linked together via a linker. Antibody variable domains or antibody variable domain like molecules can be linked to each other in different ways. Various linkers and carrier structures have been described that can bind one, two or more variable domains. An antigen-binding protein as described herein is a protein comprising at least one of such variable domain. In the case of bispecific or multispecific antigen binding proteins, such proteins comprise two or more variable domains of which at least two bind a different target. The variable domains are linked to each other via a linking portion. This is typically a stretch of 0-15, preferably 3-12, more preferably around 5-8 amino acid residues. Other examples of variable domain like molecules are so-called single domain antibody fragments. A single-domain antibody fragment (sdAb) is an antibody fragment with a single monomeric variable antibody region. Like a whole antibody, it is able to bind selectively to a specific antigen. With a molecular weight of only 12-15 kDa, single-domain antibody fragments are much smaller than common antibodies (150-160 kDa) which are composed of two heavy protein chains and two light chains, and even smaller than Fab fragments (~50 kDa, one light chain and half a heavy chain) and single-chain variable fragments (~25 kDa, two variable regions, one from a light and one from a heavy chain). Single-domain antibodies by themselves are not much smaller than normal antibodies (being typically 90-100 kDa). Single-domain antibody fragments may be engineered from heavy-chain antibodies found in camelids; these are called VHH fragments (Nanobodies®). Some fishes also have heavy-chain only antibodies (IgNAR, ‘immunoglobulin new antigen receptor’), from which single-domain antibody fragments called VNAR fragments can be obtained. An alternative approach is to split the dimeric variable domains from common immunoglobulin G (IgG) from humans or mice into monomers. Although most research into single-domain antibodies is currently based on heavy chain variable domains, nanobodies derived from light chains have also been shown to be capable of binding to target epitopes. Other non-limiting examples of variable domain-like molecules are VHH, Human Domain Antibodies (dAbs) and Unibodies. Preferred functional parts are parts that comprise variable domains comprising a heavy chain variable region and a light chain variable region. Non-limiting examples of such variable domains are F(ab)-fragments and Single chain Fv fragments. Bispecific formats for variable domain(-like) linkage are for instance Human Serum Albumin (HSA) bound to two different scFv; bispecific mini-antibodies comprising two different scFv bound together via dimerization motifs or self-associating secondary structures such as helix bundles or coiled coils to bring about dimerization of the scFv fragments (Morrison (2007) Nat. Biotechnol. 25:1233-34). Examples of suitable HSA linkers and method for coupling scFv to the linker are described in WO2009/126920.

A functional derivative can be an antibody mimetic, a polypeptide, an aptamer or a combination thereof. These proteins or aptamers typically bind to one target. The protein of the invention binds to two or more targets. It is to be understood that any combination of these antibodies, antibody mimetics, polypeptides and aptamers can be linked together by methods known in the art. For example, in some embodiments the binding molecule of the invention is a conjugate or a fusion protein.

An antibody mimetic is a polypeptide that, like antibodies, can specifically bind an antigen, but that is not structurally related to antibodies. Antibody mimetics are usually artificial peptides or proteins with a molar mass of about 3 to 20 kDa. Non-limiting examples of antibody mimetics are affibody molecules (typically based on the Z domain of Protein A); affilins (typically based on Gamma-B crystalline or Ubiquitin); affimers (typically based on Cystatin); affitins (typically based on Sac7d from Sulfolobus acidocaldarius); alphabodies (typically based on Triple helix coiled coil); anticalins (typically based on Lipocalins); avimers (typically based on A domains of various membrane receptors); DARPins (typically based on ankyrin repeat motif); fynomers (typically based on SH3 domain of Fyn 7); kunitz domain peptides (typically based on Kunitz domains of various protease inhibitors); and monobodies (typically based on type III domain of fibronectin).

Monobodies are synthetic binding proteins that are constructed using a fibronectin type Ill domain (FN3) as a molecular scaffold. Monobodies are an alternative to antibodies for creating target-binding proteins.

Monobodies and other antibody mimetics are typically generated from combinatorial libraries in which portions of the scaffold are diversified using molecular display and directed evolution technologies such as phage display, mRNA display and yeast surface display.

Aptamers are oligonucleotide or peptide molecules that bind to a specific target molecule. Aptamers are usually created by selecting them from a large random sequence pool, but natural aptamers also exist in riboswitches. Aptamers can be used for both basic research and clinical purposes as macromolecules.

Throughout the present specification and the accompanying claims, the words “comprise”, “include” and “having” and variations such as “comprises”, “comprising”, “includes” and “including” are to be interpreted inclusively. That is, these words are intended to convey the possible inclusion of other elements or integers not specifically recited, where the context allows.

The articles “a” and “an” are used herein to refer to one or to more than one (i.e. to one or at least one) of the grammatical object of the article. By way of example, “an element” may mean one element or more than one element.

An antibody of the invention is preferably a bispecific or multispecific antibody. The bispecific or multispecific antibody preferably binds at least human CD3.

An antigen-binding protein or antibody of the invention is preferably a bi- or multispecific antigen binding protein or antibody. The bi- or multispecific antigen binding protein or antibody preferably binds at least human CD3 and in addition, preferably at least a surface molecule that is expressed on human tumor cells. In a preferred embodiment the bi- or multispecific antigen binding protein or antibody binds to BCMA, CD19, CD20, CD30, CD33, CD38, CD44, CD123, CD138, CEA, CLEC12A, CS-1, EGFR, EGFRvIII, EPCAM, DLL3, LGR5, MSLN, PD-L1, FOLR1, FOLR3, HER2, HM1.24, MCSP, or PSMA. In a particularly preferred embodiment, the bispecific antibody binds to CLEC12A. In a particularly preferred embodiment, the multispecific antibody binds to CD3, PD-L1 and EGFR.

As used herein, the term “CLEC12A” refers to C type lectin domain family 12 member A. CLEC12A is also referred to as C-Type Lectin Protein CLL-1; MICL; Dendritic Cell-Associated Lectin 2; C-Type Lectin Superfamily; Myeloid Inhibitory C-Type Lectin-Like Receptor; C-Type Lectin-Like Molecule-1; DCAL2; CLL1; C-Type Lectin-Like Molecule 1; DCAL-2; Killer cell lectin like receptor subfamily L, member 1 (KLRL1); CD371 (cluster of differentiation 371) (Bakker A. et al. Cancer Res. 2004, 64, p8843 50; GenBank™ access.no: AY547296; Zhang W. et al. GenBank™ access.no: AF247788; A. S. Marshall, et al. J Biol Chem 2004, 279, p14792-802; GenBank™ access.no: AY498550; Y. Han et al. Blood 2004, 104, p2858 66; H. Floyd, et al. GenBank™ access.no: AY426759; C. H. Chen, et al. Blood 2006, 107, p1459 67). Ids: HGNC: 31713; Entrez Gene: 160364; Ensembl: ENSG00000172322; OMIM: 612088; UniProtKB: Q5QGZ9.

CLEC12A is an antigen that is expressed on leukemic blast cells and on leukemic stem cells in acute myeloid leukemia (AML), including the CD34 negative or CD34 low expressing leukemic stem cells (side population) (A. B. Bakker et al. Cancer Res 2004, 64, p8443 50; Van Rhenen et al. 2007 Blood 110:2659; Moshaver et al. 2008 Stem Cells 26:3059), as well as in myelodysplastic syndromes (MDS) (Bakker et al. 2004, supra and Toff-Peterson et al., Br. J. Haematol. 175 (3): 393-401, 2016). Expression of CLEC12A is otherwise thought to be restricted to cells of the hematopoietic lineage, particularly to myeloid lineage in peripheral blood and bone marrow, i.e., granulocytes, monocytes and dendritic cell precursors. More importantly, CLEC12A is absent on normal hematopoietic stem cells. Where reference is made to CLEC12A herein, the reference is to human CLEC12A (SEQ ID NO: 1; FIG. 19), unless specifically stated otherwise.

The term “CLEC12A” means all variants (such as splice and mutation) that are referenced herein and isoforms thereof that retain the myeloid expression profile (both at surface expression level and mRNA level) including as described in Bakker et al. Cancer Res 2004, 64, p8443-50 and Marshall 2004-J Biol Chem 279 (15), p14792-802. While accession numbers are primarily provided as a further method of identification, the actual sequence of the protein may vary, for instance because of a mutation in the encoding gene such as those occurring in some cancers or the like.

The term “CD3” (cluster of differentiation 3) refers a protein complex, which is composed of a CD3γ chain (SwissProt P09693), a CD30 chain (SwissProt P04234), CD38 chains (SwissProt P07766), and a CD3 zeta chain homodimer (SwissProt P20963). CD38 is known under various aliases some of which are: “CD3e Molecule, Epsilon (CD3-TCR Complex)”; “CD3e Antigen, Epsilon Polypeptide (TiT3 Complex)”; T-Cell Surface Antigen T3/Leu-4 Epsilon Chain; T3E; T-Cell Antigen Receptor Complex, Epsilon Subunit Of T3; CD3e Antigen; CD3-Epsilon 3; IMD18; TCRE. Ids for CD3E Gene are HGNC: 1674; Entrez Gene: 916; Ensembl: ENSG00000198851; OMIM: 186830 and UniProtKB: P07766. These chains associate with the T-cell receptor (TCR) and the Z-chain to form a TCR complex that can upon mitogenic signaling generates an activation signal in T lymphocytes. CD3 is expressed on T cells and NK T cells. Where reference is made to CD3 herein, the reference is to human CD3 (SEQ ID NOs: 2-5; FIG. 20), unless specifically stated otherwise.

BCMA is also referred to as Tumor Necrosis Factor Receptor Superfamily, Member 17 (TNFRSF17); TNFRSF13A2; B Cell Maturation Antigen; BCM; B-Cell Maturation Factor; B-Cell Maturation Protein; CD269 or CD269 Antigen. Ids: HGNC: 11913; Entrez Gene: 608; Ensembl: ENSG00000048462; OMIM: 109545; UniProtKB: Q02223.

CD19 is also referred to as CD19 Molecule; T-Cell Surface Antigen Leu-12; CD19 Antigen; CVID3; Differentiation Antigen CD19; B4; B-Lymphocyte Surface Antigen B4; B-Lymphocyte Antigen CD19. Ids: HGNC: 1633; Entrez Gene: 930; Ensembl: ENSG00000177455; OMIM: 107265; UniProtKB: P15391.

CD20 is also referred to as Membrane-Spanning 4-Domains, Subfamily A, Member 1 (MS4A1); MS4A2; CD20; S7; Leukocyte Surface Antigen Leu-16; B-Lymphocyte Antigen CD20; Bp35; B-Lymphocyte Cell-Surface Antigen B1; CD20 Antigen; CD20 Receptor; CVID5; B-Lymphocyte Surface Antigen B1; B1; Membrane-Spanning 4-Domains Subfamily A Member 1; LEU-16. Ids: HGNC: 7315; Entrez Gene: 931; Ensembl: ENSG00000156738; OMIM: 112210; UniProtKB: P11836.

CD30 is also referred to as Tumor Necrosis Factor Receptor Superfamily, Member 8 (TNFRSF8); Ki-1 Antigen; CD30; Ki-1; D1S166E; Cytokine Receptor CD30; Lymphocyte Activation Antigen CD30; Tumor Necrosis Factor Receptor Superfamily Member 8; CD30L Receptor; CD30 Antigen. Ids: HGNC: 11923; Entrez Gene: 943; Ensembl: ENSG00000120949; OMIM: 153243; UniProtKB: P28908.

CD33 is also referred to as CD33 Molecule; SIGLEC-3; CD33 Antigen (Gp67); Myeloid Cell Surface Antigen CD33; Sialic Acid Binding Ig-Like Lectin 3; Siglec-3; SIGLEC3; CD33 Antigen and gp67. Ids: HGNC: 1659; Entrez Gene: 945; Ensembl: ENSG00000105383; OMIM: 159590; UniProtKB: P20138.

CD38 is also referred to as CD38 Molecule; T10; CD38 Antigen (P45); CADPr Hydrolase 1; ADP-Ribosyl Cyclase 1; ADP-Ribosyl Cyclase/Cyclic ADP-Ribose Hydrolase; NAD(+) Nucleosidase; EC 3.2.2.5; Cyclic ADP-Ribose Hydrolase 1; CD38 Antigen. Ids: HGNC: 1667; Entrez Gene: 952; Ensembl: ENSG00000004468; OMIM: 107270; UniProtKB: P28907.

CD44 is also referred to as CD44 Molecule (Indian Blood Group); IN; MDU2; CD44 Antigen (Homing Function And Indian Blood Group System); MDU3; CDW44; MIC4; CSPG8; Chondroitin Sulfate Proteoglycan 8; HCELL; Hematopoietic Cell E- And L-Selectin Ligand; MC56; Extracellular Matrix Receptor III; Pgp1; Heparan Sulfate Proteoglycan; Cell Surface Glycoprotein CD44; Hyaluronate Receptor; epican; Phagocytic Glycoprotein 1; Homing Function And Indian Blood Group System; ECMR-III; CDw44; HUTCH-I; Epican; LHR; PGP-1; CD44 Antigen; PGP-I; CP90 Lymphocyte Homing/Adhesion Receptor; Phagocytic Glycoprotein I; Hermes Antigen. Ids: HGNC: 1681; Entrez Gene: 960; Ensembl: ENSG00000026508; OMIM: 107269; UniProtKB: P16070.

CD123 is also referred to as Cell Division Cycle 123; Cell Division Cycle 123 Homolog; C10orf7; Cell Division Cycle Protein 123 Homolog; D123; Protein D123; HT-1080; CCEP123; PZ32; CEP89; Cell Division Cycle 123 Homolog (S. Cerevisiae); FLJ14640; Chromosome 10 Open Reading Frame 7. Ids: HGNC: 16827; Entrez Gene: 8872; Ensembl: ENSG00000151465; OMIM: 615470; UniProtKB: 075794.

CD138 is also referred to as Syndecan 1 (SCD1); CD138; SDC; Heparan Sulfate Proteoglycan Fibroblast Growth Factor Receptor; Syndecan Proteoglycan 1; syndecan; SYND1; syndecan-1; CD138 Antigen. Ids: HGNC: 10658; Entrez Gene: 6382; Ensembl: ENSG00000115884; OMIM: 186355; UniProtKB: P18827.

CEA is also referred to as Carcinoembryonic Antigen-Related Cell Adhesion Molecule 5 (CEACAM5); Meconium Antigen 100; CD66e; Carcinoembryonic Antigen; CD66e Antigen. Ids: HGNC: 1817; Entrez Gene: 1048; Ensembl: ENSG00000105388; OMIM: 114890; UniProtKB: P06731.

EGFR is also referred to as Epidermal Growth Factor Receptor; Erythroblastic Leukemia Viral (V-Erb-B) Oncogene Homolog (Avian); ERBB1; PIG61; Proto-Oncogene C-ErbB-1; Avian Erythroblastic Leukemia Viral (V-Erb-B) Oncogene Homolog; Receptor Tyrosine-Protein Kinase ErbB-1; Cell Growth Inhibiting Protein 40; Cell Proliferation-Inducing Protein 61; HER1; mENA; EC 2.7.10.1; EC 2.7.10; Epidermal Growth Factor Receptor (Avian Erythroblastic Leukemia Viral (V-Erb-B) Oncogene Homolog). Ids: HGNC: 3236; Entrez Gene: 1956; Ensembl: ENSG00000146648; OMIM: 131550; UniProtKB: P00533.

EGFRvIII is a common variant of EGFR (Oncogene. 2013 May 23; 32(21):2670-81. doi: 10.1038/one.2012.280. Epub 2012 Jul. 16).

Delta like 3 (DLL3) is also referred to as Delta-Like 3); Drosophila Delta Homolog 3; Delta3; Delta (Drosophila)-Like 3; SCDO1. Ids for DLL3 are: HGNC: 2909; Entrez Gene: 10683; Ensembl: ENSG00000090932; OMIM: 602768 and UniProtKB: Q9NYJ7.

LGR5 is Leucine-Rich Repeat Containing G Protein-Coupled Receptor 5 Alternative names for the gene or protein are Leucine-Rich Repeat Containing G Protein-Coupled Receptor 5; Leucine-Rich Repeat-Containing G Protein-Coupled Receptor 5; G-Protein Coupled Receptor HG38; G-Protein Coupled Receptor 49; G-Protein Coupled Receptor 67; GPR67; GPR49; Orphan G Protein-Coupled Receptor HG38; G Protein-Coupled Receptor 49; GPR49; HG38 and FEX. A protein or antibody of the invention that binds LGR5, binds human LGR5. The LGR5 binding protein or antibody of the invention may, due to sequence and tertiary structure similarity between human and other mammalian orthologs, also bind such an ortholog but not necessarily so. Database accession numbers for the human LGR5 protein and the gene encoding it are (NC_000012.12; NT_029419.13; NC_018923.2; NP_001264155.1; NP_001264156.1; NP_003658.1).

MSLN or mesothelin is also referred to as Mesothelin; Pre-Pro-Megakaryocyte-Potentiating Factor; CAK1 Antigen; MPF; Soluble MPF Mesothelin Related Protein; Megakaryocyte Potentiating Factor and SMRP. Ids for MSLN are: HGNC: 7371; Entrez Gene: 10232; Ensembl: ENSG00000102854; OMIM: 601051; UniProtKB: Q13421.

Folate receptor 1 is also referred to as FOLR1; Folate Receptor 1; Ovarian Tumor-Associated Antigen MOv18; Adult Folate-Binding Protein; Folate Receptor, Adult; KB Cells FBP; FR-Alpha; FOLR; FBP; Folate Binding Protein; and Folate Receptor 1. Ids for FOLR1 are HGNC: 3791; Entrez Gene: 2348; Ensembl: ENSG00000110195; OMIM: 136430; UniProtKB: P15328.

Folate receptor 3 is also referred to as FOLR3; Folate Receptor 3 (Gamma); FR-Gamma; Folate Receptor 3; Gamma-HFR; and FR-G. Ids for FOLR3 are HGNC: 3795; Entrez Gene: 2352; Ensembl: ENSG00000110203; OMIM: 602469; and UniProtKB: P41439.

EPCAM is also referred to as Epithelial Cell Adhesion Molecule; EGP40; M4S1; ESA; MIC18; KS1/4; Tumor-Associated Calcium Signal Transducer 1; MK-1; TACSTD1; Human Epithelial Glycoprotein-2; TROP1; Membrane Component, Chromosome 4, Surface Marker (35 kD Glycoprotein); Adenocarcinoma-Associated Antigen; EGP; Cell Surface Glycoprotein Trop-1; Ep-CAM; Epithelial Glycoprotein 314; GA733-2; Major Gastrointestinal Tumor-Associated Protein GA733-2; M1S2; EGP314; CD326 Antigen; KSA; Epithelial Cell Surface Antigen; DIAR5; Epithelial Glycoprotein; HNPCC8; hEGP314; Antigen Identified By Monoclonal Antibody AUA1; KS 1/4 Antigen; EGP-2; ACSTD1. Ids: HGNC: 11529; Entrez Gene: 4072; Ensembl: ENSG00000119888; OMIM: 185535; UniProtKB: P16422.

HER2 is also referred to as V-Erb-B2 Avian Erythroblastic Leukemia Viral Oncogene Homolog 2; ERBB2; CD340; NGL; HER-2; HER-2/neu2; NEU2; TKR1; Neuro/Glioblastoma Derived Oncogene Homolog; C-Erb B2/Neu Protein; Metastatic Lymph Node Gene 19 Protein; herstatin; Proto-Oncogene C-ErbB-2; Neuroblastoma/Glioblastoma Derived Oncogene Homolog; Proto-Oncogene Neu; Receptor Tyrosine-Protein Kinase ErbB-2; Tyrosine Kinase-Type Cell Surface Receptor HER2; V-Erb-B2 Erythroblastic Leukemia Viral Oncogene Homolog 2, Neuro/Glioblastoma Derived Oncogene Homolog; MLN 19; MLN19; p185erbB2; CD340 Antigen; EC 2.7.10.1; EC 2.7.10; V-Erb-B2 Avian Erythroblastic Leukemia Viral Oncogene Homolog 2 (Neuro/Glioblastoma Derived Oncogene Homolog). Ids:

HGNC: 3430; Entrez Gene: 2064; Ensembl: ENSG00000141736; OMIM: 164870; UniProtKB: P04626.

HM1.24 is also referred to as BST2; Bone Marrow Stromal Cell Antigen 2; TETHERIN; BST-2; Bone Marrow Stromal Antigen 2; HM1.24 Antigen; Tetherin; CD317; CD317 Antigen; NPC-A-7. Ids: HGNC: 1119; Entrez Gene: 684; Ensembl: ENSG00000130303; OMIM: 600534; UniProtKB: Q10589.

MCSP is also referred to as Sperm Mitochondria-Associated Cysteine-Rich Protein (SMCP); MCSP; MCS; Mitochondrial Capsule Selenoprotein; HSMCSGEN1; Sperm Mitochondrial-Associated Cysteine-Rich Protein. Ids: HGNC: 6962; Entrez Gene: 4184; Ensembl: ENSG00000163206; OMIM: 601148; UniProtKB: P49901.

PD-L1 is a type 1 transmembrane protein that plays a role in suppressing an immune response during particular events such as pregnancy, tissue allografts, autoimmune disease and other disease states such as hepatitis. The binding of PDL1 to PD-1 or B7.1 (CD80) transmits an inhibitory signal which reduces the proliferation of the PD-1 expressing T cells. PD-1 is thought to be able to control the accumulation of foreign antigen specific T cells through apoptosis. PD-L1 is expressed by a variety of cancer cells and the expression thereof is thought to be at least in part responsible for a dampening of an immune response against the cancer cell. PD-L1 is a member of the B7-family of protein and is known under a variety of other names such as CD274 Molecule; CD274 Antigen; B7 Homolog 1; PDCD1 Ligand 1; PDCD1LG1; PDCD1L1; B7H1; PDL1; Programmed Cell Death 1 Ligand 1; Programmed Death Ligand 1; B7-H1; and B7-H. External Ids for CD274 are HGNC: 17635; Entrez Gene: 29126; Ensembl: ENSG00000120217; OMIM: 605402; UniProtKB: Q9NZQ7.

PSMA is also referred to as Folate Hydrolase (Prostate-Specific Membrane Antigen) 1; FOLH1; NAALAD1; FOLH; mGCP; Glutamate Carboxypeptidase II; N-Acetylated-Alpha-Linked Acidic Dipeptidase I; PSM; NAALADase I; PSMA; EC 3.4.17.21; Glutamate Carboxylase II; GCP2; Cell Growth-Inhibiting Gene 27 Protein; NAALAdase; Folylpoly-Gamma-Glutamate Carboxypeptidase; Glutamate Carboxypeptidase 2; Membrane Glutamate Carboxypeptidase; N-Acetylated Alpha-Linked Acidic Dipeptidase 1; Pteroylpoly-Gamma-Glutamate Carboxypeptidase; Prostate Specific Membrane Antigen Variant F; FGCP; Folate Hydrolase 1; GCPII; Prostate-Specific Membrane Antigen. Ids: HGNC: 3788; Entrez Gene: 2346; Ensembl: ENSG00000086205; OMIM: 600934; UniProtKB: Q04609.

PSMA is not to be confused with Proteasome (Prosome, Macropain) Subunit, Alpha Type, 1 which is also known under the alias PSMA1.

Accession numbers are primarily given to provide a further method of identification of a target, the actual sequence of the protein bound may vary, for instance because of a mutation in the encoding gene such as those occurring in some cancers or the like. The antigen binding site binds the antigen and a variety of variants thereof, such as those expressed by some antigen positive immune or tumor cells.

When herein reference is made to a gene, a protein, the reference is preferably to the human form of the gene or protein. When herein reference is made to a gene or protein reference is made to the natural gene or protein and to variant forms of the gene or protein as can be detected in tumours, cancers and the like, preferably as can be detected in human tumours, cancers and the like.

A bispecific or multispecific antibody of the invention preferably binds to the human BCMA, CD19, CD20, CD30, CD33, CD38, CD44, CD123, CD138, CEA, CLEC12A, CS-1, EGFR, EGFRVIII, EPCAM, DLL3, LGR5, MSLN, FOLR1, FOLR3, HER2, HM1.24, MCSP, PD-L1, PSMA protein or a variant thereof. The antigen binding heavy/light chain combination preferably binds the extracellular part of the antigen. A bispecific antibody according to the invention preferably binds to human CLEC12A or a variant thereof. A preferred bispecific antibody according to the invention binds to human CD3 and human CLEC12A or a variant thereof. In a preferred embodiment, the multispecific antibody binds to CD3, PD-L1 and EGFR.

HGNC stands for the HUGO Gene nomenclature committee. The number following the abbreviation is the accession number with which information on the gene and protein encoded by the gene can be retrieved from the HGNC database. Entrez Gene provides the accession number or gene ID with which information on the gene or protein encoded by the gene can be retrieved from the NCBI (National Center for Biotechnology Information) database. Ensemble provides the accession number with which information on the gene or protein encoded by the gene can be obtained from the Ensemble database. Ensembl is a joint project between EMBL-EBI and the Wellcome Trust Sanger Institute to develop a software system which produces and maintains automatic annotation on selected eukaryotic genomes.

The invention provides an antigen-binding protein, preferably an antibody, that binds human CD3 comprising an antibody variable domain comprising a heavy chain variable region and a light chain variable region wherein the heavy chain variable region comprises a CDR1, CDR2 and CDR3
comprising the amino acid sequence:

CDR1:
(SEQ ID NO 42)
SFGIS;
CDR2:
(SEQ ID NO 43)
GFIPVLGTANYAQKFQG;
CDR3:
(SEQ ID NO 44)
RGNWNPFDP

or
comprising the amino acid sequence:

CDR1:
(SEQ ID NO 6)
SX₁TFTIS;
CDR2:
(SEQ ID NO 7)
GIIPX₂FGTITYAQKFQG;
CDR3:
(SEQ ID NO 44)
RGNWNPFDP;

wherein

X₁=K or R;X₂=L or I.

In a preferred embodiment X₁=K; and X₂=L. In another preferred embodiment X₁=R; and X₂=I.

The invention provides an antigen-binding protein, preferably an antibody, that binds human CD3 comprising an antibody variable domain comprising a heavy chain variable region and a light chain variable region wherein the heavy chain variable region comprises a CDR1, CDR2 and CDR3 comprising the amino acid sequence:
CDR1: SKTLTIS; (SEQ ID NO 84) CDR2: GIIPIFGSITYAQKFQD (SEQ ID NO 85); CDR3: RGNWNPFDP (SEQ ID NO 86); or
   comprising the amino acid sequence:

CDR1:
(SEQ ID NO 100)
GSGIS;
CDR2:
(SEQ ID NO 101)
GFIPFFGSANYAQKFRD;
CDR3:
(SEQ ID NO 8)
RGNWNPX₁₃DP;

wherein

X₁₃= or L or F.

The invention further provides an antigen-binding protein, preferably an antibody, that binds human CD3 comprising an antibody variable domain comprising a heavy chain variable region and a light chain variable region wherein the heavy chain variable region comprises a CDR1, CDR2 and CDR3 comprising the amino acid sequence:

CDR1:
(SEQ ID NO 9)
RX₃WIG;
CDR2:
(SEQ ID NO 200)
IIYPGDSDTRYSPSFQG;
CDR3:
(SEQ ID NO 10)
X₄IRYFX₅WSEDYHYYX₆DV;

wherein

X₃=F or Y; X₄=H or N; X₅=D or V; and X₆=L or M.

In a preferred embodiment X₃=F; X₄=H; X₅=D; and X₆=L; or X₃=Y; X₄=N; X₅=V; and X₆=M.

The invention further provides an antigen-binding protein, preferably an antibody, that binds human CD3 comprising an antibody variable domain comprising a heavy chain variable region and a light chain variable region wherein the heavy chain variable region comprises a CDR1, CDR2 and CDR3 comprising the amino acid sequence:

CDR1:
(SEQ ID NO 117)
SYALS;
CDR2:
(SEQ ID NO 118)
GISGSGRTTWYADSVKG;
CDR3:
(SEQ ID NO 119)
DGGYSYGPYWYFDL.

The invention further provides an antigen-binding protein, preferably an antibody, that binds human CD3 comprising an antibody variable domain comprising a heavy chain variable region and a light chain variable region wherein the heavy chain variable region comprises a CDR1, CDR2 and CDR3 comprising the amino acid sequence:

CDR1:
(SEQ ID NO 117)
SYALS;
CDR2:
(SEQ ID NO 126)
AISGSGRTTWYADSVKG;
CDR3:
(SEQ ID NO 127)
DGGYTYGPYWYFDL.

The invention further provides an antigen-binding protein, preferably an antibody, that binds human CD3 comprising an antibody variable domain comprising a heavy chain variable region and a light chain variable region wherein the heavy chain variable region comprises a CDR1, CDR2 and CDR3 comprising the amino acid sequence:

CDR1:
(SEQ ID NO 2)
DYTMH;
CDR2:
(SEQ ID NO 143)
DISWSSGSIGYADSVKG;
CDR3:
(SEQ ID NO 144)
DHRGYGDYEGGGFDY.

The invention further provides an antigen-binding protein, preferably an antibody, that binds human CD3 comprising an antibody variable domain comprising a heavy chain variable region and a light chain variable region wherein the heavy chain variable region comprises a CDR1, CDR2 and CDR3 comprising the amino acid sequence:

CDR1:
(SEQ ID NO 142)
DYTMH;
CDR2:
(SEQ ID NO 11)
DISWSX₇GX₈X₉X₁₀YADSVKG;
CDR3:
(SEQ ID NO 12)
DHX₁₁GYGDYEGGGFDX₁₂;

wherein
X₇=S or G;
X₈=S or T;
X₉=I or T;
X₁₀=G or Y;
X₁₁=R or M;
X₁₂=H or Y,
preferably X₇, X₈, X₉ and X₁₀ are S, S, I and G or G, S, I and Y or S, T, T and G, and preferably X₁₁ and X₁₂ are R and H, or R and Y, or M and Y, more preferably X₇, X₈, X₉, X₁₀, X₁₁ and X₁₂ are S, S, I, G, R and H or G, S, I, Y, R and Y or S, T, T, G, M and Y, or, in other words, preferably X₇, X₈, X₉ and X₁₀ are S, S, I and G, and X₁₁ and X₁₂ are R and H; or X₇, X₈, X₉ and X₁₀ are G, S, I and Y, and X₁₁ and X₁₂ are R and Y; or X₇, X₈, X₉ and X₁₀ are S, T, T and G, and X₁₁ and X₁₂ are M and Y.

In a preferred embodiment the light chain variable region comprises the amino acid sequence of an IgVκ1-39*01 gene segment as depicted in FIG. 11A with 0-10, preferably 0-5 amino acid variations, insertions, deletions, substitutions, additions or a combination thereof. The amino acid sequence of the IgVκ1-39*01 is depicted in FIG. 11A. IgVκ1-39 is short for Immunoglobulin Variable Kappa 1-39 Gene. The gene is also known as Immunoglobulin Kappa Variable 1-39; IGKV139; IGKV1-39. External Ids for the gene are HGNC: 5740; Entrez Gene: 28930; Ensembl: ENSG00000242371. A preferred amino acid sequence for IgVκ1-39 is given in FIG. 11A. This lists the sequence of the V-region. The V-region can be combined with one of five J-regions. FIGS. 11B and 11C describe two preferred sequences for IgVκ1-39 in combination with a J-region. The joined sequences are indicated as IGKV1-39/jk1 and IGKV1-39/jk5; alternative names are IgVκ1-39*01/IGJκ1*01 or IgVκ1-39*01/IGJκ5*01 (nomenclature according to the IMGT database worldwide web at imgt.org).

It is preferred that the IgVκ1-39*01 comprising light chain variable region is a germline sequence. It is further preferred that the IGJκ1*01 or/IGJκ5*01 comprising light chain variable region is a germline sequence. In a preferred embodiment, the IGKV1-39/jk1 or IGKV1-39/jk5 light chain variable regions are germline sequences.

In a preferred embodiment the light chain variable region comprises a germline IgVκ1-39*01. In a preferred embodiment the light chain variable region comprises the kappa light chain IgVκ1-39*01/IGJκ1*01 or IgVκ1-39*01/IGJκ5*01. In a preferred embodiment a IgVκ1-39*01/IGJκ1*01. The light chain variable region preferably comprises a germline kappa light chain IgVκ1-39*01/IGJκ1*01 or germline kappa light chain IgVκ1-39*01/IGJκ5*01, preferably a germline IgVκ1-39*01/IGJκ1*01.

Mature B-cells that produce an antibody with a light chain often produce a light chain that has undergone one or more mutations with respect to the germline sequence, i.e. the normal sequence in non-lymphoid cells of the organism. The process that is responsible for these mutations is often referred to as somatic (hyper) mutation. The resulting light chain is referred to as an affinity matured light chain. Such light chains, when derived from a germline IgVκ1-39*01 sequence are IgVκ1-39*01 derived light chains. In this specification, the phrase “IgVκ1-39*01” will include IgVκ1-39*01-derived light chains, The mutations that are introduced by somatic hypermutation can also be introduced artificially in the lab. In the lab also other mutations or variations to a light chain can be introduced without affecting the properties of the light chain in kind, not necessarily in amount. A light chain is at least an IgVκ1-39*01 light chain if it comprises a sequence as depicted in FIG. 11A, FIG. 11D or FIG. 11E with 0-10, preferably 0-5 amino acid variations, insertions, deletions, substitutions, additions or a combination thereof. In a preferred embodiment the IgVκ1-39*01 light chain is a light chain comprising a sequence as depicted in FIG. 11A, FIG. 11B or FIG. 11C with 0-9, 0-8, 0-7, 0-6, 0-5, 0-4 amino acid variations, insertions, deletions, substitutions, additions or a combination thereof. In a preferred embodiment the IgVκ1-39*01 light chain is a light chain comprising a sequence as depicted in FIG. 11A, FIG. 11B or FIG. 11C with 0-5, preferably 0-4, more preferably 0-3 amino acid variations, insertions, deletions, substitutions, additions or a combination thereof. In a preferred embodiment the IgVκ1-39*01 light chain is a light chain comprising a sequence as depicted in FIG. 11A, FIG. 11B or FIG. 11C with 0-2, more preferably 0-1, most preferably 0 amino acid variations, insertions, deletions, substitutions, additions or a combination thereof. In a preferred embodiment the IgVκ1-39*01 light chain is a light chain comprising a sequence as depicted in FIG. 11A or FIG. 11B with the mentioned amino acid variations, insertions, deletions, substitutions, additions or a combination thereof. In a preferred embodiment the light chain comprises the sequence of FIG. 11B.

The light chain preferably comprises a common light chain variable region. Said common light chain variable region preferably comprises an IgVκ1-39 light chain variable region. Said light chain variable region is preferably a germline IgVκ1-39*01 variable region. Said light chain variable region preferably comprises the kappa light chain IgVκ1-39*01/IGJκ1*01 or IgVκ1-39*01/IGJκ5*01. The light chain variable region preferably comprises the germline kappa light chain IgVκ1-39*01/IGJκ1*01 or IgVκ1-39*01/IGJκ5*01. Said light chain variable region preferably comprises the amino acid sequence DIQMT QSPSS LSASV GDRVT ITCRA SQSIS SYLNW YQQKP GKAPK LLIYA ASSLQ SGVPS RFSGS GSGTD FTLTI SSLQP EDFAT YYCQQ SYSTP PTFGQ GTKVE IK (SEQ ID NO 16) or DIQMT QSPSS LSASV GDRVT ITCRA SQSIS SYLNW YQQKP GKAPK LLIYA ASSLQ SGVPS RFSGS GSGTD FTLTI SSLQP EDFAT YYCQQ SYSTP PITFG QGTRL EIK (SEQ ID NO 19) with 0-5 amino acid variations, insertions, deletions, substitutions, additions or a combination thereof.

The light chain variable region preferably comprises a CDR1, CDR2, and CDR3 region comprising the amino acid sequence CDR1-QSISSY (SEQ ID NO 21), CDR2-AAS (SEQ ID NO 22), CDR3-QQSYSTP (SEQ ID NO 23), i.e. the CDRs of IGKV1-39 (according to IMGT). The amino acid variations, insertions, deletions, substitutions, additions or combination thereof are preferably not in the CDR3 region of the light chain variable region, preferably not in the CDR1 or CDR2 region of the light chain variable region. In a preferred embodiment the light chain variable region does not comprise a deletion, addition or variations, insertion with respect to the sequence indicated. In this embodiment the light chain variable region can have 0-5 amino acid substitutions with respect to the indicated amino acid sequence. An amino acid substitution is preferably a conservative amino acid substitution. The CDR1, CDR2 and CDR3 of a light chain of an antibody of the invention preferably comprises respectively the amino acid sequence CDR1-QSISSY (SEQ ID NO 21), CDR2-AAS (SEQ ID NO 22), CDR3-QQSYSTP (SEQ ID NO 23), i.e. the CDRs of IGKV1-39 (according to IMGT).

The antigen-binding protein is preferably an antibody, preferably a bispecific or multispecific antibody. The antibody preferably comprises a common light chain including a common light variable region as defined herein and a light chain constant region as defined herein.

An antibody of the invention is, as mentioned, preferably a bispecific antibody. A “bispecific antibody” is an antibody as described herein wherein one domain of the antibody binds to a first antigen whereas a second domain of the antibody binds to a second antigen, wherein said first and second antigens are not identical. The term “bispecific antibody” also encompasses antibodies wherein one heavy chain variable region/light chain variable region (VH/VL) combination binds a first epitope on an antigen and a second VH/VL combination that binds a second epitope. The term further includes antibodies wherein VH is capable of specifically recognizing a first antigen and the VL, paired with the VH in an immunoglobulin variable region, is capable of specifically recognizing a second antigen. The resulting VH/VL pair will bind either antigen 1 or antigen 2. Such so called “two-in-one antibodies”, described in for instance WO 2008/027236, WO 2010/108127 and Schaefer et al (Cancer Cell 20, 472-486, October 2011). A bispecific antibody according to the present invention is not limited to any particular bispecific format or method of producing it.

The bispecific antibody preferably has one heavy chain variable region/light chain variable region (VH/VL) combination that binds CD3 and a second VH/VL combination that binds an antigen other than an antigen on CD3. In a preferred embodiment the antigen is a tumor antigen. In a preferred embodiment the VL in said first VH/VL combination is similar to the VL in said second VH/VL combination. In a more preferred embodiment, the VLs in the first and second VH/VL combinations are identical. In a preferred embodiment, the bispecific antibody is a full length antibody which has one heavy/light (H/L) chain combination that binds CD3 and one H/L chain combination that binds another antigen, preferably a tumor antigen. In a preferred embodiment the light chain in said first H/L chain combination is similar to the light chain in said second H/L chain combination. In a more preferred embodiment, the light chains in the first and second H/L chain combinations are identical, i.e. a similar or identical human light chain is a so-called ‘common light chain’, which is a light chain that can combine with different heavy chains to form antibodies with functional antigen binding domains. In a preferred embodiment the light chain in said first H/L chain combination comprises a light chain variable region that is similar to the light chain variable region in said second H/L chain combination. In a more preferred embodiment, the light chain variably regions in the first and second H/L chain combinations are identical, i.e. a similar or identical human light chain variable region is a so-called ‘common light chain variable region’, which is a light chain variable region that can combine with different heavy chain variable regions to form antibodies with functional antigen binding domains. The light chain comprising a common light chain variable region is preferably a common light chain. The common light chain of the bispecific antibody is preferably an IgVκ1-39 light chain as indicated herein above.

The invention also provides alternative bispecific formats, such as those described in Spiess, C., et al., (Alternative molecular formats and therapeutic applications for bispecific antibodies. Mol. Immunol. (2015) dx.doi.org/10.1016/j.molimm.2015.01.003). Bispecific antibody formats that are not classical antibodies with two H/L combinations, have at least a variable domain comprising a heavy chain variable region and a light chain variable region of the invention. This variable domain may be linked to a single chain Fv-fragment, monobody, a VHH and a Fab-fragment that provides the second binding activity.

In a bispecific antibody of the invention the light chain in the CD3-binding H/L chain combination is preferably similar to the light chain in H/L chain combination that can bind an antigen other than CD3, preferably a tumor antigen. In a more preferred embodiment, the light chain in both H/L chain combinations is identical, i.e. said human light chain is a so-called ‘common light chain’, which is a light chain that can combine with different heavy chains to form antibodies with functional antigen binding domains. Preferably, the common light chain has a germline sequence. A preferred germline sequence is a light chain variable region that is frequently used in the human repertoire and has good thermodynamic stability, yield and solubility. A preferred germline light chain is IgVκ1-39, preferably the rearranged germline human kappa light chain IgVκ1-39*01/IGJκ1*01 or a fragment or a functional equivalent (i.e. same IgVκ1-39 gene segment but different IGJκ gene segment) thereof (nomenclature according to the IMGT database worldwide web at imgt.org).

The term ‘aberrant cells’ as used herein includes tumor cells, more specifically tumor cells of hematological origin including also pre-leukemic cells such as cells that cause myelodysplastic syndromes (MDS) and leukemic cells such as acute myeloid leukemia (AML) tumor cells or chronic myelogenous leukemia (CML) cells.

The term ‘immune effector cell’ or ‘effector cell’ as used herein refers to a cell within the natural repertoire of cells in the mammalian immune system which can be activated to affect the viability of a target cell. Immune effector cells include cells of the lymphoid lineage such as natural killer (NK) cells, T cells including cytotoxic T cells, or B cells, and including cells of the myeloid lineage, such as monocytes or macrophages, dendritic cells and neutrophilic granulocytes. Hence, said effector cell is preferably an NK cell, a T cell, a B cell, a monocyte, a macrophage, a dendritic cell or a neutrophilic granulocyte. The recruitment of effector cells to aberrant cells means that immune effector cells are brought in proximity to the aberrant target cells such that the effector cells can directly kill, or indirectly initiate the killing of the aberrant cells.

As used herein, the terms “subject” and “patient” are used interchangeably and refer to a mammal such as a human, mouse, rat, hamster, guinea pig, rabbit, cat, dog, monkey, cow, horse, pig and the like (e.g., a patient, such as a human patient, having cancer).

The terms “treat,” “treating,” and “treatment,” as used herein, refer to any type of intervention or process performed on, or administering an active agent or combination of active agents to the subject with the objective of reversing, alleviating, ameliorating, inhibiting, or slowing down or preventing the progression, development, severity or recurrence of a symptom, complication, condition or biochemical indicia associated with a disease.

As used herein, “effective treatment” or “positive therapeutic response” refers to a treatment producing a beneficial effect, e.g., amelioration of at least one symptom of a disease or disorder, e.g., cancer. A beneficial effect can take the form of an improvement over baseline, including an improvement over a measurement or observation made prior to initiation of therapy according to the method. For example, a beneficial effect can take the form of slowing, stabilizing, stopping or reversing the progression of a cancer in a subject at any clinical stage, as evidenced by a decrease or elimination of a clinical or diagnostic symptom of the disease, or of a marker of cancer. Effective treatment may, for example, decrease in tumor size, decrease the presence of circulating tumor cells, reduce or prevent metastases of a tumor, slow or arrest tumor growth and/or prevent or delay tumor recurrence or relapse.

The term “therapeutic amount” refers to an amount of an agent or combination of agents that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In some embodiments, a therapeutic amount is an amount sufficient to delay tumor development. In some embodiments, a therapeutic amount is an amount sufficient to prevent or delay tumor recurrence. A therapeutic amount can be administered in one or more administrations. The therapeutic amount of the drug or composition may: (i) reduce the number of cancer cells; (ii) reduce tumor size; (iii) inhibit, retard, slow to some extent and may stop cancer cell infiltration into peripheral organs; (iv) inhibit tumor metastasis; (v) inhibit tumor growth; (vi) prevent or delay occurrence and/or recurrence of tumor; and/or (vii) relieve to some extent one or more of the symptoms associated with the cancer. In one example, an “therapeutic amount” is the amount of a CLEC12A/CD3 bispecific antibody that effects a decrease in a cancer (for example a decrease in the number of cancer cells) or slowing of progression of a cancer, such as acute myeloid leukemia, myelodysplastic syndrome or chronic myelogenous leukemia.

The invention also provides an antigen-binding protein, preferably an antibody, that binds human CD3 comprising an antibody variable domain comprising a heavy chain variable region and a light chain variable region wherein the heavy chain variable region comprises the amino acid sequence

(SEQ ID NO 45)
EVQLVQSGAEVKKPGSSVKVSCKASGGTFRSFGISWVRQAPGQGLEWMGG
FIPVLGTANYAQKFQGRVTIIADKSTNTAYMELSSLRSEDTAVYYCARRG
NWNPFDPWGQGTLVTVSS;
(SEQ ID NO 54)
QVQLVQSGAEVKKPGSSVKVSCKASGDAFKSKTFTISWVRQAPGQGLEWL
GGIIPLFGTITYAQKFQGRVTITADKSTNTAFMELSSLRSEDTAMYYCTR
RGNWNPFDPWGQGTLVTVSS;
(SEQ ID NO 63)
EVQLVQSGSELKKPGSSVKVSCKASGVTFNSRTFTISWVRQAPGQGLEWL
GSIIPIFGTITYAQKFQGRVTITADKSTSTAFMELTSLRSEDTAIYYCTR
RGNWNPFDPWGQGTLVTVSS;
(SEQ ID NO 71)
QVQLVQSGAEVKKPGSSVKVSCKASGGTFRGSGISWVRQAPGQGLEWVGG
FIPFFGSANYAQKFRDRVTITADKSATTAYMELSSLRSEDTAIYYCAKRG
NWNPLDPWGQGTLVTVSS;
(SEQ ID NO 87)
QVQLVQSGAEVKKPGSSVKVSCKASGVTFKSKTLTISWVRQAPGQGLEWL
GGIIPIFGSITYAQKFQDRVSITADKSTNTAYLELNSLRSEDTAIYYCAR
RGNWNPFDPWGQGTLVTVSS;
or
(SEQ ID NO 103)
EVQLVQSGAEVKKPGSSVKVSCKASGGTFRGSGISWVRQAPGQGLEWVGG
FIPFFGSANYAQKFRDRVTITADKSATTAYMELSSLRSEDTAIYYCAKRG
NWNPFDPWGQGTLVTVSS

with 0-10, preferably 0-5 amino acid variations, insertions, deletions, substitutions, additions or a combination thereof at one or more positions other than the CDRs.

Further provided is an antigen-binding protein, preferably an antibody, that binds human CD3 comprising an antibody variable domain comprising a heavy chain variable region and a light chain variable region wherein the heavy chain variable region comprises the amino acid sequence

(SEQ ID NO 298)
EVQLVQSGAEVKKPGESLKISCKGSGYSFTRFWIGWVRQMPGKGLEWMGI
IYPGDSDTRYSPSFQGQVTISADKSTSTAYLQWSSLKASDTGMYYCVRHI
RYFDWSEDYHYYLDVWGKGTTVTVSS;
or
(SEQ ID NO 211)
EVQLVESGAEVKKPGESLKISCKGSGYSFTRYWIGWVRQMPGKGLEWMGI
IYPGDSDTRYSPSFQGQVTISADKSISTAYLQWSSLKASDTAMYYCVRNI
RYFVWSEDYHYYMDVWGKGTTVTVSS;

with 0-10, preferably 0-5 amino acid variations, insertions, deletions, substitutions, additions or a combination thereof at one or more positions other than the CDRs.

Also provided is an antigen-binding protein, preferably an antibody, that binds human CD3 comprising an antibody variable domain comprising a heavy chain variable region and a light chain variable region wherein the heavy chain variable region comprises the amino acid sequence

(SEQ ID NO 120)
QVQLVQSGGGLVQPGGSLRLSCATSGFKFSSYALSWVRQAPGKGLEWVSG
ISGSGRTTWYADSVKGRFTISRDNSKNTLYLQMNSLRAEDTAVYYCARDG
GYSYGPYWYFDLWGRGTLVTVSS;
or
(SEQ ID NO 128)
QVQLVESGGGLVQPGGSLRLSCATSGFTFISYALSWVRQAPGKGLEWVSA
ISGSGRTTWYADSVKGRFTISRDNSKNTLFLQMNSLRAEDTAVYYCARDG
GYTYGPYWYFDLWGRGTLVTVSS

with 0-10, preferably 0-5 amino acid variations, insertions, deletions, substitutions, additions or a combination thereof at one or more positions other than the CDRs.

The invention further provides an antigen-binding protein, preferably an antibody, that binds human CD3 comprising an antibody variable domain comprising a heavy chain variable region and a light chain variable region wherein the heavy chain variable region comprises the amino acid sequence

(SEQ ID NO 145)
EVQLVESGGGLVQPGRSLRLSCATSGFNFDDYTMHWVRQAPGKGLEWVSD
ISWSSGSIGYADSVKGRFTISRDNAKNSLWLQMNSLRTEDTALYFCAKDH
RGYGDYEGGGFDYWGQGTLVTVSS;
(SEQ ID NO 153)
EVQLVESGGGLVQPGRSLRLSCATSGFTFDDYTMHWVRQAPGKGLEWVSD
ISWSSGSIGYADSVKGRFTISRDNAKNSLYLQMNSLRAEDTALYFCAKDH
RGYGDYEGGGFDHWGQGTLVTVSS;
(SEQ ID NO 185)
EVQLVESGGGLVQPGRSLRLSCVTSGFTFDDYTMHWVRQAPGKGLEWVSD
ISWSSGTTGYADSVKGRFTISRDNAKDSLYLQMNSLRTEDTALYYCAKDH
MGYGDYEGGGFDYWGQGTLVTVSS;
or
(SEQ ID NO 169)
EVQLVESGGVVVQPGGSLRLSCAASGFTFDDYTMHWVRQAPGKGLEWVSD
ISWSGGSIYYADSVKGRFTISRDNSKNSLYLQMNSLRTEDTALYYCAKDH
RGYGDYEGGGFDYWGRGTLVTVSS;

with 0-10, preferably 0-5 amino acid variations, insertions, deletions, substitutions, additions or a combination thereof at one or more positions other than the CDRs.

The amino acid variations, insertions, deletions, substitutions, additions or combination thereof are preferably not in the CDR3 region of the heavy chain variable region, preferably not in the CDR1 and/or CDR2 region of the heavy chain variable region. In a preferred embodiment the heavy chain variable region does not comprise a deletion, addition or variation, insertion with respect to the sequence indicated. In one embodiment the heavy chain variable region can have 0-10, preferably 0-5 amino acid substitutions with respect to the indicated amino acid sequence. In a preferred embodiment the heavy chain variable region comprises 0-9, 0-8, 0-7, 0-6, 0-5, 0-4, preferably 0-3, preferably 0-2, preferably 0-1 and preferably 0 amino acid variations, insertions, deletions, substitutions, additions with respect to the indicated amino acid sequence, or a combination thereof at positions other than the CDRs. A combination of an insertion, addition, deletion or substitution is a combination as claimed if aligned sequences do not differ at more than 10, preferably no more than 5 positions. A gap in one of the aligned sequences counts for as many amino acids as skipped in the other sequence. An amino acid substitution, if any, is preferably a conservative amino acid substitution.

The invention further provides an antigen-binding protein, preferably an antibody, that binds human CD3 comprising an antibody variable domain comprising a heavy chain variable region and a light chain variable region wherein the heavy chain variable region comprises the amino acid sequence of MF8057; MF8058 or MF8078 as depicted in FIG. 13.

The invention further provides an antigen-binding protein, preferably an antibody, that binds human CD3 comprising an antibody variable domain comprising a heavy chain variable region and a light chain variable region wherein the heavy chain variable region comprises the amino acid sequence of MF8397 as depicted in FIG. 13.

The invention further provides an antigen-binding protein, preferably an antibody, that binds human CD3 comprising an antibody variable domain comprising a heavy chain variable region and a light chain variable region wherein the heavy chain variable region comprises the amino acid sequence of MF8508 as depicted in FIG. 13.

The invention further provides an antigen-binding protein, preferably an antibody, that binds human CD3 comprising an antibody variable domain comprising a heavy chain variable region and a light chain variable region wherein the heavy chain variable region comprises the amino acid sequence of MF9249 or MF9267 as depicted in FIG. 13.

The light chain preferably comprises the CDR1, CDR2 and CDR3 region as defined elsewhere herein. It preferably comprises a common light chain variable region and preferably a common light chain as defined elsewhere herein. The bispecific antibody preferably further comprises a heavy chain and light chain combination that binds another antigen, preferably a tumor antigen. The light chain of the heavy chain and light chain combination that binds another antigen is preferably a common light chain as defined elsewhere herein. The heavy chain of the heavy chain and light chain combination that binds another antigen preferably comprises a heavy chain variable region comprising an amino acid sequence: MF8233 (EGFR) QVQLVQSGAEVKKPGASVKVSCKASGYTFTSYGISWVRQAPGQGLEWMGWISAYNANTNYA QKLQGRVTMTTDTSTSTAYMELRSLRSDDTAVYYCAKDRHWHWWLDAFDYWGQGTLVTVSS (SEQ ID NO 247) with 0-10, preferably 0-5 amino acid variations, insertions, deletions, substitutions, additions or a combination thereof at one or more positions other than the CDRs; or MF4327 (CLEC12A) QVQLVQSGAEVKKPGASVKVSCKASGYTFTSYYMHWVRQAPGQGLEWMGIINPSGGSTSYA QKFQGRVTMTRDTSTSTVYMELSSLRSEDTAVYYCAKGTTGDWFDYWGQGTLVTVSS (SEQ ID NO 293) with 0-10, preferably 0-5 amino acid variations, insertions, deletions, substitutions, additions or a combination thereof at one or more positions other than the CDRs.

Variable domains that bind CLEC12A that have a heavy chain variable region and a common light chain region as defined herein are described among others in WO2014/051433 and WO2017/010874 which are specifically referred to for this purpose herein and which are incorporated by reference herein. The heavy chain variable region of the heavy/light chain combination that binds human EGFR or CLEC12A can have 0-10, preferably 0-5 amino acid variations, insertions, deletions, substitutions, additions with respect to the indicated amino acid sequence, or a combination thereof. In a preferred embodiment the heavy chain variable region comprises 0-9, 0-8, 0-7, 0-6, 0-5, 0-4, preferably 0-3, preferably 0-2, preferably 0-1 and preferably 0 amino acid variations, insertions, deletions, substitutions, additions with respect to the indicated amino acid sequence, or a combination thereof. A combination of an insertion, deletion, addition or substitution is a combination as claimed if aligned sequences do not differ at more than 5 positions. A gap in one of the aligned sequences counts for as many amino acid as skipped in the other sequence.

An amino acid variation, insertion, deletion, substitution, addition or combination thereof is preferably not done/present in the binding interface of the heavy and light chain.

If an amino acid is changed in the interface of the H/L chain interaction, it is preferred that the corresponding amino acids in the other chain are changed to accommodate the change. An insertion or addition of an amino acid preferably does not entail the insertion or addition of a proline.

An addition of an amino acid can in principle be regarded to be the same as an insertion. Adding an amino acid to one of the ends of a polypeptide chain is sometimes not considered an insertion but as a strict addition (prolongation). For the present invention both an addition within a chain or to one of the ends, are considered to be an insertion.

The amino acid variations, insertions, deletions, substitutions, additions or combination thereof are preferably not in the CDR3 region of the heavy chain variable region, preferably not in the CDR1 or CDR2 region of the heavy chain variable region. In a preferred embodiment the heavy chain variable region does not comprise a deletion, addition or variations, insertion with respect to the sequence indicated. In this embodiment the heavy chain variable region can have 0-5 amino acid substitutions with respect to the indicated amino acid sequence. An amino acid substitution is preferably a conservative amino acid substitution. The CDR1, CDR2 and CDR3 of a CD3 binding VH of the invention preferably comprises a CDR1, CD2 and CDR3 combination of a CD3 binding VH depicted in FIG. 13, preferably of the VH of one of MF8057; MF8058; MF8078; MF8397; MF8508; MF9249 or MF9267.

The constant region of an antibody of the present invention, including a bispecific or multispecific antibody, is preferably a human constant region. The constant region may contain one or more, preferably not more than 10, preferably not more than 5 amino-acid differences with the constant region of a naturally occurring human antibody. Various variable domains of antibodies produced herein are derived from a human antibody variable domain library. As such these variable domains are human. The unique CDR regions may be derived from humans, be synthetic or derived from another organism. An antibody or bispecific antibody of the invention is preferably a human or humanized antibody. Suitable heavy chain constant regions are non-limitingly exemplified in FIG. 12.

In the art various methods exist to produce antibodies. Antibodies are typically produced by a cell that expresses nucleic acid encoding the antibody. Suitable cells for antibody production are a hybridoma cell, a Chinese hamster ovary (CHO) cell, an NSO cell or a PER-C6 cell. In a particularly preferred embodiment said cell is a CHO cell.

Various institutions and companies have developed cell lines for the large scale production of antibodies, for instance for clinical use. Non-limiting examples of such cell lines are CHO cells, NSO cells or PER.C6 cells. These cells are also used for other purposes such as the production of proteins. Cell lines developed for industrial scale production of proteins and antibodies are herein further referred to as industrial cell lines. In a preferred embodiment the invention provides an industrial cell line that produces and an antibody of the invention.

The invention in one embodiment provides a cell comprising an antibody according to the invention and/or a nucleic acid according to the invention. Said cell is preferably an animal cell, more preferably a mammal cell, more preferably a primate cell, most preferably a human cell. For the purposes of the invention a suitable cell is any cell capable of comprising and preferably of producing an antibody according to the invention and/or a nucleic acid according to the invention.

The invention further provides a cell comprising an antibody according to the invention. Preferably said cell (typically an in vitro, isolated or recombinant cell) produces said antibody. In a preferred embodiment said cell is a hybridoma cell, a Chinese hamster ovary (CHO) cell, an NSO cell or a PER.C6 cell. In a particularly preferred embodiment said cell is a CHO cell. Further provided is a cell culture comprising a cell according to the invention. Various institutions and companies have developed cell lines for the large scale production of antibodies, for instance for clinical use. Non-limiting examples of such cell lines are CHO cells, NSO cells or PER.C6 cells. These cells are also used for other purposes such as the production of proteins. Cell lines developed for industrial scale production of proteins and antibodies are herein further referred to as industrial cell lines. Thus in a preferred embodiment the invention provides the use of a cell line developed for the large scale production of antibody for the production of an antibody of the invention. The invention further provides a cell for producing an antibody comprising a nucleic acid molecule that codes for a VH, a VL, and/or a heavy and light chain of an antibody as claimed. Preferably said nucleic acid molecule encodes a VH identified in FIG. 13, a nucleic acid molecule encoding a VH as identified by numeral 4327 or identified by numeral 8233 or a combination thereof.

The invention further provides a method for producing an antibody comprising culturing a cell of the invention and harvesting said antibody from said culture. Preferably said cell is cultured in a serum free medium. Preferably said cell is adapted for suspension growth. Further provided is an antibody obtainable by a method for producing an antibody according to the invention. The antibody is preferably purified from the medium of the culture. Preferably said antibody is affinity purified.

A cell of the invention is for instance a hybridoma cell line, a CHO cell, a 293F cell, an NSO cell or another cell type known for its suitability for antibody production for clinical purposes. In a particularly preferred embodiment said cell is a human cell. Preferably a cell that is transformed by an adenovirus E1 region or a functional equivalent thereof. A preferred example of such a cell line is the PER.C6 cell line or equivalent thereof. In a particularly preferred embodiment said cell is a CHO cell or a variant thereof. Preferably a variant that makes use of a Glutamine synthetase (GS) vector system for expression of an antibody.

The invention further provides a method for producing an antibody comprising culturing a cell of the invention and harvesting said antibody from said culture. Preferably said cell is cultured in a serum free medium. Preferably said cell is adapted for suspension growth. Further provided is an antibody obtainable by a method for producing an antibody according to the invention. The antibody is preferably purified from the medium of the culture. Preferably said antibody is affinity purified.

Bispecific antibodies are typically also produced by cells that express nucleic acid encoding the antibody. In this case the cell expresses the different light and heavy chains that make up the bispecific antibody. To this end the cell expresses two different heavy chains and at least one light chain. As unmodified heavy chains can pair with each other to form dimers such cells typically produce the two monospecific antibodies (homodimers), in addition to the bispecific antibody (heterodimer). This principle also applies to unmodified heavy chains that comprise a first heavy chain having one heavy chain variable region and a second heavy chain having at least two heavy chain variable regions, such that cells expressing these two heavy chains produce a monospecific antibody (homodimer of the pairing of the two first heavy chain), a quadrovalent antibody (homodimer of the pairing of two of the second heavy chains) and a trispecific antibody (heterodimer of the first and second heavy chain). The number of possible heavy/light chain combinations in the produced antibodies increases when the cell expresses two or more light chains. To reduce the number of different antibody species (combinations of different heavy and light chains) produced the afore mentioned “common light chain” is preferred.

An antibody producing cell that expresses a common light chain and equal amounts of the two heavy chains typically produces 50% bispecific antibody and 25% of each of the monospecific antibodies (i.e. having identical heavy light chain combinations). Alternatively in the above, example concerning a first heavy chain having one variable region and a second heavy chain having two variable regions, the two heavy chains typically produces 50% trispecific, 25% monospecific and 25% quadrospecific.

Several methods have been published to favor the production of the bispecific antibody or vice versa, the monospecific antibodies, which can further be employed for favoring multispecific antibody production. In the present invention it is preferred that the cell favors the production of the bispecific antibody over the production of the respective monospecific antibodies. Such is typically achieved by modifying the constant region of the heavy chains such that they favor heterodimerization (i.e. dimerization with the heavy chain of the other heavy/light chain combination) over homodimerization. In a preferred embodiment the bispecific antibody of the invention comprises two different immunoglobulin heavy chains with compatible heterodimerization domains. Various compatible heterodimerization domains have been described in the art. The compatible heterodimerization domains are preferably compatible immunoglobulin heavy chain CH3 heterodimerization domains. The art describes various ways in which such hetero-dimerization of heavy chains can be achieved, including use of ‘knob into hole’ bispecific antibodies.

In U.S. Ser. No. 13/866,747 (now issued as U.S. Pat. No. 9,248,181), U.S. Ser. No. 14/081,848 (now issued as U.S. Pat. No. 9,358,286) and PCT/NL2013/050294 (published as WO2013/157954); incorporated herein by reference) methods and means are disclosed for producing bispecific antibodies using compatible heterodimerization domains. These means and methods can also be favorably employed in the present invention. Specifically, preferred mutations to produce essentially only bispecific full length IgG molecules are the amino acid substitutions L351K and T366K (according to EU numbering) in the first CH3 domain (the ‘KK-variant’ heavy chain) and the amino acid substitutions L351D and L368E in the second domain (the ‘DE-variant’ heavy chain), or vice versa. It was previously demonstrated in our US 9,248,181 and U.S. Pat. No. 9,358,286 patents as well as the WO2013/157954 PCT application that the DE-variant and KK-variant preferentially pair to form heterodimers (so-called ‘DEKK’ (SEQ ID NO 36) bispecific molecules). Homodimerization of DE-variant heavy chains (DEDE homodimers) or KK-variant heavy chains (KKKK homodimers) hardly occurs due to strong repulsion between the charged residues in the CH3-CH3 interface between identical heavy chains. In one embodiment the heavy chain/light chain combination that comprises the variable domain that binds CD3, comprises a KK variant of the heavy chain. In this embodiment the heavy chain/light chain combination that comprises the variable domain that binds an antigen other than CD3 comprises a DE variant of the heavy chain. In a preferred embodiment the antigen other than CD3 is CLEC12A. In a preferred embodiment the VH of the variable domain that binds CLEC12A is MF4327 as depicted in FIG. 13.

Some antibodies are modified in CH2/lower hinge region, for instance to reduce Fc-receptor interaction or to reduce C1q binding. In some embodiments the antibody of the invention is an IgG antibody with a mutant CH2 and/or lower hinge domain such that interaction of the bispecific IgG antibody to a Fc-gamma receptor is reduced. Such a mutant CH2 and/or lower hinge domain preferably comprise an amino substitution at position 235 and/or 236 (according to EU numbering), preferably an L235G and/or G236R substitution.

Alternatively, some antibodies are modified for instance to enhance Fc receptor interaction or enhance C1q binding. For embodiments directed to auto-immune indications, such a modification may be preferred.

The invention further provides a method of treating a subject comprising administering an antigen-binding protein, preferably an antibody of the invention to the subject in need thereof. The invention further provides an antigen-binding protein, preferably an antibody of the invention for use in the treatment of a subject in need thereof. The subject preferably has cells that are to be removed from the body. The cells can be aberrant immune cells directing an auto-immune response or cancer cells or the like. Variable domains that are suited for this purpose are among others those with a common light chain and the VH of MF9249, and MF8397. These have a suitably low affinity and low cell kill activity but are functional under auto-immune response conditions. Variable domains that are suited for this purpose are among others those with a common light chain and the VH of MF9267, MF8057, MF8058, MF8078 and MF8508. These variable domains have a suitable affinity and a suitable cell kill activity for anticancer purposes.

Further, an invention set out herein, includes an antigen-binding protein or antibody with a high affinity variable domain having a high cell kill activity, which can be administered locally or be expressed locally, including the binding domain MF8078. The invention further provides a method of treating a subject comprising administering an antigen-binding protein, preferably an antibody of the invention to the subject in need thereof through a localized means of administration, as known to those of skill in the art, including, for example, an oncolytic virus, topical treatments for melanoma or other cancers in separate compartments such as the brain.

The invention further provides an antigen-binding protein, preferably an antibody, of the invention for use in the treatment of a subject in need thereof, which preferably has moderate to high affinity and relative high cytotoxicity such as those described herein. Variable domains that are suited for this purpose are among others those with a common light chain and the VH of MF8057, MF8058, MF9267, MF8508 and MF8078.

The invention further provides a method of treating cancer or a risk of cancer in a subject comprising administering to the subject in need thereof an antigen-binding protein, preferably an antibody that binds human CD3 comprising an antibody variable domain comprising a heavy chain variable region and a light chain variable region wherein the heavy chain variable region comprises a CDR1, CDR2 and CDR3 comprising the amino acid sequence:
CDR1: SFGIS (SEQ ID NO 42)
CDR2: GFIPVLGTANYAQKFQG (SEQ ID NO 43)
CDR3: RGNWNPFDP (SEQ ID NO 44); or comprising the amino acid sequence:

CDR1:
(SEQ ID NO 6)
SX₁TFTIS;
CDR2:
(SEQ ID NO 7)
GIIPX₂FGTITYAQKFQG;
CDR3:
(SEQ ID NO 44)
RGNWNPFDP;

wherein
X₁=Kor R;
X₂=L or I.

In a preferred embodiment X₁=K; and X₂=L. In another preferred embodiment X₁=R; and X₂=I.

The invention further provides an antigen-binding protein, preferably an antibody, of the invention for use in the treatment of a subject in need thereof, which preferably has moderate to high affinity and relative high cytotoxicity such as those described herein. Variable domains that are suited for this purpose are among others those with a common light chain and the VH of MF8048, MF8056 and MF8101.

The invention further provides a method of treating cancer or a risk of cancer in a subject comprising administering to the subject in need thereof an antigen-binding protein, preferably an antibody that binds human CD3 comprising an antibody variable domain comprising a heavy chain variable region and a light chain variable region wherein the heavy chain variable region comprises aa CDR1, CDR2 and CDR3 comprising the amino acid sequence:

CDR1:
(SEQ ID NO 84)
SKTLTIS;
CDR2:
(SEQ ID NO 85)
GIIPIFGSITYAQKFQD;
CDR3:
(SEQ ID NO 44)
RGNWNPFDP;
or

comprising the amino acid sequence:

CDR1:
(SEQ ID NO 100)
GSGIS;
CDR2:
(SEQ ID NO 101)
GFIPFFGSANYAQKFRD;
CDR3:
(SEQ ID NO 8)
RGNWNPX₁₃DP;

wherein
X₁₃= or L or F.

The invention further provides a method of treating cancer or a risk of cancer in a subject comprising administering to the subject in need thereof an antigen-binding protein, preferably an antibody, that binds human CD3 comprising an antibody variable domain comprising a heavy chain variable region and a light chain variable region wherein the heavy chain variable region comprises the amino acid sequence

(SEQ ID NO 45)
EVQLVQSGAEVKKPGSSVKVSCKASGGTFRSFGISWVRQAPGQGLEWMGG
FIPVLGTANYAQKFQGRVTIIADKSTNTAYMELSSLRSEDTAVYYCARRG
NWNPFDPWGQGTLVTVSS;
(SEQ ID NO 54)
QVQLVQSGAEVKKPGSSVKVSCKASGDAFKSKTFTISWVRQAPGQGLEWL
GGIIPLFGTITYAQKFQGRVTITADKSTNTAFMELSSLRSEDTAMYYCTR
RGNWNPFDPWGQGTLVTVSS;
(SEQ ID NO 63)
EVQLVQSGSELKKPGSSVKVSCKASGVTFNSRTFTISWVRQAPGQGLEWL
GSIIPIFGTITYAQKFQGRVTITADKSTSTAFMELTSLRSEDTAIYYCTR
RGNWNPFDPWGQGTLVTVSS;
(SEQ ID NO 71)
QVQLVQSGAEVKKPGSSVKVSCKASGGTFRGSGISWVRQAPGQGLEWVGG
FIPFFGSANYAQKFRDRVTITADKSATTAYMELSSLRSEDTAIYYCAKRG
NWNPLDPWGQGTLVTVSS;
(SEQ ID NO 87)
QVQLVQSGAEVKKPGSSVKVSCKASGVTFKSKTLTISWVRQAPGQGLEWL
GGIIPIFGSITYAQKFQDRVSITADKSTNTAYLELNSLRSEDTAIYYCAR
RGNWNPFDPWGQGTLVTVSS;
or
(SEQ ID NO 103)
EVQLVQSGAEVKKPGSSVKVSCKASGGTFRGSGISWVRQAPGQGLEWVGG
FIPFFGSANYAQKFRDRVTITADKSATTAYMELSSLRSEDTAIYYCAKRG
NWNPFDPWGQGTLVTVSS;

with 0-10, preferably 0-5 amino acid variations, insertions, deletions, substitutions, additions or a combination thereof at one or more positions other than the CDRs.

The invention further provides a method of treating cancer or a risk of cancer in a subject comprising administering to the subject in need thereof an antigen-binding protein, preferably an antibody, that binds human CD3 comprising an antibody variable domain comprising a heavy chain variable region and a light chain variable region wherein the heavy chain variable region comprises a CDR1, CDR2 and CDR3 comprising the amino acid sequence:

CDR1:
(SEQ ID NO 9)
RX₃WIG;
CDR2:
(SEQ ID NO 200)
IIYPGDSDTRYSPSFQG;
CDR3:
(SEQ ID NO 10)
X₄IRYFX₅WSEDYHYYX₆DV;

wherein
X₃=F or Y;
X₄=H or N;
X₅=D or V;
X₆=L or M.
   In one embodiment X₃=F; X₄=H; X₅=D; and X₆=L. In a further embodiment X₃=Y; X₄=N; X₅=V; and X₆=M.

The invention further provides a method of treating cancer or a risk of cancer in a subject comprising administering to the subject in need thereof an antigen-binding protein, preferably an antibody, that binds human CD3 comprising an antibody variable domain comprising a heavy chain variable region and a light chain variable region wherein the heavy chain variable region comprises the amino acid sequence

(SEQ ID NO 298)
EVQLVQSGAEVKKPGESLKISCKGSGYSFTRFWIGWVRQMPGKGLEWMGI
IYPGDSDTRYSPSFQGQVTISADKSTSTAYLQWSSLKASDTGMYYCVRHI
RYFDWSEDYHYYLDVWGKGTTVTVSS;
or
(SEQ ID NO 211)
EVQLVESGAEVKKPGESLKISCKGSGYSFTRYWIGWVRQMPGKGLEWMGI
IYPGDSDTRYSPSFQGQVTISADKSISTAYLQWSSLKASDTAMYYCVRNI
RYFVWSEDYHYYMDVWGKGTTVTVSS;

with 0-10, preferably 0-5 amino acid variations, insertions, deletions, substitutions, additions or a combination thereof at one or more positions other than the CDRs.

The invention further provides a method of treating cancer or a risk of cancer in a subject comprising administering to the subject in need thereof an antigen-binding protein, preferably an antibody, that binds human CD3 comprising an antibody variable domain comprising a heavy chain variable region and a light chain variable region wherein the heavy chain variable region comprises a CDR1, CDR2 and CDR3 comprising the amino acid sequence:

CDR1:
(SEQ ID NO 117)
SYALS;
CDR2:
(SEQ ID NO 118)
GISGSGRTTWYADSVKG;
CDR3:
(SEQ ID NO 119)
DGGYSYGPYWYFDL.

The invention further provides a method of treating cancer or a risk of cancer in a subject comprising administering to the subject in need thereof an antigen-binding protein, preferably an antibody, that binds human CD3 comprising an antibody variable domain comprising a heavy chain variable region and a light chain variable region wherein the heavy chain variable region comprises a CDR1, CDR2 and CDR3 comprising the amino acid sequence:

CDR1:
(SEQ ID NO 117)
SYALS;
CDR2:
(SEQ ID NO 126)
AISGSGRTTWYADSVKG;
CDR3:
(SEQ ID NO 127)
DGGYTYGPYWYFDL.

The invention further provides a method of treating cancer or a risk of cancer in a subject comprising administering to the subject in need thereof an antigen-binding protein, preferably an antibody, that binds human CD3 comprising an antibody variable domain comprising a heavy chain variable region and a light chain variable region wherein the heavy chain variable region comprises the amino acid sequence

(SEQ ID NO 120)
QVQLVQSGGGLVQPGGSLRLSCATSGFKFSSYALSWVRQAPGKGLEWVSG
ISGSGRTTWYADSVKGRFTISRDNSKNTLYLQMNSLRAEDTAVYYCARDG
GYSYGPYWYFDLWGRGTLVTVSS;
or
(SEQ ID NO 128)
QVQLVESGGGLVQPGGSLRLSCATSGFTFISYALSWVRQAPGKGLEWVSA
ISGSGRTTWYADSVKGRFTISRDNSKNTLFLQMNSLRAEDTAVYYCARDG
GYTYGPYWYFDLWGRGTLVTVSS;

with 0-10, preferably 0-5 amino acid variations, insertions, deletions, substitutions, additions or a combination thereof at one or more positions other than the CDRs.

The invention further provides a method of treating cancer or a risk of cancer in a subject comprising administering to the subject in need thereof an antigen-binding protein, preferably an antibody that binds human CD3 comprising an antibody variable domain comprising a heavy chain variable region and a light chain variable region wherein the heavy chain variable region comprises a CDR1, CDR2 and CDR3 comprising the amino acid sequence:

CDR1:
(SEQ ID NO 142)
DYTMH;
CDR2:
(SEQ ID NO 143)
DISWSSGSIGYADSVKG;
CDR3:
(SEQ ID NO 144)
DHRGYGDYEGGGFDY.

The invention further provides a method of treating cancer or a risk of cancer in a subject comprising administering to the subject in need thereof an antigen-binding protein, preferably an antibody that binds human CD3 comprising an antibody variable domain comprising a heavy chain variable region and a light chain variable region wherein the heavy chain variable region comprises a CDR1, CDR2 and CDR3 comprising the amino acid sequence:

CDR1:
(SEQ ID NO 142)
DYTMH;
CDR2:
(SEQ ID NO 11)
DISWSX₇GX₈X₉X₁₀YADSVKG;
CDR3:
(SEQ ID NO 12)
DHX₁₁GYGDYEGGGFDX₁₂;

wherein
X₇=S or G;
X₈=S or T;
X₉=I or T;
X₁₀=G or Y;
X₁₁=R or M;
X₁₂=H or Y,
preferably X₇, X₈, X₉ and X₁₀ are S, S, I and G or G, S, I and Y or S, T, T and G, and preferably X₁₁ and X₁₂ are R and H, or R and Y, or M and Y, more preferably X₇, X₈, X₉, X₁₀, X₁₁ and X₁₂ are S, S, I, G, R and H or G, S, I, Y, R and Y or S, T, T, G, M and Y, or, in other words, preferably X₇, X₈, X₉ and X₁₀ are S, S, I and G, and X₁₁ and X₁₂ are R and H; or X₇, X₈, X₉ and X₁₀ are G, S, I and Y, and X₁₁ and X₁₂ are R and Y; or X₇, X₈, X₉ and X₁₀ are S, T, T and G, and X₁₁ and X₁₂ are M and Y.

The invention further provides a method of treating cancer or a risk of cancer in a subject comprising administering to the subject in need thereof an antigen-binding protein, preferably an antibody that binds human CD3 comprising an antibody variable domain comprising a heavy chain variable region and a light chain variable region wherein the heavy chain variable region comprises the amino acid sequence

(SEQ ID NO 145)
EVQLVESGGGLVQPGRSLRLSCATSGFNFDDYTMHWVRQAPGKGLEWVSD
ISWSSGSIGYADSVKGRFTISRDNAKNSLWLQMNSLRTEDTALYFCAKDH
RGYGDYEGGGFDYWGQGTLVTVSS;
(SEQ ID NO 153)
EVQLVESGGGLVQPGRSLRLSCATSGFTFDDYTMHWVRQAPGKGLEWVSD
ISWSSGSIGYADSVKGRFTISRDNAKNSLYLQMNSLRAEDTALYFCAKDH
RGYGDYEGGGFDHWGQGTLVTVSS;
(SEQ ID NO 185)
EVQLVESGGGLVQPGRSLRLSCVTSGFTFDDYTMHWVRQAPGKGLEWVSD
ISWSSGTTGYADSVKGRFTISRDNAKDSLYLQMNSLRTEDTALYYCAKDH
MGYGDYEGGGFDYWGQGTLVTVSS;
or
(SEQ ID NO 169)
EVQLVESGGVVVQPGGSLRLSCAASGFTFDDYTMHWVRQAPGKGLEWVSD
ISWSGGSIYYADSVKGRFTISRDNSKNSLYLQMNSLRTEDTALYYCAKDH
RGYGDYEGGGFDYWGRGTLVTVSS;

with 0-10, preferably 0-5 amino acid variations, insertions, deletions, substitutions, additions or a combination thereof at one or more positions other than the CDRs.

The antigen-binding protein, preferably an antibody in a treatment as indicated herein above preferably comprises a heavy chain-light chain (H/L) combination that binds a tumor-antigen.

The antibody is preferably a human or humanized antibody. Preferably the antibody comprises two different immunoglobulin heavy chains with compatible heterodimerization domains. Said compatible heterodimerization domains are preferably compatible immunoglobulin heavy chain CH3 heterodimerization domains. Said bispecific antibody is preferably an IgG antibody with a mutant CH2 and/or lower hinge domain such for immuno-oncology applications that interaction of the bispecific or multispecific IgG antibody to a Fc-gamma receptor is reduced. The mutant CH2 and/or lower hinge domain preferably comprise an amino substitution at position 235 and/or 236 (according to EU numbering), preferably an L235G and/or G236R substitution. Alternatively, for auto-immune applications, the interaction of the bispecific or multispecific to a Fc-gamma receptor is enhanced or ADCC and CDC is enhanced by modification to the CH2 and/or CH3 domain. For example, by engineering Fc regions (through introducing amino acid substitutions) that bind to activating receptors with greater selectivity, antibodies can be created that have greater capability to mediate cytotoxic activities desired by an anti-cancer Mab or CD3 targeting binding arm for the treatment of auto-immune related maladies. For instance, a reported technique for enhancing ADCC of an antibody is afucosylation. (See for instance Junttila, T. T., K. Parsons, et al. (2010). “Superior In vivo Efficacy of Afucosylated Trastuzumab in the Treatment of HER2-Amplified Breast Cancer.” Cancer Research 70 (11): 4481-4489). Further provided is therefore a bispecific antibody according to the invention, which is afucosylated. Alternatively, or additionally, multiple other strategies are reported to be used to achieve ADCC enhancement, for instance including glycoengineering (Kyowa Hakko/Biowa, GlycArt (Roche) and Eureka Therapeutics) and mutagenesis (Xencor and Macrogenics), all of which seek to improve Fc binding to low-affinity activating FcγRIIIa, and/or to reduce binding to the low affinity inhibitory FcγRIIb.

The antibody preferably comprises a common light chain.

The invention further provides a bispecific antigen-binding protein, preferably a bispecific antibody, that comprises a variable domain that binds a tumor-antigen and a variable domain that binds human CD3 wherein the variable domains each comprise a different heavy chain variable region and a common light chain variable region and wherein the heavy chain variable region of the variable domain that binds human CD3 comprises a CDR1, CDR2 and CDR3 comprising the amino acid sequence:

CDR1:
(SEQ ID NO 42)
SFGIS
CDR2:
(SEQ ID NO 43)
GFIPVLGTANYAQKFQG
CDR3:
(SEQ ID NO 44)
RGNWNPFDP;
or

comprising the amino acid sequence:

CDR1:
(SEQ ID NO 6)
SX₁TFIS;
CDR2:
(SEQ ID NO 7)
GIIPX₂FGTITYAQKFQG;
CDR3:
(SEQ ID NO 44)
RGNWNPFDP;

wherein
X₁=Kor R;
X₂=L or I.

In a preferred embodiment X₁=K; and X₂=L. In another preferred embodiment X₁=R; and X₂=I.

The invention further provides a bispecific antigen-binding protein, preferably a bispecific antibody, that comprises a variable domain that binds a tumor-antigen and a variable domain that binds human CD3 wherein the variable domains each comprise a different heavy chain variable region and a common light chain variable region and wherein the heavy chain variable region of the variable domain that binds human CD3 comprises a CDR1, CDR2 and CDR3 comprising the amino acid sequence:

CDR1:
(SEQ ID NO 84)
SKTLTIS;
CDR2:
(SEQ ID NO 85)
GIIPIFGSITYAQKFQD;
CDR3:
(SEQ ID NO 44)
RGNWNPFDP;
or

comprising the amino acid sequence:

CDR1:
(SEQ ID NO 100)
GSGIS;
CDR2:
(SEQ ID NO 101)
GFIPFFGSANYAQKFRD;
CDR3:
(SEQ ID NO 8)
RGNWNPX₁₃DP;

wherein
X₁₃= or L or F.

The invention further provides a bispecific antigen-binding protein, preferably a bispecific antibody, that comprises a variable domain that binds a tumor-antigen and a variable domain that binds human CD3 wherein the variable domains each comprise a different heavy chain variable region and a common light chain variable region and wherein the heavy chain variable region of the variable domain that binds human CD3 comprises the amino acid sequence

(SEQ ID NO 45)
EVQLVQSGAEVKKPGSSVKVSCKASGGTFRSFGISWVRQAPGQGLEWMGG
FIPVLGTANYAQKFQGRVTIIADKSTNTAYMELSSLRSEDTAVYYCARRG
NWNPFDPWGQGTLVTVSS;
(SEQ ID NO 54)
QVQLVQSGAEVKKPGSSVKVSCKASGDAFKSKTFTISWVRQAPGQGLEWL
GGIIPLFGTITYAQKFQGRVTITADKSTNTAFMELSSLRSEDTAMYYCTR
RGNWNPFDPWGQGTLVTVSS;
(SEQ ID NO 63)
EVQLVQSGSELKKPGSSVKVSCKASGVTFNSRTFTISWVRQAPGQGLEWL
GSIIPIFGTITYAQKFQGRVTITADKSTSTAFMELTSLRSEDTAIYYCTR
RGNWNPFDPWGQGTLVTVSS;
(SEQ ID NO 71)
QVQLVQSGAEVKKPGSSVKVSCKASGGTFRGSGISWVRQAPGQGLEWVGG
FIPFFGSANYAQKFRDRVTITADKSATTAYMELSSLRSEDTAIYYCAKRG
NWNPLDPWGQGTLVTVSS;
(SEQ ID NO 87)
QVQLVQSGAEVKKPGSSVKVSCKASGVTFKSKTLTISWVRQAPGQGLEWL
GGIIPIFGSITYAQKFQDRVSITADKSTNTAYLELNSLRSEDTAIYYCAR
RGNWNPFDPWGQGTLVTVSS;
or
(SEQ ID NO 103)
EVQLVQSGAEVKKPGSSVKVSCKASGGTFRGSGISWVRQAPGQGLEWVGG
FIPFFGSANYAQKFRDRVTITADKSATTAYMELSSLRSEDTAIYYCAKRG
NWNPFDPWGQGTLVTVSS

with 0-10, preferably 0-5 amino acid variations, insertions, deletions, substitutions, additions or a combination thereof at one or more positions other than the CDRs.

The invention further provides a bispecific antigen-binding protein, preferably a bispecific antibody, that comprises a variable domain that binds a tumor-antigen and a variable domain that binds human CD3 wherein the variable domains each comprise a different heavy chain variable region and a common light chain variable region and wherein the heavy chain variable region of the variable domain that binds human CD3 comprises a CDR1, CDR2 and CDR3 comprising the amino acid sequence:

CDR1:
(SEQ ID NO 9)
RX₃WIG;
CDR2:
(SEQ ID NO 200)
IIYPGDSDTRYSPSFQG;
CDR3:
(SEQ ID NO 10)
X₄IRYFX₅WSEDYHYYX₆DV;

wherein
X₃=F or Y;
X₄=H or N;
X₅=D or V;
X₆=L or M.
   In one embodiment X₃=F; X₄=H; X₅=D; and X₆=L. In a further embodiment X₃=Y; X₄=N; X₅=V; and X₆=M.

The invention further provides a bispecific antigen-binding protein, preferably a bispecific antibody, that comprises a variable domain that binds a tumor-antigen and a variable domain that binds human CD3 wherein the variable domains each comprise a different heavy chain variable region and a common light chain variable region and wherein the heavy chain variable region of the variable domain that binds human CD3 comprises the amino acid sequence

(SEQ ID NO 298)
EVQLVQSGAEVKKPGESLKISCKGSGYSFTRFWIGWVRQMPGKGLEWMGI
IYPGDSDTRYSPSFQGQVTISADKSTSTAYLQWSSLKASDTGMYYCVRHI
RYFDWSEDYHYYLDVWGKGTTVTVSS;
or
(SEQ ID NO 211)
EVQLVESGAEVKKPGESLKISCKGSGYSFTRYWIGWVRQMPGKGLEWMGI
IYPGDSDTRYSPSFQGQVTISADKSISTAYLQWSSLKASDTAMYYCVRNI
RYFVWSEDYHYYMDVWGKGTTVTVSS;

with 0-10, preferably 0-5 amino acid variations, insertions, deletions, substitutions, additions or a combination thereof at one or more positions other than the CDRs.

The invention further provides a bispecific antigen-binding protein, preferably a bispecific antibody, that comprises a variable domain that binds a tumor-antigen and a variable domain that binds human CD3 wherein the variable domains each comprise a different heavy chain variable region and a common light chain variable region and wherein the heavy chain variable region of the variable domain that binds human CD3 comprises a CDR1, CDR2 and CDR3 comprising the amino acid sequence:

CDR1:
(SEQ ID NO 117)
SYALS;
CDR2:
(SEQ ID NO 118)
GISGSGRTTWYADSVKG;
CDR3:
(SEQ ID NO 119)
DGGYSYGPYWYFDL.

The invention further provides a bispecific antigen-binding protein, preferably a bispecific antibody, that comprises a variable domain that binds a tumor-antigen and a variable domain that binds human CD3 comprising an antibody variable domain comprising a heavy chain variable region and a light chain variable region wherein the heavy chain variable region comprises a CDR1, CDR2 and CDR3 comprising the amino acid sequence:

CDR1:
(SEQ ID NO 117)
SYALS;
CDR2:
(SEQ ID NO 126)
AISGSGRTTWYADSVKG;
CDR3:
(SEQ ID NO 127)
DGGYTYGPYWYFDL.

The invention further provides a bispecific antigen-binding protein, preferably a bispecific antibody, that comprises a variable domain that binds a tumor-antigen and a variable domain that binds human CD3 wherein the variable domains each comprise a different heavy chain variable region and a common light chain variable region and wherein the heavy chain variable region of the variable domain that binds human CD3 comprises the amino acid sequence

(SEQ ID NO 120)
QVQLVQSGGGLVQPGGSLRLSCATSGFKFSSYALSWVRQAPGKGLEWVSG
ISGSGRTTWYADSVKGRFTISRDNSKNTLYLQMNSLRAEDTAVYYCARDG
GYSYGPYWYFDLWGRGTLVTVSS;
or
(SEQ ID NO 128)
QVQLVESGGGLVQPGGSLRLSCATSGFTFISYALSWVRQAPGKGLEWVSA
ISGSGRTTWYADSVKGRFTISRDNSKNTLFLQMNSLRAEDTAVYYCARDG
GYTYGPYWYFDLWGRGTLVTVSS

with 0-10, preferably 0-5 amino acid variations, insertions, deletions, substitutions, additions or a combination thereof at one or more positions other than the CDRs.

The invention further provides a bispecific antigen-binding protein, preferably a bispecific antibody, that comprises a variable domain that binds a tumor-antigen and a variable domain that binds human CD3 wherein the variable domains each comprise a different heavy chain variable region and a common light chain variable region and wherein the heavy chain variable region of the variable domain that binds human CD3 comprises a CDR1, CDR2 and CDR3 comprising the amino acid sequence:

CDR1:
(SEQ ID NO 142)
DYTMH;
CDR2:
(SEQ ID NO 143)
DISWSSGSIGYADSVKG;
CDR3:
(SEQ ID NO 144)
DHRGYGDYEGGGFDY.

The invention further provides a bispecific antigen-binding protein, preferably a bispecific antibody, that comprises a variable domain that binds a tumor-antigen and a variable domain that binds human CD3 comprising an antibody variable domain comprising a heavy chain variable region and a light chain variable region wherein the heavy chain variable region comprises a CDR1, CDR2 and CDR3 comprising the amino acid sequence:

CDR1:
(SEQ ID NO 142)
DYTMH;
CDR2:
(SEQ ID NO 11)
DISWSX₇GX₈X₉X₁₀YADSVKG;
CDR3:
(SEQ ID NO 12)
DHX₁₁GYGDYEGGGFDX₁₂;

wherein
X₇=S or G;
X₈=S or T;
X₉=I or T;
X₁₀=G or Y;
X₁₁=R or M;
X₁₂=H or Y,
preferably X₇, X₈, X₉ and X₁₀ are S, S, I and G or G, S, I and Y or S, T, T and G, and preferably X₁₁ and X₁₂ are R and H, or R and Y, or M and Y, more preferably X₇, X₈, X₉, X₁₀, X₁₁ and X₁₂ are S, S, I, G, R and H or G, S, I, Y, R and Y or S, T, T, G, M and Y, or, in other words, preferably X₇, X₈, X₉ and X₁₀ are S, S, I and G, and X₁₁ and X₁₂ are R and H; or X₇, X₈, X₉ and X₁₀ are G, S, I and Y, and X₁₁ and X₁₂ are R and Y; or X₇, X₈, X₉ and X₁₀ are S, T, T and G, and X₁₁ and X₁₂ are M and Y.

The invention further provides a bispecific antigen-binding protein, preferably a bispecific antibody, that comprises a variable domain that binds a tumor-antigen and a variable domain that binds human CD3 wherein the variable domains each comprise a different heavy chain variable region and a common light chain variable region and wherein the heavy chain variable region of the variable domain that binds human CD3 comprises the amino acid sequence

(SEQ ID NO 145)
EVQLVESGGGLVQPGRSLRLSCATSGFNFDDYTMHWVRQAPGKGLEWVSDI
SWSSGSIGYADSVKGRFTISRDNAKNSLWLQMNSLRTEDTALYFCAKDHRG
YGDYEGGGFDYWGQGTLVTVSS;
(SEQ ID NO 153)
EVQLVESGGGLVQPGRSLRLSCATSGFTFDDYTMHWVRQAPGKGLEWVSDI
SWSSGSIGYADSVKGRFTISRDNAKNSLYLQMNSLRAEDTALYFCAKDHRG
YGDYEGGGFDHWGQGTLVTVSS;
(SEQ ID NO 185)
EVQLVESGGGLVQPGRSLRLSCVTSGFTFDDYTMHWVRQAPGKGLEWVSDI
SWSSGTTGYADSVKGRFTISRDNAKDSLYLQMNSLRTEDTALYYCAKDHMG
YGDYEGGGFDYWGQGTLVTVSS;
or
(SEQ ID NO 169)
EVQLVESGGVVVQPGGSLRLSCAASGFTFDDYTMHWVRQAPGKGLEWVSDI
SWSGGSIYYADSVKGRFTISRDNSKNSLYLQMNSLRTEDTALYYCAKDHRG
YGDYEGGGFDYWGRGTLVTVSS;

with 0-10, preferably 0-5 amino acid variations, insertions, deletions, substitutions, additions or a combination thereof at one or more positions other than the CDRs.

In one embodiment the heavy chain variable region of the variable domain that binds a tumor antigen preferably comprises the amino acid sequence of MF8233 (EGFR)

(SEQ ID NO 247)
QVQLVQSGAEVKKPGASVKVSCKASGYTFTSYGISWVRQAPGQGLEWMGW
ISAYNANTNYAQKLQGRVTMTTDTSTSTAYMELRSLRSDDTAVYYCAKDR
HWHWWLDAFDYWGQGTLVTVSS

with 0-10, preferably 0-5 amino acid variations, insertions, deletions, substitutions, additions or a combination thereof at one or more positions other than the CDR.

In another embodiment the heavy chain variable region of the variable domain that binds a tumor antigen preferably comprises the amino acid sequence of

MF4327(CLEC12A)
(SEQ ID NO 293)
QVQLVQSGAEVKKPGASVKVSCKASGYTFTSYYMHWVRQAPGQGLEWMGI
INPSGGSTSYAQKFQGRVTMTRDTSTSTVYMELSSLRSEDTAVYYCAKGT
TGDWFDYWGQGTLVTVSS

with 0-10, preferably 0-5 amino acid variations, insertions, deletions, substitutions, additions or a combination thereof at one or more positions other than the CDRs.

The invention further provides an antibody of the invention or a derivative thereof or a pharmaceutical composition of the invention, for use in the treatment of a subject in need thereof. For the treatment of a subject that has or is at risk of having a tumor it is preferred that the antibody is a bispecific antibody of the invention. Preferably wherein the CD3 binding antibody comprises a heavy/light chain combination that binds a tumor antigen.

Provided are CD3/tumor antigen bispecific antibodies and pharmaceutical compositions comprising such bispecific antibodies for use in the treatment of solid or hematological tumors. Preferred solid tumors are of epithelial origin; gynecological cancer such as ovarian and endometrial tumors; prostate cancer, brain cancer or any other solid tumor.

Provided is also a CD3/tumor antigen bispecific antibody of the invention or a derivative thereof or pharmaceutical compositions comprising such bispecific antibody or derivative thereof for use in the treatment of various leukemias and pre-leukemic diseases of myeloid origin but also B cell lymphomas. Diseases that can be treated according to the invention include myeloid leukemias or pre-leukemic diseases such as acute myeloid leukemia (AML), myelodysplastic syndrome (MDS) and chronic myelogenous leukemia (CML), and Hodgkin's lymphomas and most non-Hodgkin's lymphomas. Also B-ALL; T-ALL, mantle cell lymphoma are also preferred targets for treatment with antibody of the invention. Thus the invention provides a bispecific full length IgG antibody according to the invention for use as a pharmaceutical in the treatment of myelodysplastic syndrome (MDS), chronic myelogenous leukemia (CML), multiple myeloma (MM) or preferably acute myeloid leukemia (AML). Also provided is a use of a bispecific IgG antibody according to the invention in the preparation of a medicament for the treatment or prevention of MDS, CML, MM or preferably AML.

The amount of antibody according to the invention to be administered to a patient is typically in the therapeutic window, meaning that a sufficient quantity is used for obtaining a therapeutic effect, while the amount does not exceed a threshold value leading to an unacceptable extent of side-effects. The lower the amount of antibody needed for obtaining a desired therapeutic effect, the larger the therapeutic window will typically be. An antibody according to the invention exerting sufficient therapeutic effects at low dosage is, therefore, preferred.

Approximately 30.000 patients are diagnosed each year with AML in Europe and US. The majority of these patients are 60 years of age or older. Older age is a major negative determinant of outcome in AML and long term survival (at 5 years) of intensively treated older AML patients is approximately 10%. In almost all patients that have achieved remission upon induction chemotherapy, disease progression is observed within 3 years. Current post remission treatment has shown limited, if any, value in older patients with AML. Therefore, a significant load of residual resistant leukemia remains, and the surviving subpopulation of drug resistant leukemic cells rapidly generates recurrence. Novel types of drugs with entirely different modes of action are needed to target these chemotherapy non responsive AML tumor cells in efforts to induce and sustain complete remissions. Although complete remission (CR) can be achieved with a number of intensive chemotherapy combinations in more than 50% of elderly AML patients and around 80% in younger patients, advancements of response or survival have remained a major investigational challenge. In a recently published network meta-analysis of 65 randomized clinical trials (15.110 patients) in older patients with AML most of the amended investigational induction regimens have similar or even worse efficacy profiles as compared to the conventional 3+7 induction regimen with daunorubicin and cytarabine. This standard treatment of AML is associated with high morbidity and even mortality. The majority of the patients in CR relapse due to remaining leukemic stem cells after chemotherapy. Further dose intensification is limited due to unacceptable toxicity. An urgent need for new treatment modalities preferably with less toxicity is thus emerging especially in elderly patients with AML.

Treatment of chemotherapy unresponsive AML could be achieved by redirecting T cells from the patient's own immune system to AML tumor cells and subsequent tumor target-specific activation of T cells using a bispecific antibody. This process is also known as a so-called “T-cell engaging approach”. In this manner, the patients' immune system is strengthened and retargeted to attack and eradicate the AML tumor cells. For example, CD3×CLEC12A bispecific IgG antibodies efficiently redirect T cells towards the AML tumor cells, thereby inducing AML tumor cell lysis.

### BRIEF DESCRIPTION OF THE DRAWINGS

FIG. 1

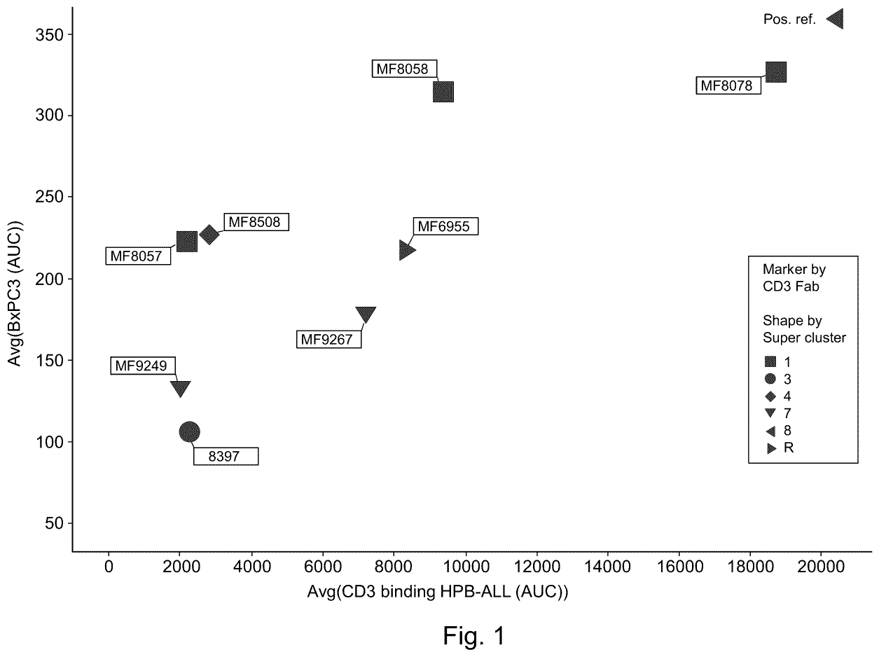

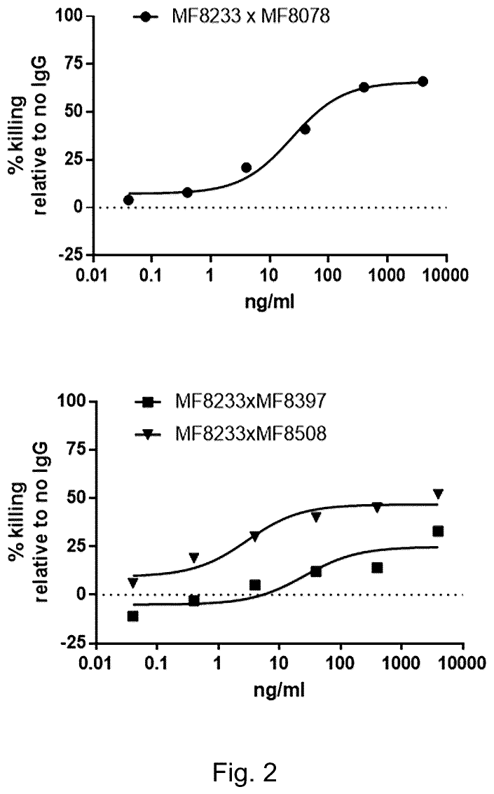

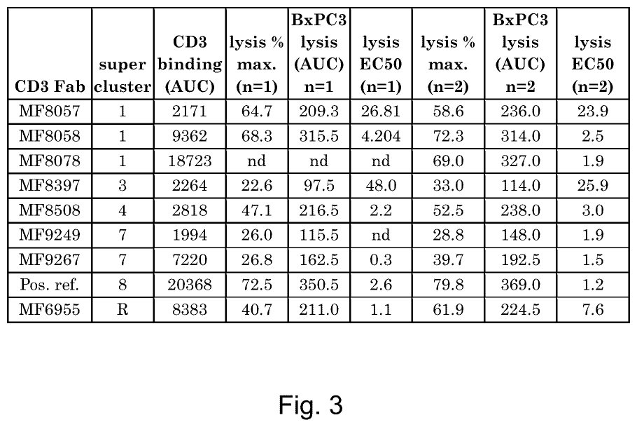

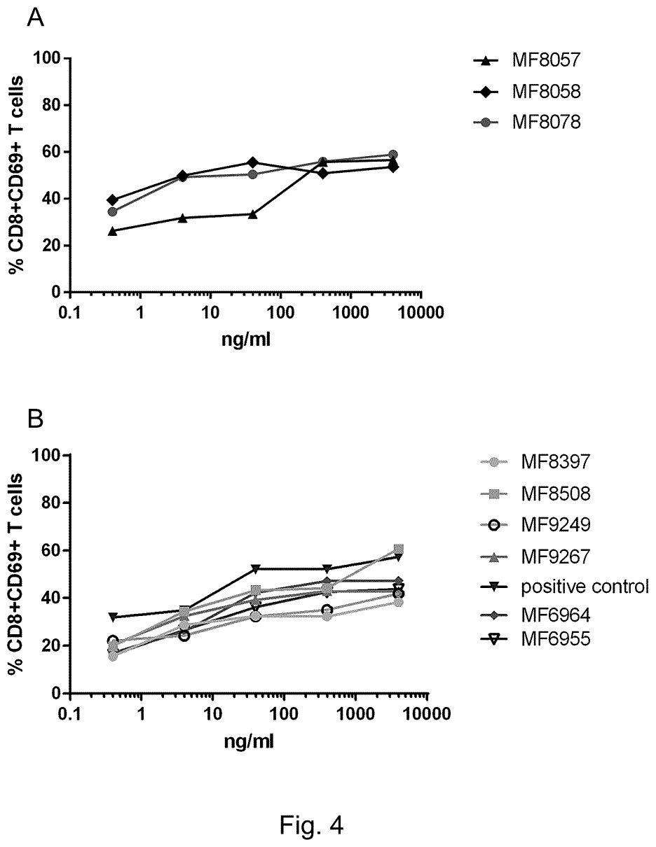

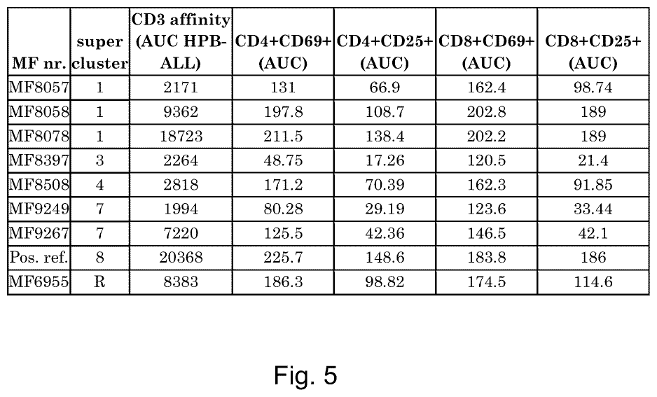

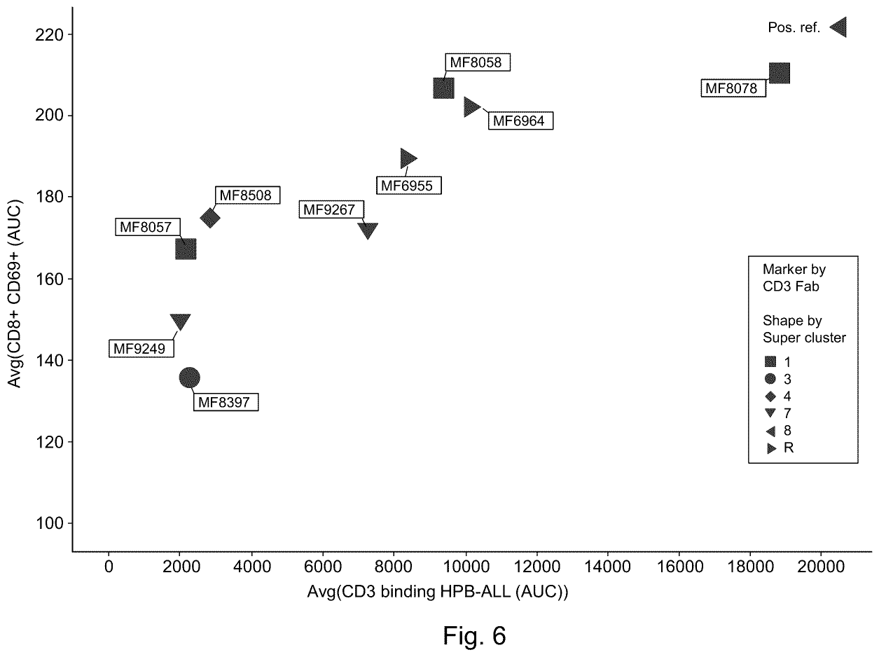

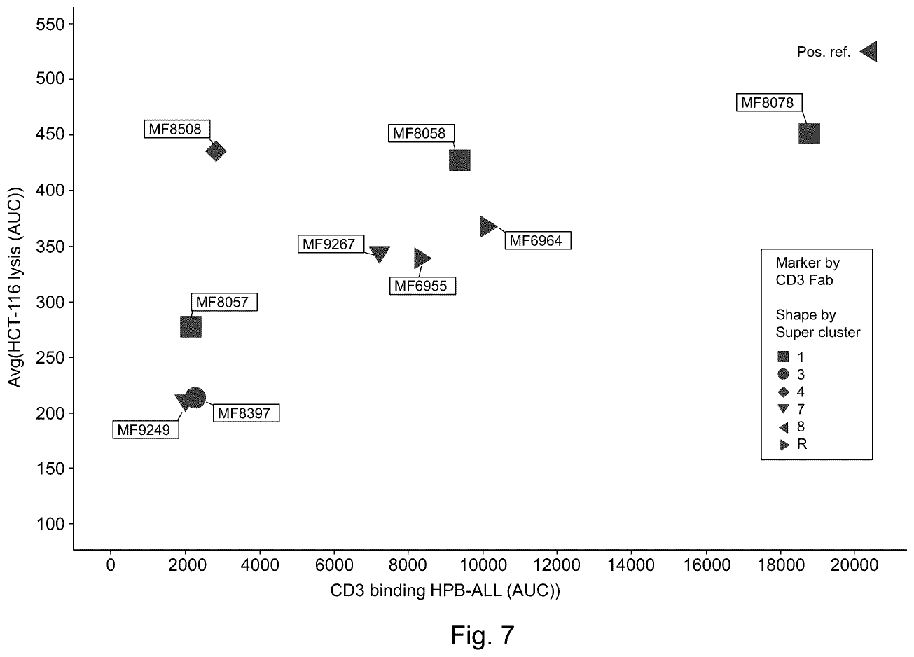

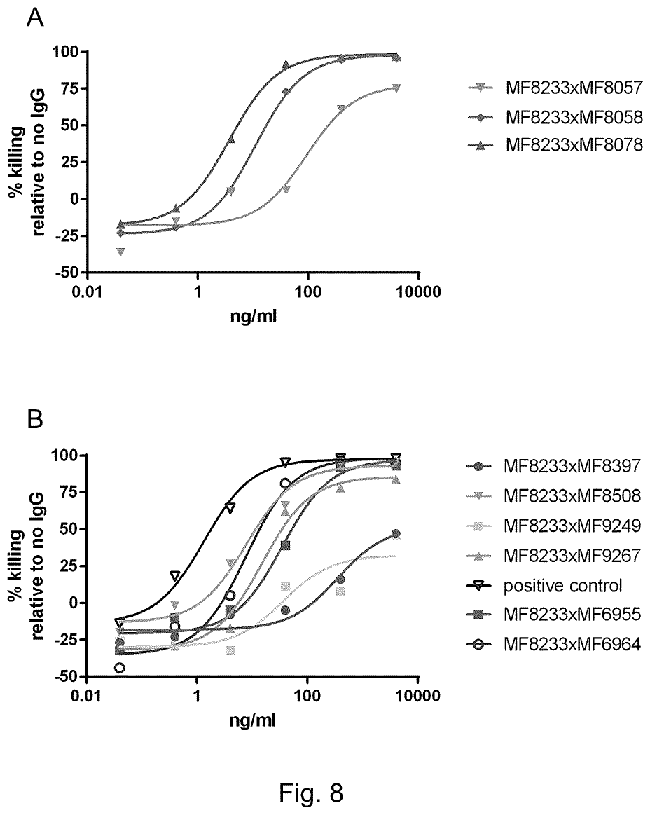

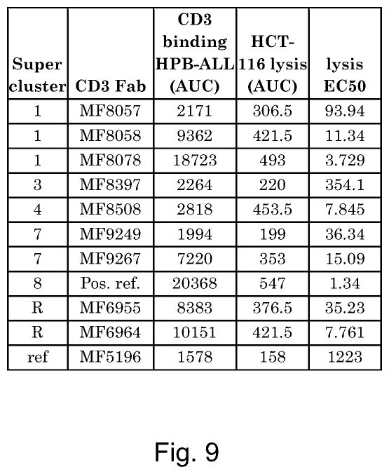

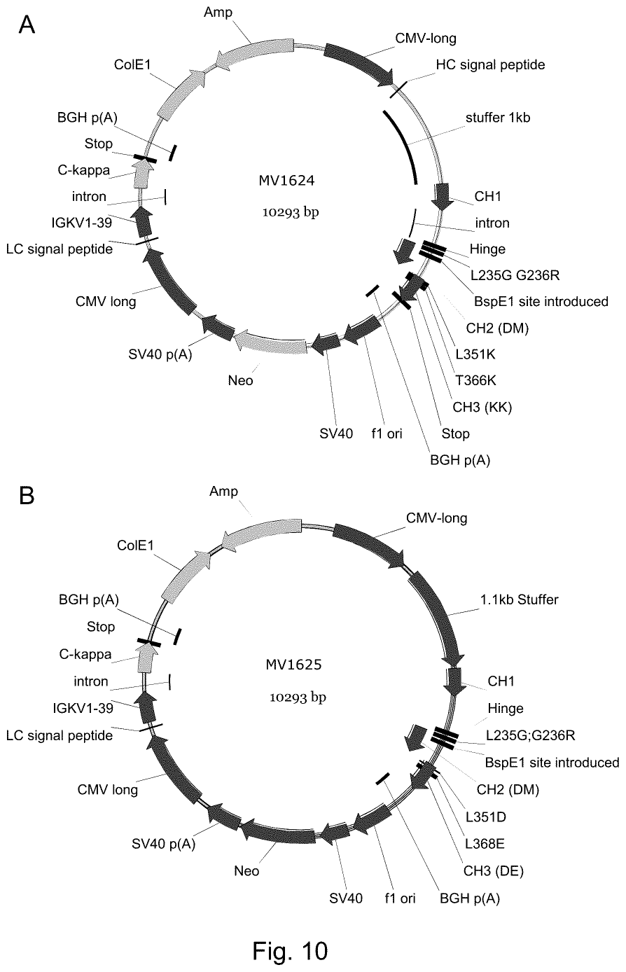

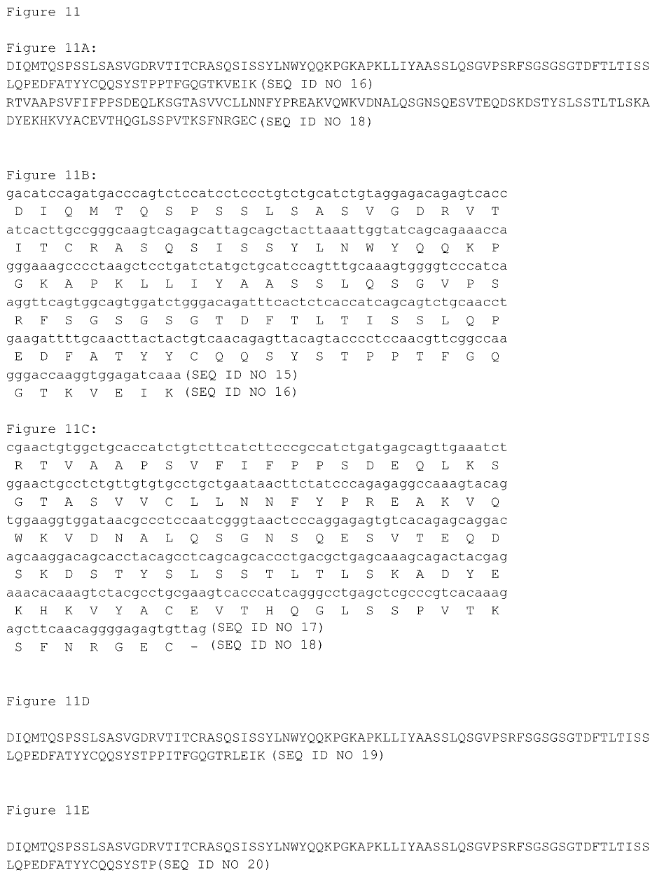

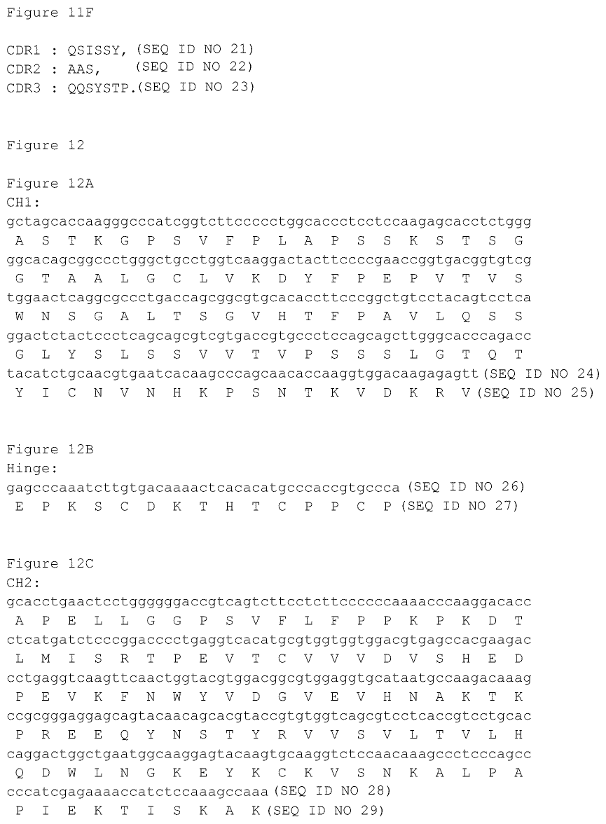

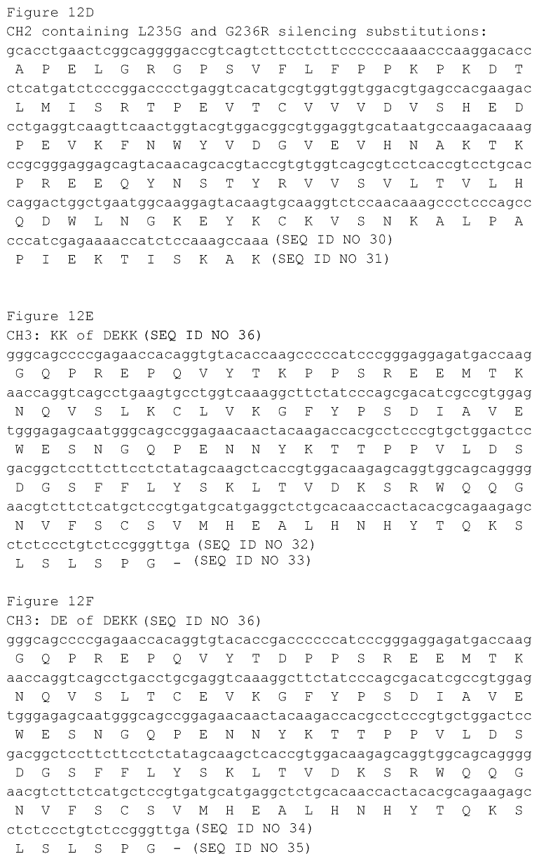

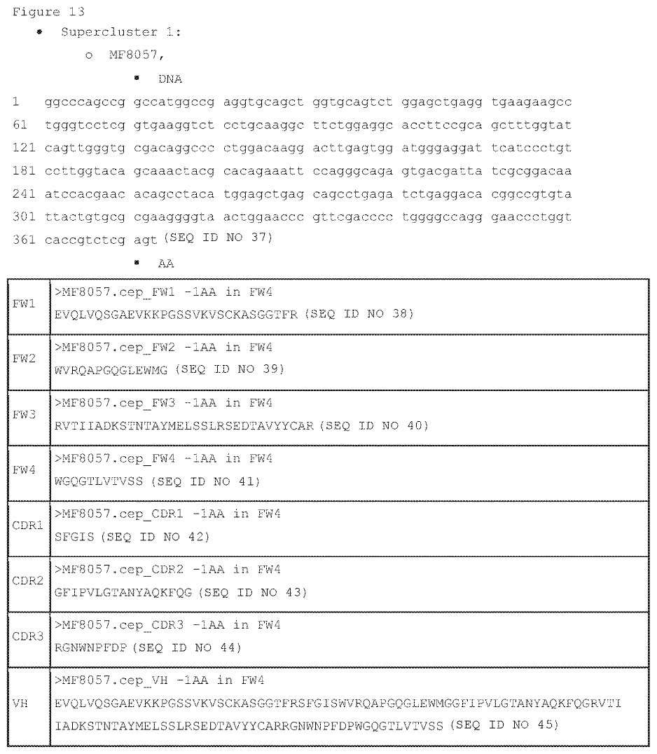

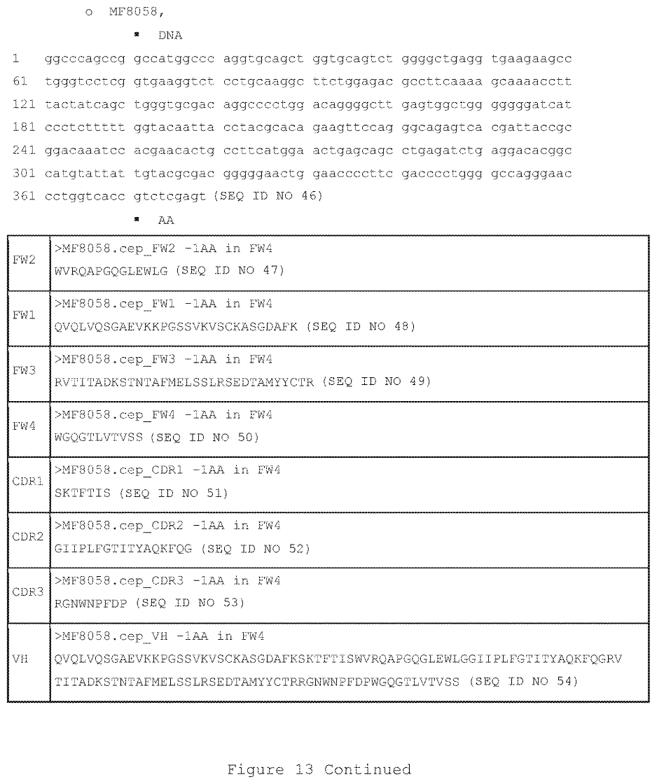

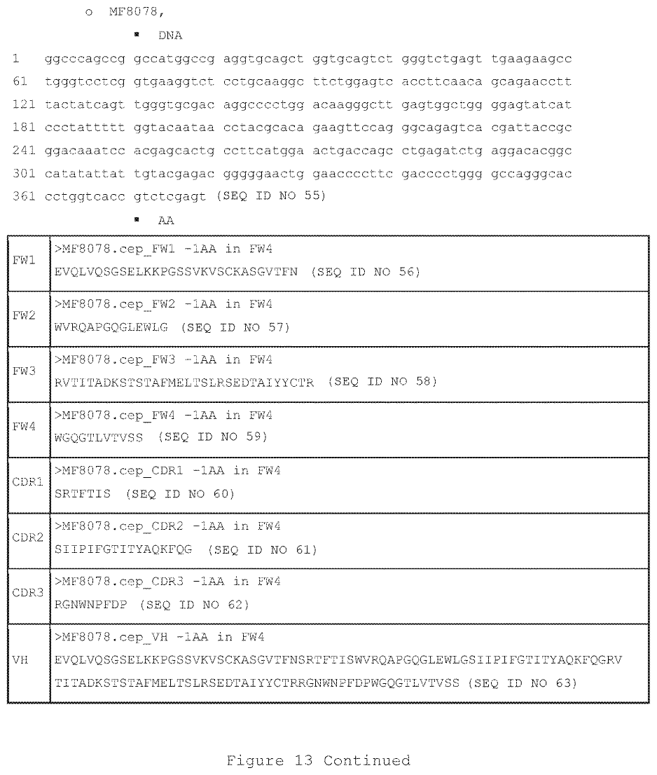

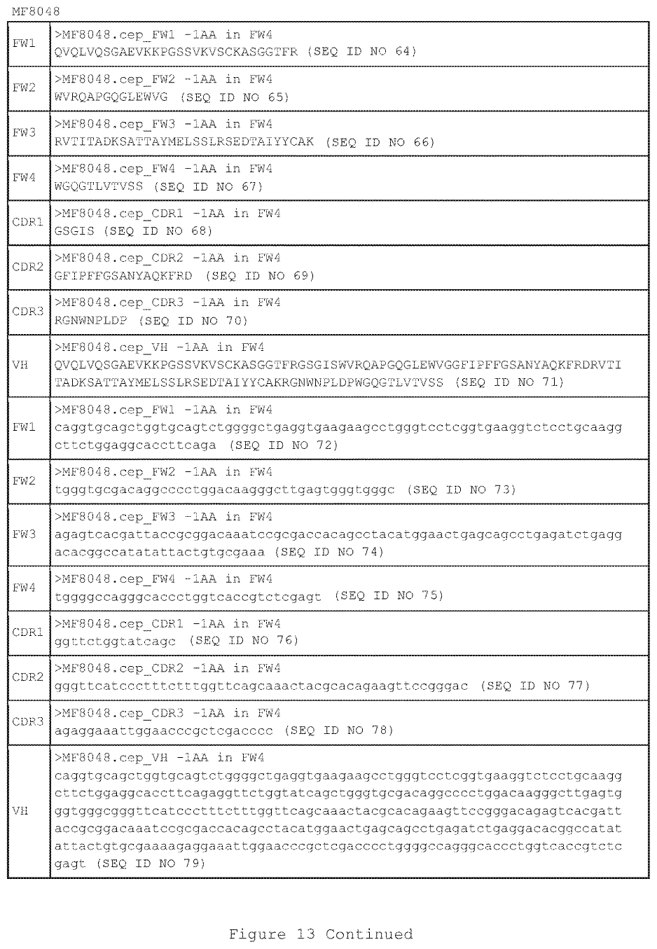

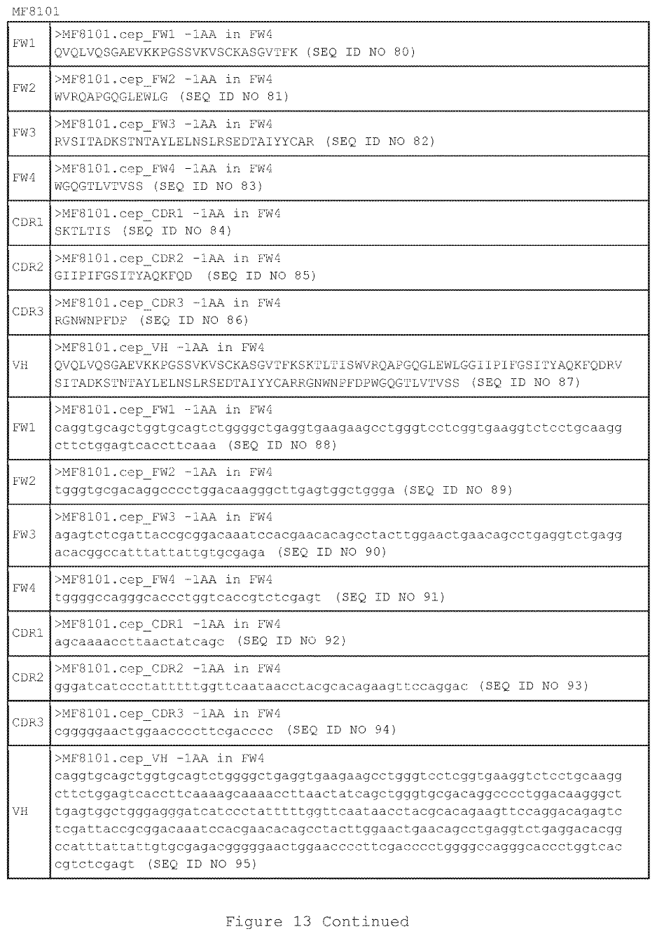

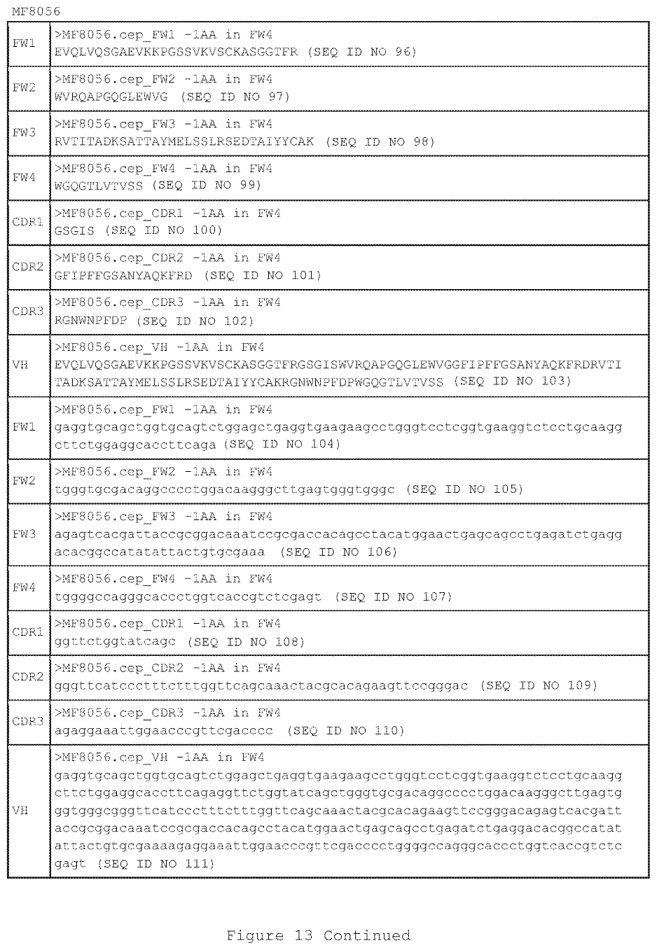

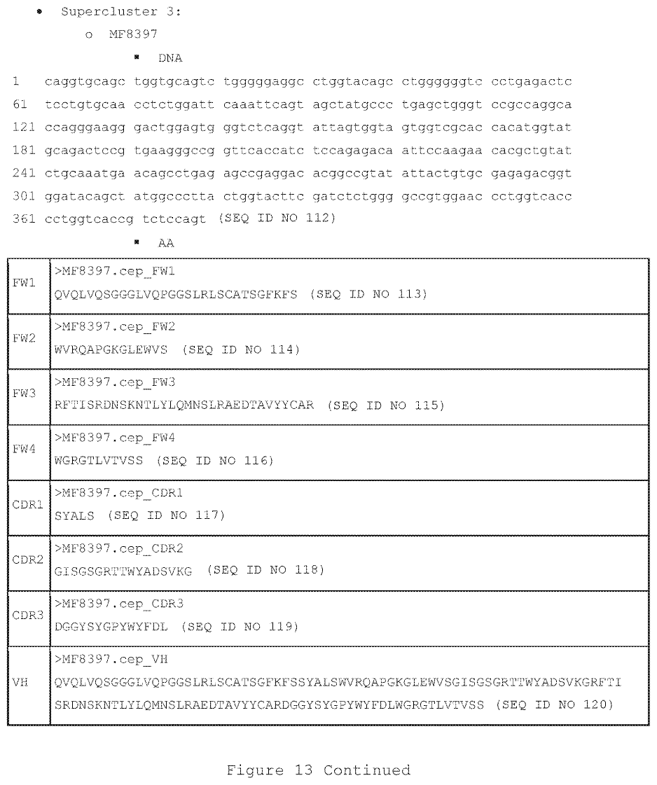

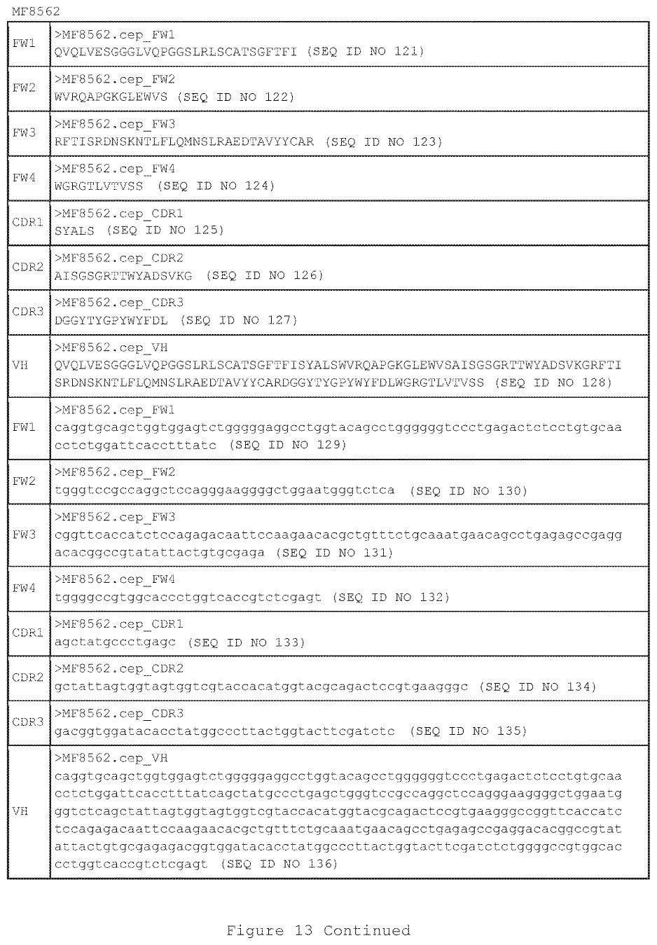

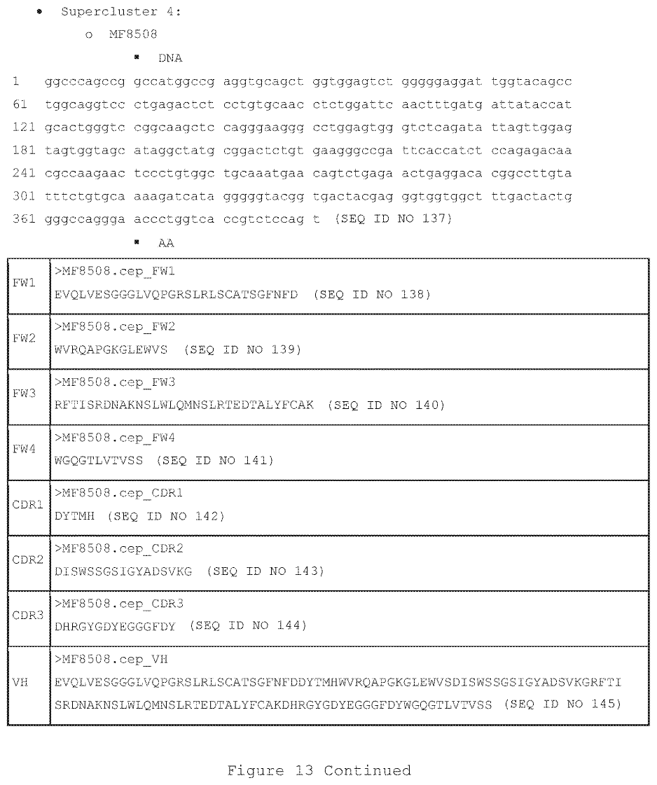

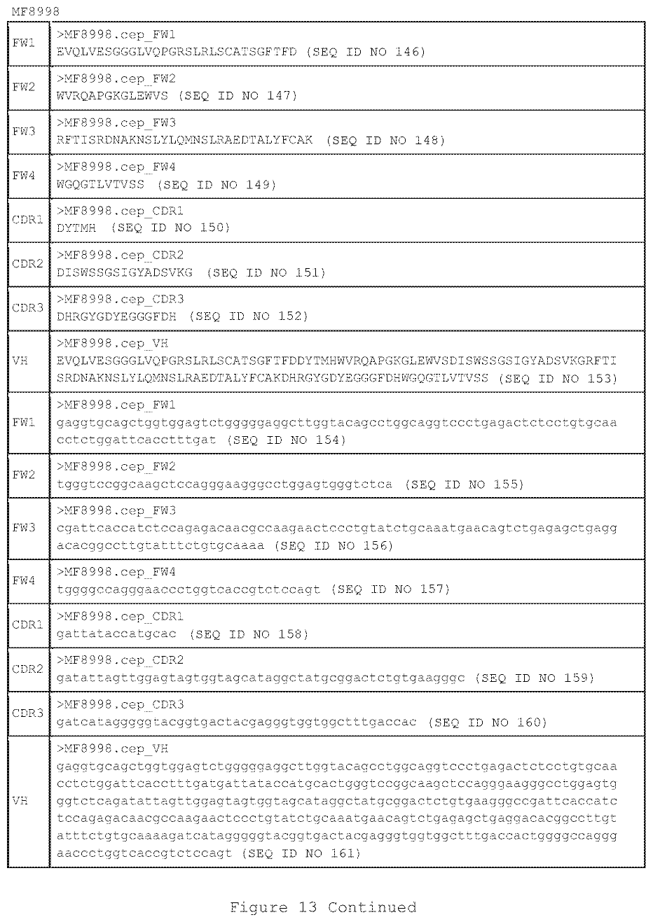

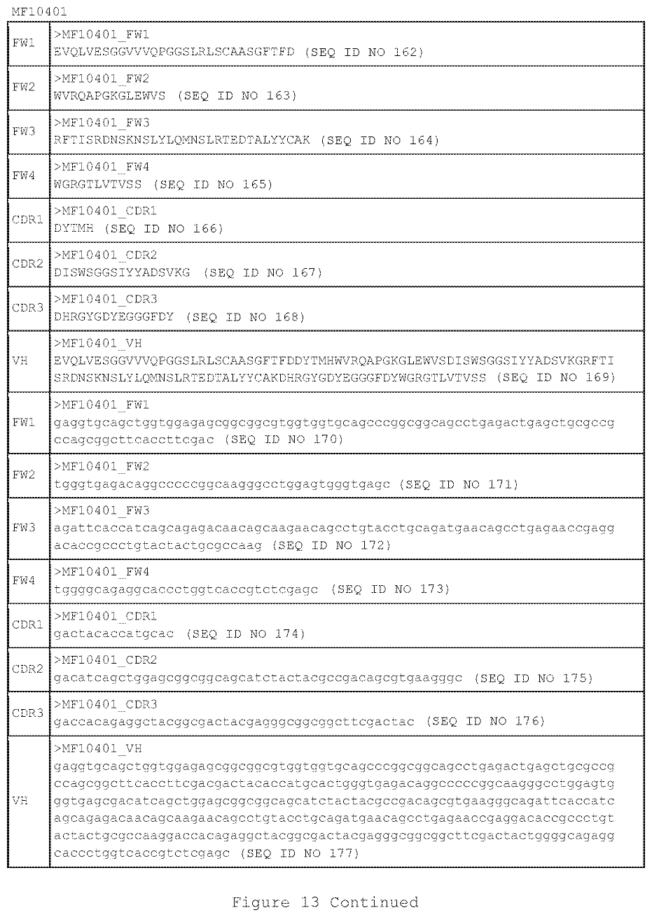

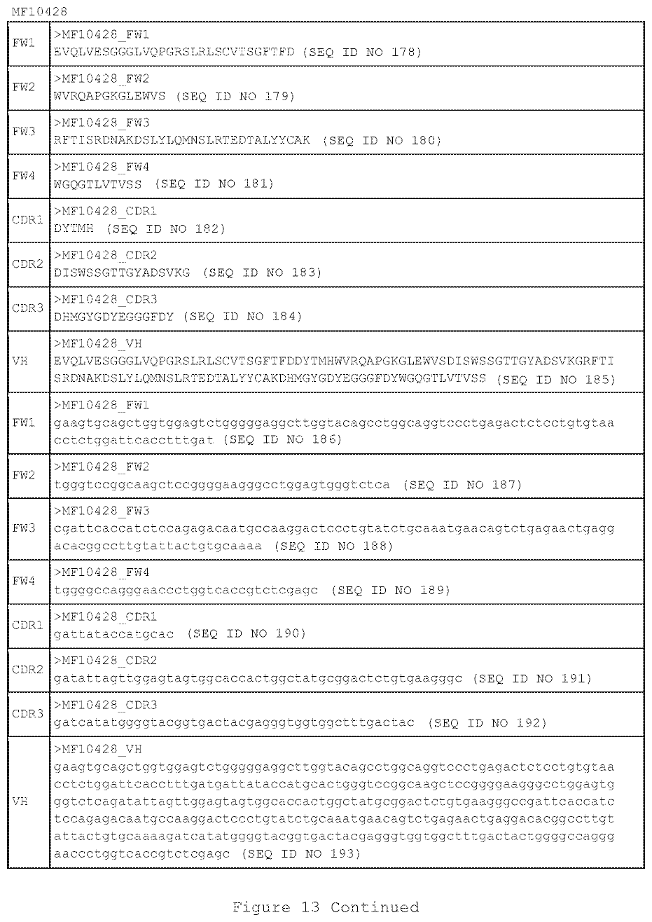

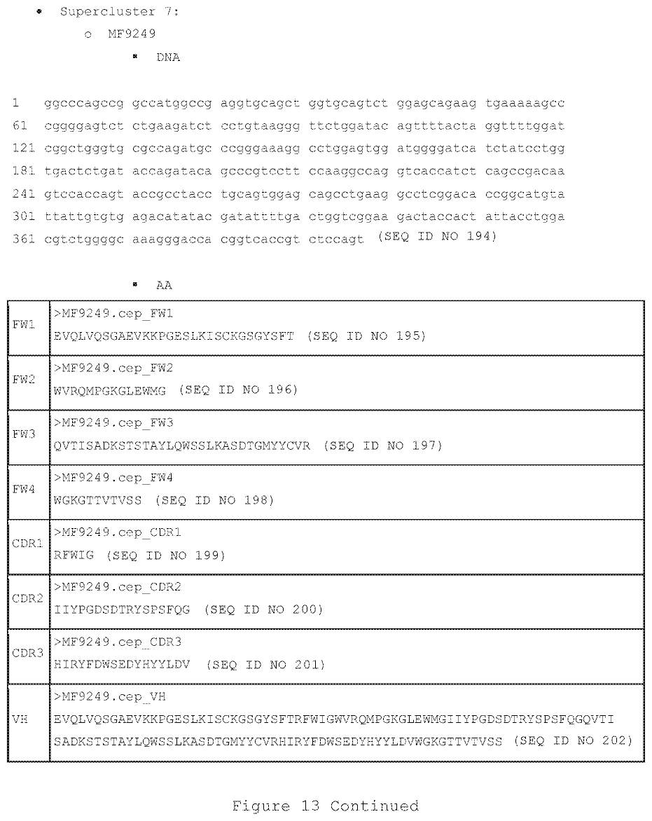

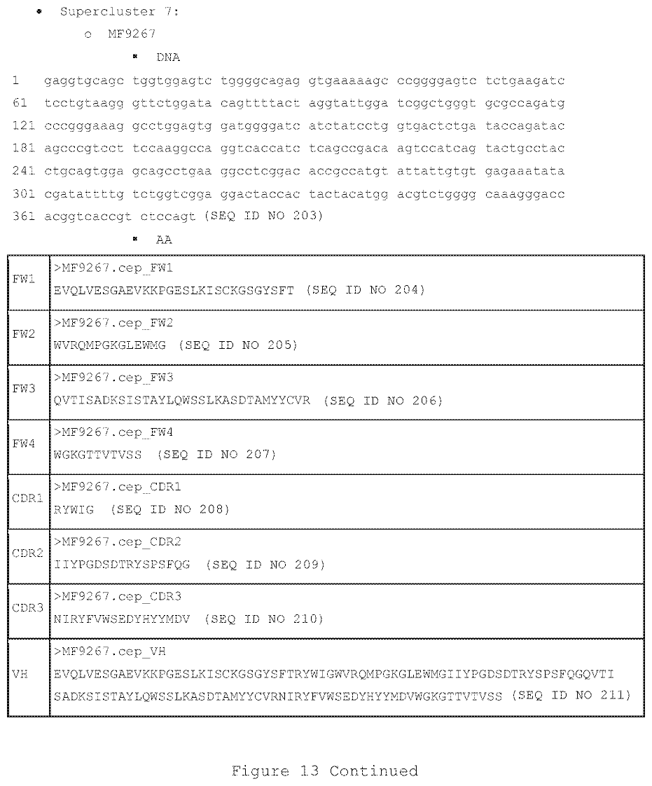

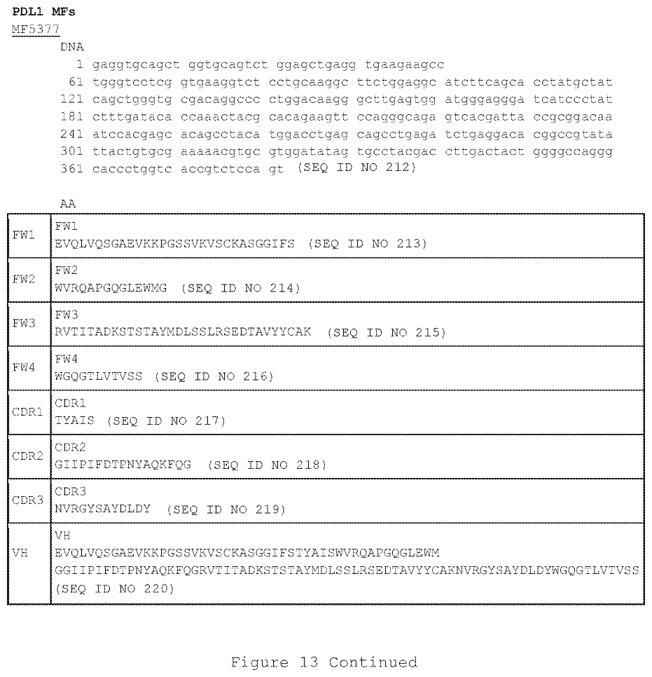

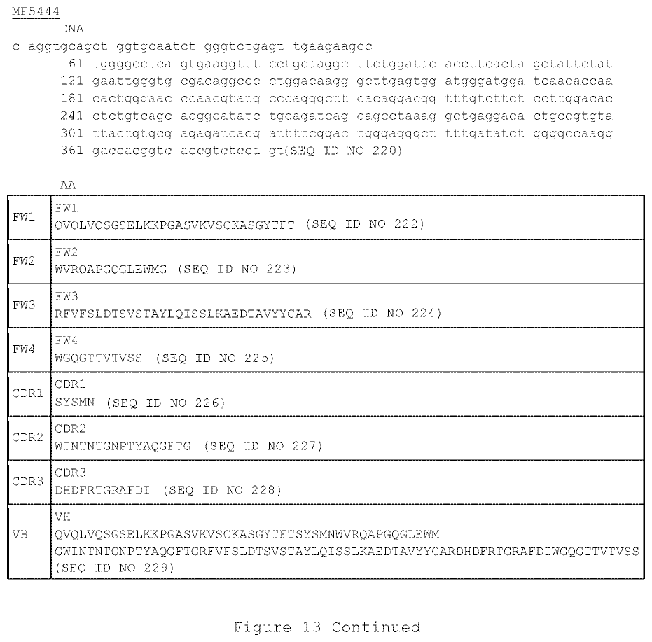

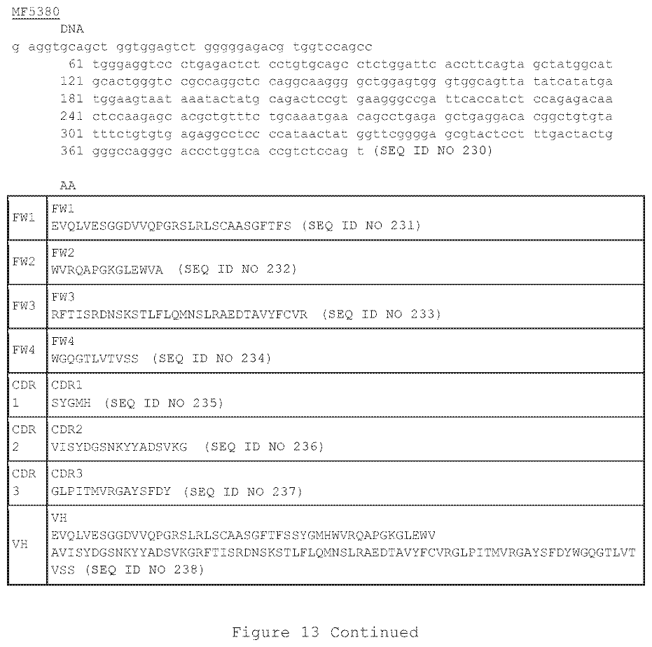

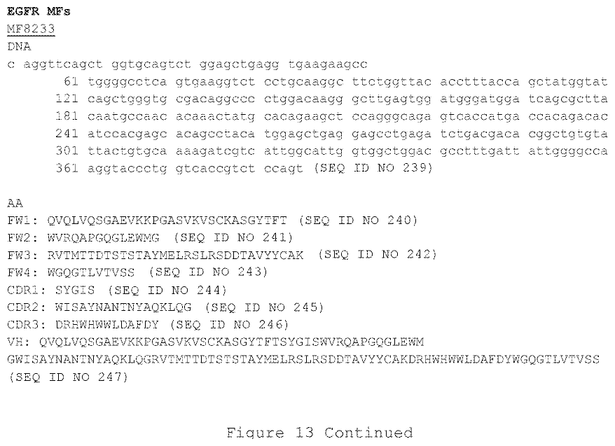

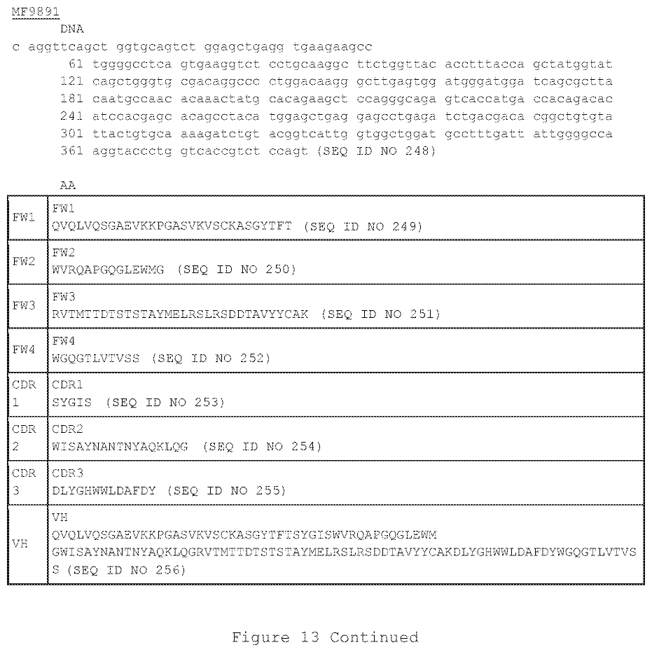

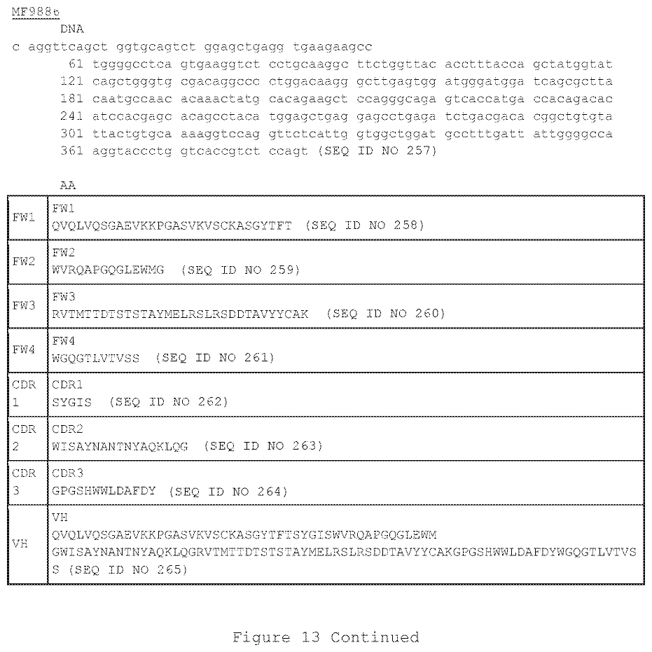

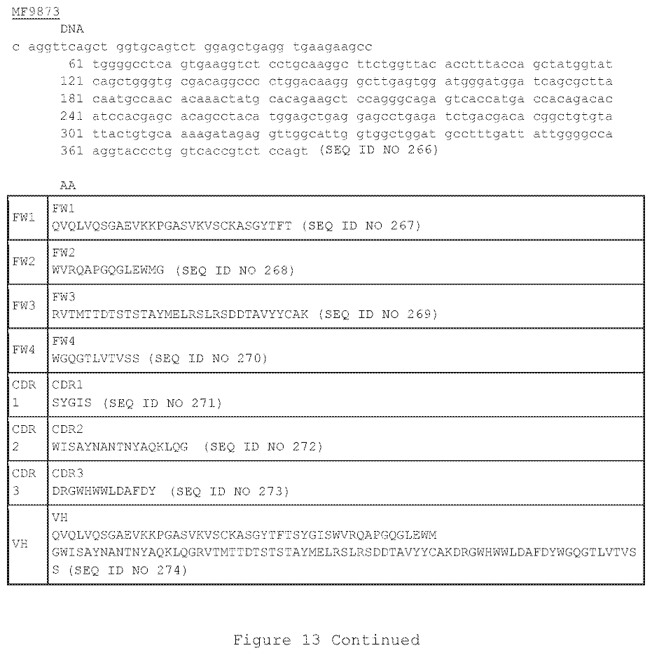

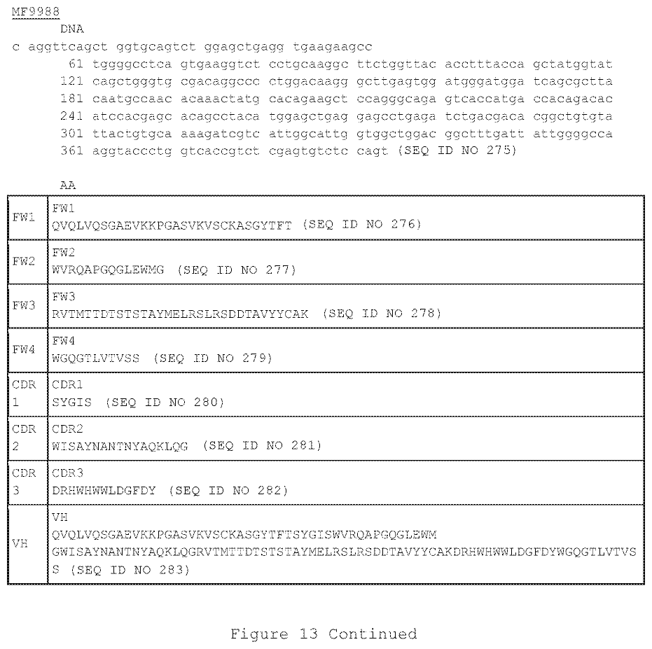

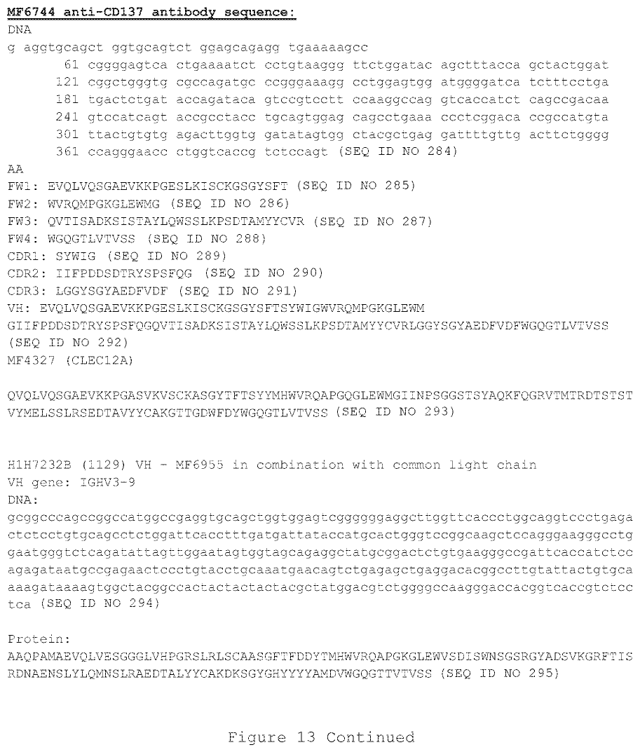

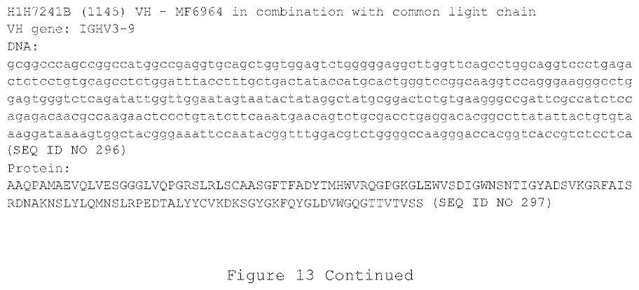

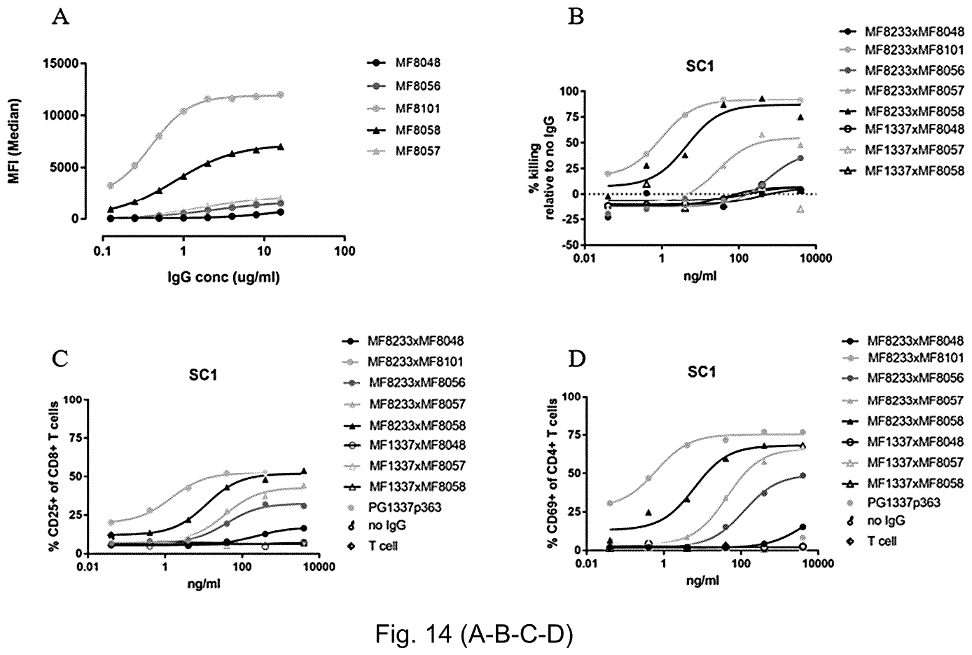

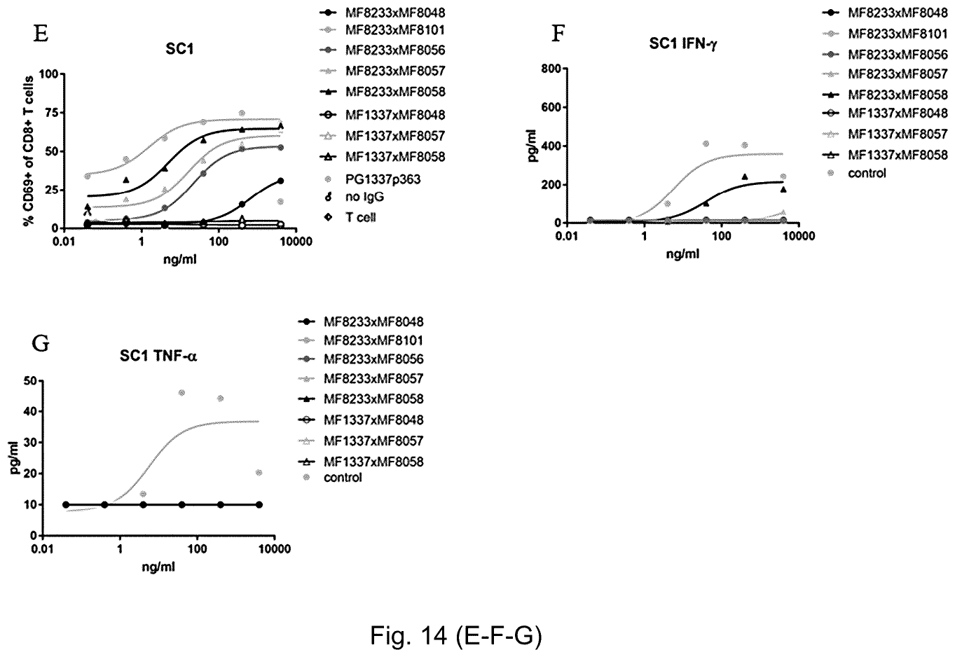

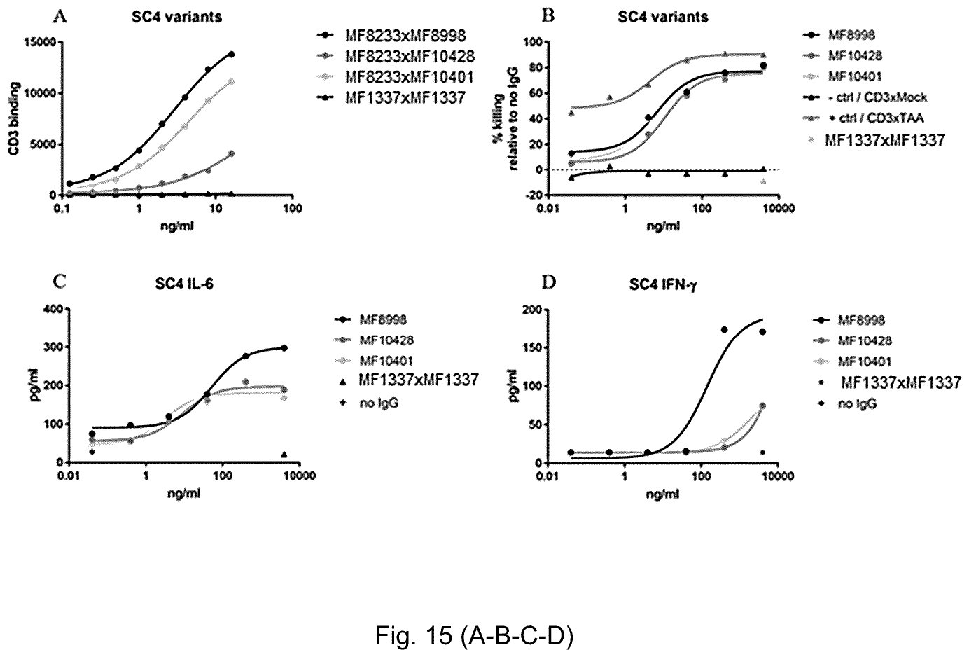

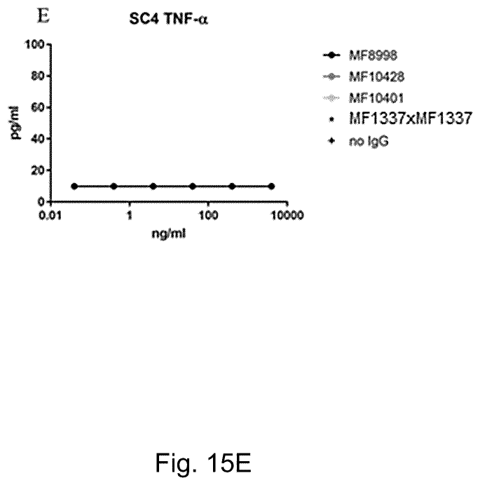

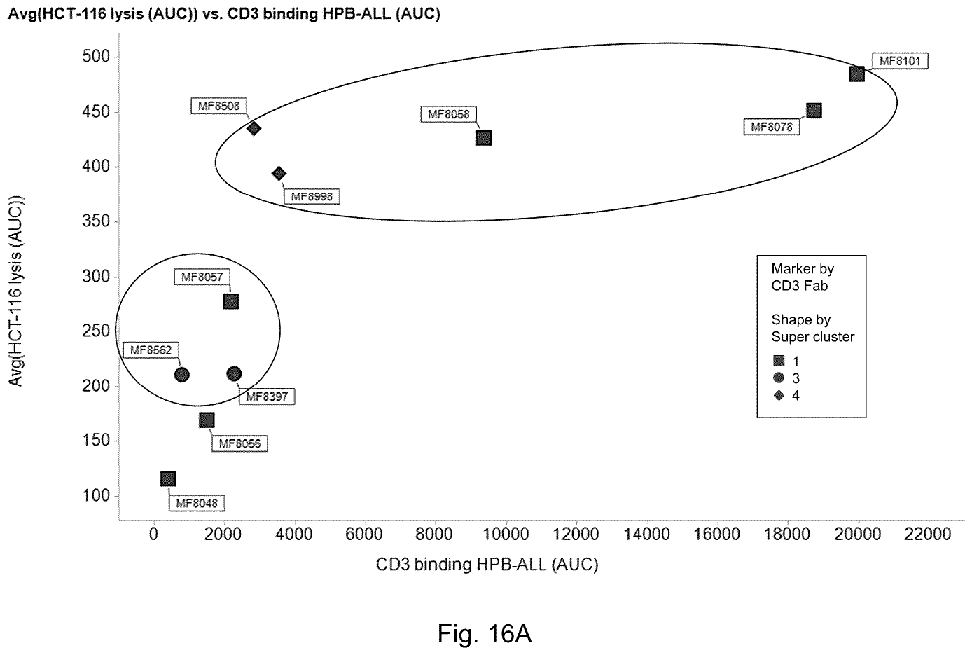

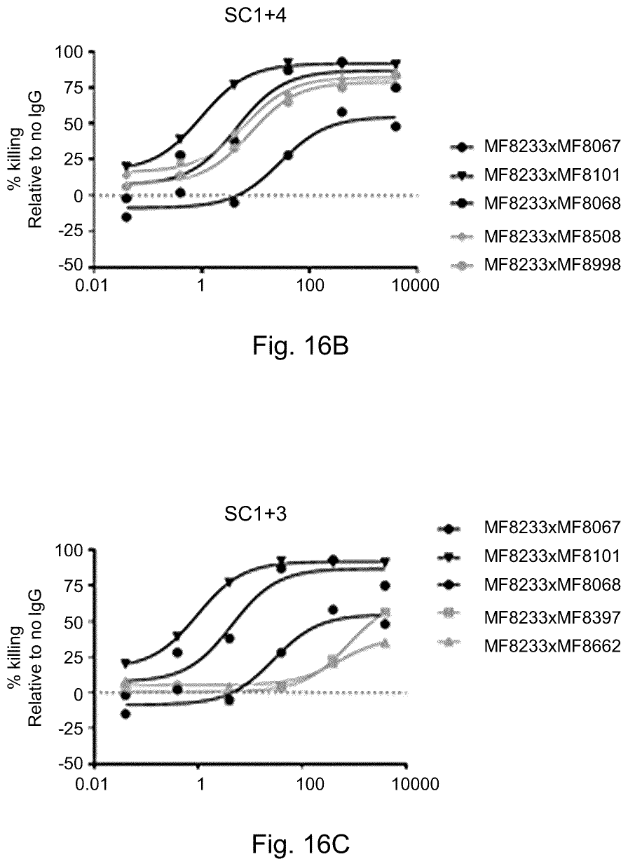

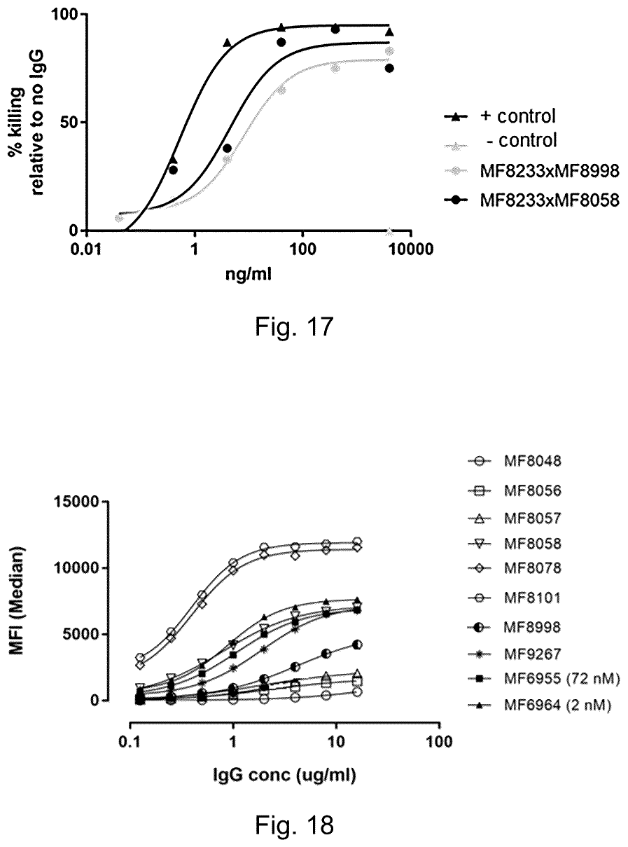

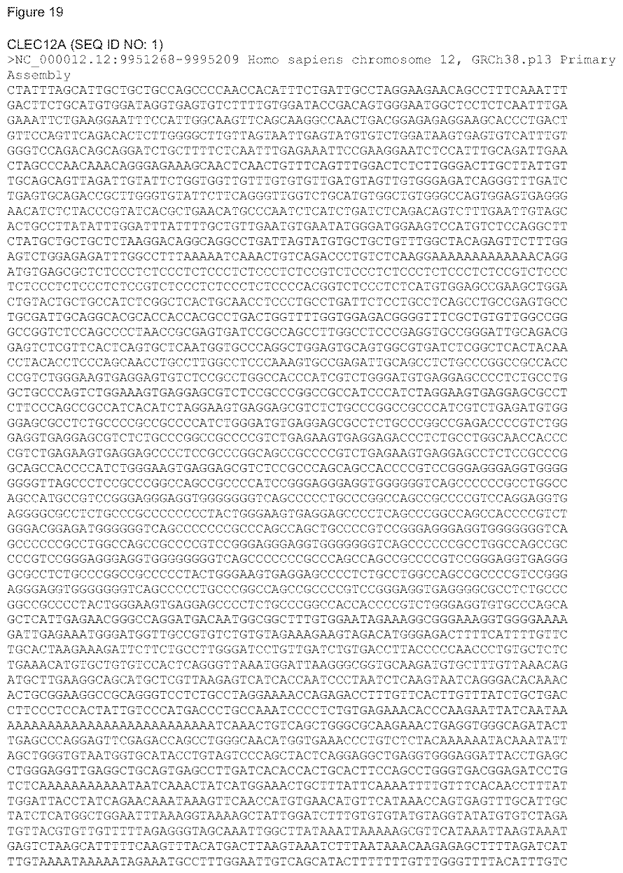

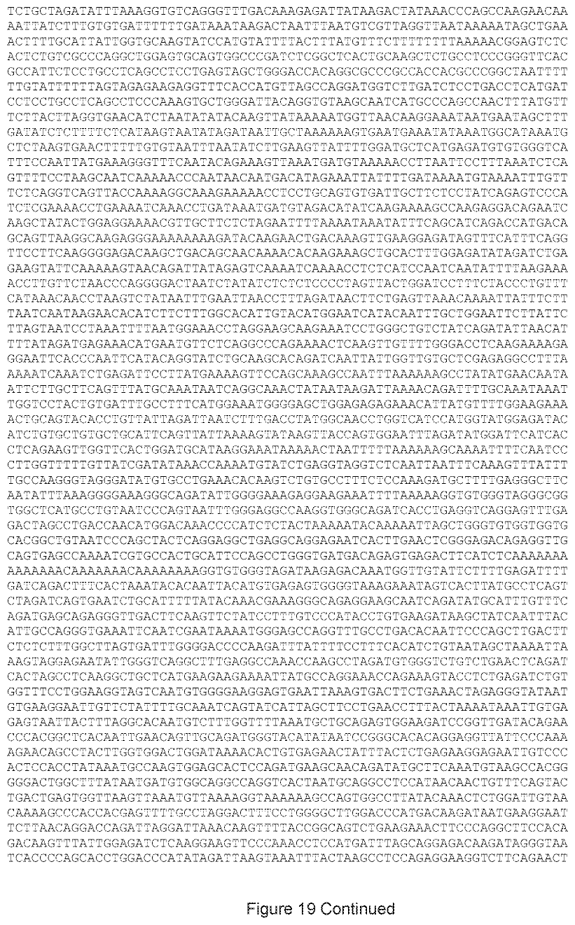

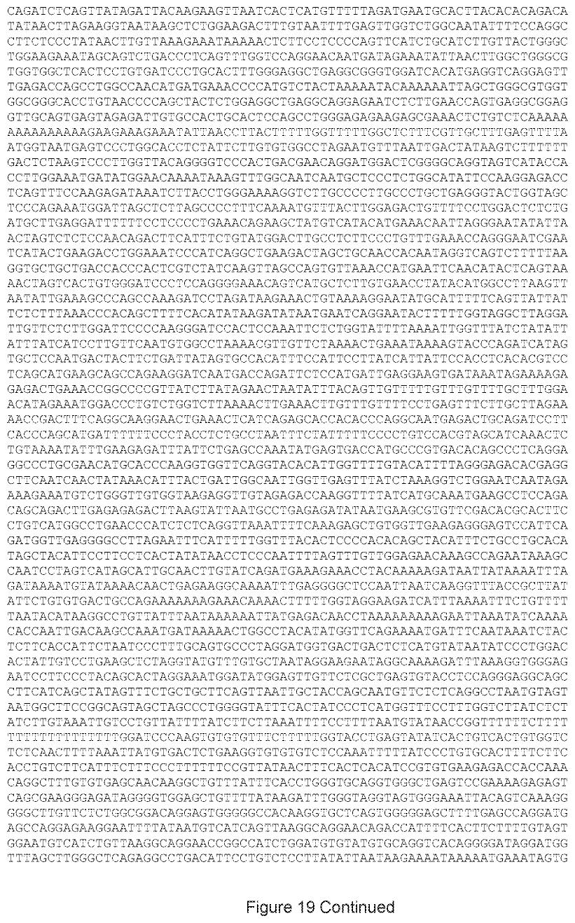

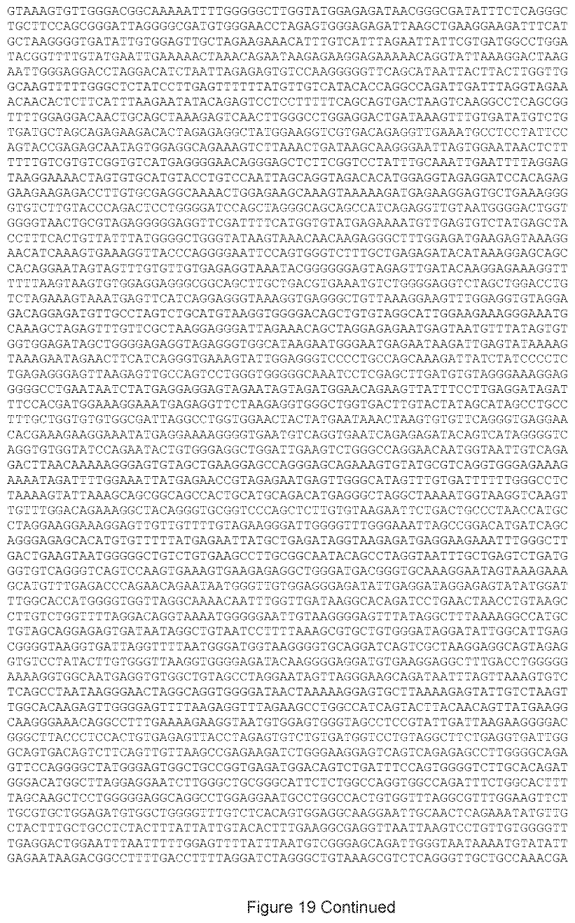

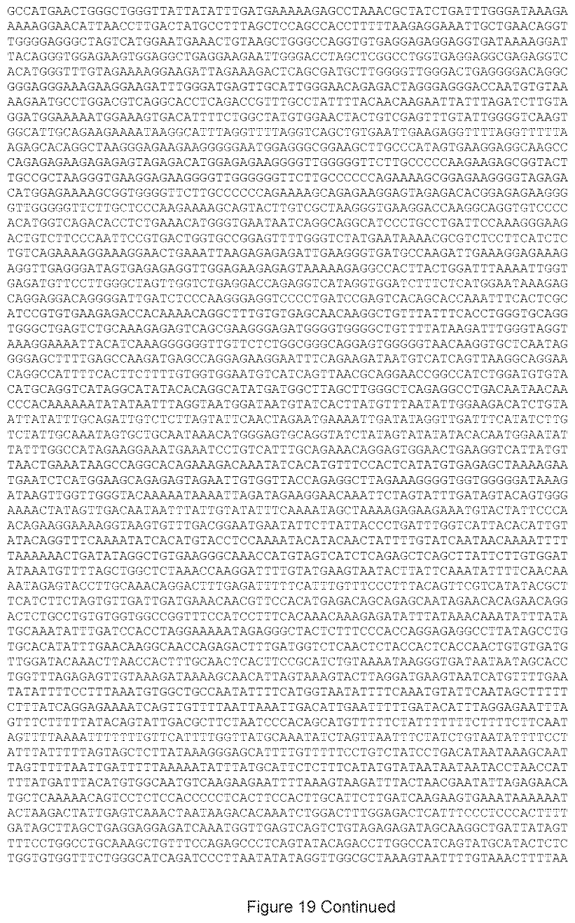

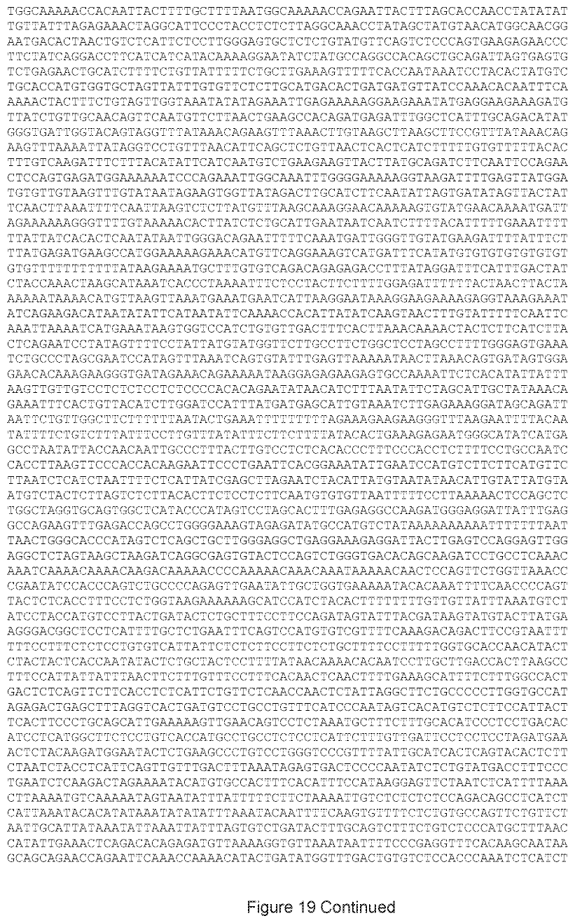

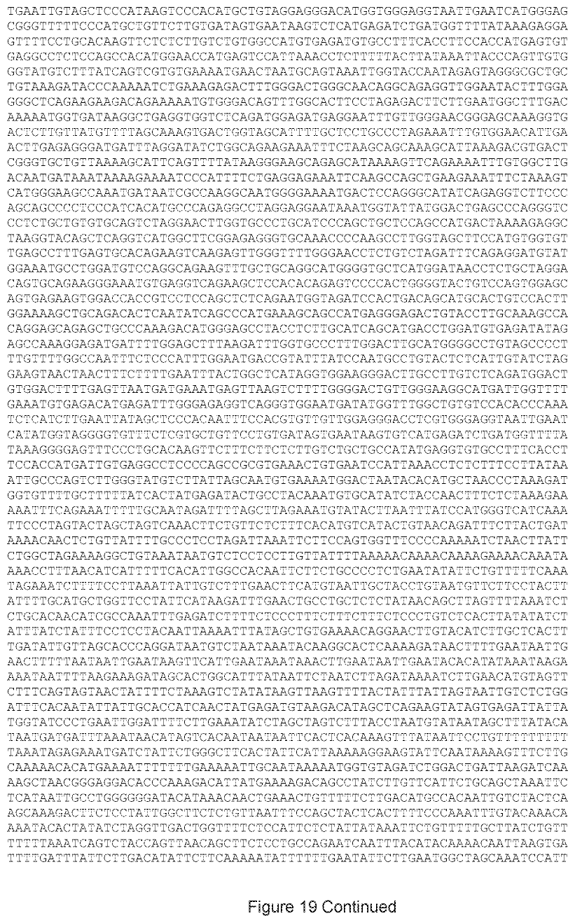

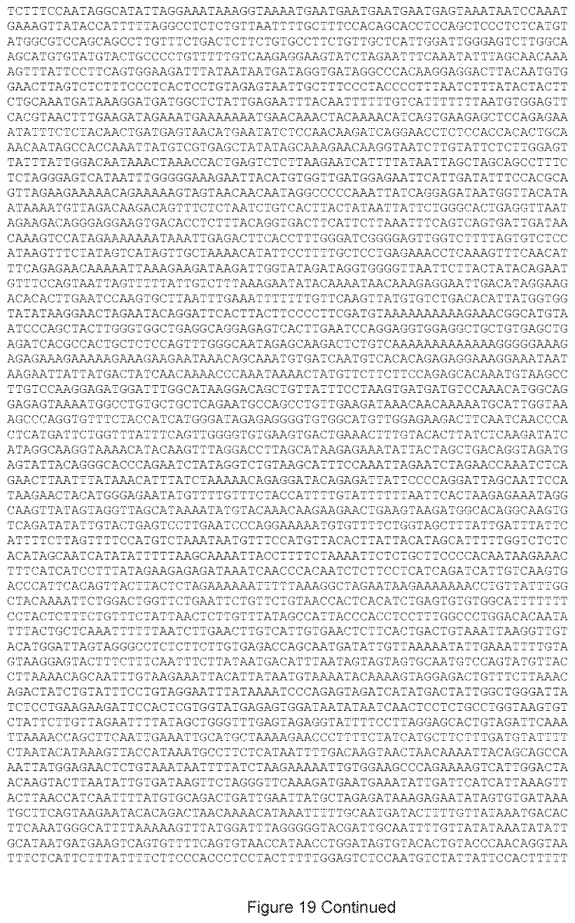

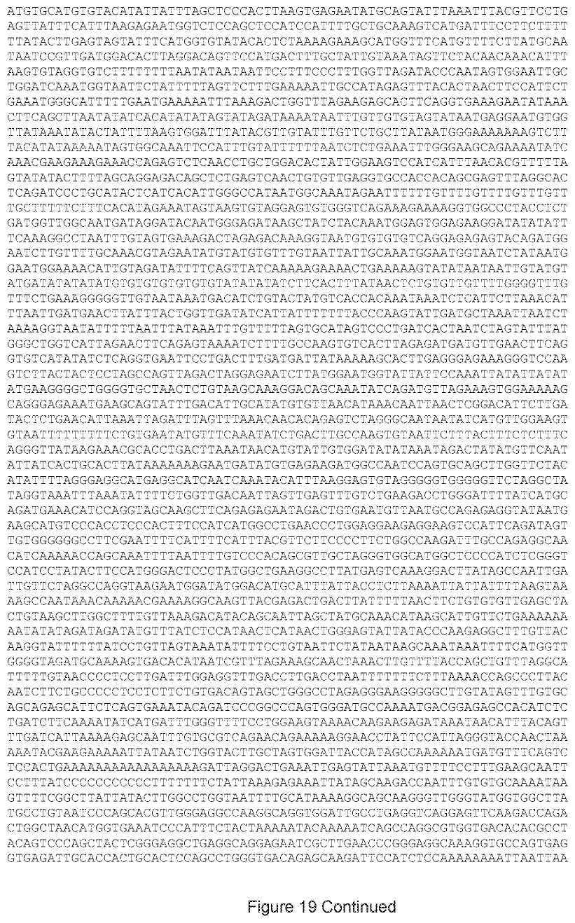

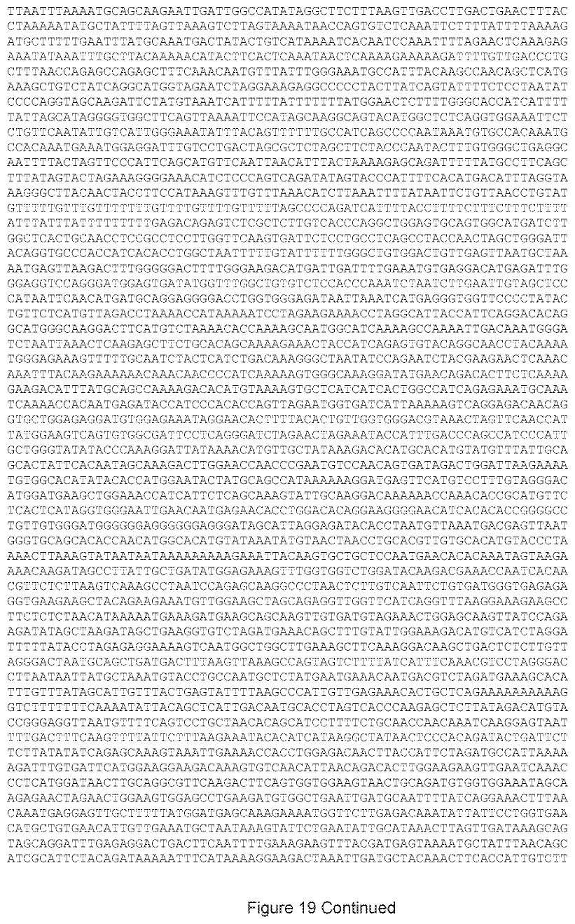

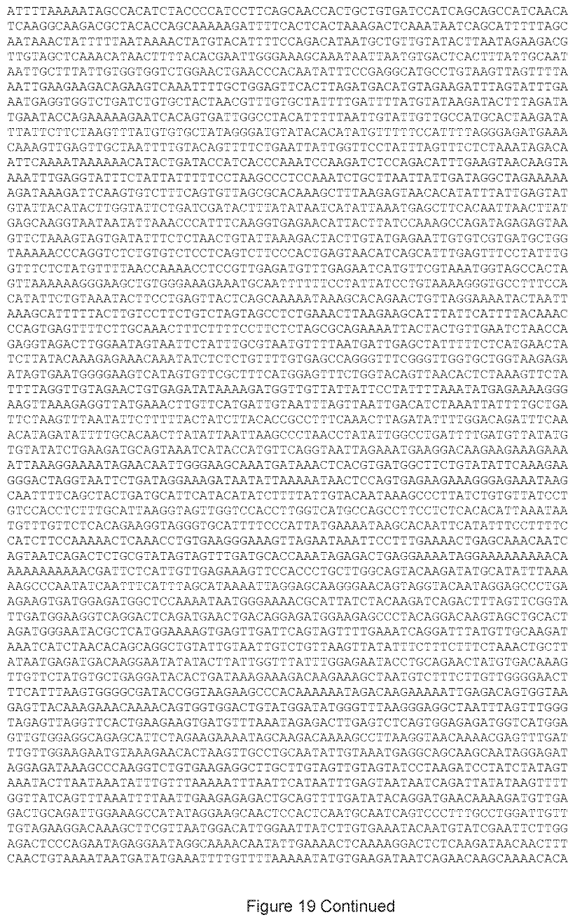

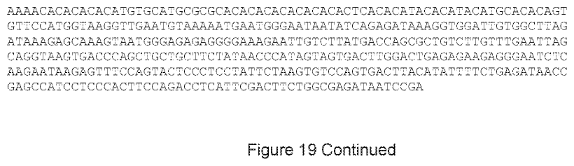

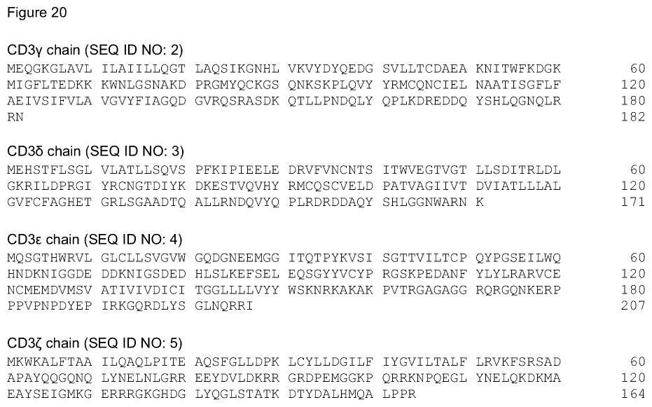

Evaluation of functional activity: T cell cytotoxicity assay with BxPC3 target cells upon treatment with EGFRxCD3 bispecific antibodies. Each bispecific antibody comprises a CD3 binding domain comprised of a heavy chain variable region designated by MF number, and an EGFR binding domain comprising a heavy chain variable region MF8233. These variable regions are paired with a common light chain to form an EGFRxCD3 bispecific antibody. Affinity (HPB-ALL binding) versus BxPC3 lysis. Certain antibodies of the invention exhibit a relative low level of binding to HPB-ALL cells indicating that the CD3 binding domain of the antibody binds human CD3 with a comparatively low affinity. It is clear that the relative lower affinity does not necessarily prohibit tumor antigen mediated T cell cytotoxicity of BxPC3 cells (BxPC3 lysis, vertical axis). The bispecific antibodies MF8233×MF8508 and MF8233×MF8057 can efficiently lyse BxPC3 cells whereas the bispecific antibodies MF8233×MF8397 and MF8233×MF9249 do not do so efficiently while having similar binding. Further, a comparison of the bispecific antibody MF8233×MF6955 binds HPB-ALL (i.e. human CD3) with a higher affinity but does not lyse BxPC3 cells more efficiently than the bispecific antibodies MF8233×MF8508 and MF8233×MF8057 that bind CD3 to a lesser extent. MF6955 is a heavy chain variable region combined with a common light chain and used as comparator sequences, and corresponds to H1H7232B (1129) VH, in US2014/0088295 A1. The comparator bispecific antibody having MF6955 and the same EGFR binding domain separately has a higher affinity for human CD3 than MF9267, and exhibits more efficient killing than MF9267. In contrast, other antibodies incorporating a CD3 binding domain of the invention, such as MF8058, have approximately the same binding activity as MF6955, yet demonstrate more efficient killing of BxPC3 cells, such as MF8233×MF8058. Other bispecific antibodies comprising a binding domain of the invention capable of binding CD3 demonstrate relative high binding and more efficient killing, such as MF8233×MF8078, which are useful for particular applications described herein. Whereas other bispecific antibodies comprising a binding domain of the invention capable of binding CD3 demonstrate relative low affinity and low killing, such as MF8233×MF9249 and MF8233×MF8397, which are useful for alternative applications described herein.

FIG. 2

Antibody titration curves indicating their capacity to induce T cell mediated % killing of BxPC3 target cells compared to no antibody control. Curves for the antibodies MF8233×MF8078, MF8233×MF8397; and MF8233×MF8508 are shown.

FIG. 3

Summary of the titration curve data of various bispecific antibodies in T cell cytotoxicity with BxPC3 target cells. The CD3 Fab column indicates the MF number of the CD3 binding arm. The EGFR arm has the indicated MF8233 number. The column indicates the supercluster numbers to set out variants based on the same VH gene segment. The column indicating CD3 binding reflect the results of the HBP-ALL binding experiment. The results of two independent cytotoxicity assays to determine capacity to induce T cell mediated lysis of BxPC3 target cells are shown.

FIG. 4

T cell activation in T cell cytotoxicity assay with BxPC3 target cells on CD8+ T cells with the expression. Antibody titration curves of various supercluster numbers, which set out variant CD3 binding domains. The other arm of the bispecific antibody has the heavy chain binding domain of MF8233. For comparison the bispecific antibodies MF8233×MF6955 and MF8233×MF6964 were tested as well, where MF6955 and MF6964 are heavy chain variable regions combined with a common light chain and used as comparator sequences, and correspond to H1H7232B (1129) VH and HH7241B (1145) respectively, in US2014/0088295 A1

FIG. 5

Summary of the titration curve data of various antibodies in T cell activation T cell cytotoxicity assay with BxPC3 target cells. The MF nr. column indicates the MF number of the CD3 binding domain. The EGFR binding domain has the indicated MF8233 number. The supercluster information of the various CD3 binding domain sequences is indicated in column “supercluster”; The column indicating CD3 affinity reflects the results of the HBP-ALL binding experiment.

The results of CD4+ and CD8+ cells are shown for the markers CD69 and CD25. The indicated bispecific antibodies are examples from a larger pool of bispecific antibodies.

FIG. 6

Evaluation of functional activity: T cell cytotoxicity assay with BxPC3 target cells. Affinity (HPB-ALL binding) versus CD8+ T cell activation measured by CD69 expression. Certain antibodies of the invention exhibit a relative low level of binding to HPB-ALL cells indicating that the CD3 binding domain of the antibody binds human CD3 with a relative low affinity. Such affinity does not necessarily prohibit tumor antigen mediated T-cell activation as exemplified by the results of the CD8 positive CD69 activation analysis. The bispecific antibodies MF8233×MF8508 and MF8233×MF8057 can efficiently activate T cells whereas the bispecific antibodies MF8233×MF8397 and MF8233×MF9249 do not do so as efficiently. Certain CD3 binding domains that do not bind efficiently to these cells also do not activate T cells (see lower left corner). Whereas other CD3 binding domains MF8508 and MF8057, which bind HPB-ALL cells less than comparator CD3 binding domain MF6955, for example, activate T cells to a similar degree. Other bispecific antibodies comprising a binding domain of the invention capable of binding CD3 demonstrate relative high binding and high levels of activation, such as MF8078, which has use in particular applications described herein. Whereas other bispecific antibodies comprising a binding domain of the invention capable of binding CD3 demonstrates relative low affinity and low activation, such as MF9249 and MF8397, which are useful for alternative applications described herein.

FIG. 7

Evaluation of functional activity: T cell cytotoxicity assay with HCT116 target cells. Affinity (HPB-ALL binding) versus HCT-116 lysis.

Certain bispecific antibodies of the invention exhibit a low level of binding to HPB-ALL cells indicating that the CD3 binding domain of the antibody binds human CD3 with a comparatively low affinity. It is clear that the low affinity does not necessarily prohibit tumor antigen mediated cell lysis of HCT-116 cells (vertical axis). The bispecific antibodies MF8233×MF8508 and MF8233×MF8057 can efficiently lyse HCT-116 cells whereas the bispecific antibodies MF8233×MF8397 and MF8233×MF9249 do not do so efficiently. For comparison the bispecific antibodies MF8233×MF6955 and MF8233×MF6964 bind HPB-ALL (i.e. human CD3) with a higher affinity than, for example, MF8233×MF8508, MF8233×MF8057 and MF8233×MF9267, but do not lyse HCT-116 cells more efficiently than MF8233×MF8508 or MF8233×MF9267, or significantly more than MF8233×MF8057 relative to the difference in binding, in a test as presented here. Another bispecific antibodies comprising a binding domain of the invention capable of binding CD3 demonstrates relative high binding and high levels of killing, such as MF8078, which has use in particular applications described herein. Whereas other bispecific antibodies comprising a binding domain of the invention capable of binding CD3 demonstrate relative low affinity and low killing, such as MF9249 and MF8397, which are useful for alternative applications described herein.

FIG. 8

Antibody titration curves in T cell cytotoxicity assay with HCT-116 target cells indicating the % killing of HCT-116 cells compared to no antibody control. Curves for various bispecific antibodies are shown.

FIG. 9

Summary of the titration curve data of various antibodies in T cell cytotoxicity assay with HCT-116 target cells. The CD3 Fab column indicates the MF number of the CD3 binding domain. The EGFR binding domain has the indicated MF8233 number. The column indicates the supercluster numbers to set out variants based on the same VH gene segment. The column indicating CD3 binding reflect the results of the HBP-ALL binding experiment. Percentage lysis of HCT-116 cells and EC50 values for lysis (ng/ml) are indicated in the next columns. The indicated bispecific antibodies are examples from a larger pool of bispecific antibodies.

FIG. 10

FIG. 10A and FIG. 10B set out a schematic diagram of the MV1624 expression vector and the MV1625 expression vector.

FIG. 11

Common light chain used in mono- and bispecific IgG.

FIG. 11A: Common light chain amino acid sequence. FIG. 11B: Common light chain variable domain DNA sequence and translation (IGKV1-39/jk1). FIG. 11C: Common light chain constant region DNA sequence and translation. FIG. 11D: IGKV1-39/jk5 common light chain variable domain translation. FIG. 11E: V-region IGKV1-39A; FIG. 11F: CDR1, CDR2 and CDR3 of the common light chain.

FIG. 12

IgG heavy chains for the generation of bispecific molecules. FIG. 12A: CH1 region. FIG. 12B: hinge region. FIG. 12C: CH2 region. FIG. 12D: CH2 containing L235G and G236R silencing substitutions. FIG. 12E: CH3 domain containing substitutions L351K and T366K (KK). FIG. 12F: CH3 domain containing substitutions L351D and L368E (DE).

FIG. 13

Sequences of various DNA encoding and amino acid sequences of the heavy chain variable regions and parts thereof described in the specification.

FIG. 14

Characterization of additional clones from supercluster 1 in comparison with clones MF8057 and MF8058. FIG. 14A: Binding of selected MF clones with HPB-ALL human cells expressing a human CD3-TCR complex in a FACS assay. FIG. 14B: T-cell cytotoxicity assay with HCT-116 cells indicating the % killing of HCT-116 cells. FIG. 14C-E: Quantification of activation markers CD25 and CD69 in FACS indicating T-cell activation. FIG. 14F-G: Cytokine production in the supernatants from the cytotoxicity assay.

FIG. 15

Characterization of clones from supercluster 4. FIG. 15A: Binding of the selected MF clones to HPB-ALL human cells. FIG. 15B: T cell cytotoxicity assay with BxPC3 cells indicating % killing of BXP3 cells. FIG. 15C-E: Cytokine production in the supernatants from the cytotoxicity assay.

FIG. 16

Evaluation of CD3 functional activity. FIG. 16A: Affinity (HPB-ALL) on X-axis versus HCT-116 Lysis on Y-axis for additional clones from supercluster 1 (MF8048, MF8101, MF8056), supercluster 3 (MF8562) and supercluster 4 (MF8998). FIG. 16B: Antibodies belonging to supercluster 1 and supercluster 4 which show similar activity in cytotoxicity assay and different binding affinity. FIG. 16C: Antibodies belonging to supercluster 1 and supercluster 3 which exhibit similar binding affinity and differential lysis activity.

FIG. 17

Activity of CD3 Fabs MF8998 and MF8058 in bispecific CD3×EGFR format.

FIG. 18

FACS binding data of a large panel of IgGs specific for CD3. For antibodies MF5196, MF6955 and MF6964, binding was determined by BIAcore™ on CD388-Fc antigen, whereas FACS binding data to HPB-ALL cells is shown for the remainder of the clones.

FIG. 19

Nucleotide sequence of human CLEC12A.

FIG. 20

Amino acid sequences of the human CD3γ-, δ-, ε- and ζ-chain.

The following Examples illustrate the invention:

### EXAMPLES

### Cell Lines

BxPC3 human pancreatic cancer cell line.

HCT-116 human colon carcinoma cell line.

Immunization of Memo® mice with CD3

For generation of human antibodies binding to CD3, mice transgenic for the human common light chain and for a human heavy chain (HC) minilocus (comprising a selection of human V gene segments, all human Ds and all human Js) (see WO2009/157771 incorporated herein by reference) were immunized with TCR/CD3 containing lipoparticles (Intergral Molecular). These mice are referred to as ‘MeMO®’ mice. For specific heavy chain variable regions, or trivalent multimers having the sequences disclosed herein, they can be produced by any means known to persons of ordinary skill in the art.

MeMo® mice were immunized with Hek293T-derived human 5D5M TCR/CD3 containing lipoparticles, followed by human T-cells for the generation of an anti-TCR/CDR3 immune response and anti-TCR/CD3 antibody panel generation.

Lipoparticles concentrate conformationally intact membrane proteins directly from the cell surface, permitting these complex proteins to be manipulated as soluble, high-concentration proteins for antibody immunization and screening

The lipoparticles used in the present study for immunisation contain the 5D5M TCRαβ combination. Vectors comprising the 5D5M TCRαβ combination were synthesized, cloned and used to generate lipoparticles containing this TCR/CD3 combination by transient transfection into HEK293T cells (Intergral Molecular).

5D5M TCRα
(SEQ ID NO 13)
MWGVFLLYVSMKMGGTTGQNIDQPTEMTATEGAIVQINCTYQTSGFNGLF
WYQQHAGEAPTFLSYNVLDGLEEKGRFSSFLSRSKGYSYLLLKELQMKDS
ASYLCAVMDSNYQLIWGAGTKLIIKPDIQNPDPAVYQLRDSKSSDKSVCL
FTDFDSQTNVSQSKDSDVYITDKTVLDMRSMDFKSNSAVAWSNKSDFACA
NAFNNSIIPEDTFFPSPESSCDVKLVEKSFETDTNLNFQNLSVIGFRILL
LKVAGFNLLMTLRLWSS
5D5M TCRβ
(SEQ ID NO 14)
MRIRLLCCVAFSLLWAGPVIAGITQAPTSQILAAGRRMTLRCTQDMRHNA
MYWYRQDLGLGLRLIHYSNTAGTTGKGEVPDGYSVSRANTDDFPLTLASA
VPSQTSVYFCASSEAGGNTGELFFGEGSRLTVLEDLNKVFPPEVAVFEPS
EAEISHTQKATLVCLATGFFPDHVELSWWVNGKEVHSGVSTDPQPLKEQP
ALNDSRYCLSSRLRVSATFWQNPRNHFRCQVQFYGLSENDEWTQDRAKPV
TQIVSAEAWGRADCGFTSVSYQQGVLSATILYEILLGKATLYAVLVSALV
LMAMVKRKDF

MeMo® mice were used for immunizations using TCR/CD3 lipoparticles and primary human T cells.

The immunization schedule contains points on day 35, 56, 77 and 98, where the antigen-specific Ig serum titer was determined by ELISA using QTG-derived 3SDX TCR/CD3 positive and −negative lipoparticles using anti mouse IgG detection and by ELISA using CD3c5E-Fc fusion protein as a positive control. The reactivity was observed in sera drawn at day 35 will determine which mice developed a relevant anti-TCR/CD3 response.

For all immunized mice, lymphoid material for antibody discovery was collected and stored when:

Titers are 1/300 for human TCR/CD3 (in ELISA using lipoparticles), or:

Titers are <1/300 and >1/100 for human TCR/CD3 and did not increase during the last booster immunization.

### Priming Immunisation Using Lipoparticles

To prime the humoral immune response in the MeMo® mice for TCR/CD3, lipoparticles containing the human 5D5M TCRαβ combination was used for immunization. Lipoparticles were used together with Gerbu adjuvant for the first and second injection.

### Booster Immunizations Using Polyclonal T-Cells

Mice were immunised by sub-cutaneous injection of cell suspension. The first booster immunisations (day 28) comprised a mix of cells in PBS with adjuvant and all subsequent injections are only composed of cells in PBS. Mice that have developed at day 35 serum IgG titers of 1/300 against human TCR/CD3 (determined by ELISA using lipoparticles) received additional injections with cells on days 42, 43 and 44. Mice that failed to meet these criteria receive booster immunisations (day 42 and 49) with cells. All subsequent immunisations are given as sub-cutaneous injections of cells in PBS. After the final immunisation, mice are sacrificed, bled for serum and the spleen and left inguinal lymph nodes are collected.

Screening Sera from Immunised Mice in ELISA

Interim serum IgG titers were screened by ELISA using TCR/CD3-containing lipoparticles and ‘null’ lipoparticles. Serum IgG titers were determined using anti-mouse IgG staining, as this staining was shown to be the most sensitive.

### Generation of ‘Immune’ Phage Antibody Repertoires by RT-PCR Cloning of VH Genes

From successfully immunized mice, the inguinal lymph nodes were used for the construction of ‘immune’ phage antibody repertoires. RNA was extracted from the lymphoid tissue using Trizol LS and 1 μg of total RNA was used in a RT reaction using an IgG-CH1 specific primer. The resulting cDNA was then used to amplify the polyclonal pool of VH-encoding cDNA using in-house developed VH-specific primers essentially as described in Marks et al. (J Mol Biol. 1991 Dec. 5; 222(3):581-97). The resulting PCR product was then cloned in a phagemid vector for the display of Fab fragments on phage, as described in de Haard et al. (J Biol Chem. 1999 Jun. 25; 274(26):18218-30) with the exception that the light chain was the same for every antibody and was encoded by the vector. After ligation, the phagemids were used to transform E. coli TG1 bacteria and transformed bacteria were plated onto LB-agar plates containing ampicillin and glucose. All phage libraries contained >10e6 transformants and had an insert frequency of >80%. Bacteria were harvested after overnight growth and used to prepare phage according to established protocols (de Haard et al., J Biol Chem. 1999 Jun. 25; 274(26):18218-30).

### Selection of Phage Carrying Fab Fragments Specifically Binding to Human CD3.

Phage libraries were rescued according to standardized procedures (J Mol Biol. 1991 Dec. 5; 222(3):581-97; J Biol Chem. 1999 Jun. 25; 274(26):18218-30) and phage were selected with one or more rounds of selection of the immune phage antibody repertoires. In the first round, recombinant CD3 protein was coated onto the wells of a maxisorp™ ELISA plate or to a NUNC immuno-tube, whereas in the second round, either recombinant CD3 protein or cells over-expressing the human CD3 protein were used. The maxisorp™ ELISA plates or immuno-tubes were blocked with 4% ELK. Phage antibody libraries were also blocked with 4% ELK and excess of human IgG to deplete for Fc region binders prior to the addition of the phage library to the coated antigen.

Incubation with the phage library with the coated protein was performed for 2 hrs at room temperature under shaking conditions. Plates or tubes were then washed with 0.05% Tween-20 in PBS followed by 5 to 10 times washing with PBS. Bound phage were eluted using 50 mM glycine (pH 2.2) and added to E. coli TG-1 and incubated at 37° C. for phage infection.

Subsequently infected bacteria were plated on agar plates containing Ampicillin, and glucose and incubated at 37° C. overnight. After the first round of selection, colonies were scraped off the plates and combined and thereafter rescued and amplified to prepare an enriched first round phage pool for the synthetic repertoires. For the ‘immune’ repertoires, single clones were screened for target binding after the first round of phage selection.

### Antibody Cloning and Production

Bispecific antibodies as used herein typically differ from each other only in the particular amino acid sequence of the heavy chain variable region of one or both variable domains. The antibodies were produced by cloning the heavy chain variable regions into expression vectors for the expression of heavy and light chains. Methods for the production of bispecific antibodies are known in the art.

Briefly, DNA encoding the heavy chain variable region for the CD3 targeted variable domain was cloned into MV1624 vector (see FIG. 10a), encoding the KK residues (L351K, T366K) in the CH3 region for the generation of IgG heavy chain heterodimers (WO2013/157954 and WO2013/157953). The Fc constant regions contains mutation in the CH2 to silence the Fc effector function. The DNA encoding the heavy chain variable region replaces the stuffer region in the construct. The variable region is preceded by an encoded HC signal peptide (not shown). The DNA encoding the heavy chain variable region for the EGFR targeted variable domain was cloned into vector MV1625 (FIG. 10b) expressing the second heavy chain of the base antibody portion of the bispecific antibodies, bearing the L351D-L368E mutations in the CH3 region (WO2013/157954 and WO2013/157953). The DNA encoding the heavy chain variable region replaces the stuffer region in the construct. The variable region is preceded by an encoded HC signal peptide (not shown). Both constructs also contain an expression cassette for expression of the IGKV1-39/jk1 light chain. Expression of the two heavy chains together with the mentioned light chain leads to the production of the bispecific antibody.

293-F cells were used for expression of the designed antibodies in a 24 wells plate format. Two days before transfection, 293-F cell stock was split in 293-F culture medium in a 1:1 ratio and incubated overnight at 37° C. and 8% CO₂ at an orbital shaking speed of 155 rpm. Cells were diluted on the day before transfection to a density of 5×10⁵ cells/mL. 4 ml of the suspension cells were seeded into a 24 deep wells plate, covered with a breathable seal and incubated overnight at 37° C. and 8% CO₂ at an orbital shaking speed of 285 rpm. On transfection day, 4.8 ml 293-F culture medium were mixed with 240 μg of polyethylenimine (PEI) linear (MW 25,000). For each IgG to be produced, 200 μL of the 293F culture medium-PEI mix was added to 8 μl of DNA (for IgG heterodimers 4 μl of DNA encoding each heavy chain). The mixture was incubated for 20 minutes at room temperature before gently adding to the cells. On the day after transfection Penicillin-Streptomycin (Pen Strep) diluted in 500 μL 293F medium was added to each well. The plates were incubated at 37° C. and 8% CO₂ at an orbital shaking speed of 285 rpm until harvest seven days after transfection. Plates were centrifuged 5 min at 500 g, supernatants containing IgGs were filtered using 10-12 μm melt blown polypropylene filter plates and stored at −20° C. prior to purification.

Purification of Antibodies from Culture Supernatant

Medium containing antibodies is harvested and centrifuged to remove the cell debris. Subsequently Protein A Sepharose beads are added to the medium. Medium and Protein A Sepharose beads are incubated with the antibodies to allow binding.

After incubation the beads are isolated from the medium and washed, by a vacuum filter. The antibodies are eluted from the beads by incubation with elution buffer. Optionally, the buffer of the purified IgG is exchanged/desalted.

### Buffer Exchange

In order to desalt the purified antibodies the antibody fraction is centrifuged using a filter plate or filter column. The plate or column is centrifuged to reduce the volume of the antibody fraction. Subsequently, PBS or the required buffer is added to the fraction to replace the buffer with a low salt buffer. Optionally this centrifugation step followed by adding buffer is repeated in order to further desalt the storage buffer of the antibodies.

### Antibody Tumor Antigen Specific T Cell Activation and Lysis of BxPC3 Cells or of HTC-116 Cells.

The capacity of the particular CD3×tumor antigen bispecific IgG combinations to induce tumor antigen-specific T cell activation and lysis of tumor antigen positive target cells in a cytotoxicity assay was tested. The effector cells were healthy donor-derived resting T cells and the target cells were BxPC3 cells or HTC-116 cells.

Using Ficoll and EasySep human T cell isolation kit according to standard techniques resting T cells were isolated from whole blood from healthy donors, checked for >95% T cell purity by anti-CD3 antibody using flow cytometric analysis and subsequently cryopreserved. For a cytotoxicity assay the cryopreserved T cells were thawed and used if their viability was >90% upon thawing, determined by standard Trypan Blue staining. Cytotoxicity assay in short, thawed resting T cells and BxPC3 or HCT116 target cells were co-cultured in an ET ratio of 5:1 for 48 hours. Antibodies were tested in a dilution range. a CD3 monospecific antibody and an EGFR monospecific antibody, as well as an irrelevant IgG1 isotype control mAb are included in the assay as controls (e.g., an antibody which binds CD3 and another antigen such as tetanus toxin (TT)). T cell activation was quantified using flow cytometry; CD8 T cells were gated based on CD8 expression and subsequently analyzed for their activation status by measuring CD69 expression on T cells. Target cell lysis was determined by measuring the fraction of alive cells by measuring ATP levels assessed by CellTiterGlo (Promega). ATP levels, measured by luminescence on an Envision Microplate reader results in Relative light unit (RLU) values, which were analyzed using GraphPad Prism.

Target cell lysis for each sample was calculated as follows:

$$\% Killing = ( 100 - ( RLU sample / RLU no IgG ) \times 100 ) .$$

In this assay, the bispecific antibodies have two binding domains. One of the binding domains is targeted towards EGFR and the other to CD3. Both binding domains have the same (common) light chain variable region (VL) and a different heavy chain variable region (VH). The EGFR targeted binding domain has a VH with the amino acid sequence of MF8233. The CD3 targeted binding domain has a VH with an amino acid sequence of one of the MFs indicated for CD3. The bispecific antibody contain mutation in the CH2 to silence the Fc effector function.

The antibody MF8233×MF8397 induced upregulation of CD69 (FIG. 4-6) and CD25 (FIG. 4-6) on CD4 and CD8 T cells, determined after 48 hours of co-culture at an E±T ratio of 5:1. T cell-mediated lysis was measured after 48 hours.

### CD3 Bispecific Antibody Characterization

A candidate EGFR/CD3 IgG bispecific antibody can be tested for binding using any suitable assay. For example, binding to membrane-expressed CD3 on HPB-ALL cells (DSMZ, ACC 483) can be assessed by flow cytometry (according to the FACS procedure as previously described in WO2014/051433). In one embodiment, the binding of a candidate EGFR/CD3 bispecific antibody to CD3 on HPB ALL cells is demonstrated by flow cytometry, performed according to standard procedures known in the art. Binding to cell expressed CD3 can be confirmed using CHO cell transfected with CD3δ/ε or CD3γ/ε. The binding of the candidate bispecific IgG1 to EGFR can be determined using BxPC3 and HCT-116 as well as CHO cells transfected with an EGFR expression construct; a CD3 monospecific antibody and an EGFR monospecific antibody, as well as an irrelevant IgG1 isotype control mAb are included in the assay as controls (e.g., an antibody which binds CD3 and another antigen such as tetanus toxin (TT)).

Generation of Further Clones from Superclusters 1, 3 and 4

From the immune phage library screening (as described in section ‘Selection of phage carrying Fab fragments specifically binding to human CD3’), additional clones were characterized carrying Fab fragments specifically binding to human CD3. From supercluster 1, additional clones were identified, including MF8048, MF8101 and MF8056. Additional clones were identified from supercluster 3 and supercluster 4, including MF8562 of supercluster 3 and MF8998 of supercluster 4.

From supercluster 4, further new clones were identified using next-generation sequencing (NGS) analysis. NGS was performed on the VH gene pools present from MeMo® mice that were used to generate the anti-CD3 panel. To this aim, the sequence data sets obtained from different mice were compared with an MF sequence belonging to supercluster 4 MF. This led to identification of sequence variants clones MF10401 and MF10428 that belong to supercluster 4. Several different mutations were found in the HCDR1 and HCDR2 for different sequences.

VH sequences of all additional clones from supercluster 1, 3 and 4 were cloned into MV1624 (DM-KK) vector and expressed as CD3×EGFR bispecific format for further characterization, as described in section ‘Antibody cloning and Production’ above.

Characterization of Further Clones from Superclusters 1 and 4

Additional clones from supercluster 1 were characterized with respect to their functional activity in a bispecific format. The EGFR binding domain of the bispecific CD3×EGFR antibody has the amino acid sequence encoded by MF8233. As a control, these CD3 clones were also tested with another antigen (e.g. Tetanus toxin) with the amino acid sequence encoded by MF1337. Reference MFs from supercluster 1 (MF8057 and MF8058) were included to directly compare the affinity of the sequence variant to that of already characterized MF clones from supercluster 1 according to sections: ‘Antibody tumor antigen specific T cell activation and lysis of BxPC3 cells or of HTC-116 Cells’ and ‘CD3 Bispecific Antibody Characterization’ as described above. Binding affinity to HPB-All cells expressing human CD3-TCR complex using flow cytometry (FIG. 14A and FIG. 18) and T-cell activation and lysis of tumor antigen positive target cells (HCT-116) in a cytotoxicity assay (FIG. 14B-E) assays were performed. No target cell lysis was observed with bispecific antibodies having the MF1337 control arm. For the different CD3 clones tested, target cell lysis was observed in a dose dependent manner. Low target cell lysis was observed for MF8048. Expression levels of activation markers CD69 and CD25 on CD4 and CD8 T cells were measured in FACS staining for the evaluation of T-cell activation. Dose-dependent T-cell activation was observed for all clones, and no T-cell activation was observed for negative control. Finally, cytokine production of IFN-γ and TNF-α was determined in the supernatant derived from the EGFRxCD3 cytotoxicity assay with HCT-116 cells after 48 hrs using Luminex® Assays (eBiosciences™) following standard manufacturer's instructions (FIG. 14F-G).

For characterization of additional clones belonging to supercluster 4, binding affinity was determined in FACS to HPB-ALL cells (FIG. 15). PG1337, a monovalent antibody with two identical MF1337 arms specific for tetanus toxin, was used as a negative control. For cytotoxicity assay, HCT-116 cells and BxPC3 cells were used as target cells to test activity of MF8998 and BxPC3 target cells were used to test activity of MF10401 and MF10428. A CD3×TAA bispecific antibody with known high activity was included as positive control. Target cell lysis was quantified using cell viability measurements. Supernatant from the cytotoxicity assay was used to measure cytokine levels for IL-6, IFN-γ and TNF-α using Luminex® assays.

The three supercluster 4 clones tested were thus found to exhibit different binding but similar lysis activity. Although the lysis activity of these clones was similar, reduced cytokine production was observed.

TABLE 1
AUC values for binding of the indicated
CD3 clones mentioned in FIG. 15A

| MF combination | AUC |
|---|---|
| MF8233 × MF8998 | 13630 |
| MF8233 × MF10428 | 2700 |
| MF8233 × MF10401 | 9646 |
| MF1337 × MF1337 | 255 |

TABLE 2
AUC and EC50 values for target cell lysis of the indicated
CD3 clones mentioned in FIG. 15A.

| MF combination | Target | AUC | EC50 (ng/mL) |
|---|---|---|---|
| MF8233 × MF8998 | EGFR | 253 | 6.9 |
| MF8233 × MF10428 | EGFR | 216 | 10.7 |
| MF8233 × MF10401 | EGFR | 241 | 4.6 |
| CD3 × Mock: −ctrl | Mock | 11 | — |
| CD3 × TAA: +ctrl | TAA | 369 | 4.5 |

TABLE 3
AUC and EC50 values for the amount of indicated cytokines
and CD3 clones mentioned in FIG. 15A.

| MF combination | Cytokine | AUC | EC50 (ng/mL) |
|---|---|---|---|
| MF8233 × MF8998 | IL-6 | 858 | 51.2 |
| MF8233 × MF10428 | IL-6 | 659 | 8.6 |
| MF8233 × MF10401 | IL-6 | 657 | 3.1 |
| MF8233 × MF8998 | IFNγ | 311 | >400 |
| MF8233 × MF10428 | IFNγ | 107 | >4000 |
| MF8233 × MF10401 | IFNγ | 114 | >4000 |
| MF8233 × MF8998 | TNFα | 50 | — |
| MF8233 × MF10428 | TNFα | 50 | — |
| MF8233 × MF10401 | TNFα | 50 | — |

As evaluated by the cytotoxicity assay, it was observed that all further identified MFs are functional. Next, a graph was plotted between lysis on Y-axis and binding affinity on X-axis (FIG. 16A) to understand the relation within and across superclusters between lysis and affinity. Overall, a diverse panel of anti-CD3 Fabs was generated composed of multiple superclusters and covering a range of affinities. Interestingly, clones were identified in supercluster 1 and 4 which exhibit similar activity but different CD3 binding (FIG. 16B). Comparison of superclusters 1 and 3 revealed clones which exhibit similar CD3 binding and differential activity (FIG. 16C).

TABLE 4
AUC and EC50 values for target cell lysis of the indicated
CD3 clones mentioned in FIGS. 16B and 16C.

| | | EC50 |
|---|---|---|
| MF combination | AUC | (ng/mL) |
| MF8233 × MF8057 | 117 | 28.5 |
| MF8233 × MF8058 | 283 | 4.4 |
| MF8233 × MF8101 | 355 | 1.0 |
| MF8233 × MF8508 | 267 | 6.4 |
| MF8233 × MF8998 | 232 | 8.0 |
| MF8233 × MF8397 | 62 | >400 |
| MF8233 × MF8562 | 57 | >400 |

### Characterization of CD3 Antigens

As described above, two clones from supercluster 1 and supercluster 4, i.e. MF8058 and MF8998 respectively, were found to have similar lysis activity in a bispecific format with clone MF8233 as Fab arm binding EGFR as the tumor-cell antigen (FIG. 17). For this experiment, lysis activity against HCT-116 cells was measured as described herein above. MF9257×MF8233 was used as a positive control and MF9257×MF1337 was the negative control. As can be seen from FIG. 17 and Table 5, dose-dependent and high killing percentage were observed for multiple tested bispecific antibodies.

TABLE 5
AUC and EC50 values for target cell lysis for the indicated bispecific
antibodies mentioned in FIG. 17. Negative controls displayed no
significant measurable activity.

| | Tumor target and | | | |
|---|---|---|---|---|
| Cytoxicity | immune cell | | | |
| assay/ | engaging target if | | | EC50 |
| target cell | applicable | MF combination | AUC | (ng/mL) |
| HCT116 | EGFR × CD3 | MF8233 × MF8058 | 283 | 4.4 |
| HCT116 | EGFR × CD3 | MF8233 × MF8998 | 232 | 8.0 |
| HCT116 | EGFR | +control | 354 | 0.5 |

### Binding Affinity

As described in section ‘CD3 Bispecific antibody characterization’, the binding affinity of additional CD3 clones was analyzed in FACS on HPB-ALL cells expressing human CD3. The affinities of MF6955 and MF6964 for CD3 were measured by surface plasmon resonance (SPR) technology using a BIAcore™ T100. An anti-human IgG mouse monoclonal antibody (Becton and Dickinson, cat. Nr. 555784) was coupled to the surfaces of a CM5 sensor chip using free amine chemistry (NHS/EDC). Then the CD3×TAA bispecific antibody was captured onto this sensor surface. Subsequently the recombinant purified antigen human CD308-Fc was run over the sensor surface in a concentration range to measure on- and off-rates. After each cycle, the sensor surface was regenerated by a pulse of HCl and CD3×TAA bispecific antibody was captured again. From the obtained sensograms, on- and off-rates were determined using the BIAevaluation software. FIG. 18 describes the binding affinity range of the CD3 panel generated.

1.-33. (canceled)

34. A method for treating a subject in need comprising administering the subject a therapeutically effective amount of an antigen-binding protein that binds human CD3 comprising an antibody variable domain comprising a heavy chain variable region and a light chain variable region wherein the heavy chain variable region comprises:
a CDR1, CDR2 and CDR3 comprising the amino acid sequence
CDR1:
(SEQ ID NO: 42)
SFGIS
CDR2:
(SEQ ID NO: 43)
GFIPVLGTANYAQKFQG
CDR3:
(SEQ ID NO: 44)
RGNWNPFDP;
or
comprising the amino acid sequence:
CDR1:
(SEQ ID NO: 51)
SKTFTIS;
CDR2:
(SEQ ID NO: 52)
GIIPLFGTITYAQKFQG;
CDR3:
(SEQ ID NO: 53)
RGNWNPFDP;
or
comprising the amino acid sequence:
CDR1:
(SEQ ID NO: 60)
SRTFTIS;
CDR2:
(SEQ ID NO: 61)
SIIPIFGTITYAQKFQG;
CDR3:
(SEQ ID NO: 62)
RGNWNPFDP;
or
comprising the amino acid sequence:
CDR1:
(SEQ ID NO: 84)
SKTLTIS;
CDR2:
(SEQ ID NO: 85)
GIIPIFGSITYAQKFQD;
CDR3:
(SEQ ID NO: 86)
RGNWNPFDP,
or
comprising the amino acid sequence:
CDR1:
(SEQ ID NO: 68)
GSGIS;
CDR2:
(SEQ ID NO: 69)
GFIPFFGSANYAQKFRD;
CDR3:
(SEQ ID NO: 70)
RGNWNPLDP;
or
comprising the amino acid sequence:
CDR1:
(SEQ ID NO: 100)
GSGIS;
CDR2:
(SEQ ID NO: 101)
GFIPFFGSANYAQKFRD;
CDR3:
(SEQ ID NO: 102)
RGNWNPFDP;
or
comprising the amino acid sequence:
CDR1:
(SEQ ID NO: 199)
RFWIG;
CDR2:
(SEQ ID NO: 200)
IIYPGDSDTRYSPSFQG;
CDR3:
(SEQ ID NO: 201)
HIRYFDWSEDYHYYLDV;
or
comprising the amino acid sequence:
CDR1:
(SEQ ID NO: 208)
RYWIG;
CDR2:
(SEQ ID NO: 209)
IIYPGDSDTRYSPSFQG;
CDR3:
(SEQ ID NO: 210)
NIRYFVWSEDYHYYMDV;
or
comprising the amino acid sequence:
CDR1:
(SEQ ID NO: 117)
SYALS;
CDR2:
(SEQ ID NO: 118)
GISGSGRTTWYADSVKG;
CDR3:
(SEQ ID NO: 119)
DGGYSYGPYWYFDL;
or
comprising the amino acid sequence:
CDR1:
(SEQ ID NO: 125)
SYALS;
CDR2:
(SEQ ID NO: 126)
AISGSGRTTWYADSVKG;
CDR3:
(SEQ ID NO: 127)
DGGYTYGPYWYFDL;
or
comprising the amino acid sequence:
CDR1:
(SEQ ID NO: 142)
DYTMH;
CDR2:
(SEQ ID NO: 143)
DISWSSGSIGYADSVKG;
CDR3:
(SEQ ID NO: 144)
DHRGYGDYEGGGFDY;
or
comprising the amino acid sequence:
CDR1:
(SEQ ID NO: 150)
DYTMH;
CDR2:
(SEQ ID NO: 151)
DISWSSGSIGYADSVKG;
CDR3:
(SEQ ID NO: 152)
DHRGYGDYEGGGFDH;
comprising the amino acid sequence:
CDR1:
(SEQ ID NO: 166)
DYTMH;
CDR2:
(SEQ ID NO: 167)
DISWSGGSIYYADSVKG;
CDR3:
(SEQ ID NO: 168)
DHRGYGDYEGGGFDY;
or
comprising the amino acid sequence:
CDR1:
(SEQ ID NO: 182)
DYTMH;
CDR2:
(SEQ ID NO: 183)
DISWSSGTTGYADSVKG;
CDR3:
(SEQ ID NO: 184)
DHMGYGDYEGGGFDY;
and wherein the light chain variable region comprises a CDR1, CDR2, and CDR3 region comprising the amino acid sequence:
CDR1:
(SEQ ID NO: 21)
QSISSY;
CDR2:
(SEQ ID NO: 22)
AAS;
CDR3:
(SEQ ID NO: 299)
QQSYSTPPT.

35. The method of claim 34, wherein the subject has cancer or is treated for cancer.

36. The method of claim 34, wherein the subject has an over-active immune system.

37. The method of claim 36, wherein the subject has an auto-immune disease.

38. The method of claim 34, wherein said light chain variable region is a germline IgVκ1-39*01 variable region.

39. The method of claim 34, wherein said light chain variable region comprises the kappa light chain IgVκ1-39*01/IGJκ1*01.

40. The method of claim 34, wherein said light chain variable region comprises the germline kappa light chain IgVκ1-39*01/IGJκ1*01.

41. The method of claim 34, wherein said light chain variable region comprises the amino acid sequence
(SEQ ID NO: 16)
DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPTFGQ
GTKVEIK
with 0-5 amino acid variations, insertions, deletions, substitutions, additions or a combination thereof at one or more positions other than the CDRs.

42. The method of claim 34, wherein the antigen-binding protein is an antibody.

43. The method of claim 42, wherein said antibody is a human or humanized antibody.

44. The method of claim 42, wherein said antibody is bispecific antibody.

45. The method of claim 44, wherein the bispecific antibody further comprises a VH/VL chain combination that binds a tumor-antigen.

46. The method of claim 45, wherein said VH/VL chain combination that binds a tumor-antigen binds human BCMA, CD19, CD20, CD30, CD33, CD38, CD44, CD123, CD138, CEA, CLEC12A, CS-1, EGFR, EGFRVIII, EPCAM, DLL3, LGR5, MSLN, FOLR1, FOLR3, HER2, HM1.24, MCSP, PD-L1, PSMA protein or a variant thereof.

47. The method of claim 44, wherein the bispecific antibody is a human or humanized antibody.

48. The method of claim 44, wherein the bispecific antibody comprises two different immunoglobulin heavy chains with compatible heterodimerization domains.

49. The method of claim 48, wherein said compatible heterodimerization domains are compatible immunoglobulin heavy chain CH3 heterodimerization domains.

50. The method of claim 44, wherein said bispecific antibody is an IgG antibody with a mutant CH2 and/or lower hinge domain such that interaction of the bispecific IgG antibody to a Fc-gamma receptor is reduced.

51. The method of claim 50, wherein said mutant CH2 and/or lower hinge domain comprise an amino substitution at position 235 and/or 236, according to EU numbering.

52. The method of claim 44, wherein the bispecific antibody comprises a common light chain.

53. The method of claim 34, wherein said heavy chain variable region comprises the amino acid sequence:
(SEQ ID NO: 45)
EVQLVQSGAEVKKPGSSVKVSCKASGGTFRSFGISWVRQAPGQGLEWMGG
FIPVLGTANYAQKFQGRVTIIADKSTNTAYMELSSLRSEDTAVYYCARRG
NWNPFDPWGQGTLVTVSS,
with 0-10 or 0-5 amino acid variations, insertions, deletions, substitutions, additions or a combination thereof at one or more positions other than the CDRs.

54. The method of claim 34, wherein said heavy chain variable region comprises the amino acid sequence:
(SEP ID NO: 54)
QVQLVQSGAEVKKPGSSVKVSCKASGDAFKSKTFTISWVRQAPGQGLEWL
GGIIPLFGTITYAQKFQGRVTITADKSTNTAFMELSSLRSEDTAMYYCTR
RGNWNPFDPWGQGTLVTVSS,
with 0-10 or 0-5 amino acid variations, insertions, deletions, substitutions, additions or a combination thereof at one or more positions other than the CDRs.

55. The method of claim 34, wherein said heavy chain variable region comprises the amino acid sequence:
(SEQ ID NO: 63)
EVQLVQSGSELKKPGSSVKVSCKASGVTGNSRTFTISWVRQAPGQGLEWL
GSIIPIFGTITYAQKFQGRVTITADKSTSTAFMELTSLRSEDTAIYYCTR
RGNWNPFDPWGQGTLVTVSS,
with 0-10 or 0-5 amino acid variations, insertions, deletions, substitutions, additions or a combination thereof at one or more positions other than the CDRs.

56. The method of claim 34, wherein said heavy chain variable region comprises the amino acid sequence:
(SEQ ID NO: 71)
QVQLVQSGAEVKKPGSSVKVSCKASGGTFRGSGISWVRQAPGQGLEWVGG
FIPFFGSANYAQKFRDRVTITADKSATTAYMELSSLRSEDTAIYYCAKRG
NWNPLDPWGQGTLVTVSS,
with 0-10 or 0-5 amino acid variations, insertions, deletions, substitutions, additions or a combination thereof at one or more positions other than the CDRs.

57. The method of claim 34, wherein said heavy chain variable region comprises the amino acid sequence:
(SEQ ID NO: 87)
QVQLVQSGAEVKKPGSSVKVSCKASGVTFKSKTLTISWVRQAPGQGLEWL
GGIIPIFGSITYAQKFQDRVSITADKSTNTAYLELNSLRSEDTAIYYCAR
RGNWNPFDPWGQGTLVTVSS,
with 0-10 or 0-5 amino acid variations, insertions, deletions, substitutions, additions or a combination thereof at one or more positions other than the CDRs.

58. The method of claim 34, wherein said heavy chain variable region comprises the amino acid sequence:
(SEQ ID NO: 103)
EVQLVQSGAEVKKPGSSVKVSCKASGGTFRGSGISWVRQAPGQGLEWVGG
FIPFFGSANYAQKFRDRVTITADKSATTAYMELSSRSEDTAIYYCAKRGN
WNPFDPWGQGTLVTVSS,
with 0-10 or 0-5 amino acid variations, insertions, deletions, substitutions, additions or a combination thereof at one or more positions other than the CDRs.

59. The method of claim 34, wherein said heavy chain variable region comprises the amino acid sequence:
(SEQ ID NO: 202)
EVQLVQSGAEVKKPGESLKISCKGSGYSFTRFWIGWVRQMPGKGLEWMGI
IYPGDSDTRYSPSFQGQVTISADKSTSTAYLQWSSLKASDTGMYYCVRHI
RYFDWSEDYHYYLDVWGKGTTVTVSS,
with 0-10 or 0-5 amino acid variations, insertions, deletions, substitutions, additions or a combination thereof at one or more positions other than the CDRs.

60. The method of claim 34, wherein said heavy chain variable region comprises the amino acid sequence:
(SEQ ID NO: 211)
EVQLVESGAEVKKPGESLKISCKGSGYSFTRYWIGWVRQMPGKGLEWMGI
IYPGDSDTRYSPSFQGQVTISADKSISTAYLQWSSLKASDTAMYYCVRNI
RYFVWSEDYHYYMDVWGKGTTVTVSS,
with 0-10 or 0-5 amino acid variations, insertions, deletions, substitutions, additions or a combination thereof at one or more positions other than the CDRs.

61. The method of claim 34, wherein said heavy chain variable region comprises the amino acid sequence:
(SEQ ID NO: 145)
EVQLVESGGGLVQPGRSLRLSCATSGFNFDDYTMHWVRQAPGKGLEWVSD
ISWSSGSIGYADSVKGRFTISRDNAKNSLWLQMNSLRTEDTALYFCAKDH
RGYGDYEGGGFDYWGQGTLVTVSS,
with 0-10 or 0-5 amino acid variations, insertions, deletions, substitutions, additions or a combination thereof at one or more positions other than the CDRs.

62. The method of claim 34, wherein said heavy chain variable region comprises the amino acid sequence:
(SEQ ID NO: 153)
EVQLVESGGGLVQPGRSLRLSCATSGFTFDDYTMHWVRQAPGKGLEWVSD
ISWSSGSIGYADSVKGRFTISRDNAKNSLYLQMNSLRAEDTALYFCAKDH
RGYGDYEGGGFDHWGQGTLVTVSS,
with 0-10 or 0-5 amino acid variations, insertions, deletions, substitutions, additions or a combination thereof at one or more positions other than the CDRs.

63. The method of claim 34, wherein said heavy chain variable region comprises the amino acid sequence:
(SEQ ID NO: 185)
EVQLVESGGGLVQPGRSLRLSCVTSGFTFDDYTMHWVRQAPGKGLEWVS
DISWSSGTTGYADSVKGRFTISRDNAKDSLYLQMNSLRTEDTALYYCAK
DHMGYGDYEGGGFDYWGQGTLVTVSS,
with 0-10 or 0-5 amino acid variations, insertions, deletions, substitutions, additions or a combination thereof at one or more positions other than the CDRs.

64. The method of claim 34, wherein said heavy chain variable region comprises the amino acid sequence:
(SEQ ID NO: 169)
EVQLVESGGVVVQPGGSLRLSCAASGFTFDDYTMHWVRQAPGKGLEWVS
DISWSGGSIYYADSVKGRFTISRDNSKNSLYLQMNSLRTEDTALYYCAK
DHRGYGDYEGGGFDYWGRGTLVTVSS,
with 0-10 or 0-5 amino acid variations, insertions, deletions, substitutions, additions or a combination thereof at one or more positions other than the CDRs.

65. The method of claim 34, wherein said heavy chain variable region comprises the amino acid sequence:
(SEQ ID NO: 120)
QVQLVQSGGGLVQPGGSLRLSCATSGFKFSSYALSWVRQAPGKGLEWVSG
ISGSGRTTWYADSVKGRFTISRDNSKNTLYLQMNSLRAEDTAVYYCARDG
GYSYGPYWYFDLWGRGTLVTVSS,
with 0-10 or 0-5 amino acid variations, insertions, deletions, substitutions, additions or a combination thereof at one or more positions other than the CDRs.

66. The method of claim 34, wherein said heavy chain variable region comprises the amino acid sequence:
(SEQ ID NO: 128)
QVQLVESGGGLVQPGGSLRLSCATSGFTFISYALSWVRQAPGKGLEWVS
AISGSGRTTWYADSVKGRFTISRDNSKNTLFLQMNSLRAEDTAVYYCAR
DGGYTYGPYWYFDLWGRGTLVTVSS,
with 0-10 or 0-5 amino acid variations, insertions, deletions, substitutions, additions or a combination thereof at one or more positions other than the CDRs.
